(19) United States
(12) Patent Application Publication
(10) Pub. No.: US 2025/0197408 A1
(43) Pub. Date: Jun. 19, 2025

(54) CRYSTALLINE FORMS AND SALTS OF A PI3K INHIBITOR AND METHODS OF MAKING AND METHODS OF USE THEREOF

(71) Applicant: Totus Medicines Inc., Emeryville, CA (US)

(72) Inventors: Neil Sonin DHAWAN, Somerville, MA (US); James Abellera BLAIR, Arlington, MA (US); Robert B. PERNI, Marlborough, MA (US); Jared Lynn RANDALL, Emeryville, CA (US); Xiaoyang WANG, Shanghai (CN); Meiqi LI, Shanghai (CN)

(21) Appl. No.: 18/849,948

(22) PCT Filed: Mar. 24, 2023
(86) PCT No.: PCT/CN2023/083760

§ 371 Date: Sep. 23, 2024

(30) Foreign Application Priority Data
Mar. 24, 2022 (WO) PCT/CN2022/082752

(51) Int. Cl. C07D 487/04; A61K 31/5377; A61P 35/00; C07C 55/10; C07C 57/145; C07C 57/15; C07C 59/255; C07C 59/265; C07C 309/04; C07C 309/30

(52) U.S. Cl. CPC C07D 487/04; A61K 31/5377; A61P 35/00; C07C 55/10; C07C 57/145; C07C 57/15; C07C 59/255; C07C 59/265; C07C 309/04; C07C 309/30

(57) ABSTRACT

Provided herein are salts and crystalline forms of Compound (I), and salts, solvates, and salt solvates thereof. Also provided herein are pharmaceutical compositions comprising the crystalline forms, and therapeutic uses of the crystalline forms and the compositions thereof.

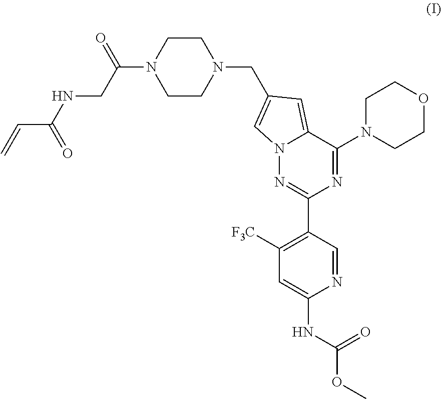 [SMILES: C=CC(=O)NCC(=O)N1CCN(Cc2cc3c(N4CCOCC4)nc(-c4cnc(NC(=O)OC)cc4C(F)(F)F)nn3c2)CC1]

### CROSS-REFERENCE TO RELATED APPLICATIONS

This application claims priority to Patent Application No. PCT/CN2022/082752, filed on Mar. 24, 2022, the contents of which is hereby incorporated by reference in its entirety for all purposes.

### BACKGROUND

Phosphatidylinositol 3-kinases (PI3Ks) comprise a family of lipid kinases that catalyze the transfer of phosphate to the 3-position of the inositol ring of phosphatidylinositol and its derivatives to produce phosphoinositol-3-phosphate (PI (3) P), phosphoinositol-3,4-diphosphate (PI (3,4) P2) and phosphoinositol-3,4,5-triphosphate (PI (3,4,5) P3) that, in turn, act as second messengers in signaling cascades by docking proteins containing pleckstrin-homology, FYVE, Phox and other phospholipid-binding domains into a variety of signaling complexes often at the plasma membrane ((Vanhaesebroeck et al., Annu. Rev. Biochem 70:535 (2001); Katso et al., Annu. Rev. Cell Dev. Biol. 17:615 (2001)). PI3Ks have been divided into three classes according to their structural characteristics and substrate specificity. Class IA PI3Ks are heterodimers composed of a p110 catalytic subunit and a p85 regulatory subunit. In mammals, there are three genes, PIK3CA, PIK3CB and PIK3CD, encoding p110 catalytic isoforms: p110α, p110β and p110δ, respectively. There are also three genes, PIK3R1, PIK3R2 and PIK3R3, encoding p85α (and its splicing variants p55α and p50 α), p85β and p55γ regulatory subunits, respectively, collectively called p85. The modular domains of the p85/55/50 subunits include Src Homology (SH2) domains that bind phosphotyrosine residues in a specific sequence context on activated receptor and cytoplasmic tyrosine kinases, resulting in activation and localization of Class 1A PI3Ks. Class IB PI3K is a heterodimer composed of a catalytic subunit p110γ and a regulatory subunit p101. p110γ is mainly expressed in leukocytes and can be activated directly by GPCRs. Class II PI3Ks are monomers with only a single catalytic subunit. Class III PI3Ks consists of a single catalytic subunit Vps34 (homolog of the yeast vacuolar protein-sorting defective 34). The phospholipid products of class I PI3K link upstream receptors with downstream cellular activities including proliferation, survival, chemotaxis, cellular trafficking, motility, metabolism, inflammatory and allergic responses, transcription and translation (Cantley et al., Cell 64:281 (1991); Escobedo and Williams, Nature 335:85 (1988); Fantl et al., Cell 69:413 (1992)). There is this interest and a current need for binding to and regulating PI3K for treatment of various diseases.

### SUMMARY

The present disclosure relates to a crystalline form of a phosphoinositide 3-kinase (PI3K) inhibitor, Compound I, or a pharmaceutically acceptable salt thereof, solvate thereof, or salt solvate thereof.

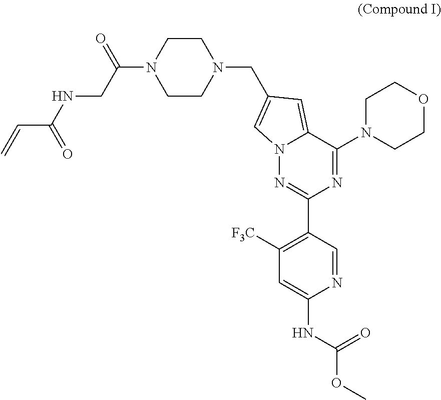 [SMILES: C=CC(=O)NCC(=O)N1CCN(Cc2cc3c(N4CCOCC4)nc(-c4cnc(NC(=O)OC)cc4C(F)(F)F)nn3c2)CC1]

In embodiments, provided herein is a crystalline form of Compound I.

In embodiments, the crystalline form of Compound I is anhydrous or non-solvated.

In embodiments, provided herein is a crystalline form of a pharmaceutically salt of Compound I or a pharmaceutically acceptable salt solvate thereof.

In embodiments, provided herein is a crystalline form of a pharmaceutically salt of Compound I.

In embodiments, provided herein is a crystalline form of a solvate of Compound I.

In embodiments, provided herein is a composition comprising a crystalline form of Compound I, or a pharmaceutically acceptable salt thereof, solvate thereof, or salt solvate thereof.

In embodiments, the present disclosure also relates to methods for treating various diseases or conditions with compound I, including diseases or conditions wherein irreversible inhibition of PI3K e.g., PIK3α provides therapeutic benefit to a subject having the disease or condition. In embodiments, the present disclosure provides a method of treating cancer in a subject in need thereof, comprising administering an effective amount of the crystalline form of Compound I, or a pharmaceutically acceptable salt thereof, solvate thereof, or salt solvate thereof, or the pharmaceutical composition comprising Compound I, or a pharmaceutically acceptable salt thereof, solvate thereof, or salt solvate thereof.

In embodiments, the present disclosure provides methods for inhibiting phosphoinositide 3-kinase (PI3K) in a subject in need thereof, comprising administering an effective amount of the crystalline form of Compound I, or a pharmaceutically acceptable salt thereof, solvate thereof, or salt solvate thereof, or the pharmaceutical composition comprising Compound I, or a pharmaceutically acceptable salt thereof, solvate thereof, or salt solvate thereof. In one embodiment, the present invention is a means for inhibiting phosphoinositide 3-kinase (PI3K). In another embodiment, the present invention may be a salt means for inhibiting PI3K. In another embodiment, the present invention may be a crystalline means for inhibiting PI3K. In another embodiment, the present invention may be an amorphous means for inhibiting PI3K. In a specific embodiment, the crystalline form may include Form A, Form A*, Form A**, Form B*, Form A-1, Form B-1, Form A-2, Form B-2, Form A-3, Form B-3, Form C-3, Form A-4, Form A-5, Form A-6, Form A-7, Form B-7, Form B-8, Form A-9, Form B, Form C, Form J, Form K, Form H2, Form E, Form F, Form G, Form I, Form H1, Form H3, Form H4, Form H5, and/or Form H6.

In one embodiment, the present invention is a pharmaceutical composition comprising a (a) means for inhibiting phosphoinositide 3-kinase (PI3K), and a pharmaceutically acceptable carrier. In a specific embodiment, the means may be in the form of a salt. In another embodiment, the composition may be in a form wherein the means is a crystalline form. In a specific embodiment, the crystalline form may include Form A, Form A*, Form A**, Form B*, Form A-1, Form B-1, Form A-2, Form B-2, Form A-3, Form B-3, Form C-3, Form A-4, Form A-5, Form A-6, Form A-7, Form B-7, Form B-8, Form A-9, Form B, Form C, Form J, Form K, Form H2, Form E, Form F, Form G, Form I, Form H1, Form H3, Form H4, Form H5, and/or Form H6.

### BRIEF DESCRIPTION OF FIGURES

FIG. 1A shows an X-ray powder diffraction (XRPD) spectrum of crystalline Form A of Compound I.

FIG. 1B shows a differential scanning calorimetry (DSC) thermogram of crystalline Form A of Compound I.

FIG. 1C shows a thermogravimetric analysis (TGA) thermogram of crystalline Form A of Compound I.

FIG. 1D shows a dynamic vapor sorption (DVS) isotherm plot of crystalline Form A of Compound I.

FIG. 2A shows an X-ray powder diffraction (XRPD) spectrum of crystalline Form A* of Compound I Phosphate salt.

FIG. 2B shows a differential scanning calorimetry (DSC) thermogram of crystalline Form A* of Compound I Phosphate salt.

FIG. 2C shows a thermogravimetric analysis (TGA) thermogram of crystalline Form A* of Compound I Phosphate salt.

FIG. 2D shows a dynamic vapor sorption (DVS) isotherm plot of crystalline Form A* of Compound I Phosphate salt.

FIG. 3A shows an X-ray powder diffraction (XRPD) spectrum of crystalline Form A** of Compound I benzenesulfonate salt.

FIG. 3B shows a differential scanning calorimetry (DSC) thermogram of crystalline Form A** of Compound I benzenesulfonate salt.

FIG. 3C shows a thermogravimetric analysis (TGA) thermogram of crystalline Form A** of Compound I benzenesulfonate salt.

FIG. 3D shows a dynamic vapor sorption (DVS) isotherm plot of crystalline Form A** of Compound I benzenesulfonate salt.

FIG. 4A shows an X-ray powder diffraction (XRPD) spectrum of crystalline Form B* of Compound I maleate salt.

FIG. 4B shows a differential scanning calorimetry (DSC) thermogram of crystalline Form B* of Compound I maleate salt.

FIG. 4C shows a thermogravimetric analysis (TGA) thermogram of crystalline Form B* of Compound I maleate salt.

FIG. 4D shows a dynamic vapor sorption (DVS) isotherm plot of crystalline Form B* of Compound I maleate salt.

FIG. 5A shows an X-ray powder diffraction (XRPD) spectrum of crystalline Form A-1 of Compound I hydrochloride salt.

FIG. 5B shows a differential scanning calorimetry (DSC) thermogram of Form A-1 of Compound I hydrochloride salt.

FIG. 6A shows an X-ray powder diffraction (XRPD) spectrum of crystalline Form B-1 of Compound I hydrochloride salt.

FIG. 6B shows a differential scanning calorimetry (DSC) thermogram of crystalline Form B-1 of Compound I hydrochloride salt.

FIG. 7A shows an X-ray powder diffraction (XRPD) spectrum of crystalline Form A-2 of Compound I sulfate salt.

FIG. 7B shows a differential scanning calorimetry (DSC) thermogram of crystalline Form A-2 of Compound I sulfate salt.

FIG. 8A shows an X-ray powder diffraction (XRPD) spectrum of crystalline Form B-2 of Compound I sulfate salt.

FIG. 8B shows a differential scanning calorimetry (DSC) thermogram of crystalline Form B-2 of Compound I sulfate salt.

FIG. 9A shows an X-ray powder diffraction (XRPD) spectrum of crystalline Form A-3 of Compound I mesylate salt.

FIG. 9B shows a differential scanning calorimetry (DSC) thermogram of crystalline Form A-3 of Compound I mesylate salt.

FIG. 10A shows an X-ray powder diffraction (XRPD) spectrum of crystalline Form B-3 of Compound I mesylate salt.

FIG. 10B shows a differential scanning calorimetry (DSC) thermogram of crystalline Form B-3 of Compound I mesylate salt.

FIG. 11A shows an X-ray powder diffraction (XRPD) spectrum of crystalline Form C-3 of Compound I mesylate salt.

FIG. 11B shows a differential scanning calorimetry (DSC) thermogram of crystalline Form C-3 of Compound I mesylate salt.

FIG. 12A shows an X-ray powder diffraction (XRPD) spectrum of crystalline Form A-4 of Compound I tosylate salt.

FIG. 12B shows a differential scanning calorimetry (DSC) thermogram of crystalline Form A-4 of Compound I tosylate salt.

FIG. 13A shows an X-ray powder diffraction (XRPD) spectrum of crystalline Form A-5 of Compound I fumarate salt.

FIG. 13B shows a differential scanning calorimetry (DSC) thermogram of crystalline Form A-5 of Compound I fumarate salt.

FIG. 14A shows an X-ray powder diffraction (XRPD) spectrum of crystalline Form A-6 of Compound I maleate salt.

FIG. 14B shows a differential scanning calorimetry (DSC) thermogram of crystalline Form A-6 of Compound I maleate salt.

FIG. 15A shows an X-ray powder diffraction (XRPD) spectrum of crystalline Form A-7 of Compound I L-tartrate salt.

FIG. 15B shows a differential scanning calorimetry (DSC) thermogram of crystalline Form A-7 of Compound I L-tartrate salt.

FIG. 16A shows an X-ray powder diffraction (XRPD) spectrum of crystalline Form B-7 of Compound I L-tartrate salt.

FIG. 16B shows a differential scanning calorimetry (DSC) thermogram of crystalline Form B-7 of Compound I L-tartrate salt.

FIG. 17A shows an X-ray powder diffraction (XRPD) spectrum of crystalline Form A-8 of Compound I citrate salt.

FIG. 17B shows a differential scanning calorimetry (DSC) thermogram of crystalline Form A-8 of Compound I citrate salt.

FIG. 18A shows an X-ray powder diffraction (XRPD) spectrum of crystalline Form B-8 of Compound I citrate salt.

FIG. 18B shows a differential scanning calorimetry (DSC) thermogram of crystalline Form B-8 of Compound I citrate salt.

FIG. 19A shows an X-ray powder diffraction (XRPD) spectrum of crystalline Form A-9 of Compound I succinate salt.

FIG. 19B shows a differential scanning calorimetry (DSC) thermogram of crystalline Form A-9 of Compound I succinate salt.

FIG. 20 shows an X-ray powder diffraction (XRPD) spectrum of crystalline Form B of Compound I phosphate salt.

FIG. 21A shows an X-ray powder diffraction (XRPD) spectrum of crystalline Form C of Compound I phosphate salt.

FIG. 21B shows a differential scanning calorimetry (DSC) thermogram of crystalline Form C of Compound I phosphate salt.

FIG. 21C shows a thermogravimetric analysis (TGA) thermogram crystalline Form C of Compound I phosphate salt.

FIG. 22A shows an X-ray powder diffraction (XRPD) spectrum of crystalline Form J of Compound I phosphate salt.

FIG. 22B shows a differential scanning calorimetry (DSC) thermogram of crystalline Form J of Compound I phosphate salt.

FIG. 22C shows a thermogravimetric analysis (TGA) thermogram crystalline Form J of Compound I phosphate salt.

FIG. 23A shows an X-ray powder diffraction (XRPD) spectrum of crystalline Form K of Compound I phosphate salt.

FIG. 23B shows a differential scanning calorimetry (DSC) thermogram of crystalline Form K of Compound I phosphate salt.

FIG. 23C shows a thermogravimetric analysis (TGA) thermogram crystalline Form K of Compound I phosphate salt.

FIG. 24 shows an X-ray powder diffraction (XRPD) spectrum of crystalline Form H2 of Compound I phosphate salt.

FIG. 25A shows an X-ray powder diffraction (XRPD) spectrum of crystalline Form E of Compound I phosphate salt.

FIG. 25B shows a differential scanning calorimetry (DSC) thermogram of crystalline Form E of Compound I phosphate salt.

FIG. 25C shows a thermogravimetric analysis (TGA) thermogram crystalline Form E of Compound I phosphate salt.

FIG. 26A shows an X-ray powder diffraction (XRPD) spectrum of crystalline Form F of Compound I phosphate salt.

FIG. 26B shows a differential scanning calorimetry (DSC) thermogram of crystalline Form F of Compound I phosphate salt.

FIG. 26C shows a thermogravimetric analysis (TGA) thermogram of crystalline Form F of Compound I phosphate salt.

FIG. 27A shows an X-ray powder diffraction (XRPD) spectrum of crystalline Form G of Compound I phosphate salt.

FIG. 27B shows a differential scanning calorimetry (DSC) thermogram of crystalline Form G of Compound I phosphate salt.

FIG. 27C shows a thermogravimetric analysis (TGA) thermogram of crystalline Form G of Compound I phosphate salt.

FIG. 28A shows an X-ray powder diffraction (XRPD) spectrum of crystalline Form I of Compound I phosphate salt.

FIG. 28B shows a differential scanning calorimetry (DSC) thermogram of crystalline Form I of Compound I phosphate salt.

FIG. 28C shows a thermogravimetric analysis (TGA) thermogram of crystalline Form I of Compound I phosphate salt.

FIG. 29A shows an X-ray powder diffraction (XRPD) spectrum of crystalline Form H1 of Compound I phosphate salt.

FIG. 29B shows a differential scanning calorimetry (DSC) thermogram of crystalline Form H1 of Compound I phosphate salt.

FIG. 29C shows a thermogravimetric analysis (TGA) thermogram of crystalline Form H1 of Compound I phosphate salt.

FIG. 30 shows an X-ray powder diffraction (XRPD) spectrum of crystalline Form H3 of Compound I phosphate salt.

FIG. 31 shows an X-ray powder diffraction (XRPD) spectrum of crystalline Form H4 of Compound I phosphate salt.

FIG. 32 shows an X-ray powder diffraction (XRPD) spectrum of crystalline Form H5 of Compound I phosphate salt.

FIG. 33 shows an X-ray powder diffraction (XRPD) spectrum of crystalline Form H6 of Compound I phosphate salt.

### Definitions

For convenience, certain terms employed in the specification, examples and claims are collected here. Unless defined otherwise, all technical and scientific terms used in this disclosure have the same meanings as commonly understood by one of ordinary skill in the art to which this disclosure belongs.

Throughout the present specification, the terms “about” and/or “approximately” may be used in conjunction with numerical values and/or ranges. The term “about” is understood to mean those values near to a recited value. Furthermore, the phrases “less than about [a value]” or “greater than about [a value]” should be understood in view of the definition of the term “about” provided herein. The terms “about” and “approximately” may be used interchangeably.

Throughout the present specification, numerical ranges are provided for certain quantities. It is to be understood that these ranges comprise all subranges therein. Thus, the range “from 50 to 80” includes all possible ranges therein (e.g., 51-79, 52-78, 53-77, 54-76, 55-75, 60-70, etc.). Furthermore, all values within a given range may be an endpoint for the range encompassed thereby (e.g., the range 50-80 includes the ranges with endpoints such as 55-80, 50-75, etc.).

The term “a” or “an” refers to one or more of that entity; for example, “a PI3-kinase (PI3K) modulator” refers to one or more PI3-kinase (PI3K) modulators or at least one PI3-kinase (PI3K) modulator. In embodiments, the PI3-kinase (PI3K) modulator is a PI3Kα modulator. As such, the terms “a” (or “an”), “one or more” and “at least one” are used interchangeably herein. In addition, reference to “an inhibitor” by the indefinite article “a” or “an” does not exclude the possibility that more than one of the inhibitors is present, unless the context clearly requires that there is one and only one of the inhibitors.

As used herein, the verb “comprise” as is used in this description and in the claims and its conjugations are used in its non-limiting sense to mean that items following the word are included, but items not specifically mentioned are not excluded. The present invention may suitably “comprise”, “consist of”, or “consist essentially of”, the steps, elements, and/or reagents described in the claims.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

Pharmaceutically acceptable salts include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. Those skilled in the art will further recognize that acid addition salts may be prepared by reaction of the compounds with the appropriate inorganic or organic acid via any of a number of known methods.

As used herein, “solvate” means a complex formed by solvation (the combination of solvent molecules with molecules or ions of the active agent of the present invention), or an aggregate that consists of a solute ion or molecule (the active agent of the present invention) with one or more solvent molecules. In embodiments, the solvate is a hydrate. Examples of hydrate include, but are not limited to, channel hydrate, hemihydrate, monohydrate, dihydrate, trihydrate, hexahydrate, sesquihydrate etc. It should be understood by one of ordinary skill in the art that the pharmaceutically acceptable salt of the present compound may also exist in a solvate form (solvate salt). The solvate is typically formed via hydration which is either part of the preparation of the present compound or through natural absorption of moisture by the anhydrous compound of the present invention. Solvates including hydrates may be consisting in stoichiometric ratios, for example, with two, three, four salt molecules per solvate or per hydrate molecule. Another possibility, for example, that two salt molecules are stoichiometric related to three, five, seven solvent or hydrate molecules. Solvents used for crystallization, such as alcohols, especially methanol and ethanol; aldehydes; ketones, especially acetone; esters, e.g. ethyl acetate; may be embedded in the crystal grating. Preferred are pharmaceutically acceptable solvents.

The term “treating” means one or more of relieving, alleviating, delaying, reducing, improving, or managing at least one symptom of a condition in a subject. The term “treating” may also mean one or more of arresting, delaying the onset (i.e., the period prior to clinical manifestation of the condition) or reducing the risk of developing or worsening a condition.

An “effective amount” means the amount of a formulation according to the invention that, when administered to a patient for treating a state, disorder or condition is sufficient to effect such treatment. The “effective amount” will vary depending on the active ingredient, the state, disorder, or condition to be treated and its severity, and the age, weight, physical condition and responsiveness of the mammal to be treated.

The term “therapeutically effective” applied to dose or amount refers to that quantity of a compound or pharmaceutical formulation that is sufficient to result in a desired clinical benefit after administration to a patient in need thereof.

As used herein, a “subject” can be a human, non-human primate, mammal, rat, mouse, cow, horse, pig, sheep, goat, dog, cat and the like. In embodiments, the subject is human. In embodiments, the subject can be suspected of having or at risk for having a cancer.

“Mammal” includes humans and both domestic animals such as laboratory animals (e.g., mice, rats, monkeys, dogs, etc.) and household pets (e.g., cats, dogs, swine, cattle, sheep, goats, horses, rabbits), and non-domestic animals such as wildlife and the like.

All weight percentages (i.e., “% by weight” and “wt. %” and w/w) referenced herein, unless otherwise indicated, are measured relative to the total weight of the pharmaceutical composition.

As used herein, “substantially” or “substantial” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking, the nearness of completion will be so as to have the same overall result as if absolute and total completion were obtained. The use of “substantially” is equally applicable when used in a negative connotation to refer to the complete or near complete lack of action, characteristic, property, state, structure, item, or result. For example, a composition that is “substantially free of” other active agents would either completely lack other active agents, or so nearly completely lack other active agents that the effect would be the same as if it completely lacked other active agents. In other words, a composition that is “substantially free of” an ingredient or element or another active agent may still contain such an item as long as there is no measurable effect thereof.

Polymorphism can be characterized as the ability of a compound to crystallize into different crystal forms, while maintaining the same chemical formula. A crystalline polymorph of a given drug substance is chemically identical to any other crystalline polymorph of that drug substance in containing the same atoms bonded to one another in the same way, but differs in its crystal forms, which can affect one or more physical properties, such as stability, solubility, melting point, bulk density, flow properties, bioavailability, etc.

The following description includes information that may be useful in understanding the present invention. It is not an admission that any of the information provided herein is prior art or relevant to the presently claimed inventions, or that any publication specifically or implicitly referenced is prior art.

### DETAILED DESCRIPTION

Throughout this disclosure, various patents, patent applications and publications are referenced. The disclosures of these patents, patent applications and publications in their entireties are incorporated into this disclosure by reference for all purposes in order to more fully describe the state of the art as known to those skilled therein as of the date of this disclosure. This disclosure will govern in the instance that there is any inconsistency between the patents, patent applications and publications cited and this disclosure.

### Compound I and Salt and Solid Forms

Compound I is methyl (5-(6-((4-(acryloylglycyl) piperazin-1-yl)methyl)-4-morpholinopyrrolo[2,1-f][1,2,4]triazin-2-yl)-4-(trifluoromethyl)pyridin-2-yl) carbamate, having the structure below. Compound I is disclosed in WO 2021/055747, which is hereby incorporated by reference in its entirety. In embodiments, Compound I is a phosphoinositide 3-kinase (PI3K) inhibitor. In embodiments, Compound I is an irreversible inhibitor of PI3K. In embodiments, Compound I is an irreversible inhibitor of PI3Kα. In embodiments, Compound I can form a covalent bond with an amino acid of a PI3K e.g., PI3Kα. In embodiments, Compound I can form a covalent bond with a cysteine in a PI3K, e.g., PI3Kα (e.g., via a Michael reaction).

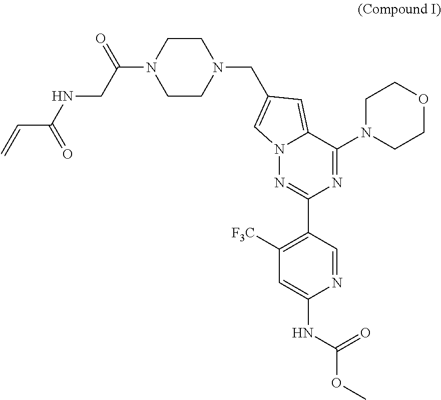 [SMILES: C=CC(=O)NCC(=O)N1CCN(Cc2cc3c(N4CCOCC4)nc(-c4cnc(NC(=O)OC)cc4C(F)(F)F)nn3c2)CC1]

In embodiments, the present disclosure relates to a salt of Compound I or a solvate thereof. In embodiments, the present disclosure relates to a crystalline form of Compound I or a pharmaceutically acceptable salt thereof, solvate thereof, or salt solvate thereof.

In one embodiment, the present disclosure relates to an anhydrous or non-solvated crystalline form of Compound I or a pharmaceutically acceptable salt thereof. In one embodiment, the present disclosure relates to an anhydrous or non-solvated crystalline form of Compound I (not a salt). In one embodiment, the present disclosure relates to a crystalline form of Compound I (not a salt). In embodiments, the present disclosure relates to a solvated crystalline form of Compound I or a pharmaceutically acceptable salt thereof. In embodiments, the present disclosure relates to a hydrated crystalline form of Compound I or a pharmaceutically acceptable salt thereof. In one embodiment, the present disclosure relates to a hydrated crystalline form of Compound I (not a salt). In one embodiment, the present disclosure relates to a hydrated crystalline form of a pharmaceutically acceptable salt of Compound I. In one embodiment, the present disclosure relates to a solvated crystalline form of Compound I (not a salt). In one embodiment, the present disclosure relates to a solvated crystalline form of a pharmaceutically acceptable salt of Compound I. In embodiments, the pharmaceutically acceptable salt of Compound I is a pharmaceutically acceptable acid. In embodiments, the pharmaceutically acceptable salt is obtained by reacting Compound I with an acid. In embodiments the acid is selected from the group consisting of phosphoric acid, hydrochloric acid, sulfuric acid, methanesulfonic acid, a benzenesulfonic acid, p-toluenesulfonic acid, fumaric acid, maleic acid, L-tartaric acid, citric acid, and succinic acid. In embodiments, the acid is phosphoric acid. In embodiments, the acid is hydrochloric acid. In embodiments, the acid is sulfuric acid. In embodiments, the acid is methanesulfonic acid. In embodiments, the acid is a benzenesulfonic acid. In embodiments, the acid is p-toluenesulfonic acid. In embodiments, the acid is fumaric acid. In embodiments, the acid is maleic acid. In embodiments, the acid is tartaric acid. In embodiments, the acid is L-tartaric acid. In embodiments, the acid is citric acid. In embodiments, the acid is succinic acid. In embodiments, the solvate of Compound I is a pharmaceutically acceptable acid. In embodiments, the solvated crystalline form of Compound I or solvated crystalline form of a pharmaceutically acceptable salt of Compound I is a solvate selected from the group consisting of water, acetone, benzyl alcohol, DMF, DMSO, THF, TFE or a combination thereof.

In embodiments, the present disclosure provides a compound of Formula (I-A):

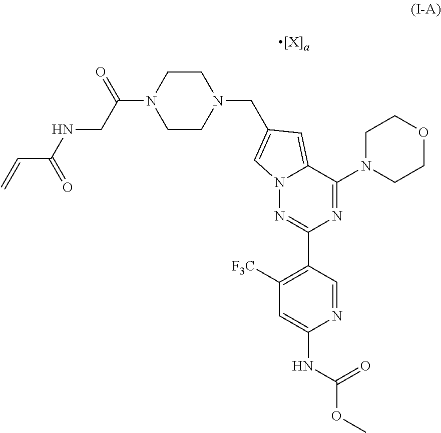

wherein X is a pharmaceutically acceptable acid; and
a is about 0.5 to about 2.

In embodiments, the present disclosure provides a compound of Formula (I-B):

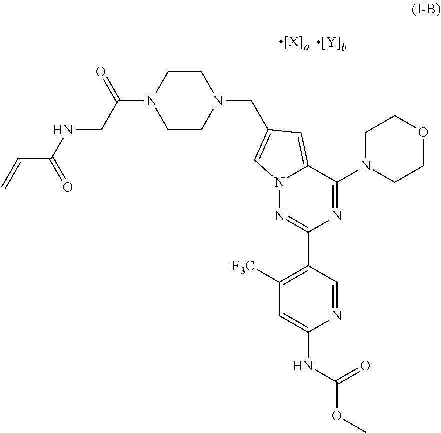

wherein:
Y is a solvent;
X is a pharmaceutically acceptable acid;
a is an integer of about 0.5 to about 2; and
b is an integer of about 0.5 to about 5.

In embodiments, the present disclosure provides a compound of Formula (I-C):

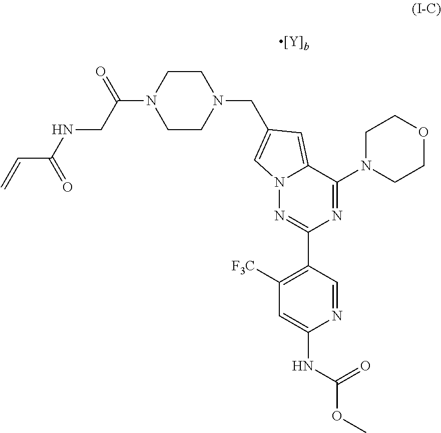

wherein Y is a solvent; and
b is about 0.5 to about 5.

In embodiments of the compound of Formula (I-A) or (I-B), a is about 1 to about 2.

In embodiments of the compound of Formula (I-A) or (I-B), a is about 0.5. In embodiments, a is about 1. In embodiments, a is about 1.5. In embodiments, a is about 2.

In embodiments of the compound of Formula (I-A) or (I-B), X is phosphoric acid, hydrochloric acid, sulfuric acid, methanesulfonic acid, benzenesulfonic acid, p-toluenesulfonic acid, fumaric acid, maleic acid, L-tartaric acid, citric acid, or succinic acid. In embodiments, X is phosphoric acid. In embodiments, X is hydrochloric acid. In embodiments, X is sulfuric acid. In embodiments, X is methanesulfonic acid. In embodiments, X is a benzenesulfonic acid. In embodiments, X is p-toluenesulfonic acid. In embodiments, X is fumaric acid. In embodiments, X is maleic acid. In embodiments, X is L-tartaric acid. In embodiments, X is citric acid. In embodiments, X is succinic acid.

In embodiments of the compound of Formula (I-B) or (I-C), the solvent is water, acetone, benzyl alcohol, DMF, DMSO, THF, TFE or a combination thereof. In embodiments of the compound of Formula (I-B) or (I-C), the solvent is water. In embodiments of the compound of Formula (I-B) or (I-C), the solvent is acetone. In embodiments of the compound of Formula (I-B) or (I-C), the solvent is benzyl alcohol. In embodiments of the compound of Formula (I-B) or (I-C), the solvent is DMF. In embodiments of the compound of Formula (I-B) or (I-C), the solvent is DMSO. In embodiments of the compound of Formula (I-B) or (I-C), the solvent is THF. In embodiments of the compound of Formula (I-B) or (I-C), the solvent is TFE.

In embodiments of the compound of Formula (I-B) or (I-C), b is about 0.5 to about 5. In embodiments, b is about 1.5 to about 2. In embodiments, b is about 0.5, about 1, about 1.5, about 2, about 2.5 about 3, about 3.5, about 4, or about 4.5.

In one embodiment, the crystalline forms are characterized by the interlattice plane intervals determined by an X-ray powder diffraction (XRPD) pattern. The spectrum of XRPD is typically represented by a diagram plotting the intensity of the peaks versus the location of the peaks, i.e., diffraction angle 20 (two-theta) in degrees. The intensities are often given in parenthesis with the following abbreviations: very strong=vst; strong=st; medium=m; weak=w; and very weak=vw. The characteristic peaks of a given XRPD can be selected according to the peak locations and their relative intensity to conveniently distinguish this crystalline structure from others. The % intensity of the peaks relative to the most intense peak may be represented as I/Io.

Those skilled in the art recognize that the measurements of the XRPD peak locations and/or intensity for a given crystalline form of the same compound will vary within a margin of error. The values of degree 20 allow appropriate error margins. Typically, the error margins are represented by “±”. For example, the degree 20 of about 17.48±0.2″ denotes a range from about 17.46 to 17.50 degree 20. Depending on the sample preparation techniques, the calibration techniques applied to the instruments, human operational variation, and etc., those skilled in the art recognize that the appropriate error of margins for a XRPD can be about ±0.7; ±0.6; ±0.5; ±0.4; ±0.3; ±0.2; ±0.1; ±0.05; or less.

Additional details of the methods and equipment used for the XRPD analysis are described in the Examples section.

In one embodiment, the crystalline forms are characterized by Differential Scanning calorimetry (DSC). The DSC thermogram is typically expressed by a diagram plotting the normalized heat flow in units of Watts/gram (“W/g”) versus the measured sample temperature in degree Celsius. The DSC thermogram is usually evaluated for extrapolated onset and end (outset) temperatures, peak temperature, and heat of fusion. A peak characteristic value of a DSC thermogram is often used as the characteristic peak to distinguish this crystalline structure from others.

Those skilled in the art recognize that the measurements of the DSC thermogram for a given crystalline form of the same compound will vary within a margin of error. The values of a single peak characteristic value, expressed in degree Celsius, allow appropriate error margins. Typically, the error margins are represented by “±”.

For example, the single peak characteristic value of about “17.48=0.2” denotes a range from about 17.46 to 17.50. Depending on the sample preparation techniques, the calibration techniques applied to the instruments, human operational variations, and etc., those skilled in the art recognize that the appropriate error of margins for a single peak characteristic value can be ±2.5; ±2.0; ±1.5; ±1.0; ±0.5; or less.

Additional details of the methods and equipment used for the DSC thermogram analysis are described in the Examples section.

In one embodiment, the crystalline forms are characterized by Dynamic Vapor Sorption (DVS). The DVS profile is typically expressed by a diagram plotting the sample relative humidity (RH) versus the change in mass (%). The DVS profile provides information on hygroscopicity of the crystalline form at different RH conditions.

Additional details of the methods and equipment used for DVS are described in the Examples section.

In embodiments, a crystalline form of Compound I, or a pharmaceutically acceptable salt thereof, solvate thereof, or salt solvate thereof (e.g., as disclosed herein) may comprise at least about 99.9%, at least about 99.8%, at least about 99.7%, at least about 99.6%, at least about 99.5%, at least about 99%, at least about 98%, at least about 97%, at least about 96%, at least about 95%, at least about 94%, at least about 93%, at least about 92%, at least about 91%, at least about 90%, at least about 85%, at least about 80%, at least about 75%, at least about 70%, at least about 65%, at least about 60%, at least about 55%, or at least about 50% of a single crystalline form (for example Form A, Form A*, Form A**, Form B*, Form A-1, Form B-1, Form A-2, Form B-2, Form A-3, Form B-3, Form C-3, Form A-4, Form A-5, Form A-6, Form A-7, Form B-7, Form A-8, Form B-8, Form A-9, Form B, Form C, Form J, Form K, Form H2, Form E, Form F, Form G, Form I, Form H1, Form H3, Form H4, Form H5, or Form H6). Polymorphic purity may be determined using methods known to those skilled in the art (including, e.g., X-ray powder crystallography).

In embodiments, the crystalline form of Compound I, or a pharmaceutically acceptable salt thereof, solvate thereof, or salt solvate thereof (e.g., as disclosed herein, for example Form A, Form A*, Form A**, Form B*, Form A-1, Form B-1, Form A-2, Form B-2, Form A-3, Form B-3, Form C-3, Form A-4, Form A-5, Form A-6, Form A-7, Form B-7, Form A-8, Form B-8, Form A-9, Form B, Form C, Form J, Form K, Form H2, Form E, Form F, Form G, Form I, Form H1, Form H3, Form H4, Form H5, or Form H6) has a purity of about 99.9% or higher, about 99.8% or higher, about 99.7% or higher, about 99.6% or higher, about 99.5% or higher, about 99% or higher, about 98% or higher, about 97% or higher, about 96% or higher, about 95% or higher, about 94% or higher, about 93% or higher, about 92% or higher, about 91%, or higher, about 90% or higher, about 85% or higher, or about 80% or higher. In embodiments, the crystalline form has a purity in the range of about 80% to about 99%. In embodiments, the crystalline form has a purity in the range of about 80% to about 99.5%. In embodiments, the crystalline form has a purity in the range of about 80% to about 99.9%. In embodiments, the crystalline form has a purity in the range of about 80% to about 100%. In embodiments, the purity is determined by HPLC.

In embodiments, the crystalline form of Compound I, or a pharmaceutically acceptable salt thereof, solvate thereof, or salt solvate thereof (as disclosed herein, for example Form A, Form A*, Form A**, Form B*, Form A-1, Form B-1, Form A-2, Form B-2, Form A-3, Form B-3, Form C-3, Form A-4, Form A-5, Form A-6, Form A-7, Form B-7, Form A-8, Form B-8, Form A-9, Form B, Form C, Form J, Form K, Form H2, Form E, Form F, Form G, Form I, Form H1, Form H3, Form H4, Form H5, or Form H6) is about 99.9% pure by weight or higher, about 99.8% pure by weight or higher, about 99.7% pure by weight or higher, about 99.6% pure by weight or higher, about 99.5% pure by weight or higher, about 99% pure by weight or higher, about 98% pure by weight or higher, about 97% pure by weight or higher, about 96% pure by weight or higher, about 95% pure by weight or higher, about 94% pure by weight or higher, about 93% pure by weight or higher, about 92% pure by weight or higher, about 91% pure by weight or higher, about 90% pure by weight or higher, about 85% pure by weight or higher, or about 80% pure by weight or higher. In embodiments, the crystalline form has a purity in the range of about 80% pure by weight to about 99% pure by weight of a single crystalline form (as disclosed herein, for example Form A, Form A*, Form A**, Form B*, Form A-1, Form B-1, Form A-2, Form B-2, Form A-3, Form B-3, Form C-3, Form A-4, Form A-5, Form A-6, Form A-7, Form B-7, Form A-8, Form B-8, Form A-9, Form B, Form C, Form J, Form K, Form H2, Form E, Form F, Form G, Form I, Form H1, Form H3, Form H4, Form H5, or Form H6). In embodiments, the purity is determined by HPLC.

In embodiments, the crystalline form of Compound I, or a pharmaceutically acceptable salt thereof, solvate thereof, or salt solvate thereof is at least about 95% pure by weight, and comprises no more than about 5% of an impurity by weight. In some embodiments, the crystalline form of Compound I, or a pharmaceutically acceptable salt thereof, solvate thereof, or salt solvate thereof is about 95.0% to 100% pure by weight, and comprises 0% to about 5% of an impurity by weight of the crystalline form of Compound I, or a pharmaceutically acceptable salt thereof, solvate thereof, or salt solvate thereof. In some embodiments, the crystalline form of Compound I, or a pharmaceutically acceptable salt thereof, solvate thereof, or salt solvate thereof is about 98% to 100% pure by weight, and comprises 0% to about 2% of an impurity by weight of the crystalline form of Compound I, or a pharmaceutically acceptable salt thereof, solvate thereof, or salt solvate thereof. In some embodiments, the crystalline form of Compound I, or a pharmaceutically acceptable salt thereof, solvate thereof, or salt solvate thereof is about 98%, about 98.5%, about 99%, about 99.5%, or 100% pure by weight, and comprises about 2%, about 1.5%, about 1%, about 0.5%, or 0%, respectively, of an impurity by weight of the crystalline form of Compound I, or a pharmaceutically acceptable salt thereof, solvate thereof, or salt solvate thereof. In some embodiments, the crystalline form of Compound I, or a pharmaceutically acceptable salt thereof, solvate thereof, or salt solvate thereof is about 99.5%, about 99.9%, or about 99.95% pure by weight, and comprises about 0.5%, about 0.1%, or about 0.05%, respectively, of an impurity by weight of the crystalline form of Compound I, or a pharmaceutically acceptable salt thereof, solvate thereof, or salt solvate thereof.

In some embodiments, the purity or the impurity are determined by high-performance liquid chromatography (HPLC).

### Compound I Form A

In embodiments, the present disclosure relates to a crystalline form of Compound I, which is Form A.

In embodiments, the present disclosure relates to Form A, which is a crystalline form of Compound I that is anhydrous or non-solvated.

In embodiments, crystalline Form A of Compound I or composition thereof comprises a mixture of one or more forms of polymorphs of Compound I. In embodiments, the crystalline form of Compound I comprises of substantially pure form of one polymorph type. In embodiment, the crystalline form of Compound I may comprise of over about 99.9%, about 99.8%, about 99.7%, about 99.6%, about 99.5%, about 99.4%, about 99.3%, about 99.2%, about 99.1%, or about 99.0% of Form A of Compound I. In another embodiment, the crystalline form of Compound I may comprise over about 99%, 98%, 97%, 96%, 95%, 94%, 93%, 92%, 91%, or 90% of Form A of Compound I. In some embodiments, the crystalline form of Compound I may comprise over about 90%, 85%, 80%, 75%, 70%, 65%, 60%, 55%, 50%, 45%, or 40% of Form A of Compound I.

In embodiments, crystalline Form A of Compound I exhibits an XRPD pattern comprising peaks at about 9.11, 22.21, and 24.99 degrees two-theta with the margin of error of about ±0.5; about ±0.4; about ±0.3; about ±0.2; about ±0.1; about ±0.05; or less. In embodiments, crystalline Form A of Compound I exhibits an XRPD pattern comprising peaks at about 9.11, 16.93, 18.70, 22.21, and 24.99 degrees two-theta, with the margin of error of about ±0.5; about ±0.4; about ±0.3; about ±0.2; about ±0.1; about ±0.05; or less. In embodiments, crystalline Form A of Compound I exhibits an XRPD pattern comprising peaks at about 9.11, 16.93, 18.70, 20.54, 20.78, 22.21, and 24.99 degrees two-theta, with the margin of error of about ±0.5; about ±0.4; about ±0.3; about ±0.2; about ±0.1; about ±0.05; or less. In embodiments, crystalline Form A of Compound I exhibits an XRPD pattern which further comprises at least two peaks selected from about 4.47, 12.45, 14.51, 22.70, and 26.54 degrees two-theta, with the margin of error of about ±0.5; about ±0.4; about ±0.3; about ±0.2; about ±0.1; about ±0.05; or less. In embodiments, crystalline Form A of Compound I exhibits an XRPD pattern which further comprises at least three peaks selected from about 4.47, 12.45, 14.51, 22.70, and 26.54 degrees two-theta, with the margin of error of about ±0.5; about ±0.4; about ±0.3; about ±0.2; about ±0.1; about ±0.05; or less. In embodiments, crystalline Form A of Compound I exhibits an XRPD pattern which further comprises at least four peaks selected from about 4.47, 12.45, 14.51, 22.70, and 26.54 degrees two-theta, with the margin of error of about ±0.5; about ±0.4; about ±0.3; about ±0.2; about ±0.1; about ±0.05; or less. In embodiments, crystalline Form A of Compound I exhibits an XRPD pattern which further comprises peaks at about 4.47, 12.45, 14.51, 22.70, and 26.54 degrees two-theta, with the margin of error of about ±0.5; about ±0.4; about ±0.3; about ±0.2; about ±0.1; about ±0.05; or less. In embodiments, crystalline Form A of Compound I exhibits an XRPD pattern comprising peaks at about 9.11, 14.51, 16.93, 18.70, 20.54, 20.78, 22.21, 22.70, 24.99, and 26.54 degrees two-theta, with the margin of error of about ±0.5; about ±0.4; about ±0.3; about ±0.2; about ±0.1; about ±0.05; or less.

In embodiments, crystalline Form A of Compound I exhibits an XRPD pattern comprising peaks at about 9.11±0.2, 22.21±0.2, and 24.99±0.2 degrees two-theta.

In embodiments, crystalline Form A of Compound I exhibits an XRPD pattern comprising peaks at about 9.11±0.2, 16.93±0.2, 18.70±0.2, 22.21±0.2, and 24.99±0.2 degrees two-theta.

In embodiments, crystalline Form A of Compound I exhibits an XRPD pattern comprising peaks at about 9.11±0.2, 16.93±0.2, 18.70±0.2, 20.54±0.2, 20.78±0.2, 22.21±0.2, and 24.99±0.2 degrees two-theta.

In embodiments, crystalline Form A of Compound I exhibits an XRPD pattern which further comprises at least two peaks selected from about 4.47±0.2, 12.45±0.2, 14.51±0.2, 22.70±0.2, and 26.54±0.2 degrees two-theta.

In embodiments, crystalline Form A of Compound I exhibits an XRPD pattern comprising peaks at about 9.11±0.2, 14.51±0.2, 16.93±0.2, 18.70±0.2, 20.54±0.2, 20.78±0.2, 22.21±0.2, 22.70±0.2, 24.99±0.2, and 26.54±0.2 degrees two-theta.

In some embodiments, the crystalline form of Compound I exhibits an XRPD pattern comprising 1 peak, 2 peaks, 3 peaks, 4 peaks, 5 peaks, 6 peaks, 7 peaks, 8 peaks, 9 peaks, or 10 peaks ±0.2 degrees two-theta of Table 1.

In an embodiment, the crystalline Form A of Compound I exhibits an XRPD comprising peaks shown in Table 1 below.

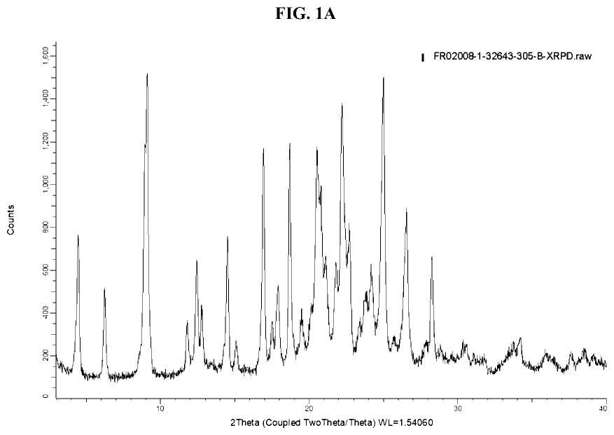

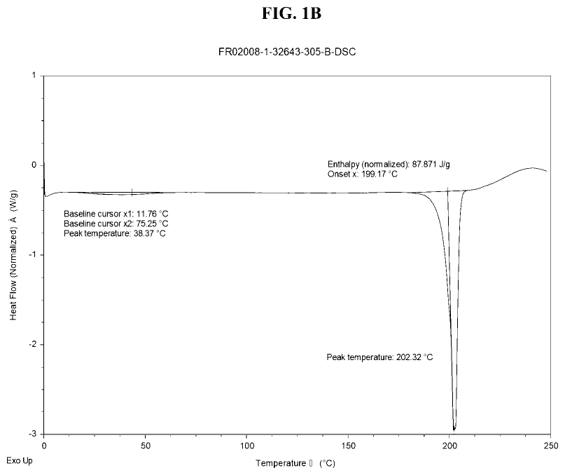

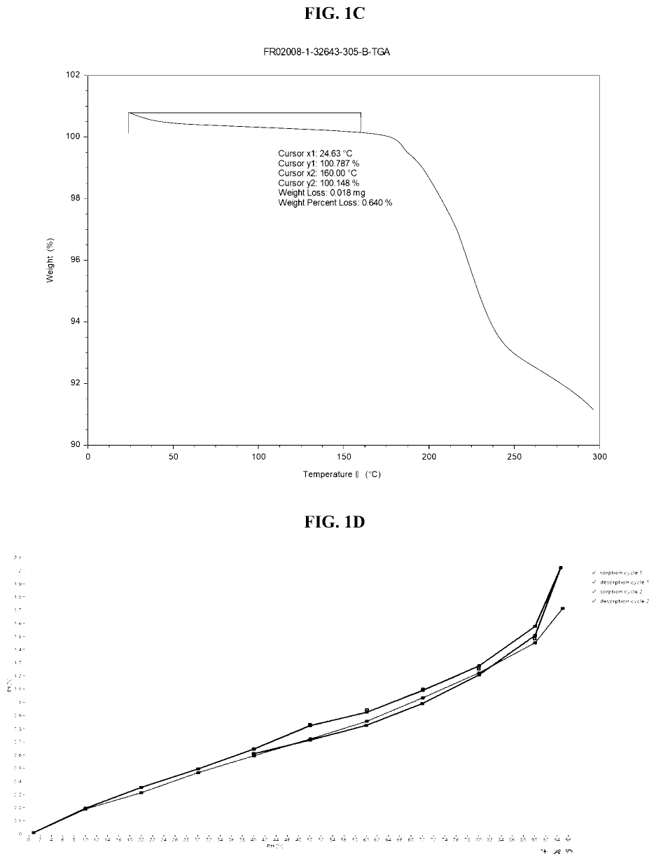

In embodiments, the crystalline Form A of Compound I exhibits an XRPD pattern that is substantially similar to FIG. 1A.

TABLE 1
Form A of Compound I

| | | | | Net | Gross | Rel. |
|---|---|---|---|---|---|---|
| Index | Angle | d Value | | Intensity | Intensity | Intensity |
| 1 | 4.465° | 19.77237 | Å | 388.314 | 499.141 | 41.4% |
| 2 | 6.251° | 14.12824 | Å | 259.625 | 360.519 | 27.7% |
| 3 | 9.106° | 9.70369 | Å | 938.077 | 1049.93 | 100.0% |
| 4 | 11.794° | 7.49753 | Å | 164.720 | 290.512 | 17.6% |
| 5 | 12.446° | 7.10625 | Å | 352.957 | 485.683 | 37.6% |
| 6 | 12.778° | 6.92249 | Å | 155.045 | 290.028 | 16.5% |
| 7 | 14.514° | 6.09782 | Å | 436.265 | 572.064 | 46.5% |
| 8 | 15.078° | 5.87132 | Å | 97.3973 | 229.875 | 10.4% |
| 9 | 16.934° | 5.23171 | Å | 720.194 | 867.742 | 76.8% |
| 10 | 17.510° | 5.06084 | Å | 131.891 | 291.642 | 14.1% |
| 11 | 17.916° | 4.94704 | Å | 242.172 | 408.968 | 25.8% |
| 12 | 18.698° | 4.74171 | Å | 653.182 | 837.786 | 69.6% |
| 13 | 19.502° | 4.54822 | Å | 170.342 | 375.188 | 18.2% |
| 14 | 20.150° | 4.40334 | Å | 149.116 | 366.632 | 15.9% |
| 15 | 20.543° | 4.31994 | Å | 566.914 | 790.519 | 60.4% |
| 16 | 20.782° | 4.27086 | Å | 519.788 | 746.501 | 55.4% |
| 17 | 21.095° | 4.20821 | Å | 285.100 | 515.213 | 30.4% |
| 18 | 21.842° | 4.06589 | Å | 241.544 | 476.690 | 25.7% |
| 19 | 22.213° | 3.99870 | Å | 774.246 | 1010.27 | 82.5% |
| 20 | 22.701° | 3.91390 | Å | 419.855 | 655.404 | 44.8% |
| 21 | 23.423° | 3.79483 | Å | 105.665 | 339.393 | 11.3% |
| 22 | 23.801° | 3.73552 | Å | 192.259 | 424.623 | 20.5% |
| 23 | 24.166° | 3.67987 | Å | 299.730 | 529.716 | 32.0% |
| 24 | 24.994° | 3.55981 | Å | 883.860 | 1104.60 | 94.2% |
| 25 | 25.709° | 3.46238 | Å | 52.5351 | 260.991 | 5.6% |
| 26 | 26.541° | 3.35567 | Å | 491.028 | 680.162 | 52.3% |
| 27 | 27.892° | 3.19613 | Å | 47.1186 | 223.505 | 5.0% |
| 28 | 28.255° | 3.15592 | Å | 348.395 | 525.383 | 37.1% |
| 29 | 28.853° | 3.09190 | Å | 52.5333 | 228.275 | 5.6% |
| 30 | 30.519° | 2.92674 | Å | 44.2586 | 201.806 | 4.7% |
| 31 | 31.101° | 2.87330 | Å | 43.7778 | 189.874 | 4.7% |
| 32 | 33.766° | 2.65237 | Å | 99.2531 | 234.180 | 10.6% |
| 33 | 34.162° | 2.62256 | Å | 81.5793 | 217.238 | 8.7% |
| 34 | 35.925° | 2.49780 | Å | 54.6594 | 190.712 | 5.8% |
| 35 | 37.572° | 2.39196 | Å | 48.8664 | 191.248 | 5.2% |
| 36 | 38.514° | 2.33562 | Å | 40.4355 | 194.727 | 4.3% |
| 37 | 39.115° | 2.30108 | Å | 24.9863 | 183.266 | 2.7% |

In embodiments, the crystalline Form A of Compound I exhibits a DSC thermogram comprising an endotherm peak at about 199° C. (onset) with the error of margin of about ±2.5; about ±2.0; about ±1.5; about ±1.0; about ±0.5; or less. In one embodiment, the crystalline Form A of Compound I exhibits a DSC thermogram comprising an endotherm peak at about 202° C. (peak) with the error of margin of about ±2.5; about ±2.0; about ±1.5; about ±1.0; about ±0.5; or less. In embodiments, the crystalline Form A of Compound I exhibits a DSC thermogram comprising an endothermic peak at 11.8° C.; with the error of margin of about ±2.5; about ±2.0; about ±1.5; about ±1.0; about ±0.5; or less.

In one embodiment, the crystalline Form A of Compound I exhibits a DSC thermogram that is substantially similar to FIG. 1B.

In embodiments, the crystalline Form A of Compound I exhibits a TGA thermogram substantially similar to FIG. 1C. In embodiments, the crystalline Form A of Compound I exhibits a weight percent loss of about 0.6% between about 25° C. to about 160° C. by a thermogravimetric analysis (TGA).

### Compound I Phosphate Salt Form A*

In one embodiment, the present disclosure relates to a Compound I phosphate salt or solvate thereof. In embodiments, the present disclosure relates to a crystalline form of Compound I phosphate salt, which is Form A*.

In embodiments, the present disclosure relates to Form A*, which is a crystalline form of Compound I phosphate salt, that is a hydrate.

In embodiments, the crystalline form of Compound I phosphate salt is a hydrate, wherein the ratio of Compound I phosphate salt molecules: water molecules in the crystalline form is about 5:1, about 4:1, about 3:1, about 2.5:1, about 2:1, about 1.5:1, about 1:1, about 1:1.5, about 1:2, about 1:2.5, about 1:3, about 1:4, or about 1:5.

In embodiments, the crystalline form of Compound I phosphate salt is a channel hydrate.

In embodiments, the crystalline form of Compound I phosphate salt comprises a mixture of one or more forms of polymorphs of Compound I phosphate salt. In embodiments, the crystalline form of Compound I phosphate salt comprises of substantially pure form of one polymorph type. In embodiment, the crystalline form of Compound I phosphate salt may comprise of over about 99.9%, about 99.8%, about 99.7%, about 99.6%, about 99.5%, about 99.4%, about 99.3%, about 99.2%, about 99.1%, or about 99.0% of Form A* of Compound I phosphate salt. In another embodiment, the crystalline form of Compound I phosphate salt may comprise over about 99%, 98%, 97%, 96%, 95%, 94%, 93%, 92%, 91%, or 90% of Form A* of Compound I phosphate salt. In some embodiments, the crystalline form of Compound I phosphate salt may comprise over about 90%, 85%, 80%, 75%, 70%, 65%, 60%, 55%, 50%, 45%, or 40% of Form A* of Compound I phosphate salt.

In embodiments, crystalline Form A* of Compound I phosphate salt exhibits an XRPD pattern comprising peaks at about 5.33, 15.97, and 22.97, degrees two-theta with the margin of error of about ±0.5; about ±0.4; about ±0.3; about ±0.2; about ±0.1; about ±0.05; or less. In embodiments, crystalline Form A* of Compound I phosphate salt exhibits an XRPD pattern comprising peaks at about 5.33, 10.63, 15.97, 20.95, and 22.97, degrees two-theta with the margin of error of about ±0.5; about ±0.4; about ±0.3; about ±0.2; about ±0.1; about ±0.05; or less. In embodiments, crystalline Form A* of Compound I phosphate salt exhibits an XRPD pattern comprising peaks at about 5.33, 10.63, 15.97, 20.26, 20.95, 22.71, and 22.97, degrees two-theta with the margin of error of about ±0.5; about ±0.4; about ±0.3; about ±0.2; about ±0.1; about ±0.05; or less. In embodiments, crystalline Form A* of Compound I phosphate salt exhibits an XRPD pattern which further comprises at least two peaks selected from about 7.24, 14.94, 18.64, 18.99, and 21.34 degrees two-theta with the margin of error of about ±0.5; about ±0.4; about ±0.3; about ±0.2; about ±0.1; about ±0.05; or less. In embodiments, crystalline Form A* of Compound I phosphate salt exhibits an XRPD pattern which further comprises at least three peaks selected from about 7.24, 14.94, 18.64, 18.99, and 21.34 degrees two-theta with the margin of error of about ±0.5; about ±0.4; about ±0.3; about ±0.2; about ±0.1; about ±0.05; or less. In embodiments, crystalline Form A* of Compound I phosphate salt exhibits an XRPD pattern which further comprises at least four peaks selected from about 7.24, 14.94, 18.64, 18.99, and 21.34 degrees two-theta with the margin of error of about ±0.5; about ±0.4; about ±0.3; about ±0.2; about ±0.1; about ±0.05; or less. In embodiments, crystalline Form A* of Compound I phosphate salt exhibits an XRPD pattern which further comprises peaks at about 7.24, 14.94, 18.64, 18.99, and 21.34 degrees two-theta with the margin of error of about ±0.5; about ±0.4; about ±0.3; about ±0.2; about ±0.1; about ±0.05; or less. In embodiments, crystalline Form A* of Compound I phosphate salt exhibits an XRPD pattern comprising peaks at about 5.33, 7.24, 10.63, 14.94, 15.97, 18.64, 20.26, 20.95, 22.71, and 22.97, degrees two-theta with the margin of error of about ±0.5; about ±0.4; about ±0.3; about ±0.2; about ±0.1; about ±0.05; or less.

In embodiments, crystalline Form A* of Compound I phosphate salt exhibits an X-ray powder diffraction (XRPD) pattern comprising peaks at about 5.33±0.2, 15.97±0.2, and 22.97±0.2, degrees two-theta.

In embodiments, crystalline Form A* of Compound I phosphate salt exhibits an X-ray powder diffraction (XRPD) pattern comprising peaks at about 5.33±0.2, 10.63±0.2, 15.97±0.2, 20.95±0.2, and 22.97±0.2, degrees two-theta.

In embodiments, crystalline Form A* of Compound I phosphate salt exhibits an X-ray powder diffraction (XRPD) pattern comprising peaks at about 5.33±0.2, 10.63±0.2, 15.97±0.2, 20.26±0.2, 20.95±0.2, 22.71±0.2, and 22.97±0.2, degrees two-theta.

In embodiments, crystalline Form A* of Compound I phosphate salt exhibits an X-ray powder diffraction (XRPD) pattern further comprises at least two peaks selected from about 7.24±0.2, 14.94±0.2, 18.64±0.2, 18.99±0.2, and 21.34±0.2 degrees two-theta.

In embodiments, crystalline Form A* of Compound I phosphate salt exhibits an X-ray powder diffraction (XRPD) pattern comprising peaks at about 5.33±0.2, 7.23±0.2, 10.63±0.2, 14.94±0.2, 15.97±0.2, 18.64±0.2, 20.26±0.2, 20.95±0.2, 22.71±0.2, and 22.97±0.2, degrees two-theta.

In some embodiments, the crystalline form of Compound I phosphate salt exhibits an XRPD pattern comprising 1 peak, 2 peaks, 3 peaks, 4 peaks, 5 peaks, 6 peaks, 7 peaks, 8 peaks, 9 peaks, or 10 peaks ±0.2 degrees two-theta of Table 2.

In an embodiment, the crystalline Form A* of Compound I phosphate salt exhibits an XRPD comprising peaks shown in Table 2 below.

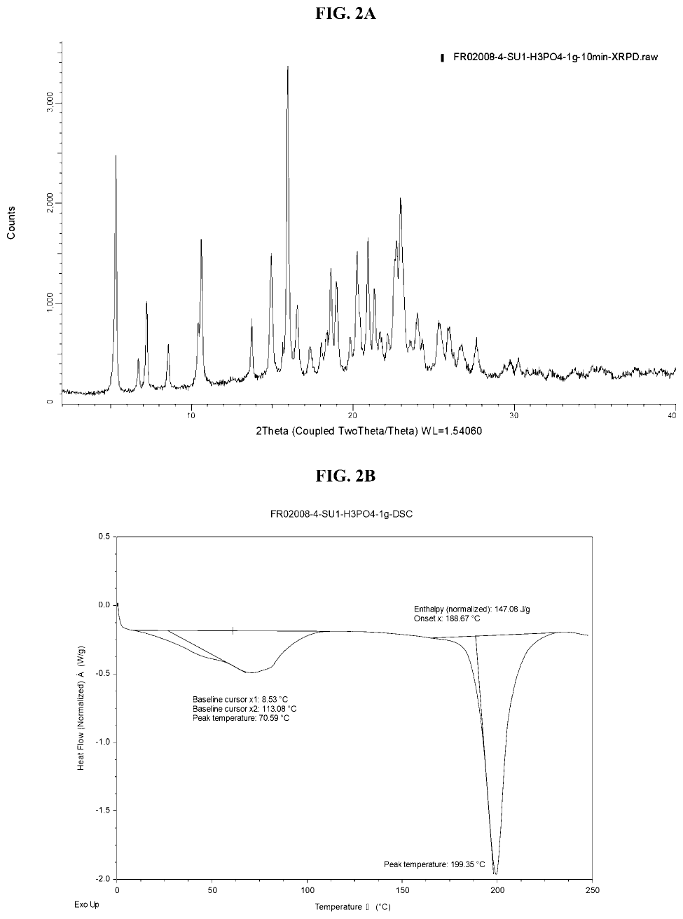

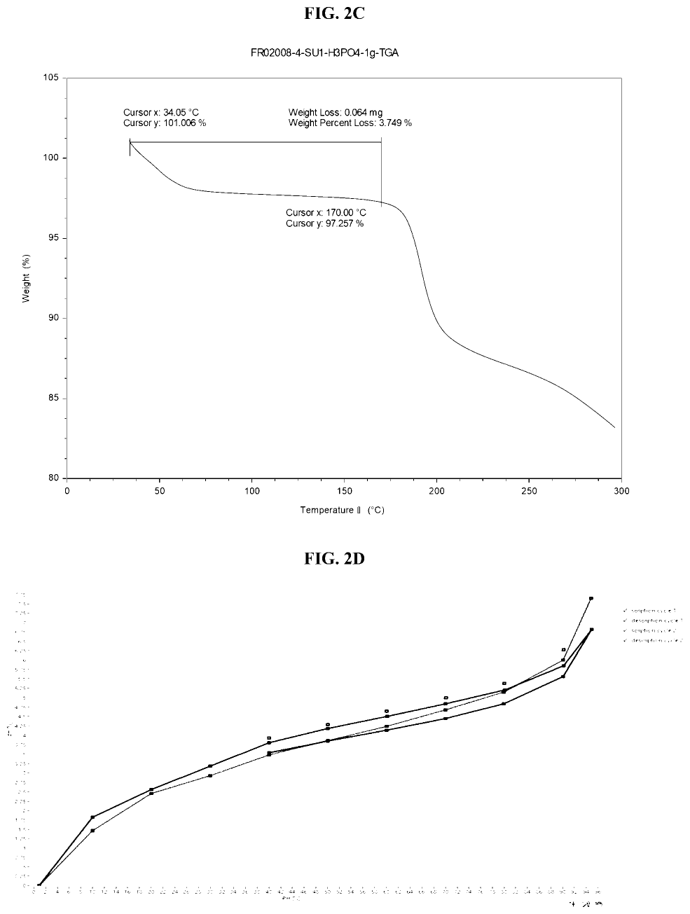

In embodiments, the crystalline Form A* of Compound I phosphate salt exhibits an XRPD pattern that is substantially similar to FIG. 2A.

TABLE 2
Form A* of Compound I phosphate salt

| Angle | d Value | | Net Intensity | Rel. Intensity |
|---|---|---|---|---|
| 5.327° | 16.57757 | Å | 1470.46 | 70.2% |
| 6.731° | 13.12119 | Å | 206.671 | 9.9% |
| 7.237° | 12.20507 | Å | 578.473 | 27.6% |
| 8.577° | 10.30081 | Å | 293.730 | 14.0% |
| 10.442° | 8.46537 | Å | 408.154 | 19.5% |
| 10.630° | 8.31607 | Å | 977.735 | 46.7% |
| 13.730° | 6.44421 | Å | 428.984 | 20.5% |
| 14.942° | 5.92430 | Å | 797.063 | 38.1% |
| 15.670° | 5.65079 | Å | 200.144 | 9.6% |
| 15.966° | 5.54643 | Å | 2094.29 | 100.0% |
| 16.556° | 5.35005 | Å | 459.591 | 21.9% |
| 17.362° | 5.10367 | Å | 147.067 | 4.0% |
| 18.043° | 4.91243 | Å | 206.090 | 9.8% |
| 18.412° | 4.81494 | Å | 244.809 | 11.7% |
| 18.641° | 4.75630 | Å | 699.260 | 33.4% |
| 18.991° | 4.66931 | Å | 576.090 | 27.5% |
| 19.815° | 4.47695 | Å | 241.758 | 11.5% |
| 20.263° | 4.37902 | Å | 802.706 | 38.3% |
| 20.947° | 4.23755 | Å | 877.919 | 41.9% |
| 21.335° | 4.16126 | Å | 543.459 | 25.9% |
| 21.687° | 4.09465 | Å | 208.404 | 10.0% |
| 22.169° | 4.00668 | Å | 214.062 | 10.2% |
| 22.712° | 3.91211 | Å | 857.532 | 40.9% |
| 22.971° | 3.86845 | Å | 1091.09 | 52.1% |
| 23.570° | 3.77155 | Å | 168.951 | 8.1% |
| 24.005° | 3.70413 | Å | 354.580 | 16.9% |
| 24.289° | 3.66150 | Å | 219.551 | 10.5% |
| 25.371° | 3.50778 | Å | 276.358 | 13.2% |
| 25.967° | 3.42860 | Å | 253.641 | 12.1% |
| 26.708° | 3.33515 | Å | 183.427 | 8.8% |
| 27.654° | 3.22317 | Å | 253.321 | 12.1% |
| 28.079° | 3.17535 | Å | 48.8649 | 2.3% |
| 29.416° | 3.03392 | Å | 67.5189 | 3.2% |
| 29.747° | 3.00099 | Å | 107.582 | 5.1% |
| 30.251° | 2.95176 | Å | 146.632 | 7.0% |
| 30.793° | 2.90134 | Å | 42.1055 | 2.0% |
| 31.537° | 2.83462 | Å | 85.5296 | 4.1% |
| 32.187° | 2.77882 | Å | 76.6406 | 3.7% |
| 34.827° | 2.57400 | Å | 78.5494 | 3.8% |
| 35.396° | 2.53386 | Å | 78.5685 | 3.8% |
| 35.831° | 2.50411 | Å | 42.6060 | 2.0% |
| 38.430° | 2.34055 | Å | 30.7115 | 1.5% |
| 38.681° | 2.32590 | Å | 67.1356 | 3.2% |

In embodiments, the crystalline Form A* of Compound I phosphate salt exhibits a DSC thermogram comprising an endotherm peak at about 8.5° C. with the error of margin of about ±2.5; about ±2.0; about ±1.5; about ±1.0; about ±0.5; or less. In embodiments, the crystalline Form A* of Compound I phosphate salt exhibits a DSC thermogram comprising an endotherm peak at about 8.5° C., which is likely due to dehydration. In embodiments, the crystalline Form A* of Compound I phosphate salt exhibits a DSC thermogram comprising an endotherm peak with an onset at about 189° C. with the error of margin of about ±2.5; about ±2.0; about ±1.5; about ±1.0; about ±0.5; or less. In embodiments, the crystalline Form A* of Compound I phosphate salt exhibits a DSC thermogram comprising an endotherm peak with an onset at about 189° C., which is likely due to decomposition. In embodiments, the crystalline Form A* of Compound I phosphate salt exhibits a DSC thermogram comprising an endotherm peak at about 199° C. (peak) with the error of margin of about ±2.5; about ±2.0; about ±1.5; about ±1.0; about ±0.5; or less.

In some embodiments, the crystalline Form A* of Compound I phosphate salt exhibits a DSC thermogram that is substantially similar to FIG. 2B.

In some embodiments, the crystalline Form A* of Compound I phosphate salt exhibits a TGA thermogram substantially similar to FIG. 2C. In embodiments, the crystalline Form A* of Compound I phosphate salt exhibits a weight percent loss of about 3.75% between about 34° C. to about 170° C. by a thermogravimetric analysis (TGA).

### Compound I Benzenesulfonate Salt Form A*

In one embodiment, the present disclosure relates to a Compound I benzenesulfonate salt, or a solvate thereof. In embodiments, the present disclosure relates to a crystalline form of Compound I benzenesulfonate salt, which is Form A**

In embodiments, the present disclosure relates to Form A**, which is a crystalline form of Compound I benzenesulfonate salt, that is an anhydrate.

In embodiments, the crystalline of Compound I benzenesulfonate salt comprises a mixture of one or more forms of polymorphs of Compound I benzenesulfonate salt. In embodiments, the crystalline form of Compound I benzenesulfonate salt comprises of substantially pure form of one polymorph type. In embodiment, the crystalline form of Compound I benzenesulfonate salt may comprise of over about 99.9%, about 99.8%, about 99.7%, about 99.6%, about 99.5%, about 99.4%, about 99.3%, about 99.2%, about 99.1%, or about 99.0% of Form A** of Compound I benzenesulfonate salt. In another embodiment, the crystalline form of Compound I benzenesulfonate salt may comprise over about 99%, 98%, 97%, 96%, 95%, 94%, 93%, 92%, 91%, or 90% of Form A** of Compound I benzenesulfonate salt. In some embodiments, the crystalline form of Compound I benzenesulfonate salt may comprise over about 90%, 85%, 80%, 75%, 70%, 65%, 60%, 55%, 50%, 45%, or 40% of Form A** of Compound I benzenesulfonate salt.

In embodiments, crystalline Form A** of Compound I benzenesulfonate salt exhibits an XRPD pattern comprising peaks at about 7.36, 18.92 and 19.54 degrees two-theta with the margin of error of about ±0.5; about ±0.4; about ±0.3; about ±0.2; about ±0.1; about ±0.05; or less. In embodiments, crystalline Form A** of Compound I benzenesulfonate salt exhibits an XRPD pattern comprising peaks at about 7.36, 14.71, 18.52, 18.92 and 19.54 degrees two-theta with the margin of error of about ±0.5; about ±0.4; about ±0.3; about ±0.2; about ±0.1; about ±0.05; or less. In embodiments, crystalline Form A** of Compound I benzenesulfonate salt exhibits an XRPD pattern comprising peaks at about 7.36, 10.08, 14.71, 18.52, 18.92, 19.54, and 21.31 degrees two-theta with the margin of error of about ±0.5; about ±0.4; about ±0.3; about ±0.2; about ±0.1; about ±0.05; or less. In embodiments, crystalline Form A** of Compound I benzenesulfonate salt exhibits an XRPD pattern, which further comprises at least two peaks selected from about 15.50±0.2, 18.23±0.2, 22.72±0.2, 23.22-0.2, and 24.63±0.2 degrees two-theta. In embodiments, crystalline Form A** of Compound I benzenesulfonate salt exhibits an XRPD pattern, which further comprises at least three peaks selected from about 15.50±0.2, 18.23-0.2, 22.72±0.2, 23.22±0.2, and 24.63±0.2 degrees two-theta. In embodiments, crystalline Form A** of Compound I benzenesulfonate salt exhibits an XRPD pattern, which further comprises at least four peaks selected from about 15.50-0.2, 18.23-0.2, 22.72±0.2, 23.22±0.2, and 24.63±0.2 degrees two-theta. In embodiments, crystalline Form A** of Compound I benzenesulfonate salt exhibits an XRPD pattern which further comprises peaks at about 15.50±0.2, 18.23-0.2, 22.72±0.2, 23.22-0.2, and 24.63±0.2 degrees two-theta. In embodiments, crystalline Form A** of Compound I benzenesulfonate salt exhibits an XRPD pattern comprising peaks at about 7.36, 10.08, 14.71, 18.52, 18.92, 19.54, 21.31, 23.22, and 24.63 degrees two-theta with the margin of error of about ±0.5; about ±0.4; about ±0.3; about ±0.2; about ±0.1; about ±0.05; or less.

In embodiments, crystalline Form A** of Compound I benzenesulfonate salt exhibits an XRPD pattern comprising peaks at about 7.36±0.2, 18.92±0.2 and 19.54±0.2 degrees two-theta.

In embodiments, crystalline Form A** of Compound I benzenesulfonate salt exhibits an XRPD pattern comprising peaks at about 7.36±0.2, 14.71±0.2, 18.52±0.2, 18.92-0.2 and 19.54±0.2 degrees two-theta.

In embodiments, crystalline Form A** of Compound I benzenesulfonate salt exhibits an XRPD pattern comprising peaks at about 7.36-0.2, 10.08±0.2, 14.71±0.2, 18.52±0.2, 18.92±0.2, 19.54±0.2, and 21.31±0.2 degrees two-theta.

In embodiments, crystalline Form A** of Compound I benzenesulfonate salt exhibits an XRPD pattern which further comprises at least two peaks selected from about 15.50±0.2, 18.23±0.2, 22.72±0.2, 23.22±0.2, and 24.63-0.2 degrees two-theta.

In embodiments, crystalline Form A** of Compound I benzenesulfonate salt exhibits an XRPD pattern comprising peaks at about 7.36±0.2, 10.08±0.2, 14.71±0.2, 18.52±0.2, 18.92±0.2, 19.54-0.2, 21.31±0.2, 23.22, and 24.63 degrees two-theta.

In some embodiments, the crystalline form of Compound I benzenesulfonate salt exhibits an XRPD pattern comprising 1 peak, 2 peaks, 3 peaks, 4 peaks, 5 peaks, 6 peaks, 7 peaks, 8 peaks, 9 peaks, or 10 peaks ±0.2 degrees two-theta of Table 3.

In embodiments, crystalline Form A** of Compound I benzenesulfonate salt exhibits an XRPD comprising peaks shown in Table 3 below.

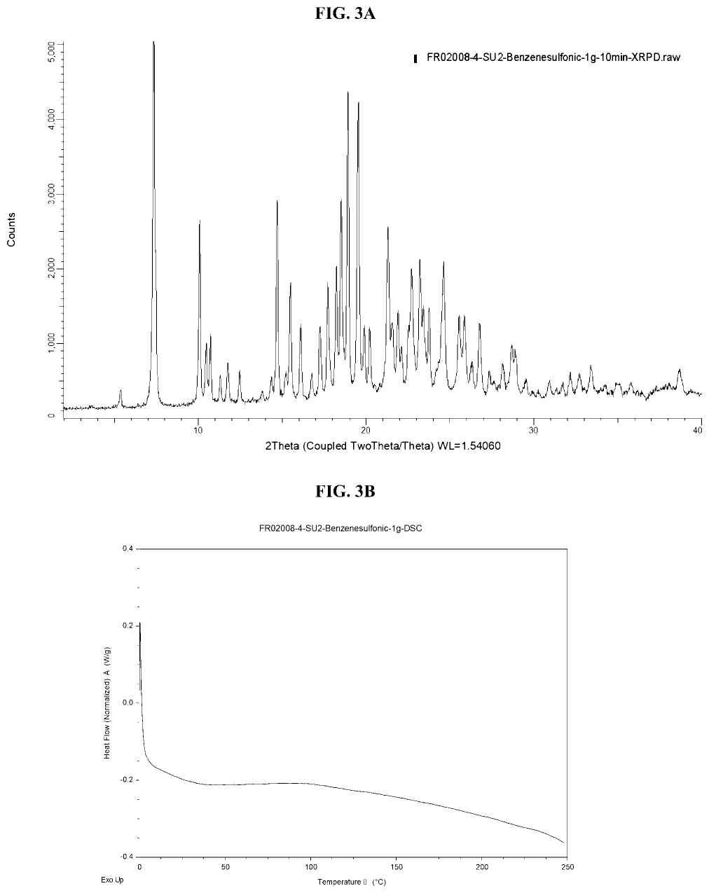

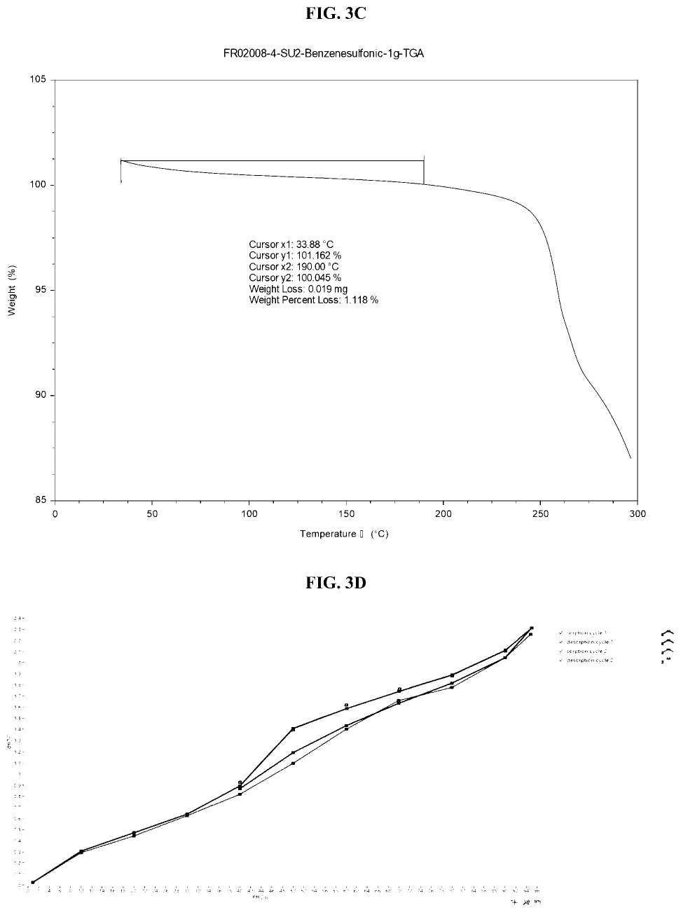

In embodiments, crystalline Form A** of Compound I benzenesulfonate salt exhibits an XRPD pattern that is substantially similar to FIG. 3A.

TABLE 3
Form A** of Compound I benzenesulfonate salt

| | d | Net | Rel. |
|---|---|---|---|
| Angle | Value | Intensity | Intensity |
| 5.378° | 16.41805 Å | 139.524 | 4.0% |
| 7.359° | 12.00373 Å | 3478.24 | 100.0% |
| 10.082° | 8.76686 Å | 1649.52 | 47.4% |
| 10.478° | 8.43623 Å | 535.089 | 15.4% |
| 10.725° | 8.24206 Å | 637.495 | 18.3% |
| 11.314° | 7.81469 Å | 241.001 | 6.9% |
| 11.767° | 7.51487 Å | 358.156 | 10.3% |
| 12.479° | 7.08773 Å | 294.248 | 8.5% |
| 13.826° | 6.39978 Å | 65.4240 | 1.9% |
| 14.364° | 6.16122 Å | 211.475 | 6.1% |
| 14.709° | 6.01768 Å | 1882.25 | 54.1% |
| 15.235° | 5.81110 Å | 240.582 | 6.9% |
| 15.497° | 5.71323 Å | 1030.04 | 29.6% |
| 16.111° | 5.49685 Å | 692.957 | 19.9% |
| 16.782° | 5.27859 Å | 214.177 | 6.2% |
| 17.254° | 5.13528 Å | 617.822 | 17.8% |
| 17.724° | 5.00010 Å | 999.823 | 28.7% |
| 18.234° | 4.86135 Å | 1201.48 | 34.5% |
| 18.519° | 4.78719 Å | 1839.38 | 52.9% |
| 18.924° | 4.68583 Å | 2886.53 | 83.0% |
| 19.540° | 4.53940 Å | 2646.97 | 76.1% |
| 19.895° | 4.45907 Å | 631.580 | 18.2% |
| 20.215° | 4.38927 Å | 598.513 | 17.2% |
| 20.511° | 4.32665 Å | 62.6740 | 1.8% |
| 20.822° | 4.26267 Å | 47.7363 | 1.4% |
| 21.311° | 4.16604 Å | 1521.93 | 43.8% |
| 21.555° | 4.11934 Å | 618.009 | 17.8% |
| 21.906° | 4.05418 Å | 709.206 | 20.4% |
| 22.121° | 4.01523 Å | 405.790 | 11.7% |
| 22.594° | 3.93229 Å | 447.401 | 12.9% |
| 22.722° | 3.91043 Å | 1124.33 | 32.3% |
| 23.218° | 3.82791 Å | 1234.22 | 35.5% |
| 23.429° | 3.79388 Å | 787.754 | 22.6% |
| 23.764° | 3.74114 Å | 796.439 | 22.9% |
| 24.237° | 3.66930 Å | 147.403 | 4.2% |
| 24.628° | 3.61188 Å | 1215.58 | 34.9% |
| 25.559° | 3.48243 Å | 699.273 | 20.1% |
| 25.874° | 3.44065 Å | 697.024 | 20.0% |
| 26.308° | 3.38490 Å | 274.552 | 7.9% |
| 26.771° | 3.32743 Å | 640.252 | 18.4% |
| 27.345° | 3.25890 Å | 211.658 | 6.1% |
| 27.620° | 3.22706 Å | 104.431 | 3.0% |
| 28.154° | 3.16701 Å | 259.356 | 7.5% |
| 28.689° | 3.10917 Å | 418.675 | 12.0% |
| 28.892° | 3.08774 Å | 424.962 | 12.2% |
| 29.523° | 3.02324 Å | 106.515 | 3.1% |
| 30.938° | 2.88811 Å | 145.997 | 4.2% |
| 31.357° | 2.85047 Å | 76.7122 | 2.2% |
| 31.726° | 2.81815 Å | 133.751 | 3.8% |
| 32.171° | 2.78013 Å | 230.792 | 6.6% |
| 32.728° | 2.73407 Å | 183.870 | 5.3% |
| 33.404° | 2.68029 Å | 308.330 | 8.9% |
| 34.272° | 2.61436 Å | 75.0350 | 2.2% |
| 34.639° | 2.58751 Å | 44.2051 | 1.3% |
| 34.969° | 2.56385 Å | 82.5067 | 2.4% |
| 35.050° | 2.55809 Å | 92.7724 | 2.7% |
| 35.466° | 2.52904 Å | 35.1568 | 1.0% |
| 35.780° | 2.50760 Å | 138.818 | 4.0% |
| 36.146° | 2.48301 Å | 62.3263 | 1.8% |
| 36.411° | 2.46555 Å | 72.3447 | 2.1% |
| 36.961° | 2.43013 Å | 68.2565 | 2.0% |
| 37.282° | 2.40993 Å | 110.053 | 3.2% |
| 37.854° | 2.37482 Å | 124.929 | 3.6% |
| 38.067° | 2.36200 Å | 126.729 | 3.6% |
| 38.710° | 2.32427 Å | 228.810 | 6.6% |

In embodiments, the crystalline Form A** of Compound I benzenesulfonate salt exhibits a DSC thermogram which shows decomposition at about 250° C. (upon melting) with the error of margin of about ±2.5; about ±2.0; about ±1.5; about ±1.0; about ±0.5; or less.

In embodiments, the crystalline Form A** of Compound I benzenesulfonate salt exhibits a TGA thermogram substantially similar to FIG. 3C. In embodiments, the crystalline Form A** of Compound I benzenesulfonate salt exhibits a weight percent loss of about 1.1% between about 34° C. to about 190° C. by a thermogravimetric analysis (TGA).

### Compound I Maleate Salt

In one embodiment, the present disclosure relates to a Compound I maleate salt, or a solvate thereof. In embodiments, the crystalline form of Compound I maleate salt comprises a mixture of one or more forms of polymorphs of Compound I maleate salt. In embodiments, the crystalline form of Compound I maleate salt comprises of substantially pure form of one polymorph type.

### Compound I Maleate Salt Form B*

In embodiments, the present disclosure relates to a crystalline form of Compound I maleate salt, which is Form B*.

In embodiments, the present disclosure relates to Form B*, which is a crystalline form of Compound I maleate salt, that is a hydrate.

In embodiment, the crystalline form of Compound I maleate salt may comprise of over about 99.9%, about 99.8%, about 99.7%, about 99.6%, about 99.5%, about 99.4%, about 99.3%, about 99.2%, about 99.1%, or about 99.0% of Form B* of Compound I maleate salt. In another embodiment, the crystalline form of Compound I maleate salt may comprise over about 99%, 98%, 97%, 96%, 95%, 94%, 93%, 92%, 91%, or 90% of Form B* of Compound I maleate salt. In some embodiments, the crystalline form of Compound I maleate salt may comprise over about 90%, 85%, 80%, 75%, 70%, 65%, 60%, 55%, 50%, 45%, or 40% of Form B* of Compound I maleate salt

In embodiments, crystalline Form B* of Compound I maleate salt exhibits an XRPD pattern comprising peaks at about 4.78, 7.07 and 19.83, degrees two-theta with the margin of error of about ±0.5; about ±0.4; about ±0.3; about ±0.2; about ±0.1; about ±0.05; or less. In embodiments, crystalline Form B* of Compound I maleate salt exhibits an XRPD pattern comprising peaks at about 4.78, 7.07, 12.27, 19.83, and 20.84 degrees two-theta with the margin of error of about ±0.5; about ±0.4; about ±0.3; about ±0.2; about ±0.1; about ±0.05; or less.

In embodiments, crystalline Form B* of Compound I maleate salt exhibits an XRPD pattern comprising peaks at about 4.78, 7.07, 12.27, 19.83, 20.84, 20.98, and 24.35 degrees two-theta with the margin of error of about ±0.5; about ±0.4; about ±0.3; about ±0.2; about ±0.1; about ±0.05; or less. In embodiments, crystalline Form B* of Compound I maleate salt exhibits an XRPD pattern which further comprises at least two peaks selected from about 18.25, 18.45, 22.88, 23.82 and 23.84 degrees two-theta with the margin of error of about ±0.5; about ±0.4; about ±0.3; about ±0.2; about ±0.1; about ±0.05; or less. In embodiments, crystalline Form B* of Compound I maleate salt exhibits an XRPD pattern which further comprises at least three peaks selected from about 18.25, 18.45, 22.88, 23.82 and 23.84 degrees two-theta with the margin of error of about ±0.5; about ±0.4; about ±0.3; about ±0.2; about ±0.1; about ±0.05; or less. In embodiments, crystalline Form B* of Compound I maleate salt exhibits an XRPD pattern which further comprises at least four peaks selected from about 18.25, 18.45, 22.88, 23.82 and 23.84 degrees two-theta with the margin of error of about ±0.5; about ±0.4; about ±0.3; about ±0.2; about ±0.1; about ±0.05; or less. In embodiments, crystalline Form B* of Compound I maleate salt exhibits an XRPD pattern which further comprises peaks at about 18.25, 18.45, 22.88, 23.82 and 23.84 degrees two-theta with the margin of error of about ±0.5; about ±0.4; about ±0.3; about ±0.2; about ±0.1; about ±0.05; or less. In embodiments, crystalline Form B* of Compound I maleate salt exhibits an XRPD pattern comprising peaks at about 4.78, 7.07, 12.27, 18.25, 19.83, 20.84, 20.98, 22.88, and 24.35 degrees two-theta with the margin of error of about ±0.5; about ±0.4; about ±0.3; about ±0.2; about ±0.1; about ±0.05; or less.

In embodiments, crystalline Form B* of Compound I maleate salt exhibits an XRPD pattern comprising peaks at

In embodiments, crystalline Form B* of Compound I maleate salt exhibits an XRPD pattern comprising peaks at about 4.78±0.2, 7.07±0.2 and 19.83±0.2 degrees two-theta.

In embodiments, crystalline Form B* of Compound I maleate salt exhibits an XRPD pattern comprising peaks at about 4.78±0.2, 7.07±0.2, 12.27±0.2, 19.83±0.2, and 20.84±0.2 degrees two-theta.

In embodiments, crystalline Form B* of Compound I maleate salt exhibits an XRPD pattern comprising peaks at about 4.78±0.2, 7.07±0.2, 12.27±0.2, 19.83±0.2, 20.84±0.2, 20.98±0.2, and 24.35±0.2 degrees two-theta.

In embodiments, crystalline Form B* of Compound I maleate salt exhibits an XRPD pattern which further comprises at least two peaks selected from about 18.25±0.2, 18.45±0.2, 22.88±0.2, 23.82±0.2 and 23.84±0.2 degrees two-theta.

In embodiments, crystalline Form B* of Compound I maleate salt exhibits an XRPD pattern comprising peaks at about 4.78±0.2, 7.07±0.2, 12.27±0.2, 18.25±0.2, 19.83±0.2, 20.84±0.2, 20.98±0.2, 22.88±0.2, and 24.35±0.2 degrees two-theta.

In some embodiments, the crystalline form of Compound I maleate salt exhibits an XRPD pattern comprising 1 peak, 2 peaks, 3 peaks, 4 peaks, 5 peaks, 6 peaks, 7 peaks, 8 peaks, 9 peaks, or 10 peaks ±0.2 degrees two-theta of Table 4.

In embodiments, crystalline Form B* of Compound I maleate salt exhibits an XRPD pattern comprising peaks shown in Table 4, below.

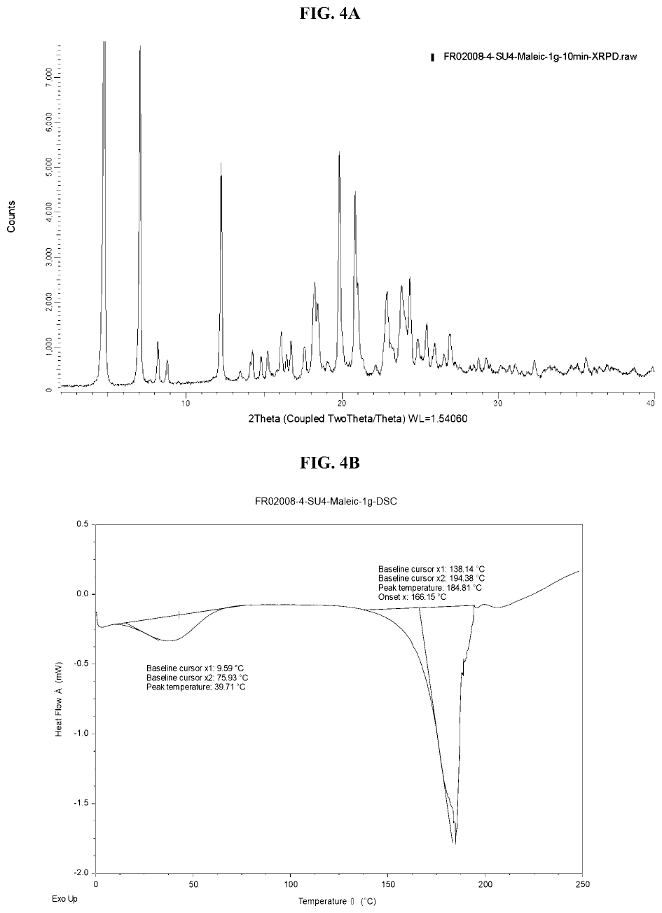

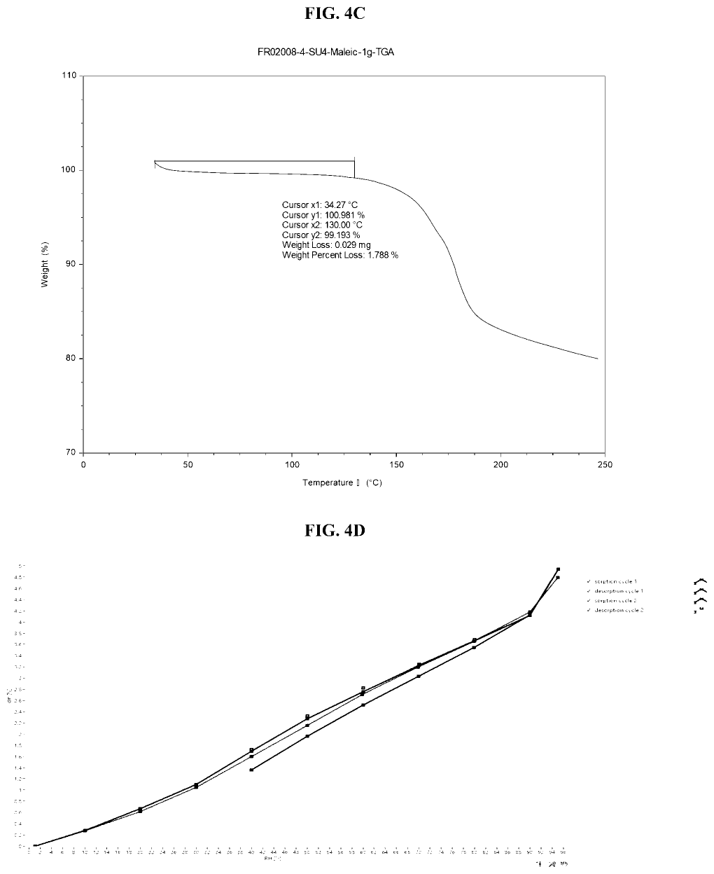

In embodiments, crystalline Form B* of Compound I maleate salt exhibits an XRPD pattern that is substantially similar to FIG. 4A.

TABLE 4
Form B* of Compound I maleate salt

| | d | Net | Rel. |
|---|---|---|---|
| Angle | Value | Intensity | Intensity |
| 4.245° | 20.80051 Å | 49.5619 | 0.7% |
| 4.778° | 18.48083 Å | 7578.79 | 100.0% |
| 7.072° | 12.48931 Å | 4892.12 | 64.6% |
| 7.713° | 11.45354 Å | 42.1818 | 0.6% |
| 8.212° | 10.75794 Å | 611.097 | 8.1% |
| 8.808° | 10.03193 Å | 339.199 | 4.5% |
| 9.494° | 9.30769 Å | 47.9951 | 0.6% |
| 11.752° | 7.52449 Å | 56.8419 | 0.8% |
| 12.269° | 7.20855 Å | 3278.49 | 43.3% |
| 13.499° | 6.55391 Å | 142.295 | 1.9% |
| 14.142° | 6.25745 Å | 268.312 | 3.5% |
| 14.273° | 6.20029 Å | 446.108 | 5.9% |
| 14.814° | 5.97523 Å | 355.057 | 4.7% |
| 15.247° | 5.80638 Å | 419.869 | 5.5% |
| 15.863° | 5.58239 Å | 126.350 | 1.7% |
| 16.112° | 5.49656 Å | 695.760 | 9.2% |
| 16.446° | 5.38580 Å | 349.976 | 4.6% |
| 16.731° | 5.29474 Å | 571.078 | 7.5% |
| 17.593° | 5.03717 Å | 455.235 | 6.0% |
| 18.251° | 4.85691 Å | 1386.55 | 18.3% |
| 18.452° | 4.80451 Å | 1109.79 | 14.6% |
| 18.802° | 4.71589 Å | 123.123 | 1.6% |
| 19.046° | 4.65598 Å | 201.980 | 2.7% |
| 19.830° | 4.47364 Å | 3500.61 | 46.2% |
| 20.837° | 4.25973 Å | 2953.76 | 39.0% |
| 20.982° | 4.23057 Å | 1432.76 | 18.9% |
| 21.276° | 4.17267 Å | 233.210 | 3.1% |
| 22.133° | 4.01303 Å | 132.529 | 1.7% |
| 22.883° | 3.88325 Å | 1227.98 | 16.2% |
| 23.271° | 3.81934 Å | 355.189 | 4.7% |
| 23.816° | 3.73308 Å | 1190.37 | 15.7% |
| 23.839° | 3.72953 Å | 1198.78 | 15.8% |
| 24.351° | 3.65238 Å | 1425.85 | 18.8% |
| 24.865° | 3.57798 Å | 414.301 | 5.5% |
| 25.420° | 3.50114 Å | 721.026 | 9.5% |
| 25.933° | 3.43296 Å | 380.291 | 5.0% |
| 26.529° | 3.35724 Å | 252.834 | 3.3% |
| 26.924° | 3.30887 Å | 481.484 | 6.4% |
| 27.217° | 3.27389 Å | 140.898 | 1.9% |
| 27.519° | 3.23869 Å | 124.124 | 1.6% |
| 28.184° | 3.16370 Å | 92.6323 | 1.2% |
| 28.447° | 3.13505 Å | 121.911 | 1.6% |
| 28.752° | 3.10253 Å | 216.740 | 2.9% |
| 29.225° | 3.05332 Å | 223.713 | 3.0% |
| 29.489° | 3.02664 Å | 131.070 | 1.7% |
| 30.155° | 2.96126 Å | 125.645 | 1.7% |
| 30.377° | 2.94014 Å | 56.1916 | 0.7% |
| 30.727° | 2.90745 Å | 129.316 | 1.7% |
| 31.102° | 2.87319 Å | 146.438 | 1.9% |
| 31.491° | 2.83862 Å | 82.5026 | 1.1% |
| 31.921° | 2.80131 Å | 48.5434 | 0.6% |
| 32.297° | 2.76959 Å | 272.241 | 3.6% |
| 32.924° | 2.71825 Å | 79.3428 | 1.0% |
| 33.272° | 2.69066 Å | 133.110 | 1.8% |
| 33.610° | 2.66436 Å | 111.949 | 1.5% |
| 34.688° | 2.58398 Å | 101.346 | 1.3% |
| 35.074° | 2.55638 Å | 179.056 | 2.4% |
| 35.608° | 2.51930 Å | 259.155 | 3.4% |
| 36.500° | 2.45972 Å | 100.752 | 1.3% |
| 36.963° | 2.42998 Å | 110.351 | 1.5% |
| 37.254° | 2.41167 Å | 116.987 | 1.5% |
| 38.700° | 2.32483 Å | 100.723 | 1.3% |
| 39.645° | 2.27158 Å | 40.6961 | 0.5% |
| 39.882° | 2.25857 Å | 101.521 | 1.3% |

In embodiments, crystalline Form B* of Compound I maleate salt exhibits a DSC thermogram comprising an endotherm peak at about 10° C. with the error of margin of about ±2.5; about ±2.0; about ±1.5; about ±1.0; about ±0.5; or less. In embodiments, the crystalline Form B* of Compound I maleate salt exhibits a DSC thermogram comprising an endotherm peak at about 10° C., which is likely due to dehydration. In embodiments, crystalline Form B* of Compound I maleate salt exhibits a DSC thermogram comprising an endotherm peak with an onset at about 166° C. with the error of margin of about ±2.5; about ±2.0; about ±1.5; about ±1.0; about ±0.5; or less. In embodiments, crystalline Form B* of Compound I maleate salt exhibits a DSC thermogram comprising an endotherm peak with an onset at about 166° C., which is likely due to decomposition. In embodiments, crystalline Form B* of Compound I maleate salt exhibits a DSC thermogram comprising an endotherm peak at about 185° C. with the error of margin of about ±2.5; about ±2.0; about ±1.5; about ±1.0; about ±0.5; or less.

In some embodiments, the crystalline Form B* of Compound I maleate salt exhibits a DSC thermogram that is substantially similar to FIG. 4B.

In some embodiments, the crystalline Form B* of Compound I maleate salt exhibits a TGA thermogram substantially similar to FIG. 4C. In embodiments, the c Form B* of Compound I maleate salt exhibits a weight percent loss of about 1.8% between about 34° C. to about 130° C. by a thermogravimetric analysis (TGA).

### Compound I Maleate Salt Form A-6

In embodiments, the present disclosure relates to a crystalline form of Compound I maleate salt, which is Form A-6.

In embodiment, the crystalline form of Compound I maleate salt may comprise of over about 99.9%, about 99.8%, about 99.7%, about 99.6%, about 99.5%, about 99.4%, about 99.3%, about 99.2%, about 99.1%, or about 99.0% of Form A-6 of Compound I maleate salt. In another embodiment, the crystalline form of Compound I maleate salt may comprise over about 99%, 98%, 97%, 96%, 95%, 94%, 93%, 92%, 91%, or 90% of Form A-6 of Compound I maleate salt. In some embodiments, the crystalline form of Compound I maleate salt may comprise over about 90%, 85%, 80%, 75%, 70%, 65%, 60%, 55%, 50%, 45%, or 40% of Form A-6 of Compound I maleate salt

In embodiments, crystalline Form A-6 of Compound I maleate salt exhibits an XRPD pattern comprising peaks at about 3.99, 23.84, and 25.40 degrees two-theta with the margin of error of about ±0.5; about ±0.4; about ±0.3; about ±0.2; about ±0.1; about ±0.05; or less. In embodiments, crystalline Form A-6 of Compound I maleate salt exhibits an XRPD pattern comprising peaks at about 3.99, 23.70, 23.74, 23.84, and 25.40 degrees two-theta with the margin of error of about ±0.5; about ±0.4; about ±0.3; about ±0.2; about ±0.1; about ±0.05; or less. In embodiments, crystalline Form A-6 of Compound I maleate salt exhibits an XRPD pattern which further comprises at least two peaks selected from about 11.84, 17.48, 18.85, 19.59, 19.97, 22.75, 24.86, and 25.97 degrees two-theta with the margin of error of about ±0.5; about ±0.4; about ±0.3; about ±0.2; about ±0.1; about ±0.05; or less. In embodiments, crystalline Form A-6 of Compound I maleate salt exhibits an XRPD pattern which further comprises at least three peaks selected from about 11.84, 17.48, 18.85, 19.59, 19.97, 22.75, 24.86, and 25.97 degrees two-theta with the margin of error of about ±0.5; about ±0.4; about ±0.3; about ±0.2; about ±0.1; about ±0.05; or less. In embodiments, crystalline Form A-6 of Compound I maleate salt exhibits an XRPD pattern which further comprises at least four peaks selected from about 11.84, 17.48, 18.85, 19.59, 19.97, 22.75, 24.86, and 25.97 degrees two-theta with the margin of error of about ±0.5; about ±0.4; about ±0.3; about ±0.2; about ±0.1; about ±0.05; or less. In embodiments, crystalline Form A-6 of Compound I maleate salt exhibits an XRPD pattern which further comprises at least five peaks selected from about 11.84, 17.48, 18.85, 19.59, 19.97, 22.75, 24.86, and 25.97 degrees two-theta with the margin of error of about ±0.5; about ±0.4; about ±0.3; about ±0.2; about ±0.1; about ±0.05; or less. In embodiments, crystalline Form A-6 of Compound I maleate salt exhibits an XRPD pattern which further comprises at least six peaks selected from about 11.84, 17.48, 18.85, 19.59, 19.97, 22.75, 24.86, and 25.97 degrees two-theta with the margin of error of about ±0.5; about ±0.4; about ±0.3; about ±0.2; about ±0.1; about ±0.05; or less. In embodiments, crystalline Form A-6 of Compound I maleate salt exhibits an XRPD pattern which further comprises at least seven peaks selected from about 11.84, 17.48, 18.85, 19.59, 19.97, 22.75, 24.86, and 25.97 degrees two-theta with the margin of error of about ±0.5; about ±0.4; about ±0.3; about ±0.2; about ±0.1; about ±0.05; or less. In embodiments, crystalline Form A-6 of Compound I maleate salt exhibits an XRPD pattern which further comprises peaks at about 11.84, 17.48, 18.85, 19.59, 19.97, 22.75, 24.86, and 25.97 degrees two-theta with the margin of error of about ±0.5; about ±0.4; about ±0.3; about ±0.2; about ±0.1; about ±0.05; or less.

In embodiments, crystalline Form A-6 of Compound I maleate salt exhibits an XRPD pattern comprising peaks at about 3.99±0.2, 23.84-0.2, and 25.40±0.2 degrees two-theta

In embodiments, crystalline Form A-6 of Compound I maleate salt exhibits an XRPD pattern comprising peaks at about 3.99±0.2, 23.70±0.2, 23.74±0.2, 23.84±0.2, and 25.40±0.2 degrees two-theta.

In embodiments, crystalline Form A-6 of Compound I maleate salt exhibits an X-ray powder diffraction (XRPD) pattern further comprising at least two peaks selected from about 11.84±0.2, 17.48±0.2, 18.85±0.2, 19.59±0.2, 19.97±0.2, 22.75±0.2, 24.86±0.2, and 25.97±0.2 degrees two-theta.

In some embodiments, the crystalline form of Compound I maleate salt exhibits an XRPD pattern comprising 1 peak, 2 peaks, 3 peaks, 4 peaks, 5 peaks, 6 peaks, 7 peaks, 8 peaks, 9 peaks, or 10 peaks ±0.2 degrees two-theta of Table 14.

In embodiments, crystalline Form A-6 of Compound I maleate salt exhibits an XRPD pattern comprising peaks shown in Table 14, below.

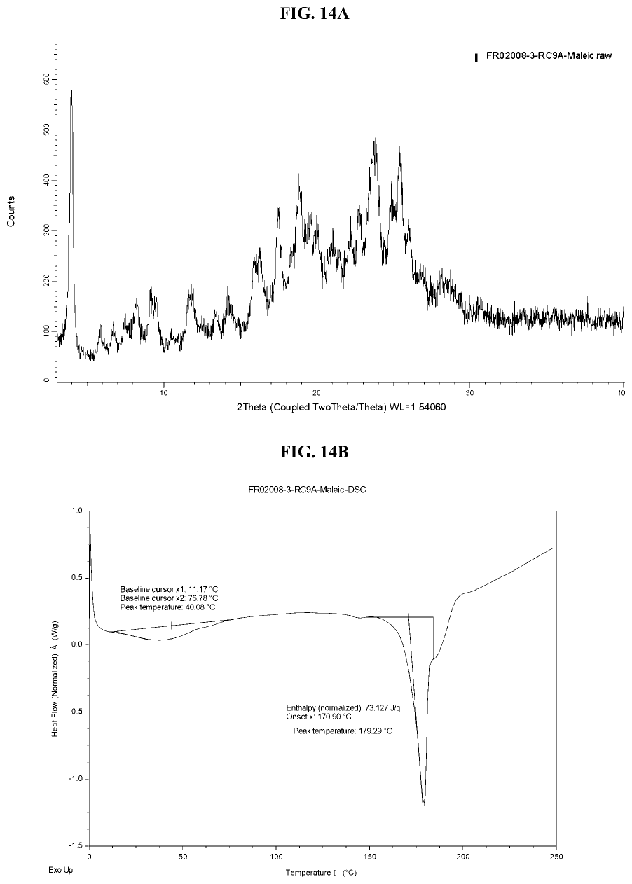

In embodiments, crystalline Form A-6 of Compound I maleate salt exhibits an XRPD pattern that is substantially similar to FIG. 14A.

TABLE 14
Form A-6 of Compound I maleate salt

| Angle | d Value | | Net Intensity | Rel. Intensity |
|---|---|---|---|---|
| 3.991° | 22.11968 | Å | 301.202 | 100.0% |
| 5.874° | 15.03396 | Å | 34.9471 | 11.6% |
| 6.712° | 13.15926 | Å | 31.9746 | 10.6% |
| 7.452° | 11.85417 | Å | 40.6166 | 13.5% |
| 8.251° | 10.70753 | Å | 65.6459 | 21.8% |
| 9.162° | 9.64456 | Å | 66.5859 | 22.1% |
| 9.541° | 9.26207 | Å | 58.4419 | 19.4% |
| 11.684° | 7.56763 | Å | 52.7770 | 17.5% |
| 11.837° | 7.47049 | Å | 84.8767 | 28.2% |
| 13.396° | 6.60428 | Å | 21.3450 | 7.1% |
| 14.196° | 6.23383 | Å | 55.8155 | 18.5% |
| 14.851° | 5.96053 | Å | 18.2990 | 6.1% |
| 15.969° | 5.54541 | Å | 71.4213 | 23.7% |
| 16.276° | 5.44154 | Å | 55.6673 | 18.5% |
| 17.484° | 5.06825 | Å | 122.931 | 40.8% |
| 18.369° | 4.82596 | Å | 41.4756 | 13.8% |
| 18.849° | 4.70421 | Å | 142.195 | 47.2% |
| 19.586° | 4.52888 | Å | 85.9061 | 28.5% |
| 19.971° | 4.44232 | Å | 92.2553 | 30.6% |
| 22.220° | 3.99763 | Å | 45.8373 | 15.2% |
| 22.753° | 3.90502 | Å | 90.9945 | 30.2% |
| 23.737° | 3.74537 | Å | 184.536 | 61.3% |
| 23.695° | 3.75191 | Å | 162.111 | 53.8% |
| 23.843° | 3.72892 | Å | 194.395 | 64.5% |
| 24.864° | 3.57812 | Å | 145.439 | 48.3% |
| 25.399° | 3.50393 | Å | 186.752 | 62.0% |
| 25.972° | 3.42799 | Å | 93.0550 | 30.9% |
| 28.087° | 3.17440 | Å | 31.7610 | 10.5% |

In embodiments, crystalline Form A-6 of Compound I maleate salt exhibits a DSC thermogram comprising an endotherm peak at about 40° C. with the error of margin of about ±2.5; about ±2.0; about ±1.5; about ±1.0; about ±0.5; or less. In embodiments, the crystalline Form A-6 of Compound I maleate salt exhibits a DSC thermogram comprising an endotherm peak with an onset at about 171° C. with the error of margin of about ±2.5; about ±2.0; about ±1.5; about ±1.0; about ±0.5; or less. In embodiments, the crystalline Form A-6 of Compound I maleate salt exhibits a DSC thermogram comprising an endotherm peak at about 179° C. with the error of margin of about ±2.5; about ±2.0; about ±1.5; about ±1.0; about ±0.5; or less.

In some embodiments, the crystalline Form A-6 of Compound I maleate salt exhibits a DSC thermogram that is substantially similar to FIG. 14B.

### Compound I Hydrochloride Salt

In one embodiment, the present disclosure relates to a Compound I hydrochloride salt, or a solvate thereof. In embodiments, the crystalline form of Compound I hydrochloride salt comprises a mixture of one or more forms of polymorphs of Compound I hydrochloride salt. In embodiments, the crystalline form of Compound I hydrochloride salt comprises of substantially pure form of one polymorph type.

### Compound I Hydrochloride Salt Form A-1

In embodiments, the present disclosure relates to a crystalline form of Compound I hydrochloride salt, which is Form A-1.

In embodiment, the crystalline form of Compound I hydrochloride salt may comprise of over about 99.9%, about 99.8%, about 99.7%, about 99.6%, about 99.5%, about 99.4%, about 99.3%, about 99.2%, about 99.1%, or about 99.0% of Form A-1 of Compound I hydrochloride salt. In another embodiment, the crystalline form of Compound I hydrochloride salt may comprise over about 99%, 98%, 97%, 96%, 95%, 94%, 93%, 92%, 91%, or 90% of Form A-1 of Compound I hydrochloride salt. In some embodiments, the crystalline form of Compound I hydrochloride salt may comprise over about 90%, 85%, 80%, 75%, 70%, 65%, 60%, 55%, 50%, 45%, or 40% of Form A-1 of Compound I hydrochloride salt.

In embodiments, crystalline Form A-1 of Compound I hydrochloride salt exhibits an XRPD pattern comprising peaks at about 4.83, 7.14 and 9.2 degrees two-theta with the margin of error of about ±0.5; about ±0.4; about ±0.3; about ±0.2; about ±0.1; about ±0.05; or less. In embodiments, crystalline Form A-1 of Compound I hydrochloride salt exhibits an XRPD pattern comprising peaks at about 4.83, 5.38, 7.14, 9.20, and 22.78 degrees two-theta with the margin of error of about ±0.5; about ±0.4; about ±0.3; about ±0.2; about ±0.1; about ±0.05; or less. In embodiments, crystalline Form A-1 of Compound I hydrochloride salt exhibits an XRPD pattern which further comprises at least two peaks selected from about 15.03±0.2, 20.32±0.2, 21.12±0.2, 22.45±0.2, 23.57±0.2, 24.66±0.2, and 27.45±0.2 degrees two-theta with the margin of error of about ±0.5; about ±0.4; about ±0.3; about ±0.2; about ±0.1; about ±0.05; or less. In embodiments, crystalline Form A-1 of Compound I hydrochloride salt exhibits an XRPD pattern which further comprises at least three peaks selected from about 15.03±0.2, 20.32±0.2, 21.12±0.2, 22.45±0.2, 23.57±0.2, 24.66±0.2, and 27.45±0.2 degrees two-theta with the margin of error of about ±0.5; about ±0.4; about ±0.3; about ±0.2; about ±0.1; about ±0.05; or less. In embodiments, crystalline Form A-1 of Compound I hydrochloride salt exhibits an XRPD pattern which further comprises at least four peaks selected from about 15.03±0.2, 20.32±0.2, 21.12±0.2, 22.45±0.2, 23.57±0.2, 24.66±0.2, and 27.45±0.2 degrees two-theta with the margin of error of about ±0.5; about ±0.4; about ±0.3; about ±0.2; about ±0.1; about ±0.05; or less. In embodiments, crystalline Form A-1 of Compound I hydrochloride salt exhibits an XRPD pattern which further comprises at least five peaks selected from about 15.03±0.2, 20.32±0.2, 21.12±0.2, 22.45±0.2, 23.57±0.2, 24.66±0.2, and 27.45±0.2 degrees two-theta with the margin of error of about ±0.5; about ±0.4; about ±0.3; about ±0.2; about ±0.1; about ±0.05; or less. In embodiments, crystalline Form A-1 of Compound I hydrochloride salt exhibits an XRPD pattern which further comprises at least six peaks selected from about 15.03±0.2, 20.32±0.2, 21.12±0.2, 22.45±0.2, 23.57±0.2, 24.66±0.2, and 27.45±0.2 degrees two-theta with the margin of error of about ±0.5; about ±0.4; about ±0.3; about ±0.2; about ±0.1; about ±0.05; or less. In embodiments, crystalline Form A-1 of Compound I hydrochloride salt exhibits an XRPD pattern which further comprises peaks at about 15.03±0.2, 20.32±0.2, 21.12±0.2, 22.45±0.2, 23.57±0.2, 24.66±0.2, and 27.45±0.2 degrees two-theta with the margin of error of about ±0.5; about ±0.4; about ±0.3; about ±0.2; about ±0.1; about ±0.05; or less.

In embodiments, crystalline Form A-1 of Compound I hydrochloride salt exhibits an XRPD pattern comprising peaks at about 4.83±0.2, 7.14±0.2 and 9.20±0.2 degrees two-theta.

In embodiments, crystalline Form A-1 of Compound I hydrochloride salt exhibits an XRPD pattern comprising peaks at about 4.83±0.2, 5.38±0.2, 7.14±0.2, 9.20±0.2, and 22.78±0.2

In embodiments, crystalline Form A-1 of Compound I hydrochloride salt exhibits an XRPD pattern which further comprises at least two peaks selected from about 15.03±0.2, 20.32±0.2, 21.12±0.2, 22.45±0.2, 23.57±0.2, 24.66±0.2, and 27.45±0.2 degrees two-theta.

In some embodiments, the crystalline form of Compound I hydrochloride salt exhibits an XRPD pattern comprising 1 peak, 2 peaks, 3 peaks, 4 peaks, 5 peaks, 6 peaks, 7 peaks, 8 peaks, 9 peaks, or 10 peaks ±0.2 degrees two-theta of Table 5.

In embodiments, crystalline Form A-1 of Compound I hydrochloride salt an XRPD pattern comprising peaks shown in Table 5, below.

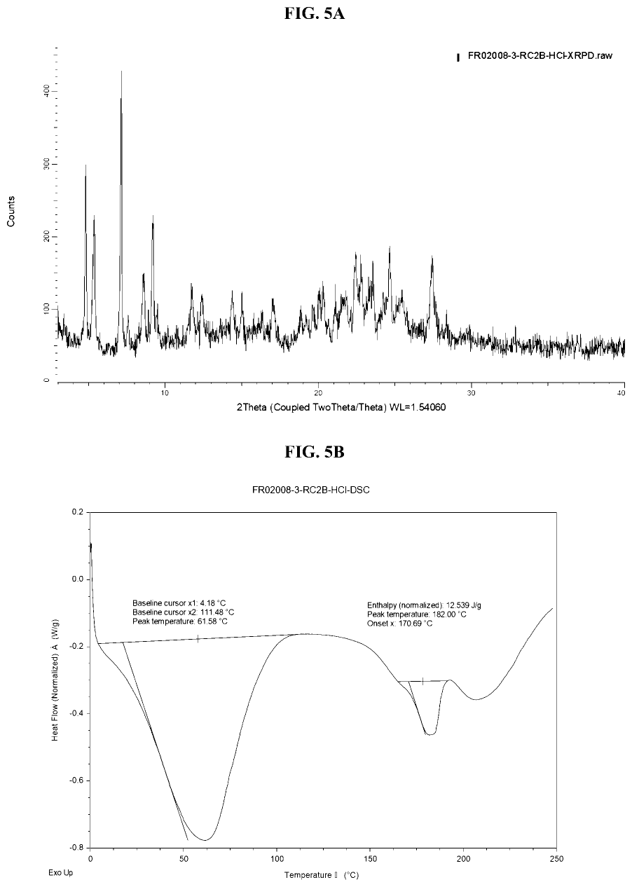

In embodiments, crystalline Form A-1 of Compound I hydrochloride salt an XRPD pattern that is substantially similar to FIG. 5A.

TABLE 5
Form A-1 of Compound I hydrochloride salt

| Angle | d Value | | Net Intensity | Rel. Intensity |
|---|---|---|---|---|
| 4.829° | 18.28287 | Å | 147.042 | 58.6% |
| 5.377° | 16.42122 | Å | 94.3328 | 37.6% |
| 7.139° | 12.37203 | Å | 251.130 | 100.0% |
| 7.588° | 11.64154 | Å | 26.0841 | 10.4% |
| 8.295° | 10.65057 | Å | 26.0380 | 10.4% |
| 8.596° | 10.27884 | Å | 51.1793 | 20.4% |
| 8.921° | 9.90482 | Å | 25.7473 | 10.3% |
| 9.198° | 9.60727 | Å | 102.606 | 40.9% |
| 9.463° | 9.33863 | Å | 22.6961 | 9.0% |
| 10.185° | 8.67780 | Å | 13.9846 | 5.6% |
| 11.542° | 7.66039 | Å | 15.6705 | 6.2% |
| 11.750° | 7.52583 | Å | 34.6740 | 13.8% |
| 12.414° | 7.12462 | Å | 36.4821 | 14.5% |
| 13.595° | 6.50826 | Å | 25.1475 | 10.0% |
| 14.388° | 6.15104 | Å | 42.4346 | 16.9% |
| 15.027° | 5.89115 | Å | 48.2592 | 19.2% |
| 17.042° | 5.19862 | Å | 33.9670 | 13.5% |
| 17.396° | 5.09370 | Å | 14.6679 | 5.8% |
| 18.869° | 4.69915 | Å | 29.7123 | 11.8% |
| 19.660° | 4.51189 | Å | 26.2829 | 10.5% |
| 20.061° | 4.42269 | Å | 36.7904 | 14.6% |
| 20.318° | 4.36717 | Å | 47.8925 | 19.1% |
| 20.649° | 4.29808 | Å | 18.8861 | 7.5% |
| 21.120° | 4.20327 | Å | 56.5909 | 22.5% |
| 21.816° | 4.07066 | Å | 38.5157 | 15.3% |
| 22.450° | 3.95713 | Å | 67.7554 | 27.0% |
| 22.783° | 3.89997 | Å | 70.5562 | 28.1% |
| 23.385° | 3.80097 | Å | 49.6445 | 19.8% |
| 23.574° | 3.77089 | Å | 65.2396 | 26.0% |
| 24.248° | 3.66762 | Å | 40.4197 | 16.1% |
| 24.661° | 3.60714 | Å | 56.0820 | 22.3% |
| 25.445° | 3.49774 | Å | 27.0321 | 10.8% |
| 27.446° | 3.24704 | Å | 79.3609 | 31.6% |
| 28.378° | 3.14257 | Å | 35.4266 | 14.1% |
| 37.131° | 2.41935 | Å | 23.3905 | 9.3% |

In embodiments, crystalline Form A-1 of Compound I hydrochloride salt exhibits a DSC thermogram comprising an endotherm peak at about 4.2° C. with the error of margin of about ±2.5; about ±2.0; about ±1.5; about ±1.0; about ±0.5; or less. In embodiments, the crystalline Form A-1 of Compound I hydrochloride salt exhibits a DSC thermogram comprising an endotherm peak with an onset at about 171° C. with the error of margin of about ±2.5; about ±2.0; about ±1.5; about ±1.0; about ±0.5; or less. In embodiments, the crystalline Form A-1 of Compound I hydrochloride salt exhibits a DSC thermogram comprising an endotherm peak at about 182° C. with the error of margin of about ±2.5; about ±2.0; about ±1.5; about ±1.0; about ±0.5; or less.

In some embodiments, the crystalline Form A-1 of Compound I hydrochloride salt exhibits a DSC thermogram that is substantially similar to FIG. 5B.

### Compound I Hydrochloride Salt Form B-1

In embodiments, the present disclosure relates to a crystalline form of Compound I hydrochloride salt, which is Form B-1.

In embodiment, the crystalline form of Compound I hydrochloride salt may comprise of over about 99.9%, about 99.8%, about 99.7%, about 99.6%, about 99.5%, about 99.4%, about 99.3%, about 99.2%, about 99.1%, or about 99.0% of Form B-1 of Compound I hydrochloride salt. In another embodiment, the crystalline form of Compound I hydrochloride salt may comprise over about 99%, 98%, 97%, 96%, 95%, 94%, 93%, 92%, 91%, or 90% of Form B-1 of Compound I hydrochloride salt. In some embodiments, the crystalline form of Compound I hydrochloride salt may comprise over about 90%, 85%, 80%, 75%, 70%, 65%, 60%, 55%, 50%, 45%, or 40% of Form B-1 of Compound I hydrochloride salt.

In embodiments, crystalline Form B-1 of Compound I hydrochloride salt exhibits an XRPD pattern comprising peaks at about 7.40, 23.26, and 24.21 degrees two-theta with the margin of error of about ±0.5; about ±0.4; about ±0.3; about ±0.2; about ±0.1; about ±0.05; or less. In embodiments, crystalline Form B-1 of Compound I hydrochloride salt exhibits an XRPD pattern comprising peaks at about 4.48, 7.40, 7.79, 23.26 and 24.21 degrees two-theta with the margin of error of about ±0.5; about ±0.4; about ±0.3; about ±0.2; about ±0.1; about ±0.05; or less. In embodiments, crystalline Form B-1 of Compound I hydrochloride salt exhibits an XRPD pattern which further comprises at least two peaks selected from about 9.73, 10.12, 12.93, 13.96, 16.08, 18.93, 20.79, and 22.33 degrees two-theta with the margin of error of about ±0.5; about ±0.4; about ±0.3; about ±0.2; about ±0.1; about ±0.05; or less. In embodiments, crystalline Form B-1 of Compound I hydrochloride salt exhibits an XRPD pattern further comprises at least three peaks selected from about 9.73, 10.12, 12.93, 13.96, 16.08, 18.93, 20.79, and 22.33 degrees two-theta with the margin of error of about ±0.5; about ±0.4; about ±0.3; about ±0.2; about ±0.1; about ±0.05; or less. In embodiments, crystalline Form B-1 of Compound I hydrochloride salt exhibits an XRPD pattern further comprises at least four peaks selected from about 9.73, 10.12, 12.93, 13.96, 16.08, 18.93, 20.79, and 22.33 degrees two-theta with the margin of error of about ±0.5; about ±0.4; about ±0.3; about ±0.2; about ±0.1; about ±0.05; or less. In embodiments, crystalline Form B-1 of Compound I hydrochloride salt exhibits an XRPD pattern further comprises at least five peaks selected from about 9.73, 10.12, 12.93, 13.96, 16.08, 18.93, 20.79, and 22.33 degrees two-theta with the margin of error of about ±0.5; about ±0.4; about ±0.3; about ±0.2; about ±0.1; about ±0.05; or less. In embodiments, crystalline Form B-1 of Compound I hydrochloride salt exhibits an XRPD pattern further comprises at least six peaks selected from about 9.73, 10.12, 12.93, 13.96, 16.08, 18.93, 20.79, and 22.33 degrees two-theta with the margin of error of about ±0.5; about ±0.4; about ±0.3; about ±0.2; about ±0.1; about ±0.05; or less. In embodiments, crystalline Form B-1 of Compound I hydrochloride salt exhibits an XRPD pattern further comprises at least seven peaks selected from about 9.73, 10.12, 12.93, 13.96, 16.08, 18.93, 20.79, and 22.33 degrees two-theta with the margin of error of about ±0.5; about ±0.4; about ±0.3; about ±0.2; about ±0.1; about ±0.05; or less. In embodiments, crystalline Form B-1 of Compound I hydrochloride salt exhibits an XRPD pattern further comprises peaks selected from about 9.73, 10.12, 12.93, 13.96, 16.08, 18.93, 20.79, and 22.33 degrees two-theta with the margin of error of about ±0.5; about ±0.4; about ±0.3; about ±0.2; about ±0.1; about ±0.05; or less.

In embodiments, crystalline Form B-1 of Compound I hydrochloride salt exhibits an XRPD pattern comprising peaks at about 7.40±0.2, 23.26±0.2 and 24.21±0.2 degrees two-theta.

In embodiments, crystalline Form B-1 of Compound I hydrochloride salt exhibits an XRPD pattern comprising peaks at about 4.48-0.2, 7.40±0.2, 7.79±0.2, 23.26-0.2 and 24.21±0.2 degrees two-theta.

In embodiments, crystalline Form B-1 of Compound I hydrochloride salt exhibits an XRPD pattern which further comprises at least two peaks selected from about 9.73±0.2, 10.12±0.2, 12.93±0.2, 13.96±0.2, 16.08±0.2, 18.93±0.2, 20.79±0.2, and 22.33±0.2 degrees two-theta.

In some embodiments, the crystalline form of Compound I hydrochloride salt exhibits an XRPD pattern comprising 1 peak, 2 peaks, 3 peaks, 4 peaks, 5 peaks, 6 peaks, 7 peaks, 8 peaks, 9 peaks, or 10 peaks ±0.2 degrees two-theta of Table 6.

In embodiments, crystalline Form B-1 of Compound I hydrochloride salt an XRPD pattern comprising peaks shown in Table 6, below.

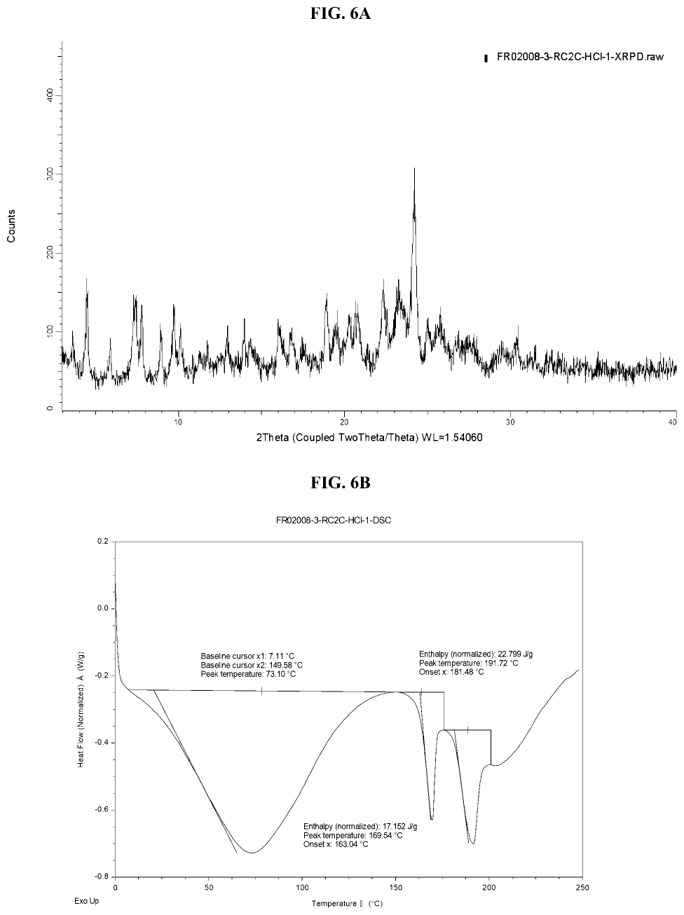

In embodiments, crystalline Form B-1 of Compound I hydrochloride salt an XRPD pattern that is substantially similar to FIG. 6A.

TABLE 6
Form B-1 of Compound I hydrochloride salt

| Angle | d Value | | Net Intensity | Rel. Intensity |
|---|---|---|---|---|
| 3.621° | 24.38084 | Å | 21.3348 | 12.2% |
| 4.482° | 19.69726 | Å | 58.3215 | 33.4% |
| 5.846° | 15.10529 | Å | 18.2784 | 10.5% |
| 7.399° | 11.93786 | Å | 67.1692 | 38.5% |
| 7.790° | 11.34045 | Å | 64.4153 | 36.9% |
| 8.946° | 9.87660 | Å | 30.7737 | 17.6% |
| 9.733° | 9.07964 | Å | 47.8500 | 27.4% |
| 10.123° | 8.73119 | Å | 43.1906 | 24.7% |
| 12.932° | 6.84010 | Å | 36.0207 | 20.6% |
| 13.959° | 6.33937 | Å | 49.3384 | 28.3% |
| 14.329° | 6.17633 | Å | 29.3393 | 16.8% |
| 16.079° | 5.50797 | Å | 41.4735 | 23.8% |
| 16.801° | 5.27265 | Å | 16.4943 | 9.5% |
| 18.928° | 4.68476 | Å | 50.7367 | 29.1% |
| 19.517° | 4.54474 | Å | 25.9046 | 14.8% |
| 20.297° | 4.37170 | Å | 31.7002 | 18.2% |
| 20.789° | 4.26937 | Å | 53.2726 | 30.5% |
| 22.330° | 3.97810 | Å | 56.3678 | 32.3% |
| 23.255° | 3.82199 | Å | 66.9458 | 38.4% |
| 24.209° | 3.67336 | Å | 174.538 | 100.0% |
| 25.047° | 3.55245 | Å | 30.7876 | 17.6% |
| 30.400° | 2.93792 | Å | 30.7297 | 17.6% |

In embodiments, crystalline Form B-1 of Compound I hydrochloride salt exhibits a DSC thermogram comprising an endotherm peak at about 73° C. with the error of margin of about ±2.5; about ±2.0; about ±1.5; about ±1.0; about ±0.5; or less. In embodiments, the crystalline Form B-1 of Compound I hydrochloride salt exhibits a DSC thermogram comprising an endotherm peak with an onset at about 170° C. with the error of margin of about ±2.5; about ±2.0; about ±1.5; about ±1.0; about ±0.5; or less. In embodiments, the crystalline Form B-1 of Compound I hydrochloride salt exhibits a DSC thermogram comprising an endotherm peak at about 163° C. with the error of margin of about ±2.5; about ±2.0; about ±1.5; about ±1.0; about ±0.5; or less. In embodiments, the crystalline Form B-1 of Compound I hydrochloride salt exhibits a DSC thermogram comprising an endotherm peak with an onset at about 182° C. with the error of margin of about ±2.5; about ±2.0; about ±1.5; about ±1.0; about ±0.5; or less. In embodiments, the crystalline Form B-1 of Compound I hydrochloride salt exhibits a DSC thermogram comprising an endotherm peak at about 192° C. with the error of margin of about ±2.5; about ±2.0; about ±1.5; about ±1.0; about ±0.5; or less.

In some embodiments, the crystalline Form B-1 of Compound I hydrochloride salt exhibits a DSC thermogram that is substantially similar to FIG. 6B.

### Compound I Sulfate Salt

In one embodiment, the present disclosure relates to a Compound I sulfate salt, or a solvate thereof. In embodiments, the crystalline form of Compound I sulfate salt comprises a mixture of one or more forms of polymorphs of Compound I sulfate salt. In embodiments, the crystalline form of Compound I sulfate salt comprises of substantially pure form of one polymorph type.

### Compound I Sulfate Salt Form A-2

In embodiments, the present disclosure relates to a crystalline form of Compound I sulfate salt, which is Form A-2.

In embodiments, the crystalline form of Compound I sulfate salt may comprise of over about 99.9%, about 99.8%, about 99.7%, about 99.6%, about 99.5%, about 99.4%, about 99.3%, about 99.2%, about 99.1%, or about 99.0% of Form A-2 of Compound I sulfate salt. In another embodiment, the crystalline form of Compound I sulfate salt may comprise over about 99%, 98%, 97%, 96%, 95%, 94%, 93%, 92%, 91%, or 90% of Form A-2 of Compound I sulfate salt. In some embodiments, the crystalline form of Compound I sulfate salt may comprise over about 90%, 85%, 80%, 75%, 70%, 65%, 60%, 55%, 50%, 45%, or 40% of Form A-2 of Compound I sulfate salt.

In embodiments, crystalline Form A-2 of Compound I sulfate salt exhibits an XRPD pattern comprising peaks at about 6.95, 9.52 and 9.82 degrees two-theta with the margin of error of about ±0.5; about ±0.4; about ±0.3; about ±0.2; about ±0.1; about ±0.05; or less. In embodiments, crystalline Form A-2 of Compound I sulfate salt exhibits an XRPD pattern comprising peaks at about 6.95, 9.52, 9.82, 12.92 and 19.46 degrees two-theta with the margin of error of about ±0.5; about ±0.4; about ±0.3; about ±0.2; about ±0.1; about ±0.05; or less. In embodiments, crystalline Form A-2 of Compound I sulfate salt exhibits an XRPD pattern which further comprises at least two peaks selected from about 13.77±0.2, 15.30±0.2, 25.63±0.2 degrees two-theta with the margin of error of about ±0.5; about ±0.4; about ±0.3; about ±0.2; about ±0.1; about ±0.05; or less. In embodiments, crystalline Form A-2 of Compound I sulfate salt exhibits an XRPD pattern which further comprises three peaks selected at about 13.77±0.2, 15.30±0.2, 25.63±0.2 degrees two-theta with the margin of error of about ±0.5; about ±0.4; about ±0.3; about ±0.2; about ±0.1; about ±0.05; or less.

In embodiments, crystalline Form A-2 of Compound I sulfate salt exhibits an X-ray powder diffraction (XRPD) pattern comprising peaks at about 6.95±0.2, 9.52±0.2 and 9.82±0.2 degrees two-theta.

In embodiments, crystalline Form A-2 of Compound I sulfate salt exhibits an X-ray powder diffraction (XRPD) pattern comprising peaks at about 6.95±0.2, 9.52±0.2, 9.82±0.2, 12.92±0.2 and 19.46±0.2 degrees two-theta.

In embodiments, crystalline Form A-2 of Compound I sulfate salt exhibits an XRPD pattern which further comprises at least two peaks selected from about 13.77±0.2, 15.30-0.2, 25.63±0.2 degrees two-theta.

In some embodiments, the crystalline form of Compound I sulfate salt exhibits an XRPD pattern comprising 1 peak, 2 peaks, 3 peaks, 4 peaks, 5 peaks, 6 peaks, 7 peaks, 8 peaks, 9 peaks, or 10 peaks ±0.2 degrees two-theta of Table 7.

In embodiments, crystalline Form A-2 of Compound I sulfate salt exhibits an XRPD pattern comprising peaks shown in Table 7, below.

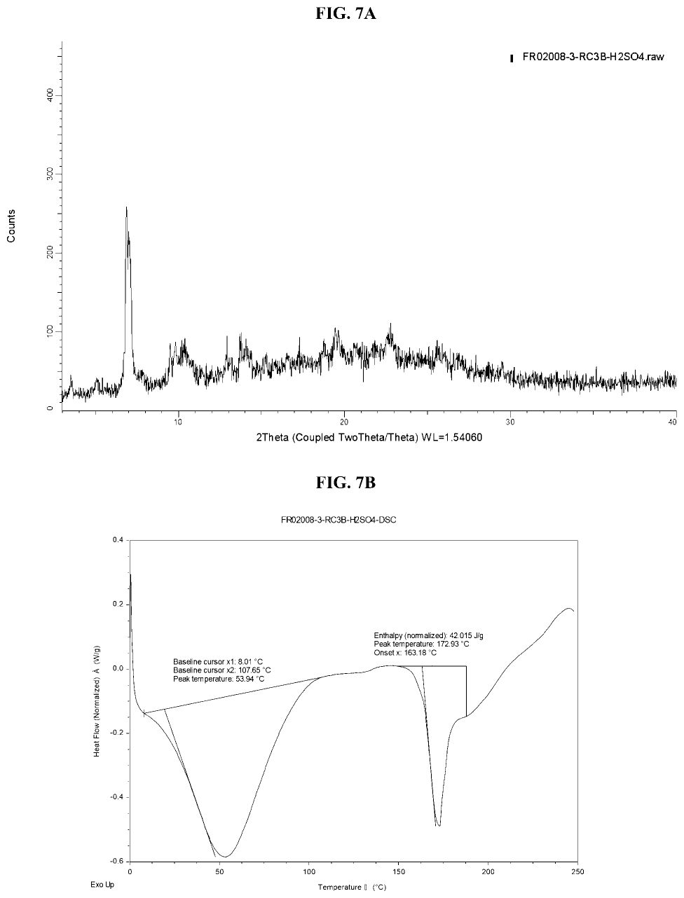

In embodiments, crystalline Form A-2 of Compound I sulfate salt an XRPD pattern that is substantially similar to FIG. 7A.

TABLE 7
Form A-2 of Compound I sulfate salt

| Angle | d Value | | Net Intensity | Rel. Intensity |
|---|---|---|---|---|
| 3.533° | 24.98572 | Å | 13.9594 | 13.2% |
| 5.091° | 17.34271 | Å | 9.56325 | 9.0% |
| 6.952° | 12.70455 | Å | 105.783 | 100.0% |
| 9.515° | 9.28727 | Å | 26.6312 | 25.2% |
| 9.816° | 9.00331 | Å | 33.5591 | 31.7% |
| 12.919° | 6.84683 | Å | 27.7389 | 26.2% |
| 13.771° | 6.42506 | Å | 24.9194 | 23.6% |
| 15.300° | 5.78651 | Å | 20.8213 | 19.7% |
| 19.455° | 4.55906 | Å | 25.3789 | 24.0% |
| 22.872° | 3.88500 | Å | 19.3881 | 18.3% |
| 25.626° | 3.47341 | Å | 20.6408 | 19.5% |
| 29.487° | 3.02679 | Å | 18.3251 | 17.3% |

In embodiments, crystalline Form A-2 of Compound I sulfate salt exhibits a DSC thermogram comprising an endotherm peak at about 54° C. with the error of margin of about ±2.5; about ±2.0; about ±1.5; about ±1.0; about ±0.5; or less. In embodiments, the crystalline Form A-2 of Compound I sulfate salt exhibits a DSC thermogram comprising an endotherm peak with an onset at about 163° C. with the error of margin of about ±2.5; about ±2.0; about ±1.5; about ±1.0; about ±0.5; or less. In embodiments, the crystalline Form A-2 of Compound I sulfate salt exhibits a DSC thermogram comprising an endotherm peak at about 173° C. with the error of margin of about ±2.5; about ±2.0; about ±1.5; about ±1.0; about ±0.5; or less.

In some embodiments, the crystalline Form A-2 of Compound I sulfate salt exhibits a DSC thermogram that is substantially similar to FIG. 7B.

### Compound I Sulfate Salt Form B-2

In embodiments, the present disclosure relates to a crystalline form of Compound I sulfate salt, which is Form B-2.

In embodiments, the crystalline form of Compound I sulfate salt may comprise of over about 99.9%, about 99.8%, about 99.7%, about 99.6%, about 99.5%, about 99.4%, about 99.3%, about 99.2%, about 99.1%, or about 99.0% of Form B-2 of Compound I sulfate salt. In another embodiment, the crystalline form of Compound I sulfate salt may comprise over about 99%, 98%, 97%, 96%, 95%, 94%, 93%, 92%, 91%, or 90% of Form B-2 of Compound I sulfate salt. In some embodiments, the crystalline form of Compound I sulfate salt may comprise over about 90%, 85%, 80%, 75%, 70%, 65%, 60%, 55%, 50%, 45%, or 40% of Form B-2 of Compound I sulfate salt.

In embodiments, crystalline Form B-2 of Compound I sulfate salt exhibits an XRPD pattern comprising peaks at about 17.85, 20.29, and 24.63 degrees two-theta with the margin of error of about +0.5; about +0.4; about +0.3; about =0.2; about +0.1; about +0.05; or less. In embodiments, crystalline Form B-2 of Compound I sulfate salt exhibits an XRPD pattern comprising peaks at about 17.85, 20.29, 23.26, 24.63, and 24.74 degrees two-theta with the margin of error of about +0.5; about +0.4; about +0.3; about +0.2; about +0.1; about +0.05; or less. In embodiments, crystalline Form B-2 of Compound I sulfate salt exhibits an XRPD pattern which further comprises at least two peaks selected from about 8.96, 12.77, 15.31, 16.68, 19.14, 20.95, 20.96, and 27.78 degrees two-theta with the margin of error of about +0.5; about +0.4; about +0.3; about +0.2; about +0.1; about +0.05; or less. In embodiments, crystalline Form B-2 of Compound I sulfate salt exhibits an XRPD pattern which further comprises at least three peaks selected from about 8.96, 12.77, 15.31, 16.68, 19.14, 20.95, 20.96, and 27.78 degrees two-theta with the margin of error of about +0.5; about +0.4; about +0.3; about +0.2; about +0.1; about +0.05; or less. In embodiments, crystalline Form B-2 of Compound I sulfate salt exhibits an XRPD pattern which further comprises at least four peaks selected from about 8.96, 12.77, 15.31, 16.68, 19.14, 20.95, 20.96, and 27.78 degrees two-theta with the margin of error of about +0.5; about +0.4; about +0.3; about =0.2; about +0.1; about +0.05; or less. In embodiments, crystalline Form B-2 of Compound I sulfate salt exhibits an XRPD pattern which further comprises at least five peaks selected from about 8.96, 12.77, 15.31, 16.68, 19.14, 20.95, 20.96, and 27.78 degrees two-theta with the margin of error of about +0.5; about +0.4; about =0.3; about +0.2; about +0.1; about +0.05; or less. In embodiments, crystalline Form B-2 of Compound I sulfate salt exhibits an XRPD pattern which further comprises at least six peaks selected from about 8.96, 12.77, 15.31, 16.68, 19.14, 20.95, 20.96, and 27.78 degrees two-theta with the margin of error of about ±0.5; about ±0.4; about ±0.3; about ±0.2; about ±0.1; about ±0.05; or less. In embodiments, crystalline Form B-2 of Compound I sulfate salt exhibits an XRPD pattern which further comprises at least seven peaks selected from about 8.96, 12.77, 15.31, 16.68, 19.14, 20.95, 20.96, and 27.78 degrees two-theta with the margin of error of about ±0.5; about ±0.4; about ±0.3; about ±0.2; about ±0.1; about ±0.05; or less. In embodiments, crystalline Form B-2 of Compound I sulfate salt exhibits an XRPD pattern which further comprises peaks at 8.96, 12.77, 15.31, 16.68, 19.14, 20.95, 20.96, and 27.78 degrees two-theta with the margin of error of about ±0.5; about ±0.4; about ±0.3; about ±0.2; about ±0.1; about ±0.05; or less.

In embodiments, crystalline Form B-2 of Compound I sulfate salt exhibits an XRPD pattern comprising peaks at about 17.85±0.2, 20.29±0.2, and 24.63±0.2 degrees two-theta.

In embodiments, crystalline Form B-2 of Compound I sulfate salt exhibits an XRPD pattern comprising peaks at about 17.85±0.2, 20.29±0.2, 23.26±0.2, 24.63±0.2, and 24.74±0.2 degrees two-theta.

In embodiments, crystalline Form B-2 of Compound I sulfate salt exhibits an XRPD pattern which further comprises at least two peaks selected from about 8.96±0.2, 12.77±0.2, 15.31±0.2, 16.68±0.2, 19.14±0.2, 20.95±0.2, 20.96±0.2, and 27.78±0.2 degrees two-theta.

In some embodiments, the crystalline form of Compound I sulfate salt exhibits an XRPD pattern comprising 1 peak, 2 peaks, 3 peaks, 4 peaks, 5 peaks, 6 peaks, 7 peaks, 8 peaks, 9 peaks, or 10 peaks ±0.2 degrees two-theta of Table 8.

In embodiments, crystalline Form B-2 of Compound I sulfate salt exhibits an XRPD pattern comprising peaks shown in Table 8, below.

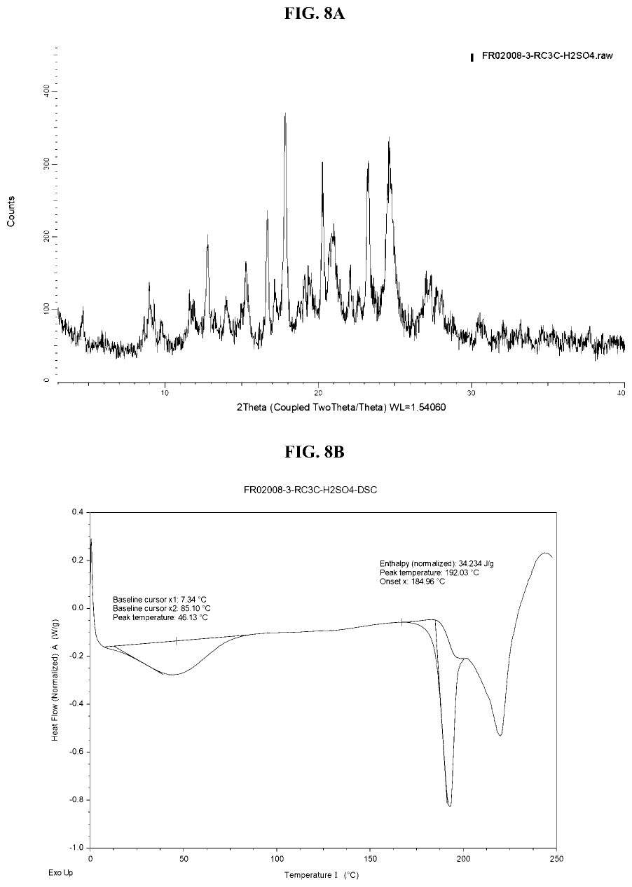

In embodiments, crystalline Form B-2 of Compound I sulfate salt an XRPD pattern that is substantially similar to FIG. 8A.

TABLE 8
Form B-2 of Compound I sulfate salt

| Angle | d Value | | Net Intensity | Rel. Intensity |
|---|---|---|---|---|
| 4.661° | 18.94436 | Å | 23.0564 | 12.0% |
| 8.611° | 10.26084 | Å | 19.0304 | 9.9% |
| 8.961° | 9.86083 | Å | 56.8513 | 29.5% |
| 9.289° | 9.51263 | Å | 38.3208 | 19.9% |
| 9.774° | 9.04234 | Å | 18.3658 | 9.5% |
| 11.663° | 7.58158 | Å | 29.2309 | 15.2% |
| 12.770° | 6.92659 | Å | 91.7794 | 47.7% |
| 14.007° | 6.31767 | Å | 25.6641 | 13.3% |
| 15.305° | 5.78470 | Å | 62.6691 | 32.5% |
| 16.676° | 5.31205 | Å | 105.828 | 55.0% |
| 17.181° | 5.15700 | Å | 35.4937 | 18.4% |
| 17.846° | 4.96615 | Å | 192.561 | 100.0% |
| 19.142° | 4.63288 | Å | 41.9335 | 21.8% |
| 19.463° | 4.55726 | Å | 33.0789 | 17.2% |
| 20.287° | 4.37384 | Å | 136.225 | 70.7% |
| 20.946° | 4.23770 | Å | 65.8775 | 34.2% |
| 20.956° | 4.23575 | Å | 72.4681 | 37.6% |
| 22.082° | 4.02222 | Å | 39.7926 | 20.7% |
| 23.261° | 3.82100 | Å | 135.286 | 70.3% |
| 24.628° | 3.61189 | Å | 175.408 | 91.1% |
| 24.738° | 3.59613 | Å | 114.766 | 59.6% |
| 27.284° | 3.26603 | Å | 47.7528 | 24.8% |
| 27.778° | 3.20905 | Å | 33.5533 | 17.4% |
| 28.068° | 3.17649 | Å | 29.4597 | 15.3% |
| 30.461° | 2.93217 | Å | 18.1841 | 9.4% |
| 33.166° | 2.69896 | Å | 19.3101 | 10.0% |
| 34.603° | 2.59014 | Å | 12.2306 | 6.4% |
| 37.691° | 2.38470 | Å | 22.5338 | 11.7% |

In embodiments, crystalline Form B-2 of Compound I sulfate salt exhibits a DSC thermogram comprising an endotherm peak at about 46° C. with the error of margin of about ±2.5; about ±2.0; about ±1.5; about ±1.0; about ±0.5; or less. In embodiments, the crystalline Form B-2 of Compound I sulfate salt exhibits a DSC thermogram comprising an endotherm peak with an onset at about 185° C. with the error of margin of about ±2.5; about ±2.0; about ±1.5; about ±1.0; about ±0.5; or less. In embodiments, the crystalline Form B-2 of Compound I sulfate salt exhibits a DSC thermogram comprising an endotherm peak at about 192° C. with the error of margin of about ±2.5; about ±2.0; about ±1.5; about ±1.0; about ±0.5; or less.

In some embodiments, the crystalline Form B-2 of Compound I sulfate salt exhibits a DSC thermogram that is substantially similar to FIG. 8B.

### Compound I Mesylate Salt

In one embodiment, the present disclosure relates to a Compound I mesylate salt, or a solvate thereof. In embodiments, the crystalline form of Compound I mesylate salt comprises a mixture of one or more forms of polymorphs of Compound I mesylate salt. In embodiments, the crystalline form of Compound I mesylate salt comprises of substantially pure form of one polymorph type.

### Compound I Mesylate Salt Form A-3

In embodiments, the present disclosure relates to a crystalline form of Compound I mesylate salt, which is Form A-3.

In embodiments, the crystalline form of Compound I mesylate salt may comprise of over about 99.9%, about 99.8%, about 99.7%, about 99.6%, about 99.5%, about 99.4%, about 99.3%, about 99.2%, about 99.1%, or about 99.0% of Form A-3 of Compound I mesylate salt. In another embodiment, the crystalline form of Compound I mesylate salt may comprise over about 99%, 98%, 97%, 96%, 95%, 94%, 93%, 92%, 91%, or 90% of Form A-3 of Compound I mesylate salt. In some embodiments, the crystalline form of Compound I mesylate salt may comprise over about 90%, 85%, 80%, 75%, 70%, 65%, 60%, 55%, 50%, 45%, or 40% of Form A-3 of Compound I mesylate salt.

In embodiments, crystalline Form A-3 of Compound I mesylate salt exhibits an XRPD pattern comprising peaks at about 10.23, 14.18 and 18.56 degrees two-theta with the margin of error of about ±0.5; about ±0.4; about ±0.3; about ±0.2; about ±0.1; about ±0.05; or less. In embodiments, crystalline Form A-3 of Compound I mesylate salt exhibits an XRPD pattern comprising peaks at about 5.41, 7.09, 10.23, 14.18 and 18.56 degrees two-theta with the margin of error of about ±0.5; about ±0.4; about ±0.3; about ±0.2; about ±0.1; about ±0.05; or less. In embodiments, crystalline Form A-3 of Compound I mesylate salt exhibits an XRPD pattern which further comprises at least two peaks at about 3.55, 10.78, 12.45, 18.76, 19.82, 21.89, 22.32, and 23.24 degrees two-theta with the margin of error of about ±0.5; about ±0.4; about ±0.3; about ±0.2; about ±0.1; about ±0.05; or less. In embodiments, crystalline Form A-3 of Compound I mesylate salt exhibits an XRPD pattern which further comprises at least three peaks at about 3.55, 10.78, 12.45, 18.76, 19.82, 21.89, 22.32, and 23.24 degrees two-theta with the margin of error of about ±0.5; about ±0.4; about ±0.3; about ±0.2; about ±0.1; about ±0.05; or less. In embodiments, crystalline Form A-3 of Compound I mesylate salt exhibits an XRPD pattern which further comprises at least four peaks at about 3.55, 10.78, 12.45, 18.76, 19.82, 21.89, 22.32, and 23.24 degrees two-theta with the margin of error of about ±0.5; about ±0.4; about ±0.3; about ±0.2; about ±0.1; about ±0.05; or less. In embodiments, crystalline Form A-3 of Compound I mesylate salt exhibits an XRPD pattern which further comprises at least five peaks at about 3.55, 10.78, 12.45, 18.76, 19.82, 21.89, 22.32, and 23.24 degrees two-theta with the margin of error of about ±0.5; about ±0.4; about ±0.3; about ±0.2; about ±0.1; about ±0.05; or less. In embodiments, crystalline Form A-3 of Compound I mesylate salt exhibits an XRPD pattern which further comprises at least six peaks at about 3.55, 10.78, 12.45, 18.76, 19.82, 21.89, 22.32, and 23.24 degrees two-theta with the margin of error of about ±0.5; about ±0.4; about ±0.3; about ±0.2; about ±0.1; about ±0.05; or less. In embodiments, crystalline Form A-3 of Compound I mesylate salt exhibits an XRPD pattern which further comprises at least seven peaks at about 3.55, 10.78, 12.45, 18.76, 19.82, 21.89, 22.32, and 23.24 degrees two-theta with the margin of error of about ±0.5; about ±0.4; about ±0.3; about ±0.2; about ±0.1; about ±0.05; or less. In embodiments, crystalline Form A-3 of Compound I mesylate salt exhibits an XRPD pattern which further comprises peaks at about 3.55, 10.78, 12.45, 18.76, 19.82, 21.89, 22.32, and 23.24 degrees two-theta with the margin of error of about ±0.5; about ±0.4; about ±0.3; about ±0.2; about ±0.1; about ±0.05; or less.

In embodiments, crystalline Form A-3 of Compound I mesylate salt exhibits an XRPD pattern comprising peaks at about 10.23±0.2, 14.18±0.2 and 18.56±0.2 degrees two-theta.

In embodiments, crystalline Form A-3 of Compound I mesylate salt exhibits an XRPD pattern comprising peaks at about 5.41±0.2, 7.09±0.2, 10.23±0.2, 14.18±0.2 and 18.56±0.2 degrees two-theta.

In embodiments, crystalline Form A-3 of Compound I mesylate salt exhibits an XRPD pattern which further comprises peaks at least two peaks selected from about 3.55±0.2, 10.78±0.2, 12.45±0.2, 18.76±0.2, 19.82±0.2, 21.89±0.2, 22.32±0.2, and 23.24±0.2 degrees two-theta

In some embodiments, the crystalline form of Compound I mesylate salt exhibits an XRPD pattern comprising 1 peak, 2 peaks, 3 peaks, 4 peaks, 5 peaks, 6 peaks, 7 peaks, 8 peaks, 9 peaks, or 10 peaks ±0.2 degrees two-theta of Table 9.

In embodiments, crystalline Form A-3 of Compound I mesylate salt exhibits an XRPD pattern comprising peaks shown in Table 9, below.

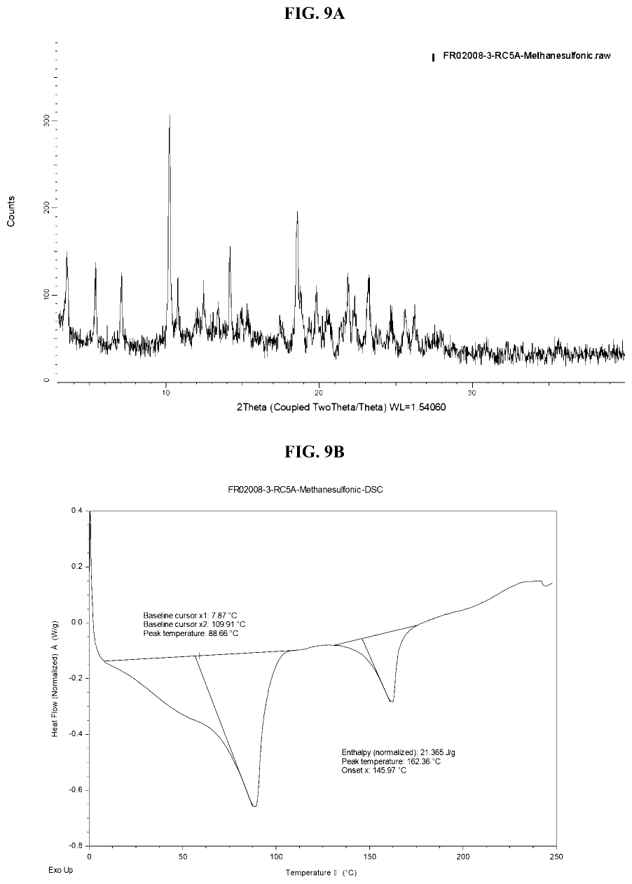

In embodiments, crystalline Form A-3 of Compound I mesylate salt exhibits an XRPD pattern that is substantially similar to FIG. 9A.

TABLE 9
Form A-3 of Compound I mesylate salt

| Angle | d Value | | Net Intensity | Rel. Intensity |
|---|---|---|---|---|
| 3.550° | 24.86704 | Å | 48.6552 | 28.3% |
| 5.409° | 16.32629 | Å | 57.9899 | 33.7% |
| 7.089° | 12.45948 | Å | 58.2853 | 33.9% |
| 10.229° | 8.64046 | Å | 171.994 | 100.0% |
| 10.777° | 8.20289 | Å | 48.2462 | 28.1% |
| 12.451° | 7.10311 | Å | 46.3511 | 26.9% |
| 13.396° | 6.60415 | Å | 24.1937 | 14.1% |
| 14.182° | 6.23981 | Å | 68.1007 | 39.6% |
| 17.448° | 5.07857 | Å | 22.6146 | 13.1% |
| 18.562° | 4.77615 | Å | 92.1941 | 53.6% |
| 18.760° | 4.72642 | Å | 44.7154 | 26.0% |
| 19.362° | 4.58076 | Å | 28.6202 | 16.6% |
| 19.820° | 4.47580 | Å | 44.3185 | 25.8% |
| 20.574° | 4.31351 | Å | 24.7953 | 14.4% |
| 20.652° | 4.29729 | Å | 20.0119 | 11.6% |
| 21.408° | 4.14724 | Å | 20.7227 | 12.0% |
| 21.894° | 4.05628 | Å | 55.4023 | 32.2% |
| 22.321° | 3.97962 | Å | 47.6172 | 27.7% |
| 23.243° | 3.82389 | Å | 38.2285 | 22.2% |
| 24.709° | 3.60020 | Å | 20.0773 | 11.7% |
| 25.611° | 3.47548 | Å | 32.9831 | 19.2% |
| 26.241° | 3.39336 | Å | 32.3912 | 18.8% |

In embodiments, crystalline Form A-3 of Compound I mesylate salt exhibits a DSC thermogram comprising an endotherm peak at about 89° C. with the error of margin of about ±2.5; about ±2.0; about ±1.5; about ±1.0; about ±0.5; or less. In embodiments, the crystalline Form A-3 of Compound I mesylate salt exhibits a DSC thermogram comprising an endotherm peak with an onset at about 146° C. with the error of margin of about ±2.5; about ±2.0; about ±1.5; about ±1.0; about ±0.5; or less. In embodiments, the crystalline Form A-3 of Compound I mesylate salt exhibits a DSC thermogram comprising an endotherm peak at about 162° C. with the error of margin of about ±2.5; about ±2.0; about ±1.5; about ±1.0; about ±0.5; or less.

In some embodiments, the crystalline Form A-3 of Compound I mesylate salt exhibits a DSC thermogram that is substantially similar to FIG. 9B.

### Compound I Mesylate Salt Form B-3

In embodiments, the present disclosure relates to a crystalline form of Compound I mesylate salt, which is Form B-3.

In embodiments, the crystalline form of Compound I mesylate salt may comprise of over about 99.9%, about 99.8%, about 99.7%, about 99.6%, about 99.5%, about 99.4%, about 99.3%, about 99.2%, about 99.1%, or about 99.0% of Form B-3 of Compound I mesylate salt. In another embodiment, the crystalline form of Compound I mesylate salt may comprise over about 99%, 98%, 97%, 96%, 95%, 94%, 93%, 92%, 91%, or 90% of Form B-3 of Compound I mesylate salt. In some embodiments, the crystalline form of Compound I mesylate salt may comprise over about 90%, 85%, 80%, 75%, 70%, 65%, 60%, 55%, 50%, 45%, or 40% of Form B-3 of Compound I mesylate salt.

In embodiments, crystalline Form B-3 of Compound I mesylate salt exhibits an XRPD pattern comprising peaks at about 4.80, 7.20 and 19.93 degrees two-theta with the margin of error of about ±0.5; about ±0.4; about ±0.3; about ±0.2; about ±0.1; about ±0.05; or less. In embodiments, crystalline Form B-3 of Compound I mesylate salt exhibits an XRPD pattern comprising peaks at about 4.80, 7.20, 18.28, 19.93, and 21.17 degrees two-theta with the margin of error of about ±0.5; about ±0.4; about ±0.3; about ±0.2; about ±0.1; about ±0.05; or less. In embodiments, crystalline Form B-3 of Compound I mesylate salt exhibits an XRPD pattern which further comprises at least two peaks selected from about 12.43, 14.77, 16.08, 18.56, 22.77, 23.04, 23.86, and 24.43 degrees two-theta with the margin of error of about ±0.5; about ±0.4; about ±0.3; about ±0.2; about ±0.1; about ±0.05; or less. In embodiments, crystalline Form B-3 of Compound I mesylate salt exhibits an XRPD pattern which further comprises at least three peaks selected from about 12.43, 14.77, 16.08, 18.56, 22.77, 23.04, 23.86, and 24.43 degrees two-theta with the margin of error of about ±0.5; about ±0.4; about ±0.3; about ±0.2; about ±0.1; about ±0.05; or less. In embodiments, crystalline Form B-3 of Compound I mesylate salt exhibits an XRPD pattern which further comprises at least four peaks selected from about 12.43, 14.77, 16.08, 18.56, 22.77, 23.04, 23.86, and 24.43 degrees two-theta with the margin of error of about ±0.5; about ±0.4; about ±0.3; about ±0.2; about ±0.1; about ±0.05; or less. In embodiments, crystalline Form B-3 of Compound I mesylate salt exhibits an XRPD pattern which further comprises at least five peaks selected from about 12.43, 14.77, 16.08, 18.56, 22.77, 23.04, 23.86, and 24.43 degrees two-theta with the margin of error of about ±0.5; about ±0.4; about ±0.3; about ±0.2; about ±0.1; about ±0.05; or less. In embodiments, crystalline Form B-3 of Compound I mesylate salt exhibits an XRPD pattern which further comprises at least six peaks selected from about 12.43, 14.77, 16.08, 18.56, 22.77, 23.04, 23.86, and 24.43 degrees two-theta with the margin of error of about ±0.5; about ±0.4; about ±0.3; about ±0.2; about ±0.1; about ±0.05; or less. In embodiments, crystalline Form B-3 of Compound I mesylate salt exhibits an XRPD pattern which further comprises at least seven peaks selected from about 12.43, 14.77, 16.08, 18.56, 22.77, 23.04, 23.86, and 24.43 degrees two-theta with the margin of error of about ±0.5; about ±0.4; about ±0.3; about ±0.2; about ±0.1; about ±0.05; or less. In embodiments, crystalline Form B-3 of Compound I mesylate salt exhibits an XRPD pattern which further comprises peaks at about 12.43, 14.77, 16.08, 18.56, 22.77, 23.04, 23.86, and 24.43 degrees two-theta with the margin of error of about ±0.5; about ±0.4; about ±0.3; about ±0.2; about ±0.1; about ±0.05; or less.

In embodiments, crystalline Form B-3 of Compound I mesylate salt exhibits an X-ray powder diffraction (XRPD) pattern comprising peaks at about 4.80±0.2, 7.20±0.2 and 19.93±0.2 degrees two-theta.

In embodiments, crystalline Form B-3 of Compound I mesylate salt exhibits an X-ray powder diffraction (XRPD) pattern comprising peaks at about 4.80±0.2, 7.200.2, 18.28±0.2, 19.93±0.2, and 21.17±0.2 degrees two-theta.

In embodiments, crystalline Form B-3 of Compound I mesylate salt exhibits an X-ray powder diffraction (XRPD) pattern further comprising at least two peaks selected from about 12.43±0.2, 14.77±0.2, 16.08±0.2, 18.56±0.2, 22.77±0.2, 23.04±0.2, 23.86±0.2, and 24.43±0.2 degrees two-theta.

In some embodiments, the crystalline form of Compound I mesylate salt exhibits an XRPD pattern comprising 1 peak, 2 peaks, 3 peaks, 4 peaks, 5 peaks, 6 peaks, 7 peaks, 8 peaks, 9 peaks, or 10 peaks ±0.2 degrees two-theta of Table 10.

In embodiments, crystalline Form B-3 of Compound I mesylate salt exhibits an XRPD pattern comprising peaks in Table 10, below.

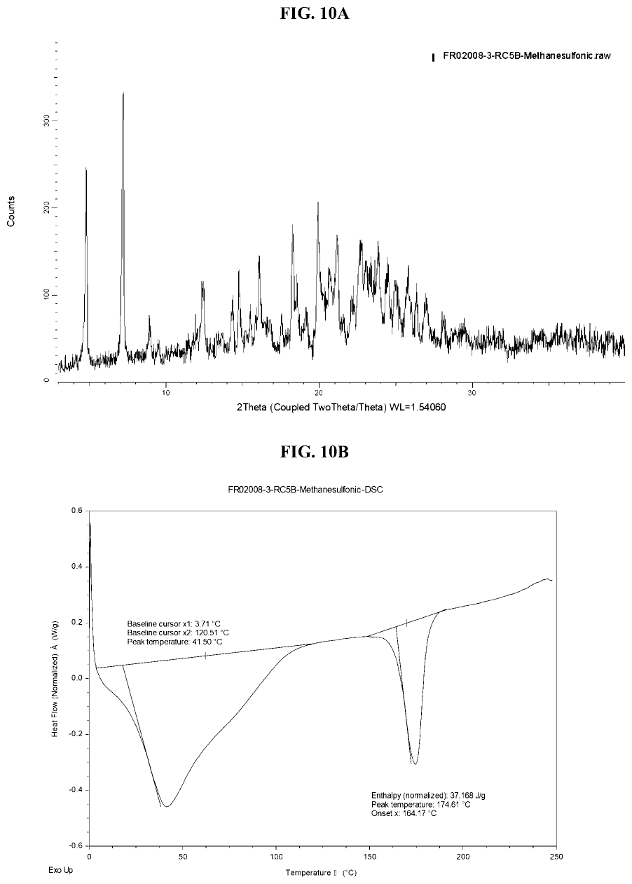

In embodiments, crystalline Form B-3 of Compound I mesylate salt exhibits an XRPD pattern that is substantially similar to FIG. 10A.

TABLE 10
Form B-3 of Compound I mesylate salt

| Angle | d Value | | Net Intensity | Rel. Intensity |
|---|---|---|---|---|
| 4.795° | 18.41525 | Å | 139.716 | 69.2% |
| 7.197° | 12.27365 | Å | 201.899 | 100.0% |
| 8.920° | 9.90550 | Å | 35.3057 | 17.5% |
| 12.434° | 7.11314 | Å | 57.2520 | 28.4% |
| 14.353° | 6.16609 | Å | 42.8039 | 21.2% |
| 14.774° | 5.99130 | Å | 53.7044 | 26.6% |
| 15.511° | 5.70821 | Å | 24.4426 | 12.1% |
| 16.078° | 5.50807 | Å | 53.2311 | 26.4% |
| 16.804° | 5.27168 | Å | 17.1684 | 8.5% |
| 17.541° | 5.05194 | Å | 27.3069 | 13.5% |
| 18.281° | 4.84898 | Å | 100.606 | 49.8% |
| 18.562° | 4.77631 | Å | 58.9034 | 29.2% |
| 19.154° | 4.62985 | Å | 28.9927 | 14.4% |
| 19.925° | 4.45250 | Å | 108.505 | 53.7% |
| 20.656° | 4.29662 | Å | 47.2103 | 23.4% |
| 21.168° | 4.19381 | Å | 79.8443 | 39.5% |
| 22.173° | 4.00587 | Å | 33.6967 | 16.7% |
| 22.208° | 3.99969 | Å | 19.9888 | 9.9% |
| 22.768° | 3.90263 | Å | 65.4686 | 32.4% |
| 23.043° | 3.85664 | Å | 60.9786 | 30.2% |
| 23.341° | 3.80810 | Å | 30.1561 | 14.9% |
| 23.859° | 3.72649 | Å | 69.8369 | 34.6% |
| 24.434° | 3.64014 | Å | 65.4595 | 32.4% |
| 24.949° | 3.56619 | Å | 41.0277 | 20.3% |
| 25.802° | 3.45010 | Å | 50.7735 | 25.1% |
| 26.349° | 3.37978 | Å | 44.0883 | 21.8% |
| 27.010° | 3.29853 | Å | 22.7964 | 11.3% |

In embodiments, crystalline Form B-3 of Compound I mesylate salt exhibits a DSC thermogram comprising an endotherm peak at about 42° C. with the error of margin of about ±2.5; about ±2.0; about ±1.5; about ±1.0; about ±0.5; or less. In embodiments, the crystalline Form B-3 of Compound I mesylate salt exhibits a DSC thermogram comprising an endotherm peak with an onset at about 164° C. with the error of margin of about ±2.5; about ±2.0; about ±1.5; about ±1.0; about ±0.5; or less. In embodiments, the crystalline Form B-3 of Compound I mesylate salt exhibits a DSC thermogram comprising an endotherm peak at about 175° C. with the error of margin of about ±2.5; about ±2.0; about ±1.5; about ±1.0; about ±0.5; or less.

In some embodiments, the crystalline Form B-3 of Compound I mesylate salt exhibits a DSC thermogram that is substantially similar to FIG. 10B.

### Compound I Mesylate Salt Form C-3

In embodiments, the present disclosure relates to a crystalline form of Compound I mesylate salt, which is Form C-3.

In embodiments, the crystalline form of Compound I mesylate salt may comprise of over about 99.9%, about 99.8%, about 99.7%, about 99.6%, about 99.5%, about 99.4%, about 99.3%, about 99.2%, about 99.1%, or about 99.0% of Form C-3 of Compound I mesylate salt. In another embodiment, the crystalline form of Compound I mesylate salt may comprise over about 99%, 98%, 97%, 96%, 95%, 94%, 93%, 92%, 91%, or 90% of Form C-3 of Compound I mesylate salt. In some embodiments, the crystalline form of Compound I mesylate salt may comprise over about 90%, 85%, 80%, 75%, 70%, 65%, 60%, 55%, 50%, 45%, or 40% of Form C-3 of Compound I mesylate salt.

In embodiments, crystalline Form C-3 of Compound I mesylate salt exhibits an XRPD pattern comprising peaks at about 7.09, 16.73 and 22.68 degrees two-theta with the margin of error of about ±0.5; about ±0.4; about ±0.3; about ±0.2; about ±0.1; about ±0.05; or less. In embodiments, crystalline Form C-3 of Compound I mesylate salt exhibits an XRPD pattern comprising peaks at about 6.89, 7.09, 16.73, 22.34, and 22.68 degrees two-theta with the margin of error of about ±0.5; about ±0.4; about ±0.3; about ±0.2; about ±0.1; about ±0.05; or less. In embodiments, crystalline Form C-3 of Compound I mesylate salt exhibits an XRPD pattern which further comprises at least two peaks selected from about 9.24, 16.02, 16.73, 19.86, 21.29, 21.81, 23.93, 24.54, and 27.40 degrees two-theta with the margin of error of about ±0.5; about ±0.4; about ±0.3; about ±0.2; about ±0.1; about ±0.05; or less. In embodiments, crystalline Form C-3 of Compound I mesylate salt exhibits an XRPD pattern which further comprises at least three peaks selected from about 9.24, 16.02, 16.73, 19.86, 21.29, 21.81, 23.93, 24.54, and 27.40 degrees two-theta with the margin of error of about ±0.5; about ±0.4; about ±0.3; about ±0.2; about ±0.1; about ±0.05; or less. In embodiments, crystalline Form C-3 of Compound I mesylate salt exhibits an XRPD pattern which further comprises at least four peaks selected from about 9.24, 16.02, 16.73, 19.86, 21.29, 21.81, 23.93, 24.54, and 27.40 degrees two-theta with the margin of error of about ±0.5; about ±0.4; about ±0.3; about ±0.2; about ±0.1; about ±0.05; or less. In embodiments, crystalline Form C-3 of Compound I mesylate salt exhibits an XRPD pattern which further comprises at least five peaks selected from about 9.24, 16.02, 16.73, 19.86, 21.29, 21.81, 23.93, 24.54, and 27.40 degrees two-theta with the margin of error of about ±0.5; about ±0.4; about ±0.3; about ±0.2; about ±0.1; about ±0.05; or less. In embodiments, crystalline Form C-3 of Compound I mesylate salt exhibits an XRPD pattern which further comprises at least six peaks selected from about 9.24, 16.02, 16.73, 19.86, 21.29, 21.81, 23.93, 24.54, and 27.40 degrees two-theta with the margin of error of about ±0.5; about ±0.4; about ±0.3; about ±0.2; about ±0.1; about ±0.05; or less. In embodiments, crystalline Form C-3 of Compound I mesylate salt exhibits an XRPD pattern which further comprises at least seven peaks selected from about 9.24, 16.02, 16.73, 19.86, 21.29, 21.81, 23.93, 24.54, and 27.40 degrees two-theta with the margin of error of about ±0.5; about ±0.4; about ±0.3; about ±0.2; about ±0.1; about ±0.05; or less. In embodiments, crystalline Form C-3 of Compound I mesylate salt exhibits an XRPD pattern which further comprises peaks at about 9.24, 16.02, 16.73, 19.86, 21.29, 21.81, 23.93, 24.54, and 27.40 degrees two-theta with the margin of error of about ±0.5; about ±0.4; about ±0.3; about ±0.2; about ±0.1; about ±0.05; or less.

In embodiments, crystalline Form C-3 of Compound I mesylate salt exhibits an XRPD pattern comprising peaks at about 7.09±0.2, 16.73±0.2 and 22.68±0.2 degrees two-theta.

In embodiments, crystalline Form C-3 of Compound I mesylate salt exhibits an XRPD pattern comprising peaks at about 6.89±0.2, 7.09±0.2, 16.73±0.2, 22.34±0.2, and 22.68±0.2

In embodiments, crystalline Form C-3 of Compound I mesylate salt exhibits an XRPD pattern comprising peaks at about 9.24±0.2, 16.02±0.2, 16.73±0.2, 19.86±0.2, 21.29±0.2, 21.81±0.2, 23.93±0.2, 24.54±0.2, and 27.40±0.2 degrees two-theta.

In some embodiments, the crystalline form of Compound I mesylate salt exhibits an XRPD pattern comprising 1 peak, 2 peaks, 3 peaks, 4 peaks, 5 peaks, 6 peaks, 7 peaks, 8 peaks, 9 peaks, or 10 peaks ±0.2 degrees two-theta of Table 11.

In embodiments, crystalline Form C-3 of Compound I mesylate salt exhibits an XRPD pattern comprising peaks in Table 11, below.

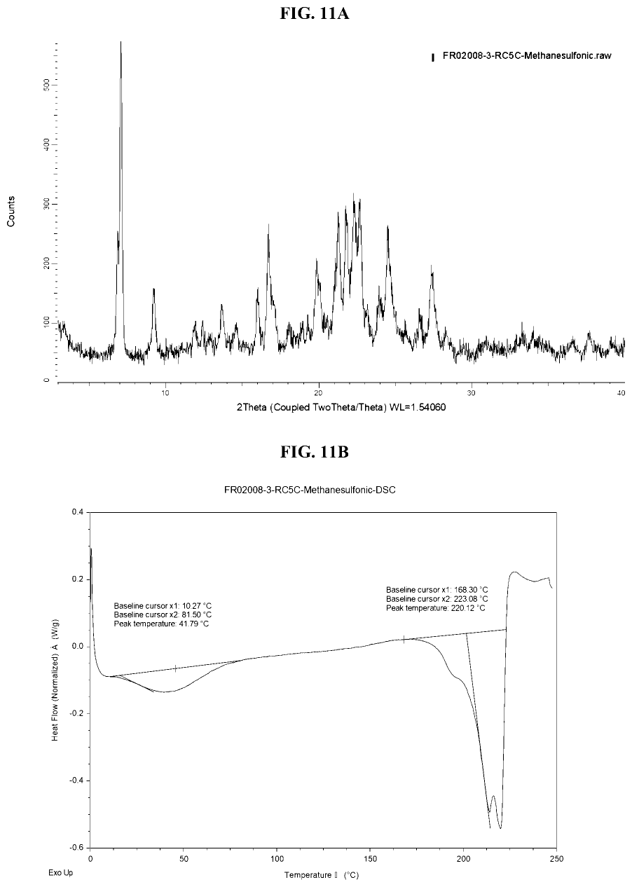

In embodiments, crystalline Form C-3 of Compound I mesylate salt exhibits an XRPD pattern that is substantially similar to FIG. 11A.

TABLE 11
Form C-3 of Compound I mesylate salt

| Angle | d Value | | Net Intensity | Rel. Intensity |
|---|---|---|---|---|
| 6.891° | 12.81759 | Å | 133.916 | 36.9% |
| 7.091° | 12.45583 | Å | 362.946 | 100.0% |
| 9.240° | 9.56294 | Å | 82.8898 | 22.8% |
| 11.911° | 7.42411 | Å | 15.3116 | 4.2% |
| 12.423° | 7.11934 | Å | 30.4547 | 8.4% |
| 13.696° | 6.46049 | Å | 38.7574 | 10.7% |
| 14.629° | 6.05021 | Å | 26.3748 | 7.3% |
| 16.016° | 5.52921 | Å | 60.6772 | 16.7% |
| 16.731° | 5.29466 | Å | 146.424 | 40.3% |
| 17.029° | 5.20272 | Å | 45.5790 | 12.6% |
| 18.968° | 4.67486 | Å | 15.8339 | 4.4% |
| 19.303° | 4.59453 | Å | 27.9978 | 7.7% |
| 19.861° | 4.46672 | Å | 87.8637 | 24.2% |
| 21.164° | 4.19450 | Å | 39.0375 | 10.8% |
| 21.293° | 4.16938 | Å | 126.072 | 34.7% |
| 21.810° | 4.07174 | Å | 116.097 | 32.0% |
| 22.336° | 3.97698 | Å | 143.890 | 39.6% |
| 22.683° | 3.91691 | Å | 151.844 | 41.8% |
| 23.173° | 3.83521 | Å | 41.3479 | 11.4% |
| 23.933° | 3.71520 | Å | 61.6919 | 17.0% |
| 24.536° | 3.62520 | Å | 131.606 | 36.3% |
| 25.677° | 3.46666 | Å | 20.5503 | 5.7% |
| 26.635° | 3.34409 | Å | 23.6148 | 6.5% |
| 27.398° | 3.25271 | Å | 76.2094 | 21.0% |
| 28.339° | 3.14678 | Å | 26.5161 | 7.3% |
| 33.966° | 2.63721 | Å | 13.6938 | 3.8% |
| 36.558° | 2.45599 | Å | 13.8146 | 3.8% |
| 37.633° | 2.38822 | Å | 15.2070 | 4.2% |

In embodiments, crystalline Form C-3 of Compound I mesylate salt exhibits a DSC thermogram comprising an endotherm peak at about 42° C. with the error of margin of about ±2.5; about ±2.0; about ±1.5; about ±1.0; about ±0.5; or less. In embodiments, the crystalline Form C-3 of Compound I mesylate salt exhibits a DSC thermogram comprising an endotherm peak at about 220° C. with the error of margin of about ±2.5; about ±2.0; about ±1.5; about ±1.0; about ±0.5; or less.

In some embodiments, the crystalline Form C-3 of Compound I mesylate salt exhibits a DSC thermogram that is substantially similar to FIG. 11B.

### Compound I Tosylate Salt

In one embodiment, the present disclosure relates to a Compound I tosylate salt, or a solvate thereof. In embodiments, the crystalline form of Compound I tosylate salt comprises a mixture of one or more forms of polymorphs of Compound I tosylate salt. In embodiments, the crystalline form of Compound I tosylate salt comprises of substantially pure form of one polymorph type.

### Compound I Tosylate Salt Form A-4

In embodiments, the present disclosure relates to a crystalline form of Compound I tosylate salt, which is Form A-4.

In embodiments, the crystalline form of Compound I tosylate salt may comprise of over about 99.9%, about 99.8%, about 99.7%, about 99.6%, about 99.5%, about 99.4%, about 99.3%, about 99.2%, about 99.1%, or about 99.0% of Form A-4 of Compound I tosylate salt. In another embodiment, the crystalline form of Compound I tosylate salt may comprise over about 99%, 98%, 97%, 96%, 95%, 94%, 93%, 92%, 91%, or 90% of Form A-4 of Compound I tosylate salt. In some embodiments, the crystalline form of Compound I tosylate salt may comprise over about 90%, 85%, 80%, 75%, 70%, 65%, 60%, 55%, 50%, 45%, or 40% of Form A-4 of Compound I tosylate salt

In embodiments, crystalline Form A-4 of Compound I tosylate salt exhibits an XRPD pattern comprising peaks at about 7.46, 9.99 and 19.09 degrees two-theta with the margin of error of about ±0.5; about ±0.4; about ±0.3; about ±0.2; about ±0.1; about ±0.05; or less. In embodiments, crystalline Form A-4 of Compound I tosylate salt exhibits an XRPD pattern comprising peaks at about 7.46, 9.99, 14.89, 19.09, and 22.39 degrees two-theta with the margin of error of about ±0.5; about ±0.4; about ±0.3; about ±0.2; about ±0.1; about ±0.05; or less. In embodiments, crystalline Form A-4 of Compound I tosylate salt exhibits an XRPD pattern which further comprises at least two peaks selected from about 10.61, 15.36, 17.64, 18.27, 19.65, 19.97, 23.10, and 25.30 degrees two-theta with the margin of error of about ±0.5; about ±0.4; about ±0.3; about ±0.2; about ±0.1; about ±0.05; or less. In embodiments, crystalline Form A-4 of Compound I tosylate salt exhibits an XRPD pattern which further comprises at least three peaks selected from about 10.61, 15.36, 17.64, 18.27, 19.65, 19.97, 23.10, and 25.30 degrees two-theta with the margin of error of about ±0.5; about ±0.4; about ±0.3; about ±0.2; about ±0.1; about ±0.05; or less. In embodiments, crystalline Form A-4 of Compound I tosylate salt exhibits an XRPD pattern which further comprises at least four peaks selected from about 10.61, 15.36, 17.64, 18.27, 19.65, 19.97, 23.10, and 25.30 degrees two-theta with the margin of error of about ±0.5; about ±0.4; about ±0.3; about ±0.2; about ±0.1; about ±0.05; or less. In embodiments, crystalline Form A-4 of Compound I tosylate salt exhibits an XRPD pattern which further comprises at least five peaks selected from about 10.61, 15.36, 17.64, 18.27, 19.65, 19.97, 23.10, and 25.30 degrees two-theta with the margin of error of about ±0.5; about ±0.4; about ±0.3; about ±0.2; about ±0.1; about ±0.05; or less. In embodiments, crystalline Form A-4 of Compound I tosylate salt exhibits an XRPD pattern which further comprises at least six peaks selected from about 10.61, 15.36, 17.64, 18.27, 19.65, 19.97, 23.10, and 25.30 degrees two-theta with the margin of error of about ±0.5; about ±0.4; about ±0.3; about ±0.2; about ±0.1; about ±0.05; or less. In embodiments, crystalline Form A-4 of Compound I tosylate salt exhibits an XRPD pattern which further comprises at least seven peaks selected from about 10.61, 15.36, 17.64, 18.27, 19.65, 19.97, 23.10, and 25.30 degrees two-theta with the margin of error of about ±0.5; about ±0.4; about ±0.3; about ±0.2; about ±0.1; about ±0.05; or less. In embodiments, crystalline Form A-4 of Compound I tosylate salt exhibits an XRPD pattern which further comprises peaks at 10.61, 15.36, 17.64, 18.27, 19.65, 19.97, 23.10, and 25.30 degrees two-theta with the margin of error of about ±0.5; about ±0.4; about ±0.3; about ±0.2; about ±0.1; about ±0.05; or less.

In embodiments, crystalline Form A-4 of Compound I tosylate salt exhibits an XRPD pattern comprising peaks at about 7.46±0.2, 9.99±0.2 and 19.09±0.2 degrees two-theta.

In embodiments, crystalline Form A-4 of Compound I tosylate salt exhibits an XRPD pattern comprising peaks at about 7.46±0.2, 9.99±0.2, 14.89±0.2, 19.09±0.2, and 22.39±0.2 degrees two-theta.

In embodiments, crystalline Form A-4 of Compound I tosylate salt exhibits an XRPD pattern which further comprises at least two peaks selected from about 10.61±0.2, 15.36±0.2, 17.64±0.2, 18.27±0.2, 19.65±0.2, 19.97±0.2, 23.10±0.2, and 25.30±0.2 degrees two-theta.

In some embodiments, the crystalline form of Compound I tosylate salt exhibits an XRPD pattern comprising 1 peak, 2 peaks, 3 peaks, 4 peaks, 5 peaks, 6 peaks, 7 peaks, 8 peaks, 9 peaks, or 10 peaks ±0.2 degrees two-theta of Table 12.

In embodiments, crystalline Form A-4 of Compound I tosylate salt exhibits an XRPD pattern comprising peaks in Table 12.

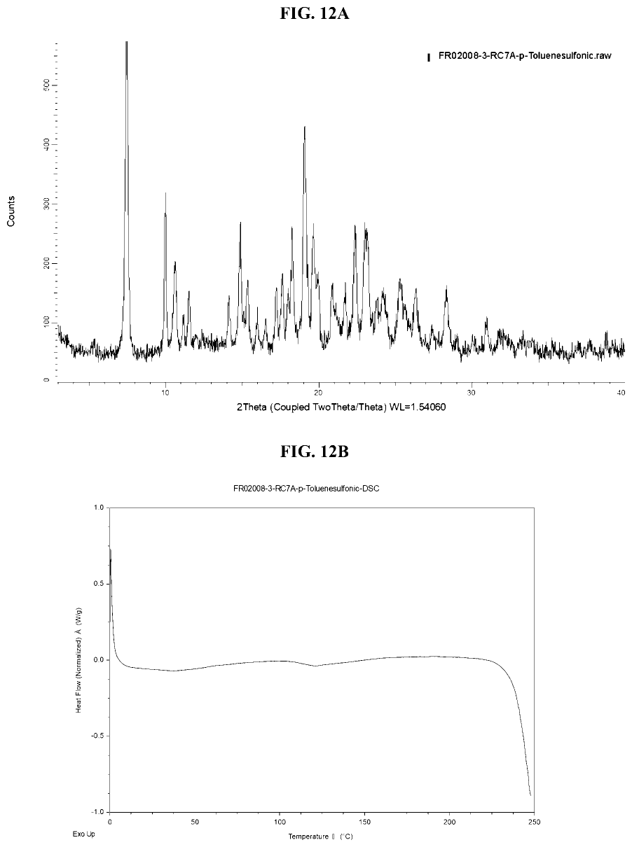

In embodiments, crystalline Form A-4 of Compound I tosylate salt exhibits an XRPD pattern that is substantially similar to FIG. 12A.

TABLE 12
Form A-4 of Compound I tosylate salt

| Angle | d Value | | Net Intensity | Rel. Intensity | h, k, l |
|---|---|---|---|---|---|
| 7.456° | 11.84655 | Å | 373.580 | 100.0% | n.a. |
| 9.989° | 8.84789 | Å | 178.627 | 47.8% | n.a. |
| 10.613° | 8.32875 | Å | 88.2962 | 23.6% | n.a. |
| 11.192° | 7.89954 | Å | 34.6693 | 9.3% | n.a. |
| 11.534° | 7.66597 | Å | 58.9717 | 15.8% | n.a. |
| 14.148° | 6.25476 | Å | 57.4970 | 15.4% | n.a. |
| 14.886° | 5.94654 | Å | 135.028 | 36.1% | n.a. |
| 15.356° | 5.76559 | Å | 79.9504 | 21.4% | n.a. |
| 16.008° | 5.53207 | Å | 58.3128 | 15.6% | n.a. |
| 16.549° | 5.35229 | Å | 38.7512 | 10.4% | n.a. |
| 17.239° | 5.13967 | Å | 61.2088 | 16.4% | n.a. |
| 17.639° | 5.02401 | Å | 81.4934 | 21.8% | n.a. |
| 18.025° | 4.91732 | Å | 65.9356 | 17.6% | n.a. |
| 18.274° | 4.85077 | Å | 129.001 | 34.5% | n.a. |
| 19.085° | 4.64660 | Å | 235.966 | 63.2% | n.a. |
| 19.652° | 4.51372 | Å | 114.148 | 30.6% | n.a. |
| 19.974° | 4.44172 | Å | 75.1744 | 20.1% | n.a. |
| 20.870° | 4.25304 | Å | 68.7504 | 18.4% | n.a. |
| 21.723° | 4.08794 | Å | 37.4362 | 10.0% | n.a. |
| 22.388° | 3.96796 | Å | 130.793 | 35.0% | n.a. |
| 23.097° | 3.84773 | Å | 104.286 | 27.9% | n.a. |
| 23.817° | 3.73301 | Å | 43.5751 | 11.7% | n.a. |
| 24.230° | 3.67023 | Å | 52.3532 | 14.0% | n.a. |
| 25.300° | 3.51747 | Å | 72.3767 | 19.4% | n.a. |
| 26.344° | 3.38033 | Å | 56.2048 | 15.0% | n.a. |
| 27.400° | 3.25241 | Å | 29.6865 | 7.9% | n.a. |
| 28.326° | 3.14813 | Å | 55.3924 | 14.8% | n.a. |
| 30.967° | 2.88543 | Å | 18.6466 | 5.0% | n.a. |

In some embodiments, the crystalline Form A-4 of Compound I tosylate salt exhibits a DSC thermogram that is substantially similar to FIG. 12B.

### Compound I Fumarate Salt

In one embodiment, the present disclosure relates to a Compound I fumarate salt, or a solvate thereof. In embodiments, the crystalline form of Compound I fumarate salt comprises a mixture of one or more forms of polymorphs of Compound I fumarate salt. In embodiments, the crystalline form of Compound I fumarate salt comprises of substantially pure form of one polymorph type.

### Compound I Fumarate Salt Form A-5

In embodiments, the present disclosure relates to a crystalline form of Compound I fumarate salt, which is Form A-5.

In embodiments, the crystalline form of Compound I fumarate salt may comprise of over about 99.9%, about 99.8%, about 99.7%, about 99.6%, about 99.5%, about 99.4%, about 99.3%, about 99.2%, about 99.1%, or about 99.0% of Form A-5 of Compound I fumarate salt. In another embodiment, the crystalline form of Compound I fumarate salt may comprise over about 99%, 98%, 97%, 96%, 95%, 94%, 93%, 92%, 91%, or 90% of Form A-5 of Compound I fumarate salt. In some embodiments, the crystalline form of Compound I fumarate salt may comprise over about 90%, 85%, 80%, 75%, 70%, 65%, 60%, 55%, 50%, 45%, or 40% of Form A-5 of Compound I fumarate salt.

In embodiments, crystalline Form A-5 of Compound I fumarate salt exhibits an XRPD pattern comprising peaks at about 18.12, 23.11, and 23.59 degrees two-theta with the margin of error of about ±0.5; about ±0.4; about ±0.3; about ±0.2; about ±0.1; about ±0.05; or less. In embodiments, crystalline Form A-5 of Compound I fumarate salt exhibits an XRPD pattern comprising peaks at about 5.31, 18.12, 19.79, 23.11, and 23.59 degrees two-theta with the margin of error of about ±0.5; about ±0.4; about ±0.3; about ±0.2; about ±0.1; about ±0.05; or less. In embodiments, crystalline Form A-5 of Compound I fumarate salt exhibits an XRPD pattern which further comprises at least two peaks selected from about 8.50, 9.42, 13.23, 19.12, 21.16, 25.17, 25.68 and 28.82 degrees two-theta with the margin of error of about ±0.5; about ±0.4; about ±0.3; about ±0.2; about ±0.1; about ±0.05; or less. In embodiments, crystalline Form A-5 of Compound I fumarate salt exhibits an XRPD pattern which further comprises at least three peaks selected from about 8.50, 9.42, 13.23, 19.12, 21.16, 25.17, 25.68 and 28.82 degrees two-theta with the margin of error of about ±0.5; about ±0.4; about ±0.3; about ±0.2; about ±0.1; about ±0.05; or less. In embodiments, crystalline Form A-5 of Compound I fumarate salt exhibits an XRPD pattern which further comprises at least four peaks selected from about 8.50, 9.42, 13.23, 19.12, 21.16, 25.17, 25.68 and 28.82 degrees two-theta with the margin of error of about ±0.5; about ±0.4; about ±0.3; about ±0.2; about ±0.1; about ±0.05; or less. In embodiments, crystalline Form A-5 of Compound I fumarate salt exhibits an XRPD pattern which further comprises at least two five selected from about 8.50, 9.42, 13.23, 19.12, 21.16, 25.17, 25.68 and 28.82 degrees two-theta with the margin of error of about ±0.5; about ±0.4; about ±0.3; about ±0.2; about ±0.1; about ±0.05; or less. In embodiments, crystalline Form A-5 of Compound I fumarate salt exhibits an XRPD pattern which further comprises at least six peaks selected from about 8.50, 9.42, 13.23, 19.12, 21.16, 25.17, 25.68 and 28.82 degrees two-theta with the margin of error of about ±0.5; about ±0.4; about ±0.3; about ±0.2; about ±0.1; about ±0.05; or less. In embodiments, crystalline Form A-5 of Compound I fumarate salt exhibits an XRPD pattern which further comprises at least seven peaks selected from about 8.50, 9.42, 13.23, 19.12, 21.16, 25.17, 25.68 and 28.82 degrees two-theta with the margin of error of about ±0.5; about ±0.4; about ±0.3; about ±0.2; about ±0.1; about ±0.05; or less. In embodiments, crystalline Form A-5 of Compound I fumarate salt exhibits an XRPD pattern which further peaks at about 8.50, 9.42, 13.23, 19.12, 21.16, 25.17, 25.68 and 28.82 degrees two-theta with the margin of error of about ±0.5; about ±0.4; about ±0.3; about ±0.2; about ±0.1; about ±0.05; or less.

In embodiments, crystalline Form A-5 of Compound I fumarate salt exhibits an XRPD pattern comprising peaks at about 18.12±0.2, 23.11±0.2, and 23.59±0.2 degrees two-theta.

In embodiments, crystalline Form A-5 of Compound I fumarate salt exhibits an XRPD pattern comprising peaks at about 5.31±0.2, 18.12±0.2, 19.79±0.2, 23.11±0.2, and 23.59±0.2 degrees two-theta.

In embodiments, crystalline Form A-5 of Compound I fumarate salt exhibits an XRPD pattern which further comprises at least two peaks selected from about 8.50±0.2, 9.42±0.2, 13.23±0.2, 19.12±0.2, 21.16±0.2, 25.17±0.2, 25.68±0.2 and 28.82±0.2 degrees two-theta.

In some embodiments, the crystalline form of Compound I fumarate salt exhibits an XRPD pattern comprising 1 peak, 2 peaks, 3 peaks, 4 peaks, 5 peaks, 6 peaks, 7 peaks, 8 peaks, 9 peaks, or 10 peaks ±0.2 degrees two-theta of Table 13.

In embodiments, crystalline Form A-5 of Compound I fumarate salt exhibits an XRPD pattern comprising peaks shown in Table 13, below.

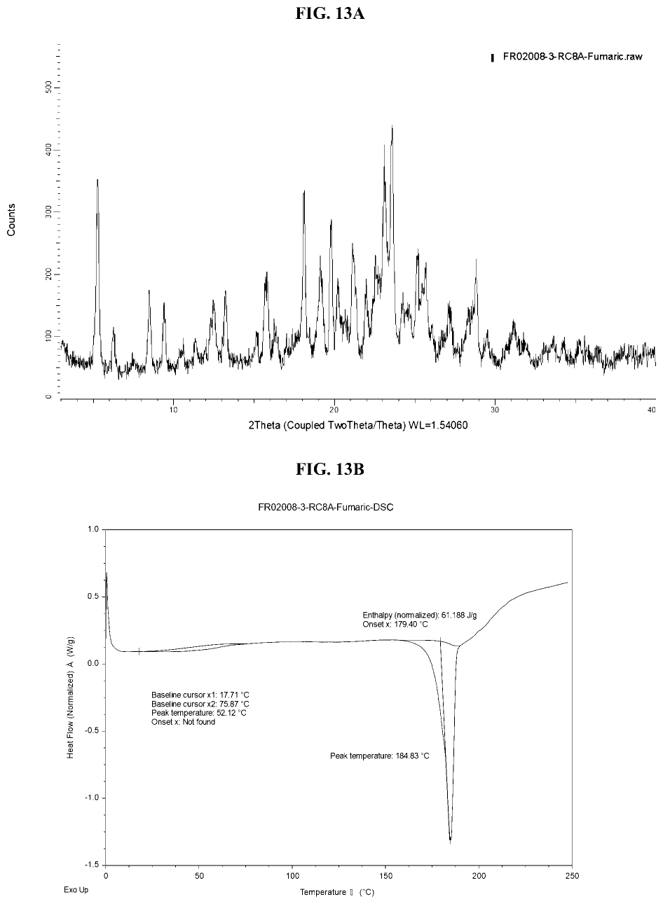

In embodiments, crystalline Form A-5 of Compound I fumarate salt exhibits an XRPD pattern that is substantially similar to FIG. 13A.

TABLE 13
Form A-5 of Compound I fumarate salt

| Angle | d Value | | Net Intensity | Rel. Intensity |
|---|---|---|---|---|
| 5.306° | 16.64265 | Å | 175.511 | 74.1% |
| 6.256° | 14.11598 | Å | 35.9078 | 15.2% |
| 8.495° | 10.39977 | Å | 85.2226 | 36.0% |
| 9.420° | 9.38127 | Å | 77.6747 | 32.8% |
| 10.529° | 8.39536 | Å | 13.4606 | 5.7% |
| 11.366° | 7.77878 | Å | 18.9852 | 8.0% |
| 12.484° | 7.08481 | Å | 66.8012 | 28.2% |
| 13.225° | 6.68925 | Å | 80.7785 | 34.1% |
| 15.172° | 5.83509 | Å | 25.0285 | 10.6% |
| 15.771° | 5.61471 | Å | 58.9621 | 24.9% |
| 16.339° | 5.42063 | Å | 44.5116 | 18.8% |
| 18.119° | 4.89211 | Å | 181.758 | 76.7% |
| 19.123° | 4.63743 | Å | 96.4633 | 40.7% |
| 19.789° | 4.48284 | Å | 128.653 | 54.3% |
| 20.209° | 4.39056 | Å | 66.9878 | 28.3% |
| 21.156° | 4.19607 | Å | 104.070 | 43.9% |
| 21.970° | 4.04244 | Å | 62.3233 | 26.3% |
| 22.641° | 3.92415 | Å | 77.5668 | 32.7% |
| 23.106° | 3.84622 | Å | 201.575 | 85.1% |
| 23.588° | 3.76867 | Å | 236.911 | 100.0% |
| 24.272° | 3.66403 | Å | 66.4168 | 28.0% |
| 24.575° | 3.61961 | Å | 53.0785 | 22.4% |
| 25.174° | 3.53481 | Å | 100.335 | 42.4% |
| 25.683° | 3.46581 | Å | 83.9560 | 35.4% |
| 26.096° | 3.41192 | Å | 32.6019 | 13.8% |
| 27.167° | 3.27985 | Å | 53.4042 | 22.5% |
| 28.360° | 3.14450 | Å | 49.2602 | 20.8% |
| 28.823° | 3.09503 | Å | 106.306 | 44.9% |
| 29.495° | 3.02599 | Å | 32.4296 | 13.7% |
| 31.158° | 2.86822 | Å | 39.8069 | 16.8% |
| 33.168° | 2.69886 | Å | 23.2008 | 9.8% |
| 34.220° | 2.61825 | Å | 21.8609 | 9.2% |
| 35.270° | 2.54264 | Å | 25.7470 | 10.9% |
| 36.498° | 2.45988 | Å | 20.1993 | 8.5% |

In embodiments, crystalline Form A-5 of Compound I fumarate salt exhibits a DSC thermogram comprising an endotherm peak at about 52° C. with the error of margin of about ±2.5; about ±2.0; about ±1.5; about ±1.0; about ±0.5; or less. In embodiments, the crystalline Form A-5 of Compound I fumarate salt exhibits a DSC thermogram comprising an endotherm peak with an onset at about 179° C. with the error of margin of about ±2.5; about ±2.0; about ±1.5; about ±1.0; about ±0.5; or less. In embodiments, the crystalline Form A-5 of Compound I fumarate salt exhibits a DSC thermogram comprising an endotherm peak at about 184° C. with the error of margin of about ±2.5; about ±2.0; about ±1.5; about ±1.0; about ±0.5; or less.

In some embodiments, the crystalline Form A-5 of Compound I fumarate salt exhibits a DSC thermogram that is substantially similar to FIG. 13B.

### Compound I L-Tartrate Salt

In one embodiment, the present disclosure relates to a Compound I L-tartrate salt, or a solvate thereof. In embodiments, the crystalline form of Compound I L-tartrate comprises a mixture of one or more forms of polymorphs of Compound I L-tartrate. In embodiments, the crystalline form of Compound I L-tartrate salt comprises of substantially pure form of one polymorph type.

### Compound I L-Tartrate Salt Form A-7

In embodiments, the present disclosure relates to a crystalline form of Compound I L-tartrate salt, which is Form A-7.

In embodiments, the crystalline form of Compound I L-tartrate salt may comprise of over about 99.9%, about 99.8%, about 99.7%, about 99.6%, about 99.5%, about 99.4%, about 99.3%, about 99.2%, about 99.1%, or about 99.0% of Form A-7 of Compound I L-tartrate salt. In another embodiment, the crystalline form of Compound I L-tartrate salt may comprise over about 99%, 98%, 97%, 96%, 95%, 94%, 93%, 92%, 91%, or 90% of Form A-7 of Compound I L-tartrate salt. In some embodiments, the crystalline form of Compound I L-tartrate salt may comprise over about 90%, 85%, 80%, 75%, 70%, 65%, 60%, 55%, 50%, 45%, or 40% of Form A-7 of Compound I L-tartrate salt.

In embodiments, crystalline Form A-7 of Compound I L-tartrate salt exhibits an XRPD pattern comprising peaks at about 5.61, 6.93, and 19.66 degrees two-theta with the margin of error of about ±0.5; about ±0.4; about ±0.3; about ±0.2; about ±0.1; about ±0.05; or less. In embodiments, crystalline Form A-7 of Compound I L-tartrate salt exhibits an XRPD pattern comprising peaks at about 5.61, 6.93, 17.51, 19.66, and 21.75 degrees two-theta with the margin of error of about ±0.5; about ±0.4; about ±0.3; about ±0.2; about ±0.1; about ±0.05; or less.

In embodiments, crystalline Form A-7 of Compound I L-tartrate salt exhibits an XRPD pattern comprising peaks at about 5.61±0.2, 6.93±0.2, and 19.66±0.2 degrees two-theta.

In embodiments, crystalline Form A-7 of Compound I L-tartrate salt exhibits an XRPD pattern comprising peaks at about 5.61±0.2, 6.93±0.2, 17.51±0.2, 19.66±0.2, and 21.75±0.2 degrees two-theta.

In some embodiments, the crystalline form of Compound I L-tartrate salt exhibits an XRPD pattern comprising 1 peak, 2 peaks, 3 peaks, 4 peaks, 5 peaks, 6 peaks, 7 peaks, 8 peaks, 9 peaks, or 10 peaks ±0.2 degrees two-theta of Table 15.

In embodiments, crystalline Form A-7 of Compound I L-tartrate salt exhibits an XRPD pattern comprising peaks shown in Table 15, below.

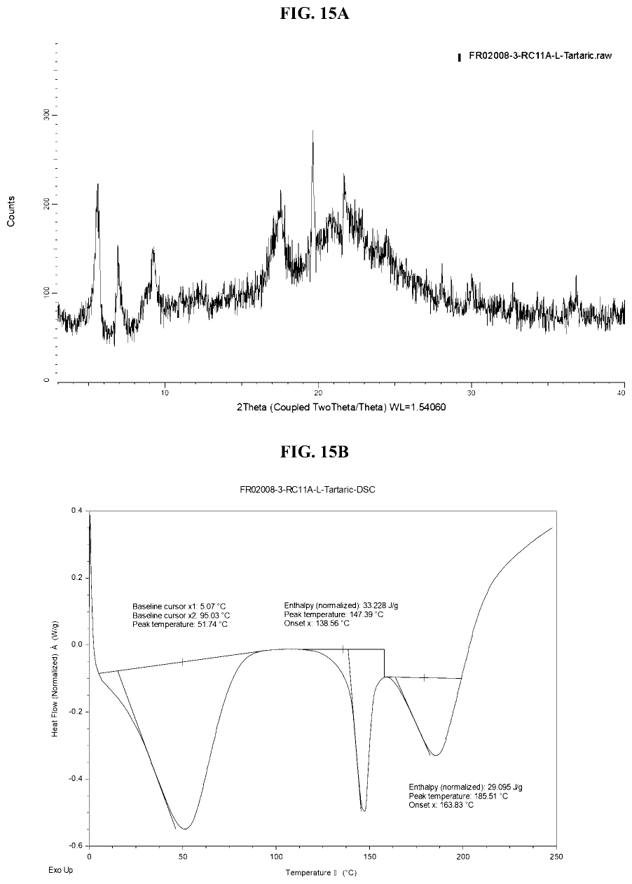

In embodiments, crystalline Form A-7 of Compound I L-tartrate salt exhibits an XRPD pattern that is substantially similar to FIG. 15A.

TABLE 15
Form A-7 of Compound I L-tartrate

| Angle | d Value | | Net Intensity | Rel. Intensity |
|---|---|---|---|---|
| 5.606° | 15.75112 | Å | 99.7855 | 100.0% |
| 6.932° | 12.74201 | Å | 56.3552 | 56.5% |
| 9.281° | 9.52133 | Å | 44.3864 | 44.5% |
| 17.514° | 5.05974 | Å | 46.4117 | 46.5% |
| 19.658° | 4.51243 | Å | 92.5735 | 92.8% |
| 21.750° | 4.08277 | Å | 44.8981 | 45.0% |
| 28.085° | 3.17468 | Å | 34.2472 | 34.3% |
| 36.826° | 2.43868 | Å | 43.9121 | 44.0% |

In embodiments, crystalline Form A-7 of Compound I L-tartrate salt exhibits a DSC thermogram comprising an endotherm peak at about 52° C. with the error of margin of about ±2.5; about ±2.0; about ±1.5; about ±1.0; about ±0.5; or less. In embodiments, the crystalline Form A-7 of Compound I L-tartrate salt exhibits a DSC thermogram comprising an endotherm peak with an onset at about 139° C. with the error of margin of about ±2.5; about ±2.0; about ±1.5; about ±1.0; about ±0.5; or less. In embodiments, the crystalline Form A-7 of Compound I L-tartrate salt exhibits a DSC thermogram comprising an endotherm peak at about 147° C. with the error of margin of about ±2.5; about ±2.0; about ±1.5; about ±1.0; about ±0.5; or less. In embodiments, the crystalline Form A-7 of Compound I L-tartrate salt exhibits a DSC thermogram comprising an endotherm peak with an onset at about 164° C. with the error of margin of about ±2.5; about ±2.0; about ±1.5; about ±1.0; about ±0.5; or less. In embodiments, the crystalline Form A-7 of Compound I L-tartrate salt exhibits a DSC thermogram comprising an endotherm peak at about 186° C. with the error of margin of about ±2.5; about ±2.0; about ±1.5; about ±1.0; about ±0.5; or less.

In some embodiments, the crystalline Form A-1 of Compound I hydrochloride salt exhibits a DSC thermogram that is substantially similar to FIG. 15B.

### Compound I L-Tartrate Salt Form B-7

In embodiments, the present disclosure relates to a crystalline form of Compound I L-tartrate salt, which is Form B-7.

In embodiments, the crystalline form of Compound I L-tartrate salt may comprise of over about 99.9%, about 99.8%, about 99.7%, about 99.6%, about 99.5%, about 99.4%, about 99.3%, about 99.2%, about 99.1%, or about 99.0% of Form B-7 of Compound I L-tartrate salt. In another embodiment, the crystalline form of Compound I L-tartrate salt may comprise over about 99%, 98%, 97%, 96%, 95%, 94%, 93%, 92%, 91%, or 90% of Form B-7 of Compound I L-tartrate salt. In some embodiments, the crystalline form of Compound I L-tartrate salt may comprise over about 90%, 85%, 80%, 75%, 70%, 65%, 60%, 55%, 50%, 45%, or 40% of Form B-7 of Compound I L-tartrate salt.

In embodiments, crystalline Form B-7 of Compound I L-tartrate salt exhibits an XRPD pattern comprising peaks at about 5.36 degrees two-theta with the margin of error of about ±0.5; about ±0.4; about ±0.3; about ±0.2; about ±0.1; about ±0.05; or less.

In embodiments, crystalline Form B-7 of Compound I L-tartrate salt exhibits an XRPD pattern comprising peaks at about 5.36±0.2 degrees two-theta.

In some embodiments, the crystalline form of Compound I L-tartrate salt exhibits an XRPD pattern comprising 1 peak, 2 peaks, 3 peaks, 4 peaks, 5 peaks, 6 peaks, 7 peaks, 8 peaks, 9 peaks, or 10 peaks ±0.2 degrees two-theta of Table 16.

In embodiments, crystalline Form B-7 of Compound I L-tartrate salt exhibits an XRPD pattern comprising peaks shown in Table 16, below.

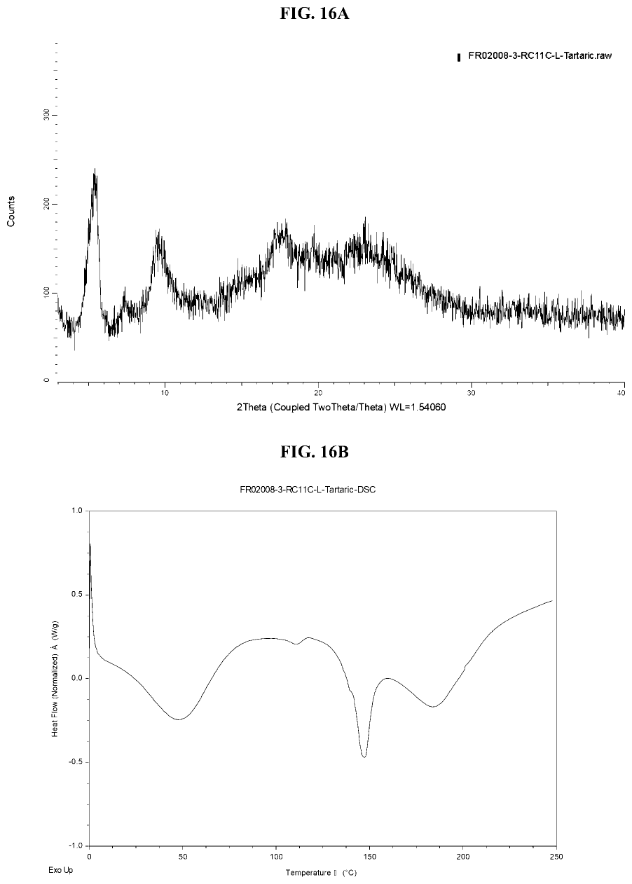

In embodiments, crystalline Form B-7 of Compound I L-tartrate salt exhibits an XRPD pattern that is substantially similar to FIG. 16A.

TABLE 16
Form B-7 of Compound I L-tartrate

| Angle | d Value | Net Intensity | Rel. Intensity |
|---|---|---|---|
| 5.362° | 16.46962 Å | 109.185 | 100.0% |

In some embodiments, the crystalline Form B-7 of Compound I L-tartrate salt exhibits a DSC thermogram that is substantially similar to FIG. 16B.

### Compound I Citrate Salt

In one embodiment, the present disclosure relates to a Compound I citrate salt, or a solvate thereof. In embodiments, the crystalline form of Compound I citrate salt comprises a mixture of one or more forms of polymorphs of Compound I citrate salt. In embodiments, the crystalline form of Compound I citrate salt comprises of substantially pure form of one polymorph type.

### Compound I Citrate Salt Form A-8

In embodiments, the present disclosure relates to a crystalline form of Compound I citrate salt, which is Form A-8.

In embodiments, the crystalline form of Compound I citrate salt may comprise of over about 99.9%, about 99.8%, about 99.7%, about 99.6%, about 99.5%, about 99.4%, about 99.3%, about 99.2%, about 99.1%, or about 99.0% of Form A-8 of Compound I citrate salt. In another embodiment, the crystalline form of Compound I citrate salt may comprise over about 99%, 98%, 97%, 96%, 95%, 94%, 93%, 92%, 91%, or 90% of Form A-8 of Compound I citrate salt. In some embodiments, the crystalline form of Compound I citrate salt may comprise over about 90%, 85%, 80%, 75%, 70%, 65%, 60%, 55%, 50%, 45%, or 40% of Form A-8 of Compound I citrate salt.

In embodiments, crystalline Form A-8 of Compound I citrate salt exhibits an XRPD pattern comprising peaks at about 4.56, 9.15, and 12.05 degrees two-theta with the margin of error of about ±0.5; about ±0.4; about ±0.3; about ±0.2; about ±0.1; about ±0.05; or less. In embodiments, crystalline Form A-8 of Compound I citrate salt exhibits an XRPD pattern comprising peaks at about 4.56, 9.15, 12.05, 17.43, and 18.63 degrees two-theta with the margin of error of about ±0.5; about ±0.4; about ±0.3; about ±0.2; about ±0.1; about ±0.05; or less. In embodiments, crystalline Form A-8 of Compound I citrate salt exhibits an XRPD pattern further comprises at least two peaks selected from about 7.94±0.2, 15.77±0.2, 18.27±0.2, 19.10±0.2, 20.23±0.2, 20.88±0.2, 22.31±0.2, and 24.02±0.2 degrees two-theta with the margin of error of about ±0.5; about ±0.4; about ±0.3; about ±0.2; about ±0.1; about ±0.05; or less. In embodiments, crystalline Form A-8 of Compound I citrate salt exhibits an XRPD pattern further comprises at least three peaks selected from about 7.94±0.2, 15.77±0.2, 18.27±0.2, 19.10±0.2, 20.23±0.2, 20.88±0.2, 22.31±0.2, and 24.02±0.2 degrees two-theta with the margin of error of about ±0.5; about ±0.4; about ±0.3; about ±0.2; about ±0.1; about ±0.05; or less. In embodiments, crystalline Form A-8 of Compound I citrate salt exhibits an XRPD pattern further comprises at least four peaks selected from about 7.94±0.2, 15.77±0.2, 18.27±0.2, 19.10±0.2, 20.23±0.2, 20.88±0.2, 22.31±0.2, and 24.02±0.2 degrees two-theta with the margin of error of about ±0.5; about ±0.4; about ±0.3; about ±0.2; about ±0.1; about ±0.05; or less. In embodiments, crystalline Form A-8 of Compound I citrate salt exhibits an XRPD pattern further comprises at least five peaks selected from about 7.94±0.2, 15.77±0.2, 18.27±0.2, 19.10±0.2, 20.23±0.2, 20.88±0.2, 22.31±0.2, and 24.02±0.2 degrees two-theta with the margin of error of about ±0.5; about ±0.4; about ±0.3; about ±0.2; about ±0.1; about ±0.05; or less. In embodiments, crystalline Form A-8 of Compound I citrate salt exhibits an XRPD pattern further comprises at least six peaks selected from about 7.94±0.2, 15.77±0.2, 18.27±0.2, 19.10±0.2, 20.23±0.2, 20.88±0.2, 22.31±0.2, and 24.02±0.2 degrees two-theta with the margin of error of about ±0.5; about ±0.4; about ±0.3; about ±0.2; about ±0.1; about ±0.05; or less. In embodiments, crystalline Form A-8 of Compound I citrate salt exhibits an XRPD pattern further comprises at least seven peaks selected from about 7.94±0.2, 15.77±0.2, 18.27±0.2, 19.10±0.2, 20.23±0.2, 20.88±0.2, 22.31±0.2, and 24.02±0.2 degrees two-theta with the margin of error of about ±0.5; about ±0.4; about ±0.3; about ±0.2; about ±0.1; about ±0.05; or less. In embodiments, crystalline Form A-8 of Compound I citrate salt exhibits an XRPD pattern further comprises peaks at about 7.94±0.2, 15.77±0.2, 18.27±0.2, 19.10±0.2, 20.23±0.2, 20.88±0.2, 22.31±0.2, and 24.02±0.2 degrees two-theta with the margin of error of about ±0.5; about ±0.4; about ±0.3; about ±0.2; about ±0.1; about ±0.05; or less.

In embodiments, crystalline Form A-8 of Compound I citrate salt exhibits an XRPD pattern comprising peaks at about 4.56±0.2, 9.15±0.2, and 12.05±0.2 degrees two-theta.

In embodiments, crystalline Form A-8 of Compound I citrate salt exhibits an XRPD pattern comprising peaks at about 4.56±0.2, 9.15±0.2, 12.05±0.2, 17.43±0.2, and 18.63±0.2 degrees two-theta.

In embodiments, crystalline Form A-8 of Compound I citrate salt exhibits an XRPD pattern further comprises at least two peaks selected from about 7.94±0.2, 15.77±0.2, 18.27±0.2, 19.10±0.2, 20.23±0.2, 20.88±0.2, 22.31±0.2, and 24.02±0.2 degrees two-theta.

In some embodiments, the crystalline form of Compound I citrate salt exhibits an XRPD pattern comprising 1 peak, 2 peaks, 3 peaks, 4 peaks, 5 peaks, 6 peaks, 7 peaks, 8 peaks, 9 peaks, or 10 peaks ±0.2 degrees two-theta of Table 17.

In embodiments, crystalline Form A-8 of Compound I citrate salt exhibits an XRPD pattern comprising peaks shown in Table 17, below.

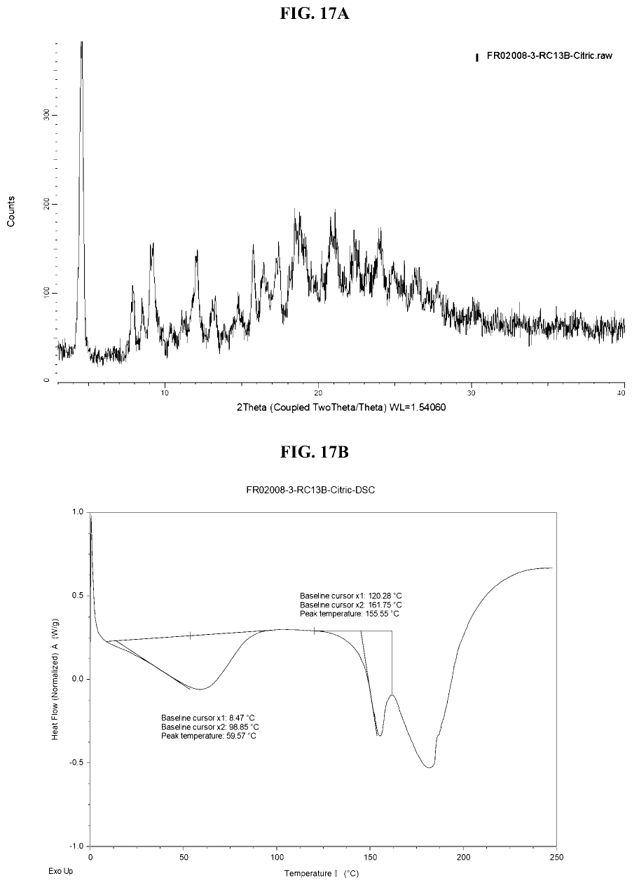

In embodiments, crystalline Form A-8 of Compound I citrate salt exhibits an XRPD pattern that is substantially similar to FIG. 17A.

TABLE 17
Form A-8 of Compound I citrate salt

| Angle | d Value | | Net Intensity | Rel. Intensity |
|---|---|---|---|---|
| 4.556° | 19.38114 | Å | 232.479 | 100.0% |
| 7.940° | 11.12564 | Å | 46.2643 | 19.9% |
| 8.525° | 10.36365 | Å | 28.4360 | 12.2% |
| 9.154° | 9.65285 | Å | 72.9070 | 31.4% |
| 9.842° | 8.97951 | Å | 14.6396 | 6.3% |
| 12.050° | 7.33912 | Å | 62.9606 | 27.1% |
| 13.181° | 6.71156 | Å | 17.4981 | 7.5% |
| 15.774° | 5.61353 | Å | 51.7948 | 22.3% |
| 16.405° | 5.39901 | Å | 34.0732 | 14.7% |
| 17.426° | 5.08512 | Å | 56.5565 | 24.3% |
| 18.269° | 4.85217 | Å | 36.0159 | 15.5% |
| 18.628° | 4.75956 | Å | 60.1131 | 25.9% |
| 19.103° | 4.64221 | Å | 45.1427 | 19.4% |
| 20.229° | 4.38635 | Å | 34.2751 | 14.7% |
| 20.876° | 4.25182 | Å | 49.0140 | 21.1% |
| 22.309° | 3.98180 | Å | 40.0847 | 17.2% |
| 24.015° | 3.70268 | Å | 35.6779 | 15.3% |
| 27.817° | 3.20465 | Å | 28.8130 | 12.4% |

In embodiments, crystalline Form A-8 of Compound I citrate salt exhibits a.

In embodiments, crystalline Form A-8 of Compound I citrate salt exhibits a DSC thermogram comprising an endotherm peak at about 60° C. with the error of margin of about ±2.5; about ±2.0; about ±1.5; about ±1.0; about ±0.5; or less. In embodiments, the crystalline Form A-8 of Compound I citrate salt exhibits a DSC thermogram comprising an endotherm peak at about 156° C. with the error of margin of about ±2.5; about ±2.0; about ±1.5; about ±1.0; about ±0.5; or less.

In some embodiments, the crystalline Form A-8 of Compound I citrate salt exhibits a DSC thermogram that is substantially similar to FIG. 17B.

### Compound I Citrate Salt Form B-8

In embodiments, the present disclosure relates to a crystalline form of Compound I citrate salt, which is Form B-8.

In embodiments, the crystalline form of Compound I citrate salt may comprise of over about 99.9%, about 99.8%, about 99.7%, about 99.6%, about 99.5%, about 99.4%, about 99.3%, about 99.2%, about 99.1%, or about 99.0% of Form B-8 of Compound I citrate salt. In another embodiment, the crystalline form of Compound I citrate salt may comprise over about 99%, 98%, 97%, 96%, 95%, 94%, 93%, 92%, 91%, or 90% of Form B-8 of Compound I citrate salt. In some embodiments, the crystalline form of Compound I citrate salt may comprise over about 90%, 85%, 80%, 75%, 70%, 65%, 60%, 55%, 50%, 45%, or 40% of Form B-8 of Compound I citrate salt.

In embodiments, crystalline Form B-8 of Compound I citrate salt exhibits an XRPD pattern comprising peaks at about 5.36, 6.85, and 20.59 degrees two-theta with the margin of error of about ±0.5; about ±0.4; about ±0.3; about ±0.2; about ±0.1; about ±0.05; or less. In embodiments, crystalline Form B-8 of Compound I citrate salt exhibits an XRPD pattern comprising peaks at about 5.36, 6.85, 17.81, 20.59, and 22.81 degrees two-theta with the margin of error of about ±0.5; about ±0.4; about ±0.3; about ±0.2; about ±0.1; about ±0.05; or less. In embodiments, crystalline Form B-8 of Compound I citrate salt exhibits an XRPD pattern which further comprises at least two peaks selected from about 16.08, 21.20, 25.81, and 27.02 degrees two-theta with the margin of error of about ±0.5; about ±0.4; about ±0.3; about ±0.2; about ±0.1; about ±0.05; or less. In embodiments, crystalline Form B-8 of Compound I citrate salt exhibits an XRPD pattern which further comprises at least three peaks selected from about 16.08, 21.20, 25.81, and 27.02 degrees two-theta with the margin of error of about ±0.5; about ±0.4; about ±0.3; about ±0.2; about ±0.1; about ±0.05; or less. In embodiments, crystalline Form B-8 of Compound I citrate salt exhibits an XRPD pattern which further comprises peaks at about 16.08, 21.20, 25.81, and 27.02 degrees two-theta with the margin of error of about ±0.5; about ±0.4; about ±0.3; about ±0.2; about ±0.1; about ±0.05; or less.

In embodiments, crystalline Form B-8 of Compound I citrate salt exhibits an XRPD pattern comprising peaks at about 5.36±0.2, 6.85±0.2, and 20.59±0.2 degrees two-theta.

In embodiments, crystalline Form B-8 of Compound I citrate salt exhibits an XRPD pattern comprising peaks at about 5.36±0.2, 6.85±0.2, 17.81±0.2, 20.59±0.2, and 22.81±0.2 degrees two-theta.

In embodiments, crystalline Form B-8 of Compound I citrate salt exhibits an XRPD pattern which further comprises at least two peaks selected from about 16.08±0.2, 21.20±0.2, 25.81±0.2, and 27.02±0.2 degrees two-theta.

In some embodiments, the crystalline form of Compound I citrate salt exhibits an XRPD pattern comprising 1 peak, 2 peaks, 3 peaks, 4 peaks, 5 peaks, 6 peaks, 7 peaks, 8 peaks, 9 peaks, or 10 peaks ±0.2 degrees two-theta of Table 18.

In embodiments, crystalline Form B-8 of Compound I citrate salt exhibits an XRPD pattern comprising peaks shown in Table 18, below.

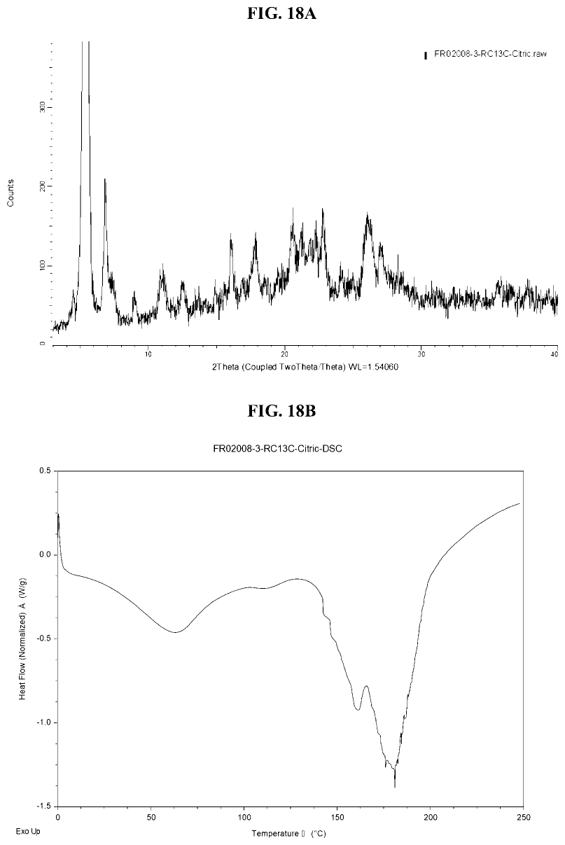

In embodiments, crystalline Form B-8 of Compound I citrate salt exhibits an XRPD pattern that is substantially similar to FIG. 18A.

TABLE 18
Form B-8 of Compound I citrate salt

| Angle | d Value | | Net Intensity | Rel. Intensity |
|---|---|---|---|---|
| 4.489° | 19.66764 | Å | 22.1149 | 2.9% |
| 5.359° | 16.47773 | Å | 770.679 | 100.0% |
| 6.850° | 12.89304 | Å | 104.000 | 13.5% |
| 7.362° | 11.99746 | Å | 22.7685 | 3.0% |
| 8.983° | 9.83620 | Å | 28.0863 | 3.6% |
| 11.009° | 8.03052 | Å | 24.1355 | 3.1% |
| 12.551° | 7.04724 | Å | 18.1809 | 2.4% |
| 16.081° | 5.50707 | Å | 46.6443 | 6.1% |
| 17.808° | 4.97665 | Å | 52.1114 | 6.8% |
| 20.588° | 4.31063 | Å | 70.7939 | 9.2% |
| 21.196° | 4.18830 | Å | 47.8422 | 6.2% |
| 22.257° | 3.99091 | Å | 22.2716 | 2.9% |
| 22.810° | 3.89551 | Å | 56.4444 | 7.3% |
| 24.209° | 3.67337 | Å | 20.0818 | 2.6% |
| 25.810° | 3.44904 | Å | 49.3728 | 6.4% |
| 27.022° | 3.29712 | Å | 42.7260 | 5.5% |

In some embodiments, the crystalline Form B-8 of Compound I citrate salt exhibits a DSC thermogram that is substantially similar to FIG. 18B.

### Compound I Succinate Salt Form A-9

In one embodiment, the present disclosure relates to a Compound I succinate salt, or a solvate thereof. In embodiments, the present disclosure relates to a crystalline form of Compound I succinate salt, which is Form A-9.

In embodiments, the crystalline form of Compound I succinate salt may comprise of over about 99.9%, about 99.8%, about 99.7%, about 99.6%, about 99.5%, about 99.4%, about 99.3%, about 99.2%, about 99.1%, or about 99.0% of Form A-9 of Compound I succinate salt. In another embodiment, the crystalline form of Compound I succinate salt may comprise over about 99%, 98%, 97%, 96%, 95%, 94%, 93%, 92%, 91%, or 90% of Form A-9 of Compound I succinate salt. In some embodiments, the crystalline form of Compound I succinate salt may comprise over about 90%, 85%, 80%, 75%, 70%, 65%, 60%, 55%, 50%, 45%, or 40% of Form A-9 of Compound I succinate salt.

In embodiments, crystalline Form A-9 of Compound I succinate salt exhibits an XRPD pattern comprising peaks at about 8.73, 20.05, and 26.15 degrees two-theta with the margin of error of about ±0.5; about ±0.4; about ±0.3; about ±0.2; about ±0.1; about ±0.05; or less. In embodiments, crystalline Form A-9 of Compound I succinate salt exhibits an XRPD pattern comprising peaks at about 4.08, 8.16, 8.73, 20.05, and 26.15 degrees two-theta with the margin of error of about ±0.5; about ±0.4; about ±0.3; about ±0.2; about ±0.1; about ±0.05; or less. In embodiments, crystalline Form A-9 of Compound I succinate salt exhibits an XRPD pattern which further comprises at least two peaks selected from about 6.76, 8.96, 12.30, 19.63, 21.10, 22.76, 25.88, and 31.55 degrees two-theta with the margin of error of about ±0.5; about ±0.4; about ±0.3; about ±0.2; about ±0.1; about ±0.05; or less. In embodiments, crystalline Form A-9 of Compound I succinate salt exhibits an XRPD pattern which further comprises at least three peaks selected from about 6.76, 8.96, 12.30, 19.63, 21.10, 22.76, 25.88, and 31.55 degrees two-theta with the margin of error of about ±0.5; about ±0.4; about ±0.3; about ±0.2; about ±0.1; about ±0.05; or less. In embodiments, crystalline Form A-9 of Compound I succinate salt exhibits an XRPD pattern which further comprises at least four peaks selected from about 6.76, 8.96, 12.30, 19.63, 21.10, 22.76, 25.88, and 31.55 degrees two-theta with the margin of error of about ±0.5; about ±0.4; about ±0.3; about ±0.2; about ±0.1; about ±0.05; or less. In embodiments, crystalline Form A-9 of Compound I succinate salt exhibits an XRPD pattern which further comprises at least five peaks selected from about 6.76, 8.96, 12.30, 19.63, 21.10, 22.76, 25.88, and 31.55 degrees two-theta with the margin of error of about ±0.5; about ±0.4; about ±0.3; about ±0.2; about ±0.1; about ±0.05; or less. In embodiments, crystalline Form A-9 of Compound I succinate salt exhibits an XRPD pattern which further comprises at least six peaks selected from about 6.76, 8.96, 12.30, 19.63, 21.10, 22.76, 25.88, and 31.55 degrees two-theta with the margin of error of about ±0.5; about ±0.4; about ±0.3; about ±0.2; about ±0.1; about ±0.05; or less. In embodiments, crystalline Form A-9 of Compound I succinate salt exhibits an XRPD pattern which further comprises at least seven peaks selected from about 6.76, 8.96, 12.30, 19.63, 21.10, 22.76, 25.88, and 31.55 degrees two-theta with the margin of error of about ±0.5; about ±0.4; about ±0.3; about ±0.2; about ±0.1; about ±0.05; or less. In embodiments, crystalline Form A-9 of Compound I succinate salt exhibits an XRPD pattern which further comprises peaks at 6.76, 8.96, 12.30, 19.63, 21.10, 22.76, 25.88, and 31.55 degrees two-theta with the margin of error of about ±0.5; about ±0.4; about ±0.3; about ±0.2; about ±0.1; about ±0.05; or less.

In embodiments, crystalline Form A-9 of Compound I succinate salt exhibits an XRPD pattern comprising peaks at about 8.73±0.2, 20.05±0.2, and 26.15±0.2 degrees two-theta.

In embodiments, crystalline Form A-9 of Compound I succinate salt exhibits an XRPD pattern comprising peaks at about 4.08±0.2, 8.16±0.2, 8.73±0.2, 20.05±0.2, and 26.15±0.2 degrees two-theta.

In embodiments, crystalline Form A-9 of Compound I succinate salt exhibits an XRPD pattern which further comprises at least two peaks selected from about 6.76±0.2, 8.96±0.2, 12.30±0.2, 19.63±0.2, 21.10±0.2, 22.76±0.2, 25.88±0.2, and 31.55±0.2 degrees two-theta.

In some embodiments, the crystalline form of Compound I succinate salt exhibits an XRPD pattern comprising 1 peak, 2 peaks, 3 peaks, 4 peaks, 5 peaks, 6 peaks, 7 peaks, 8 peaks, 9 peaks, or 10 peaks ±0.2 degrees two-theta of Table 19.

In embodiments, crystalline Form A-9 of Compound I succinate salt exhibits an XRPD pattern comprising peaks shown in Table 19, below.

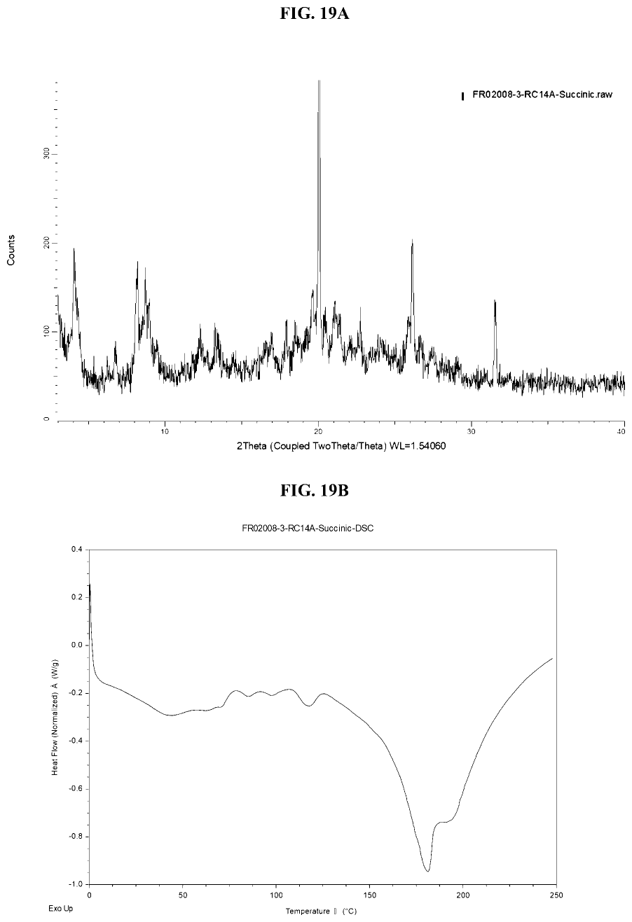

In embodiments, crystalline Form A-9 of Compound I succinate salt exhibits an XRPD pattern that is substantially similar to FIG. 19A.

TABLE 19
Form A-9 of Compound I succinate salt

| Angle | d Value | | Net Intensity | Rel. Intensity |
|---|---|---|---|---|
| 4.078° | 21.65238 | Å | 68.1927 | 19.7% |
| 6.763° | 13.06035 | Å | 34.3929 | 9.9% |
| 8.160° | 10.82603 | Å | 74.4621 | 21.5% |
| 8.725° | 10.12708 | Å | 80.0239 | 23.1% |
| 8.958° | 9.86355 | Å | 54.6964 | 15.8% |
| 9.494° | 9.30766 | Å | 24.3384 | 7.0% |
| 11.800° | 7.49393 | Å | 12.1565 | 3.5% |
| 12.303° | 7.18866 | Å | 37.8964 | 10.9% |
| 13.264° | 6.66985 | Å | 32.3373 | 9.3% |
| 17.905° | 4.94996 | Å | 31.6999 | 9.2% |
| 19.628° | 4.51911 | Å | 37.0157 | 10.7% |
| 20.053° | 4.42437 | Å | 346.167 | 100.0% |
| 20.460° | 4.33734 | Å | 28.6267 | 8.3% |
| 21.099° | 4.20728 | Å | 47.1351 | 13.6% |
| 21.347° | 4.15899 | Å | 27.2311 | 7.9% |
| 22.755° | 3.90469 | Å | 55.1880 | 15.9% |
| 25.875° | 3.44054 | Å | 43.2737 | 12.5% |
| 26.150° | 3.40501 | Å | 103.082 | 29.8% |
| 26.701° | 3.33599 | Å | 14.1631 | 4.1% |
| 31.546° | 2.83380 | Å | 62.3345 | 18.0% |

In embodiments, crystalline Form A-9 of Compound I succinate salt exhibits a DSC thermogram that is substantially similar to FIG. 19B.

### Compound I Phosphate Salt Form B

In embodiments, the present disclosure relates to a crystalline form of Compound I phosphate salt, which is Form B.

In embodiments, the crystalline form of Compound I phosphate salt may comprise of over about 99.9%, about 99.8%, about 99.7%, about 99.6%, about 99.5%, about 99.4%, about 99.3%, about 99.2%, about 99.1%, or about 99.0% of Form B of Compound I phosphate salt. In another embodiment, the crystalline form of Compound I phosphate salt may comprise over about 99%, 98%, 97%, 96%, 95%, 94%, 93%, 92%, 91%, or 90% of Form B of Compound I phosphate salt. In some embodiments, the crystalline form of Compound I phosphate salt may comprise over about 90%, 85%, 80%, 75%, 70%, 65%, 60%, 55%, 50%, 45%, or 40% of Form B of Compound I phosphate salt.

In embodiments, crystalline Form B of Compound I phosphate salt exhibits an XRPD pattern comprising peaks at about 5.73, 17.02, and 23.23 degrees two-theta with the margin of error of about ±0.5; about ±0.4; about ±0.3; about ±0.2; about ±0.1; about ±0.05; or less. In embodiments, crystalline Form B of Compound I phosphate salt exhibits an XRPD pattern comprising peaks at about 5.73, 11.37, 17.02, 22.70 and 23.23 degrees two-theta with the margin of error of about ±0.5; about ±0.4; about ±0.3; about ±0.2; about ±0.1; about ±0.05; or less. In embodiments, crystalline Form B of Compound I phosphate salt exhibits an XRPD pattern which further comprises at least two peaks selected from about 6.85, 7.39, 10.90, 14.65, 16.13, 19.77, 19.99, and 20.40 degrees two-theta with the margin of error of about ±0.5; about ±0.4; about ±0.3; about ±0.2; about ±0.1; about ±0.05; or less. In embodiments, crystalline Form B of Compound I phosphate salt exhibits an XRPD pattern which further comprises at least three peaks selected from about 6.85, 7.39, 10.90, 14.65, 16.13, 19.77, 19.99, and 20.40 degrees two-theta with the margin of error of about ±0.5; about ±0.4; about ±0.3; about ±0.2; about ±0.1; about ±0.05; or less. In embodiments, crystalline Form B of Compound I phosphate salt exhibits an XRPD pattern which further comprises at least four peaks selected from about 6.85, 7.39, 10.90, 14.65, 16.13, 19.77, 19.99, and 20.40 degrees two-theta with the margin of error of about ±0.5; about ±0.4; about ±0.3; about ±0.2; about ±0.1; about ±0.05; or less. In embodiments, crystalline Form B of Compound I phosphate salt exhibits an XRPD pattern which further comprises at least five peaks selected from about 6.85, 7.39, 10.90, 14.65, 16.13, 19.77, 19.99, and 20.40 degrees two-theta with the margin of error of about ±0.5; about ±0.4; about ±0.3; about ±0.2; about ±0.1; about ±0.05; or less. In embodiments, crystalline Form B of Compound I phosphate salt exhibits an XRPD pattern which further comprises at least six peaks selected from about 6.85, 7.39, 10.90, 14.65, 16.13, 19.77, 19.99, and 20.40 degrees two-theta with the margin of error of about ±0.5; about ±0.4; about ±0.3; about ±0.2; about ±0.1; about ±0.05; or less. In embodiments, crystalline Form B of Compound I phosphate salt exhibits an XRPD pattern which further comprises at least seven peaks selected from about 6.85, 7.39, 10.90, 14.65, 16.13, 19.77, 19.99, and 20.40 degrees two-theta with the margin of error of about ±0.5; about ±0.4; about ±0.3; about ±0.2; about ±0.1; about ±0.05; or less. In embodiments, crystalline Form B of Compound I phosphate salt exhibits an XRPD pattern which further comprises peaks at about 6.85, 7.39, 10.90, 14.65, 16.13, 19.77, 19.99, and 20.40 degrees two-theta with the margin of error of about ±0.5; about ±0.4; about ±0.3; about ±0.2; about ±0.1; about ±0.05; or less.

In embodiments, crystalline Form B of Compound I phosphate salt exhibits an XRPD pattern comprising peaks at about 5.73±0.2, 17.02±0.2, and 23.23±0.2 degrees two-theta.

In embodiments, crystalline Form B of Compound I phosphate salt exhibits an XRPD pattern comprising peaks at about 5.73±0.2, 11.37±0.2, 17.02±0.2, 22.70±0.2 and 23.23±0.2 degrees two-theta.

In embodiments, crystalline Form B of Compound I phosphate salt exhibits an XRPD pattern which further comprises at least two peaks selected from about 6.85±0.2, 7.39±0.2, 10.90±0.2, 14.65±0.2, 16.13±0.2, 19.77±0.2, 19.99±0.2, and 20.40±0.2 degrees two-theta degrees.

In some embodiments, the crystalline form of Compound I phosphate salt exhibits an XRPD pattern comprising 1 peak, 2 peaks, 3 peaks, 4 peaks, 5 peaks, 6 peaks, 7 peaks, 8 peaks, 9 peaks, or 10 peaks ±0.2 degrees two-theta of Table 20.

In embodiments, crystalline Form B of Compound I phosphate salt exhibits an XRPD pattern comprising peaks shown in Table 20, below.

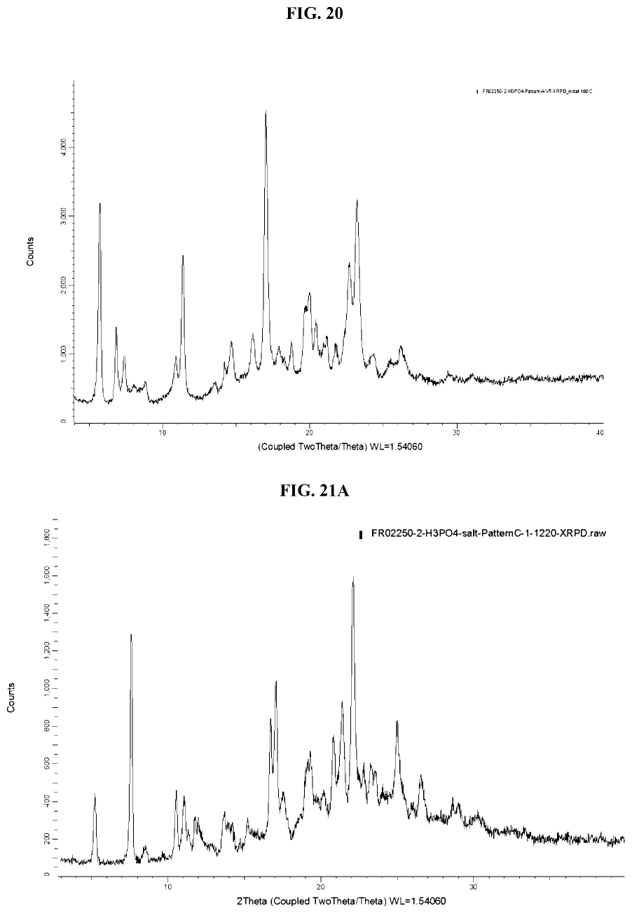

In embodiments, crystalline Form B of Compound I phosphate salt exhibits an XRPD pattern that is substantially similar to FIG. 20.

TABLE 20
Form B of Compound I phosphate salt

| | | | | Net | Gross | Rel. |
|---|---|---|---|---|---|---|
| Index | Angle | d Value | | Intensity | Intensity | Intensity |
| 1 | 5.732° | 15.40541 | Å | 1814.92 | 2127.44 | 68.1% |
| 2 | 6.848° | 12.89790 | Å | 693.171 | 1012.23 | 26.0% |
| 3 | 7.390° | 11.95220 | Å | 429.554 | 750.755 | 16.1% |
| 4 | 8.088° | 10.92227 | Å | 150.319 | 468.442 | 5.6% |
| 5 | 8.802° | 10.03841 | Å | 197.971 | 506.165 | 7.4% |
| 6 | 10.901° | 8.10953 | Å | 433.882 | 775.208 | 16.3% |
| 7 | 11.373° | 7.77379 | Å | 1392.64 | 1742.93 | 52.2% |
| 8 | 13.579° | 6.51578 | Å | 99.1167 | 557.144 | 3.7% |
| 9 | 14.277° | 6.19864 | Å | 164.847 | 681.312 | 6.2% |
| 10 | 14.652° | 6.04068 | Å | 441.657 | 986.696 | 16.6% |
| 11 | 16.134° | 5.48933 | Å | 469.826 | 1109.09 | 17.6% |
| 12 | 17.020° | 5.20550 | Å | 2666.64 | 3348.16 | 100.0% |
| 13 | 17.906° | 4.94984 | Å | 276.128 | 989.318 | 10.4% |
| 14 | 18.757° | 4.72695 | Å | 313.559 | 1047.23 | 11.8% |
| 15 | 19.767° | 4.48781 | Å | 629.075 | 1384.49 | 23.6% |
| 16 | 19.988° | 4.43856 | Å | 759.152 | 1518.59 | 28.5% |
| 17 | 20.398° | 4.35031 | Å | 492.787 | 1257.93 | 18.5% |
| 18 | 21.131° | 4.20103 | Å | 360.137 | 1129.83 | 13.5% |
| 19 | 21.779° | 4.07744 | Å | 171.995 | 939.694 | 6.4% |
| 20 | 22.701° | 3.91395 | Å | 1025.64 | 1780.75 | 38.5% |
| 21 | 23.229° | 3.82614 | Å | 1665.92 | 2408.66 | 62.5% |
| 22 | 24.329° | 3.65557 | Å | 191.058 | 895.972 | 7.2% |
| 23 | 25.520° | 3.48765 | Å | 204.644 | 878.427 | 7.7% |
| 24 | 26.163° | 3.40329 | Å | 311.866 | 977.305 | 11.7% |
| 25 | 26.327° | 3.38245 | Å | 170.418 | 832.844 | 6.4% |
| 26 | 27.523° | 3.23819 | Å | 53.8812 | 683.356 | 2.0% |
| 27 | 29.469° | 3.02857 | Å | 62.4851 | 659.900 | 2.3% |
| 28 | 31.079° | 2.87531 | Å | 97.1135 | 697.778 | 3.6% |

### Compound I Phosphate Salt Form C

In embodiments, the present disclosure relates to a crystalline form of Compound I phosphate salt, which is Form C.

In embodiments, the present disclosure relates to Form C, which is a crystalline form of Compound I phosphate salt, that is a hydrate.

In embodiments, the crystalline form of Compound I phosphate salt may comprise of over about 99.9%, about 99.8%, about 99.7%, about 99.6%, about 99.5%, about 99.4%, about 99.3%, about 99.2%, about 99.1%, or about 99.0% of Form C of Compound I phosphate salt. In another embodiment, the crystalline form of Compound I phosphate salt may comprise over about 99%, 98%, 97%, 96%, 95%, 94%, 93%, 92%, 91%, or 90% of Form C of Compound I phosphate salt. In some embodiments, the crystalline form of Compound I phosphate salt may comprise over about 90%, 85%, 80%, 75%, 70%, 65%, 60%, 55%, 50%, 45%, or 40% of Form C of Compound I phosphate salt.

In embodiments, crystalline Form C of Compound I phosphate salt exhibits an XRPD pattern comprising peaks at about 7.66, 17.15, and 22.09 degrees two-theta with the margin of error of about ±0.5; about ±0.4; about ±0.3; about ±0.2; about ±0.1; about ±0.05; or less. In embodiments, crystalline Form C of Compound I phosphate salt exhibits an XRPD pattern comprising peaks at about 5.22, 7.66, 17.15, 22.09, and 24.96 degrees two-theta with the margin of error of about ±0.5; about ±0.4; about ±0.3; about ±0.2; about ±0.1; about ±0.05; or less. In embodiments, crystalline Form C of Compound I phosphate salt exhibits an X-ray powder diffraction (XRPD) pattern which further comprises at least two peaks selected from about 10.55, 11.06, 16.81, 17.60, 19.32, 20.88, 21.39, and 26.46 degrees two-theta with the margin of error of about ±0.5; about ±0.4; about ±0.3; about ±0.2; about ±0.1; about ±0.05; or less. In embodiments, crystalline Form C of Compound I phosphate salt exhibits an X-ray powder diffraction (XRPD) pattern which further comprises at least three peaks selected from about 10.55, 11.06, 16.81, 17.60, 19.32, 20.88, 21.39, and 26.46 degrees two-theta with the margin of error of about ±0.5; about ±0.4; about ±0.3; about ±0.2; about ±0.1; about ±0.05; or less. In embodiments, crystalline Form C of Compound I phosphate salt exhibits an X-ray powder diffraction (XRPD) pattern which further comprises at least four peaks selected from about 10.55, 11.06, 16.81, 17.60, 19.32, 20.88, 21.39, and 26.46 degrees two-theta with the margin of error of about ±0.5; about ±0.4; about ±0.3; about ±0.2; about ±0.1; about ±0.05; or less. In embodiments, crystalline Form C of Compound I phosphate salt exhibits an X-ray powder diffraction (XRPD) pattern which further comprises at least five peaks selected from about 10.55, 11.06, 16.81, 17.60, 19.32, 20.88, 21.39, and 26.46 degrees two-theta with the margin of error of about ±0.5; about ±0.4; about ±0.3; about ±0.2; about ±0.1; about ±0.05; or less. In embodiments, crystalline Form C of Compound I phosphate salt exhibits an X-ray powder diffraction (XRPD) pattern which further comprises at least six peaks selected from about 10.55, 11.06, 16.81, 17.60, 19.32, 20.88, 21.39, and 26.46 degrees two-theta with the margin of error of about ±0.5; about ±0.4; about ±0.3; about ±0.2; about ±0.1; about ±0.05; or less. In embodiments, crystalline Form C of Compound I phosphate salt exhibits an X-ray powder diffraction (XRPD) pattern which further comprises at least seven peaks selected from about 10.55, 11.06, 16.81, 17.60, 19.32, 20.88, 21.39, and 26.46 degrees two-theta with the margin of error of about ±0.5; about ±0.4; about ±0.3; about ±0.2; about ±0.1; about ±0.05; or less. In embodiments, crystalline Form C of Compound I phosphate salt exhibits an X-ray powder diffraction (XRPD) pattern which further comprises peaks at about 10.55, 11.06, 16.81, 17.60, 19.32, 20.88, 21.39, and 26.46 degrees two-theta with the margin of error of about ±0.5; about ±0.4; about ±0.3; about ±0.2; about ±0.1; about ±0.05; or less.

In embodiments, crystalline Form C of Compound I phosphate salt exhibits an XRPD pattern comprising peaks at about 7.66±0.2, 17.15±0.2, and 22.09±0.2 degrees two-theta.

In embodiments, crystalline Form C of Compound I phosphate salt exhibits an XRPD pattern comprising peaks at about 5.22±0.2, 7.66±0.2, 17.15±0.2, 22.09±0.2, and 24.96±0.2 degrees two-theta.

In embodiments, crystalline Form C of Compound I phosphate salt exhibits an X-ray powder diffraction (XRPD) pattern which further comprises at least two peaks selected from about 10.55±0.2, 11.06±0.2, 16.81±0.2, 17.60±0.2, 19.32±0.2, 20.88±0.2, 21.39±0.2, and 26.46±0.2 degrees two-theta.

In some embodiments, the crystalline form of Compound I phosphate salt exhibits an XRPD pattern comprising 1 peak, 2 peaks, 3 peaks, 4 peaks, 5 peaks, 6 peaks, 7 peaks, 8 peaks, 9 peaks, or 10 peaks ±0.2 degrees two-theta of Table 21.

In embodiments, crystalline Form C of Compound I phosphate salt exhibits an XRPD pattern comprising peaks shown in Table 21, below.

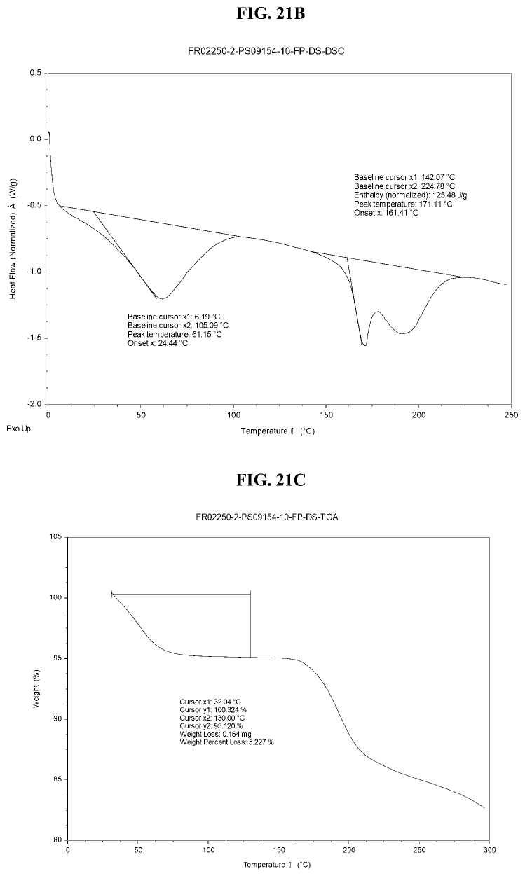

In embodiments, crystalline Form C of Compound I phosphate salt exhibits an XRPD pattern that is substantially similar to FIG. 21A.

TABLE 21
Form C of Compound I phosphate salt

| | | | | Net | Gross | Rel. |
|---|---|---|---|---|---|---|
| Index | Angle | d Value | | Intensity | Intensity | Intensity |
| 1 | 3.863° | 22.85681 | Å | 10.8975 | 41.5625 | 2.2% |
| 2 | 5.218° | 16.92231 | Å | 190.290 | 229.317 | 37.9% |
| 3 | 7.664° | 11.52621 | Å | 436.863 | 488.627 | 87.0% |
| 4 | 8.496° | 10.39867 | Å | 24.7273 | 80.7389 | 4.9% |
| 5 | 10.553° | 8.37662 | Å | 134.482 | 207.887 | 26.8% |
| 6 | 11.057° | 7.99521 | Å | 136.926 | 216.121 | 27.3% |
| 7 | 12.817° | 6.90147 | Å | 35.6759 | 122.909 | 7.1% |
| 8 | 13.690° | 6.46330 | Å | 74.9312 | 166.384 | 14.9% |
| 9 | 14.072° | 6.28831 | Å | 25.8905 | 119.288 | 5.2% |
| 10 | 14.181° | 6.24064 | Å | 61.5020 | 155.287 | 12.2% |
| 11 | 15.312° | 5.78201 | Å | 48.0351 | 153.456 | 9.6% |
| 12 | 15.650° | 5.65793 | Å | 47.9859 | 156.920 | 9.6% |
| 13 | 16.814° | 5.26881 | Å | 181.166 | 304.518 | 36.1% |
| 14 | 17.148° | 5.16683 | Å | 350.973 | 477.711 | 69.9% |
| 15 | 17.603° | 5.03414 | Å | 89.7759 | 220.028 | 17.9% |
| 16 | 18.752° | 4.72825 | Å | 32.1080 | 179.330 | 6.4% |
| 17 | 19.320° | 4.59056 | Å | 166.487 | 327.558 | 33.1% |
| 18 | 19.695° | 4.50393 | Å | 66.8592 | 236.001 | 13.3% |
| 19 | 20.197° | 4.39323 | Å | 63.6863 | 242.274 | 12.7% |
| 20 | 20.881° | 4.25073 | Å | 156.055 | 344.916 | 31.1% |
| 21 | 21.394° | 4.15003 | Å | 175.156 | 369.736 | 34.9% |
| 22 | 22.086° | 4.02145 | Å | 502.240 | 702.003 | 100.0% |
| 23 | 23.297° | 3.81520 | Å | 63.1589 | 264.959 | 12.6% |
| 24 | 23.659° | 3.75755 | Å | 72.7541 | 273.427 | 14.5% |
| 25 | 24.962° | 3.56424 | Å | 193.610 | 383.612 | 38.5% |
| 26 | 26.455° | 3.36639 | Å | 96.3986 | 270.699 | 19.2% |
| 27 | 28.947° | 3.08204 | Å | 28.3126 | 185.560 | 5.6% |

In embodiments, the crystalline Form C of Compound I phosphate salt exhibits a DSC thermogram comprising an endotherm peak at about 6° C. with the error of margin of about ±2.5; about ±2.0; about ±1.5; about ±1.0; about ±0.5; or less. In embodiments, the crystalline Form C of Compound I phosphate salt exhibits a DSC thermogram comprising an endotherm peak with an onset at about 161° C. with the error of margin of about ±2.5; about ±2.0; about ±1.5; about ±1.0; about ±0.5; or less. In embodiments, the crystalline Form C of Compound I phosphate salt exhibits a DSC thermogram comprising an endotherm peak at about 171° C. with the error of margin of about ±2.5; about ±2.0; about ±1.5; about ±1.0; about ±0.5; or less.

In some embodiments, the crystalline Form C of Compound I phosphate salt exhibits a DSC thermogram that is substantially similar to FIG. 21B.

In embodiments, the crystalline Form C of Compound I phosphate salt exhibits a TGA thermogram substantially similar to FIG. 21C. In embodiments, the crystalline Form C of Compound I phosphate salt exhibits a weight percent loss of about 5% between about 32° C. to about 130° C. by a thermogravimetric analysis (TGA).

### Compound I Phosphate Salt Form J

In embodiments, the present disclosure relates to a crystalline form of Compound I phosphate salt, which is Form J.

In embodiments, the present disclosure relates to Form J, which is a crystalline form of Compound I phosphate salt, that is a hydrate.

In embodiments, the crystalline form of Compound I phosphate salt may comprise of over about 99.9%, about 99.8%, about 99.7%, about 99.6%, about 99.5%, about 99.4%, about 99.3%, about 99.2%, about 99.1%, or about 99.0% of Form J of Compound I phosphate salt. In another embodiment, the crystalline form of Compound I phosphate salt may comprise over about 99%, 98%, 97%, 96%, 95%, 94%, 93%, 92%, 91%, or 90% of Form J of Compound I phosphate salt. In some embodiments, the crystalline form of Compound I phosphate salt may comprise over about 90%, 85%, 80%, 75%, 70%, 65%, 60%, 55%, 50%, 45%, or 40% of Form J of Compound I phosphate salt.

In embodiments, crystalline Form J of Compound I phosphate salt exhibits an XRPD pattern comprising peaks at about 4.97, 8.09, and 23.89 degrees two-theta with the margin of error of about ±0.5; about ±0.4; about ±0.3; about ±0.2; about ±0.1; about ±0.05; or less. In embodiments, crystalline Form J of Compound I phosphate salt exhibits an XRPD pattern comprising peaks at about 4.97, 8.09, 17.46, 23.89, and 30.74 degrees two-theta with the margin of error of about ±0.5; about ±0.4; about ±0.3; about ±0.2; about ±0.1; about ±0.05; or less.

In embodiments, crystalline Form J of Compound I phosphate salt exhibits an XRPD pattern comprising peaks at about 4.97±0.2, 8.09±0.2, and 23.89±0.2 degrees two-theta.

In embodiments, crystalline Form J of Compound I phosphate salt exhibits an XRPD pattern comprising peaks at about 4.97±0.2, 8.09±0.2, 17.46±0.2, 23.89±0.2, and 30.74±0.2 degrees two-theta.

In some embodiments, the crystalline form of Compound I phosphate salt exhibits an XRPD pattern comprising 1 peak, 2 peaks, 3 peaks, 4 peaks, 5 peaks, 6 peaks, 7 peaks, 8 peaks, 9 peaks, or 10 peaks ±0.2 degrees two-theta of Table 22.

In embodiments, crystalline Form J of Compound I phosphate salt exhibits an XRPD pattern comprising peaks shown in Table 22, below.

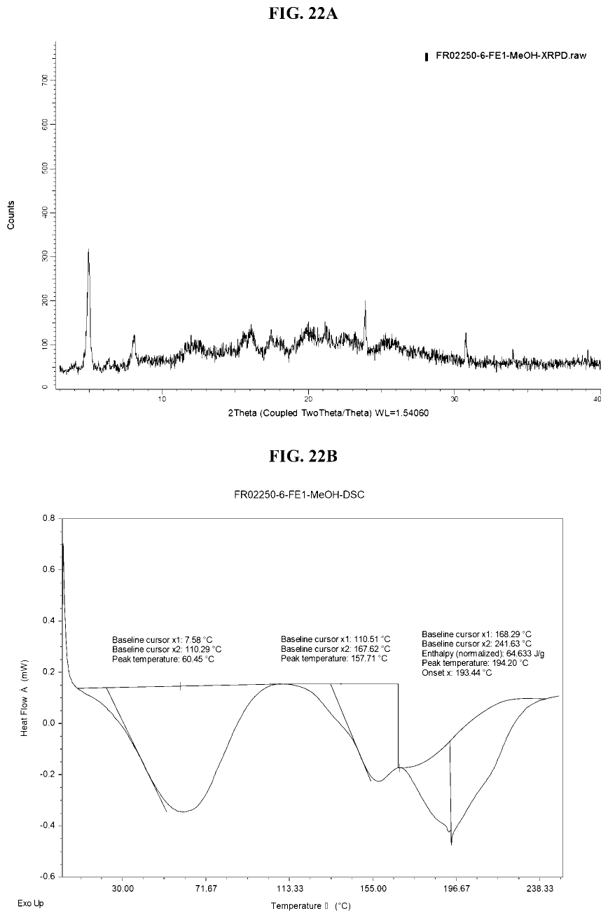

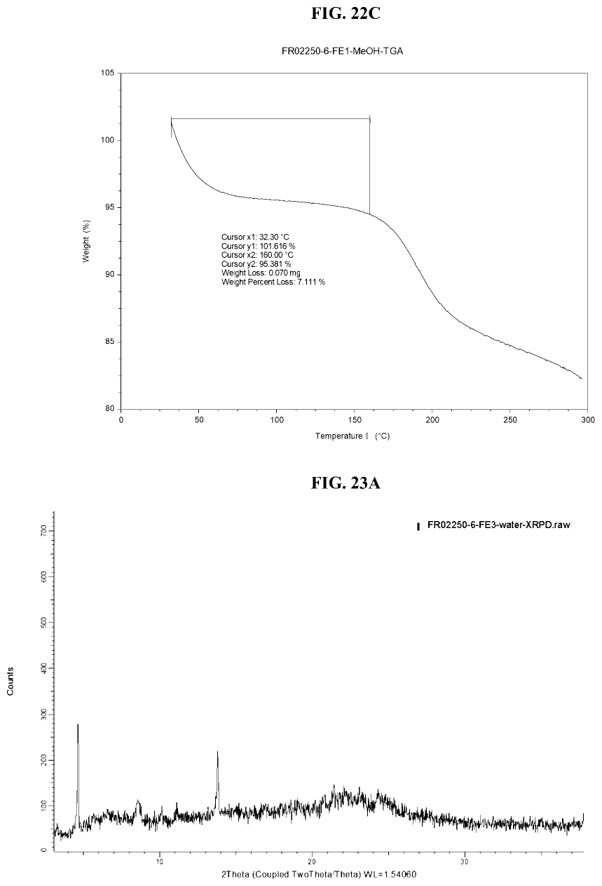

In embodiments, crystalline Form J of Compound I phosphate salt exhibits an XRPD pattern that is substantially similar to FIG. 22A.

TABLE 22
Form J of Compound I phosphate salt

| | | | | Net | Gross | Rel. |
|---|---|---|---|---|---|---|
| Index | Angle | d Value | | Intensity | Intensity | Intensity |
| 1 | 4.971° | 17.76248 | Å | 170.110 | 213.145 | 100.0% |
| 2 | 6.294° | 14.03161 | Å | 11.6388 | 55.8010 | 6.8% |
| 3 | 8.087° | 10.92346 | Å | 49.3497 | 100.247 | 29.0% |
| 4 | 16.095° | 5.50228 | Å | 24.8346 | 103.799 | 14.6% |
| 5 | 17.456° | 5.07644 | Å | 41.8344 | 119.775 | 24.6% |
| 6 | 23.886° | 3.72239 | Å | 84.3154 | 175.983 | 49.6% |
| 7 | 30.738° | 2.90639 | Å | 45.7147 | 105.760 | 26.9% |

In embodiments, the crystalline Form J of Compound I phosphate salt exhibits a DSC thermogram comprising an endotherm peak at about 60.5° C. with the error of margin of about ±2.5; about ±2.0; about ±1.5; about ±1.0; about ±0.5; or less. In embodiments, the crystalline Form J of Compound I phosphate salt exhibits a DSC thermogram comprising an endotherm peak at about 157.7° C. with the error of margin of about ±2.5; about ±2.0; about ±1.5; about ±1.0; about ±0.5; or less. In embodiments, crystalline Form J of Compound I phosphate salt exhibits a DSC thermogram comprising an endotherm peak with an onset at about 193° C. with the error of margin of about ±2.5; about ±2.0; about ±1.5; about ±1.0; about ±0.5; or less. In embodiments, the crystalline Form J of Compound I phosphate salt exhibits a DSC thermogram comprising an endotherm peak at about 194° C. with the error of margin of about ±2.5; about ±2.0; about ±1.5; about ±1.0; about ±0.5; or less.

In some embodiments, the crystalline Form J of Compound I phosphate salt exhibits a DSC thermogram that is substantially similar to FIG. 22B.

In embodiments, the crystalline Form J of Compound I phosphate salt exhibits a TGA thermogram substantially similar to FIG. 22C. In embodiments, the crystalline Form J of Compound I phosphate salt exhibits a weight percent loss of about 7.1% between about 32° C. to about 160° C. by a thermogravimetric analysis (TGA).

### Compound I Phosphate Salt Form K

In embodiments, the present disclosure relates to a crystalline form of Compound I phosphate salt, which is Form K.

In embodiments, the present disclosure relates to Form K, which is a crystalline form of Compound I phosphate salt, that is a hydrate.

In embodiments, the crystalline form of Compound I phosphate salt may comprise of over about 99.9%, about 99.8%, about 99.7%, about 99.6%, about 99.5%, about 99.4%, about 99.3%, about 99.2%, about 99.1%, or about 99.0% of Form K of Compound I phosphate salt. In another embodiment, the crystalline form of Compound I phosphate salt may comprise over about 99%, 98%, 97%, 96%, 95%, 94%, 93%, 92%, 91%, or 90% of Form K of Compound I phosphate salt. In some embodiments, the crystalline form of Compound I phosphate salt may comprise over about 90%, 85%, 80%, 75%, 70%, 65%, 60%, 55%, 50%, 45%, or 40% of Form K of Compound I phosphate salt.

In embodiments, crystalline Form K of Compound I phosphate salt exhibits an XRPD pattern comprising peaks at about 4.59, 13.75, and 21.37 degrees two-theta with the margin of error of about ±0.5; about ±0.4; about ±0.3; about ±0.2; about ±0.1; about ±0.05; or less. In embodiments, crystalline Form K of Compound I phosphate salt exhibits an XRPD pattern comprising peaks at about 4.59, 8.53, 13.75, 21.37, and 23.02 degrees two-theta with the margin of error of about ±0.5; about ±0.4; about ±0.3; about ±0.2; about ±0.1; about ±0.05; or less. In embodiments, crystalline Form K of Compound I phosphate salt exhibits an XRPD pattern which further comprises at least two peaks selected from about 6.58±0.2, 10.08±0.2, 11.08±0.2, 24.21±0.2, and 31.80±0.2 degrees two-theta with the margin of error of about ±0.5; about ±0.4; about ±0.3; about ±0.2; about ±0.1; about ±0.05; or less. In embodiments, crystalline Form K of Compound I phosphate salt exhibits an XRPD pattern which further comprises at least three peaks selected from about 6.58±0.2, 10.08±0.2, 11.08±0.2, 24.21±0.2, and 31.80±0.2 degrees two-theta with the margin of error of about ±0.5; about ±0.4; about ±0.3; about ±0.2; about ±0.1; about ±0.05; or less. In embodiments, crystalline Form K of Compound I phosphate salt exhibits an XRPD pattern which further comprises at least four peaks selected from about 6.58±0.2, 10.08±0.2, 11.08±0.2, 24.21±0.2, and 31.80±0.2 degrees two-theta with the margin of error of about ±0.5; about ±0.4; about ±0.3; about ±0.2; about ±0.1; about ±0.05; or less. In embodiments, crystalline Form K of Compound I phosphate salt exhibits an XRPD pattern which further comprises peaks at about 6.58±0.2, 10.08±0.2, 11.08±0.2, 24.21±0.2, and 31.80±0.2 degrees two-theta with the margin of error of about ±0.5; about ±0.4; about ±0.3; about ±0.2; about ±0.1; about ±0.05; or less.

In embodiments, crystalline Form K of Compound I phosphate salt exhibits an XRPD pattern comprising peaks at about 4.59±0.2, 13.75±0.2, and 21.37±0.2 degrees two-theta.

In embodiments, crystalline Form K of Compound I phosphate salt exhibits an XRPD pattern comprising peaks at about 4.59±0.2, 8.53±0.2, 13.75±0.2, 21.37±0.2, and 23.02±0.2 degrees two-theta.

In embodiments, crystalline Form K of Compound I phosphate salt exhibits an XRPD pattern which further comprises at least two peaks selected from about 6.58±0.2, 10.08±0.2, 11.08±0.2, 24.21±0.2, and 31.80±0.2 degrees two-theta.

In some embodiments, the crystalline form of Compound I phosphate salt exhibits an XRPD pattern comprising 1 peak, 2 peaks, 3 peaks, 4 peaks, 5 peaks, 6 peaks, 7 peaks, 8 peaks, 9 peaks, or 10 peaks ±0.2 degrees two-theta of Table 23.

In embodiments, crystalline Form K of Compound I phosphate salt exhibits an XRPD pattern comprising peaks shown in Table 23, below.

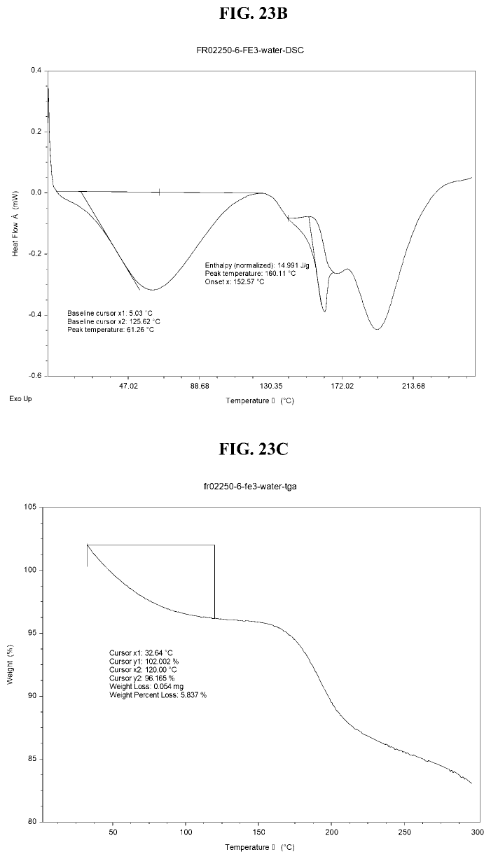

In embodiments, crystalline Form K of Compound I phosphate salt exhibits an XRPD pattern that is substantially similar to FIG. 23A.

TABLE 23
Form K of Compound I phosphate salt

| | | | | Net | Gross | Rel. |
|---|---|---|---|---|---|---|
| Index | Angle | d Value | | Intensity | Intensity | Intensity |
| 1 | 4.593° | 19.22149 | Å | 152.089 | 195.063 | 100.0% |
| 2 | 5.000° | 17.65923 | Å | 13.3543 | 60.5840 | 8.8% |
| 3 | 6.577° | 13.42803 | Å | 19.4621 | 77.9802 | 12.8% |
| 4 | 8.534° | 10.35299 | Å | 33.7122 | 94.7070 | 22.2% |
| 5 | 10.082° | 8.76680 | Å | 24.8007 | 86.3072 | 16.3% |
| 6 | 11.078° | 7.98054 | Å | 27.8654 | 94.4804 | 18.3% |
| 7 | 13.754° | 6.43298 | Å | 100.084 | 177.889 | 65.8% |
| 8 | 21.373° | 4.15399 | Å | 40.2217 | 134.063 | 26.4% |
| 9 | 23.024° | 3.85966 | Å | 38.7429 | 136.276 | 25.5% |
| 10 | 24.205° | 3.67399 | Å | 27.2155 | 121.814 | 17.9% |
| 11 | 31.799° | 2.81181 | Å | 21.1067 | 77.7338 | 13.9% |
| 12 | 38.756° | 2.32160 | Å | 18.5494 | 65.3235 | 12.2% |
| 13 | 39.110° | 2.30140 | Å | 13.1796 | 61.9240 | 8.7% |

In embodiments, the crystalline Form K of Compound I phosphate salt exhibits a DSC thermogram comprising an endotherm peak at about 61.3° C. with the error of margin of about ±2.5; about ±2.0; about ±1.5; about ±1.0; about ±0.5; or less. In embodiments, the crystalline Form K of Compound I phosphate salt exhibits a DSC thermogram comprising an endotherm peak with an onset at about 152.6° C. with the error of margin of about ±2.5; about ±2.0; about ±1.5; about ±1.0; about ±0.5; or less.

In some embodiments, the crystalline Form K of Compound I phosphate salt exhibits a DSC thermogram that is substantially similar to FIG. 23B.

In embodiments, the crystalline Form K of Compound I phosphate salt exhibits a TGA thermogram substantially similar to FIG. 23C. In embodiments, the crystalline Form K of Compound I phosphate salt exhibits a weight percent loss of about 5.8% between about 32° C. to about 120° C. by a thermogravimetric analysis (TGA).

### Compound I Phosphate Salt Form H2

In embodiments, the present disclosure relates to a crystalline form of Compound I phosphate salt, which is Form H2.

In embodiments, the present disclosure relates to Form H2, which is a crystalline form of Compound I phosphate salt, that is a hydrate.

In embodiments, the crystalline form of Compound I phosphate salt may comprise of over about 99.9%, about 99.8%, about 99.7%, about 99.6%, about 99.5%, about 99.4%, about 99.3%, about 99.2%, about 99.1%, or about 99.0% of Form H2 of Compound I phosphate salt. In another embodiment, the crystalline form of Compound I phosphate salt may comprise over about 99%, 98%, 97%, 96%, 95%, 94%, 93%, 92%, 91%, or 90% of Form H2 of Compound I phosphate salt. In some embodiments, the crystalline form of Compound I phosphate salt may comprise over about 90%, 85%, 80%, 75%, 70%, 65%, 60%, 55%, 50%, 45%, or 40% of Form H2 of Compound I phosphate salt.

In embodiments, crystalline Form H2 of Compound I phosphate salt exhibits an XRPD pattern comprising peaks at about 10.66, 21.99, and 22.38 degrees two-theta with the margin of error of about ±0.5; about ±0.4; about ±0.3; about ±0.2; about ±0.1; about ±0.05; or less. In embodiments, crystalline Form H2 of Compound I phosphate salt exhibits an XRPD pattern comprising peaks at about 10.66, 18.78, 21.99, 22.38, and 23.56 degrees two-theta with the margin of error of about ±0.5; about ±0.4; about ±0.3; about ±0.2; about ±0.1; about ±0.05; or less. In embodiments, crystalline Form H2 of Compound I phosphate salt exhibits an XRPD pattern which further comprises at least two peaks selected from about 8.49, 11.51, 13.62, 14.02, 15.37, 21.35, 23.20 and 24.13 degrees two-theta with the margin of error of about ±0.5; about ±0.4; about ±0.3; about ±0.2; about ±0.1; about ±0.05; or less. In embodiments, crystalline Form H2 of Compound I phosphate salt exhibits an XRPD pattern which further comprises at least three peaks selected from about 8.49, 11.51, 13.62, 14.02, 15.37, 21.35, 23.20 and 24.13 degrees two-theta with the margin of error of about ±0.5; about ±0.4; about ±0.3; about ±0.2; about ±0.1; about ±0.05; or less. In embodiments, crystalline Form H2 of Compound I phosphate salt exhibits an XRPD pattern which further comprises at least four peaks selected from about 8.49, 11.51, 13.62, 14.02, 15.37, 21.35, 23.20 and 24.13 degrees two-theta with the margin of error of about ±0.5; about ±0.4; about ±0.3; about ±0.2; about ±0.1; about ±0.05; or less. In embodiments, crystalline Form H2 of Compound I phosphate salt exhibits an XRPD pattern which further comprises at least five peaks selected from about 8.49, 11.51, 13.62, 14.02, 15.37, 21.35, 23.20 and 24.13 degrees two-theta with the margin of error of about ±0.5; about ±0.4; about ±0.3; about ±0.2; about ±0.1; about ±0.05; or less. In embodiments, crystalline Form H2 of Compound I phosphate salt exhibits an XRPD pattern which further comprises at least six peaks selected from about 8.49, 11.51, 13.62, 14.02, 15.37, 21.35, 23.20 and 24.13 degrees two-theta with the margin of error of about ±0.5; about ±0.4; about ±0.3; about ±0.2; about ±0.1; about ±0.05; or less. In embodiments, crystalline Form H2 of Compound I phosphate salt exhibits an XRPD pattern which further comprises at least seven peaks selected from about 8.49, 11.51, 13.62, 14.02, 15.37, 21.35, 23.20 and 24.13 degrees two-theta with the margin of error of about ±0.5; about ±0.4; about ±0.3; about ±0.2; about ±0.1; about ±0.05; or less. In embodiments, crystalline Form H2 of Compound I phosphate salt exhibits an XRPD pattern which further comprises peaks at about 8.49, 11.51, 13.62, 14.02, 15.37, 21.35, 23.20 and 24.13 degrees two-theta with the margin of error of about ±0.5; about ±0.4; about ±0.3; about ±0.2; about ±0.1; about ±0.05; or less.

In embodiments, crystalline Form H2 of Compound I phosphate salt exhibits an XRPD pattern comprising peaks at about 10.66±0.2, 21.99±0.2, and 22.38±0.2 degrees two-theta.

In embodiments, crystalline Form H2 of Compound I phosphate salt exhibits an XRPD pattern comprising peaks at about 10.66±0.2, 18.78±0.2, 21.99±0.2, 22.38±0.2, and 23.56±0.2 degrees two-theta.

In embodiments, crystalline Form H2 of Compound I phosphate salt exhibits an XRPD pattern which further comprises at least two peaks selected from about 8.49±0.2, 11.51±0.2, 13.62±0.2, 14.02±0.2, 15.37±0.2, 21.35±0.2, 23.20±0.2 and 24.13±0.2 degrees two-theta.

In some embodiments, the crystalline form of Compound I phosphate salt exhibits an XRPD pattern comprising 1 peak, 2 peaks, 3 peaks, 4 peaks, 5 peaks, 6 peaks, 7 peaks, 8 peaks, 9 peaks, or 10 peaks ±0.2 degrees two-theta of Table 24.

In embodiments, crystalline Form H2 of Compound I phosphate salt exhibits an XRPD pattern comprising peaks shown in Table 24, below.

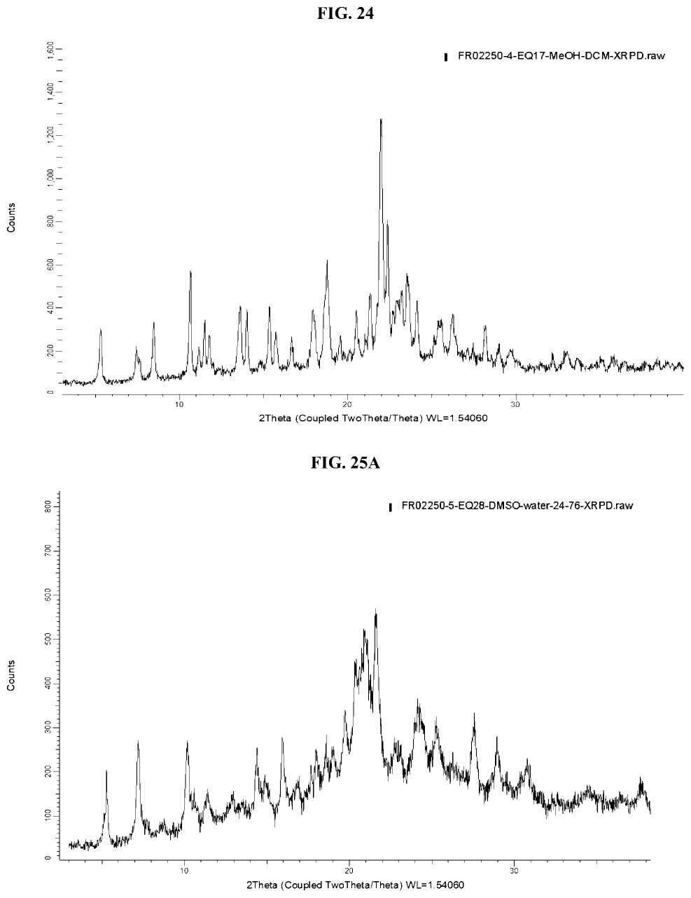

In embodiments, crystalline Form H2 of Compound I phosphate salt exhibits an XRPD pattern that is substantially similar to FIG. 24.

TABLE 24
Form H2 of Compound I phosphate salt

| | | | | Net | Gross | Rel. |
|---|---|---|---|---|---|---|
| Index | Angle | d Value | | Intensity | Intensity | Intensity |
| 1 | 5.340° | 16.53524 | Å | 144.852 | 198.003 | 20.3% |
| 2 | 7.470° | 11.82489 | Å | 75.7841 | 144.322 | 10.6% |
| 3 | 8.489° | 10.40719 | Å | 173.770 | 245.460 | 24.3% |
| 4 | 10.659° | 8.29318 | Å | 340.305 | 424.240 | 47.6% |
| 5 | 11.165° | 7.91840 | Å | 87.6299 | 177.082 | 12.3% |
| 6 | 11.510° | 7.68175 | Å | 170.192 | 262.358 | 23.8% |
| 7 | 11.807° | 7.48951 | Å | 110.896 | 204.713 | 15.5% |
| 8 | 12.347° | 7.16281 | Å | 20.7277 | 115.940 | 2.9% |
| 9 | 13.619° | 6.49659 | Å | 209.293 | 310.069 | 29.3% |
| 10 | 14.024° | 6.31000 | Å | 189.714 | 293.722 | 26.6% |
| 11 | 14.803° | 5.97966 | Å | 20.0038 | 130.590 | 2.8% |
| 12 | 15.366° | 5.76185 | Å | 202.239 | 316.863 | 28.3% |
| 13 | 15.738° | 5.62622 | Å | 127.052 | 243.104 | 17.8% |
| 14 | 16.669° | 5.31405 | Å | 112.780 | 232.502 | 15.8% |
| 15 | 17.947° | 4.93863 | Å | 160.903 | 299.130 | 22.5% |
| 16 | 18.780° | 4.72127 | Å | 326.608 | 478.731 | 45.7% |
| 17 | 19.572° | 4.53210 | Å | 85.7423 | 246.467 | 12.0% |
| 18 | 20.517° | 4.32532 | Å | 158.668 | 331.831 | 22.2% |
| 19 | 21.349° | 4.15862 | Å | 180.830 | 364.493 | 25.3% |
| 20 | 21.993° | 4.03825 | Å | 714.190 | 902.590 | 100.0% |
| 21 | 22.377° | 3.96978 | Å | 439.431 | 629.243 | 61.5% |
| 22 | 23.203° | 3.83041 | Å | 199.573 | 388.852 | 27.9% |
| 23 | 23.559° | 3.77330 | Å | 222.554 | 410.100 | 31.2% |
| 24 | 24.127° | 3.68576 | Å | 176.691 | 359.601 | 24.7% |
| 25 | 25.480° | 3.49304 | Å | 80.8916 | 261.199 | 11.3% |
| 26 | 26.254° | 3.39172 | Å | 137.329 | 314.361 | 19.2% |
| 27 | 27.122° | 3.28513 | Å | 27.2561 | 195.529 | 3.8% |
| 28 | 27.424° | 3.24965 | Å | 29.7431 | 193.705 | 4.2% |
| 29 | 28.185° | 3.16357 | Å | 107.094 | 257.715 | 15.0% |
| 30 | 28.963° | 3.08036 | Å | 43.5768 | 176.421 | 6.1% |
| 31 | 29.683° | 3.00725 | Å | 59.7715 | 183.429 | 8.4% |
| 32 | 32.196° | 2.77802 | Å | 41.7925 | 159.812 | 5.9% |
| 33 | 33.053° | 2.70792 | Å | 46.8397 | 168.193 | 6.6% |
| 34 | 33.640° | 2.66205 | Å | 28.3829 | 151.565 | 4.0% |
| 35 | 35.765° | 2.50861 | Å | 30.1015 | 148.413 | 4.2% |
| 36 | 35.830° | 2.50417 | Å | 28.8500 | 147.354 | 4.0% |
| 37 | 36.428° | 2.46446 | Å | 23.6911 | 142.559 | 3.3% |

### Compound I Phosphate Salt Form E

In embodiments, the present disclosure relates to a crystalline form of Compound I phosphate salt, which is Form E.

In embodiments of the present disclosure, crystalline Form E of Compound I phosphate salt is a solvate.

In embodiments of the present disclosure, crystalline Form E of Compound I phosphate salt is a DMSO solvate.

In embodiments, the crystalline form of Compound I phosphate salt may comprise of over about 99.9%, about 99.8%, about 99.7%, about 99.6%, about 99.5%, about 99.4%, about 99.3%, about 99.2%, about 99.1%, or about 99.0% of Form E of Compound I phosphate salt. In another embodiment, the crystalline form of Compound I phosphate salt may comprise over about 99%, 98%, 97%, 96%, 95%, 94%, 93%, 92%, 91%, or 90% of Form E of Compound I phosphate salt. In some embodiments, the crystalline form of Compound I phosphate salt may comprise over about 90%, 85%, 80%, 75%, 70%, 65%, 60%, 55%, 50%, 45%, or 40% of Form E of Compound I phosphate salt.

In embodiments, crystalline Form E of Compound I phosphate salt exhibits an XRPD pattern comprising peaks at about 20.42, 20.94, and 21.65 degrees two-theta with the margin of error of about ±0.5; about ±0.4; about ±0.3; about ±0.2; about ±0.1; about ±0.05; or less. In embodiments, crystalline Form E of Compound I phosphate salt exhibits an XRPD pattern comprising peaks at about 7.21, 10.18, 20.42, 20.94, and 21.65 degrees two-theta with the margin of error of about ±0.5; about ±0.4; about ±0.3; about ±0.2; about ±0.1; about ±0.05; or less. In embodiments, crystalline Form E of Compound I phosphate salt exhibits an XRPD pattern which further comprises at least two peaks selected from about 5.30, 14.41, 15.95, 19.76, 24.22, 25.28, 27.56, and 28.97 degrees two-theta with the margin of error of about ±0.5; about ±0.4; about ±0.3; about ±0.2; about ±0.1; about ±0.05; or less. In embodiments, crystalline Form E of Compound I phosphate salt exhibits an XRPD pattern which further comprises at least three peaks selected from about 5.30, 14.41, 15.95, 19.76, 24.22, 25.28, 27.56, and 28.97 degrees two-theta with the margin of error of about ±0.5; about ±0.4; about ±0.3; about ±0.2; about ±0.1; about ±0.05; or less. In embodiments, crystalline Form E of Compound I phosphate salt exhibits an XRPD pattern which further comprises at least four peaks selected from about 5.30, 14.41, 15.95, 19.76, 24.22, 25.28, 27.56, and 28.97 degrees two-theta with the margin of error of about ±0.5; about ±0.4; about ±0.3; about ±0.2; about ±0.1; about ±0.05; or less. In embodiments, crystalline Form E of Compound I phosphate salt exhibits an XRPD pattern which further comprises at least five peaks selected from about 5.30, 14.41, 15.95, 19.76, 24.22, 25.28, 27.56, and 28.97 degrees two-theta with the margin of error of about ±0.5; about ±0.4; about ±0.3; about ±0.2; about ±0.1; about ±0.05; or less. In embodiments, crystalline Form E of Compound I phosphate salt exhibits an XRPD pattern which further comprises at least six peaks selected from about 5.30, 14.41, 15.95, 19.76, 24.22, 25.28, 27.56, and 28.97 degrees two-theta with the margin of error of about ±0.5; about ±0.4; about ±0.3; about ±0.2; about ±0.1; about ±0.05; or less. In embodiments, crystalline Form E of Compound I phosphate salt exhibits an XRPD pattern which further comprises at least seven peaks selected from about 5.30, 14.41, 15.95, 19.76, 24.22, 25.28, 27.56, and 28.97 degrees two-theta with the margin of error of about ±0.5; about ±0.4; about ±0.3; about ±0.2; about ±0.1; about ±0.05; or less. In embodiments, crystalline Form E of Compound I phosphate salt exhibits an XRPD pattern which further comprises peaks at about 5.30, 14.41, 15.95, 19.76, 24.22, 25.28, 27.56, and 28.97 degrees two-theta with the margin of error of about ±0.5; about ±0.4; about ±0.3; about ±0.2; about ±0.1; about ±0.05; or less.

In embodiments, crystalline Form E of Compound I phosphate salt exhibits an XRPD pattern comprising peaks at about 20.42±0.2, 20.94±0.2, and 21.65±0.2 degrees two-theta.

In embodiments, crystalline Form E of Compound I phosphate salt exhibits an XRPD pattern comprising peaks at about 7.21±0.2, 10.18±0.2, 20.42±0.2, 20.94±0.2, and 21.65±0.2 degrees two-theta.

In embodiments, crystalline Form E of Compound I phosphate salt exhibits an XRPD pattern which further comprises at least two peaks selected from about 5.30±0.2, 14.41±0.2, 15.95±0.2, 19.76±0.2, 24.22±0.2, 25.28±0.2, 27.56±0.2, and 28.97±0.2 degrees two-theta.

In some embodiments, the crystalline form of Compound I phosphate salt exhibits an XRPD pattern comprising 1 peak, 2 peaks, 3 peaks, 4 peaks, 5 peaks, 6 peaks, 7 peaks, 8 peaks, 9 peaks, or 10 peaks ±0.2 degrees two-theta of Table 25.

In embodiments, crystalline Form E of Compound I phosphate salt exhibits an XRPD pattern comprising peaks shown in Table 25, below.

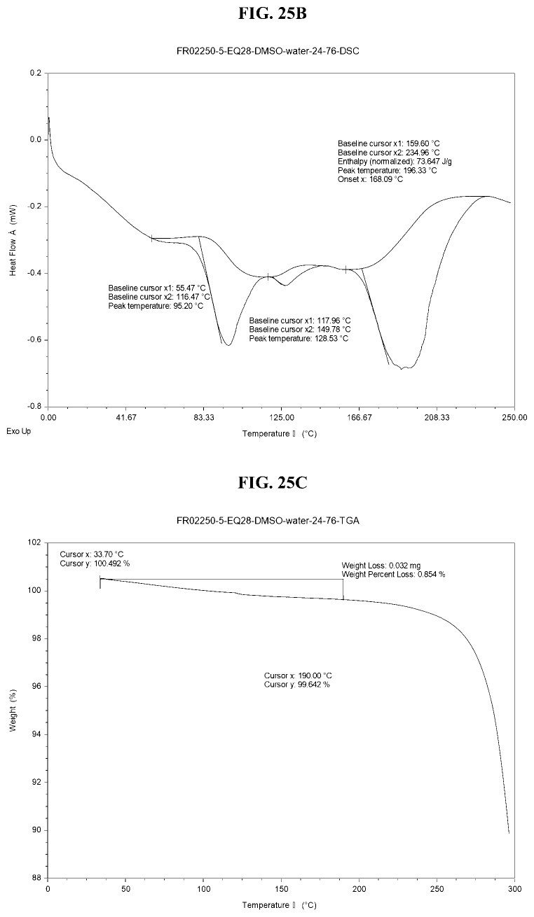

In embodiments, crystalline Form E of Compound I phosphate salt exhibits an XRPD pattern that is substantially similar to FIG. 25A.

TABLE 25
Form E of Compound I phosphate salt

| | | d | Net | Gross | Rel. |
|---|---|---|---|---|---|
| Index | Angle | Value | Intensity | Intensity | Intensity |
| 1 | 5.298° | 16.66737 Å | 101.856 | 136.726 | 43.0% |
| 2 | 7.213° | 12.24563 Å | 138.891 | 188.088 | 58.7% |
| 3 | 7.730° | 11.42769 Å | 23.7538 | 77.7986 | 10.0% |
| 4 | 10.179° | 8.68305 Å | 128.177 | 201.538 | 54.1% |
| 5 | 10.610° | 8.33167 Å | 54.3158 | 134.091 | 22.9% |
| 6 | 11.409° | 7.74968 Å | 39.7023 | 129.039 | 16.8% |
| 7 | 12.897° | 6.85882 Å | 20.1155 | 121.589 | 8.5% |
| 8 | 14.406° | 6.14348 Å | 96.3404 | 205.456 | 40.7% |
| 9 | 14.893° | 5.94363 Å | 55.8280 | 166.977 | 23.6% |
| 10 | 15.950° | 5.55193 Å | 98.0827 | 216.203 | 41.4% |
| 11 | 18.009° | 4.92179 Å | 62.2723 | 209.997 | 26.3% |
| 12 | 18.595° | 4.76776 Å | 73.3536 | 231.939 | 31.0% |
| 13 | 19.041° | 4.65714 Å | 70.9229 | 236.659 | 30.0% |
| 14 | 19.760° | 4.48926 Å | 112.462 | 287.733 | 47.5% |
| 15 | 20.423° | 4.34509 Å | 188.500 | 370.372 | 79.6% |
| 16 | 20.939° | 4.23906 Å | 229.170 | 414.734 | 96.8% |
| 17 | 21.645° | 4.10239 Å | 236.757 | 425.306 | 100.0% |
| 18 | 22.799° | 3.89727 Å | 37.4372 | 225.742 | 15.8% |
| 19 | 24.218° | 3.67205 Å | 98.9525 | 284.646 | 41.8% |
| 20 | 25.281° | 3.52000 Å | 86.6381 | 268.632 | 36.6% |
| 21 | 27.559° | 3.23405 Å | 98.7062 | 254.605 | 41.7% |
| 22 | 28.972° | 3.07943 Å | 106.257 | 250.527 | 44.9% |
| 23 | 30.763° | 2.90407 Å | 47.0895 | 182.410 | 19.9% |
| 24 | 37.717° | 2.38312 Å | 26.3436 | 144.989 | 11.1% |

In embodiments, the crystalline Form E of Compound I phosphate salt exhibits a DSC thermogram comprising an endotherm peak at about 95.2° C. with the error of margin of about ±2.5; about ±2.0; about ±1.5; about ±1.0; about ±0.5; or less. In embodiments, the crystalline Form E of Compound I phosphate salt exhibits a DSC thermogram comprising an endotherm peak at about 128.6° C. with the error of margin of about ±2.5; about ±2.0; about ±1.5; about ±1.0; about ±0.5; or less. In embodiments, the crystalline Form E of Compound I phosphate salt exhibits a DSC thermogram comprising an endotherm peak with an onset at about 168° C. with the error of margin of about ±2.5; about ±2.0; about ±1.5; about ±1.0; about ±0.5; or less.

In some embodiments, the crystalline Form E of Compound I phosphate salt exhibits a DSC thermogram that is substantially similar to FIG. 25B.

In embodiments, the crystalline Form E of Compound I phosphate salt exhibits a TGA thermogram substantially similar to FIG. 25C. In embodiments, the crystalline Form E of Compound I phosphate salt exhibits a weight percent loss of about 0.9% between about 34° C. to about 190° C. by a thermogravimetric analysis (TGA).

### Compound I Phosphate Salt Form F

In embodiments, the present disclosure relates to a crystalline form of Compound I phosphate salt, which is Form F.

In embodiments of the present disclosure, crystalline Form F of Compound I phosphate salt is a solvate.

In embodiments of the present disclosure, crystalline Form F of Compound I phosphate salt is a DMSO solvate.

In embodiments, the crystalline form of Compound I phosphate salt may comprise of over about 99.9%, about 99.8%, about 99.7%, about 99.6%, about 99.5%, about 99.4%, about 99.3%, about 99.2%, about 99.1%, or about 99.0% of Form F of Compound I phosphate salt. In another embodiment, the crystalline form of Compound I phosphate salt may comprise over about 99%, 98%, 97%, 96%, 95%, 94%, 93%, 92%, 91%, or 90% of Form F of Compound I phosphate salt. In some embodiments, the crystalline form of Compound I phosphate salt may comprise over about 90%, 85%, 80%, 75%, 70%, 65%, 60%, 55%, 50%, 45%, or 40% of Form F of Compound I phosphate salt.

In embodiments, crystalline Form F of Compound I phosphate salt exhibits an XRPD pattern comprising peaks at about 11.11, 20.77, and 21.32 degrees two-theta with the margin of error of about ±0.5; about ±0.4; about ±0.3; about ±0.2; about ±0.1; about ±0.05; or less. In embodiments, crystalline Form F of Compound I phosphate salt exhibits an XRPD pattern comprising peaks at about 10.10, 11.11, 20.77, 21.32, and 24.20 degrees two-theta with the margin of error of about ±0.5; about ±0.4; about ±0.3; about ±0.2; about ±0.1; about ±0.05; or less. In embodiments, crystalline Form F of Compound I phosphate salt exhibits an XRPD pattern which further comprises at least two peaks selected from about 15.84, 16.81, 20.19, 22.57, 22.71, 23.15, 25.23 and 25.60 degrees two-theta with the margin of error of about ±0.5; about ±0.4; about ±0.3; about ±0.2; about ±0.1; about ±0.05; or less. In embodiments, crystalline Form F of Compound I phosphate salt exhibits an XRPD pattern which further comprises at least three peaks selected from about 15.84, 16.81, 20.19, 22.57, 22.71, 23.15, 25.23 and 25.60 degrees two-theta with the margin of error of about ±0.5; about ±0.4; about ±0.3; about ±0.2; about ±0.1; about ±0.05; or less. In embodiments, crystalline Form F of Compound I phosphate salt exhibits an XRPD pattern which further comprises at least four peaks selected from about 15.84, 16.81, 20.19, 22.57, 22.71, 23.15, 25.23 and 25.60 degrees two-theta with the margin of error of about ±0.5; about ±0.4; about ±0.3; about ±0.2; about ±0.1; about ±0.05; or less. In embodiments, crystalline Form F of Compound I phosphate salt exhibits an XRPD pattern which further comprises at least five peaks selected from about 15.84, 16.81, 20.19, 22.57, 22.71, 23.15, 25.23 and 25.60 degrees two-theta with the margin of error of about ±0.5; about ±0.4; about ±0.3; about ±0.2; about ±0.1; about ±0.05; or less. In embodiments, crystalline Form F of Compound I phosphate salt exhibits an XRPD pattern which further comprises at least six peaks selected from about 15.84, 16.81, 20.19, 22.57, 22.71, 23.15, 25.23 and 25.60 degrees two-theta with the margin of error of about ±0.5; about ±0.4; about ±0.3; about ±0.2; about ±0.1; about ±0.05; or less. In embodiments, crystalline Form F of Compound I phosphate salt exhibits an XRPD pattern which further comprises at least seven peaks selected from about 15.84, 16.81, 20.19, 22.57, 22.71, 23.15, 25.23 and 25.60 degrees two-theta with the margin of error of about ±0.5; about ±0.4; about ±0.3; about ±0.2; about ±0.1; about ±0.05; or less. In embodiments, crystalline Form F of Compound I phosphate salt exhibits an XRPD pattern which further comprises peaks at about 15.84, 16.81, 20.19, 22.57, 22.71, 23.15, 25.23 and 25.60 degrees two-theta with the margin of error of about ±0.5; about ±0.4; about ±0.3; about ±0.2; about ±0.1; about ±0.05; or less.

In embodiments, crystalline Form F of Compound I phosphate salt exhibits an XRPD pattern comprising peaks at about 11.11±0.2, 20.77±0.2, and 21.32±0.2 degrees two-theta.

In embodiments, crystalline Form F of Compound I phosphate salt exhibits an XRPD pattern comprising peaks at about 10.10±0.2, 11.11±0.2, 20.77±0.2, 21.32±0.2, and 24.20±0.2 degrees two-theta.

In embodiments, crystalline Form F of Compound I phosphate salt exhibits an XRPD pattern which further comprises at least two peaks selected from about 15.84±0.2, 16.81±0.2, 20.19±0.2, 22.57±0.2, 22.71±0.2, 23.15±0.2, 25.23±0.2 and 25.60±0.2 degrees two-theta.

In some embodiments, the crystalline form of Compound I phosphate salt exhibits an XRPD pattern comprising 1 peak, 2 peaks, 3 peaks, 4 peaks, 5 peaks, 6 peaks, 7 peaks, 8 peaks, 9 peaks, or 10 peaks ±0.2 degrees two-theta of Table 26.

In embodiments, crystalline Form F of Compound I phosphate salt exhibits an XRPD pattern comprising peaks shown in Table 26, below.

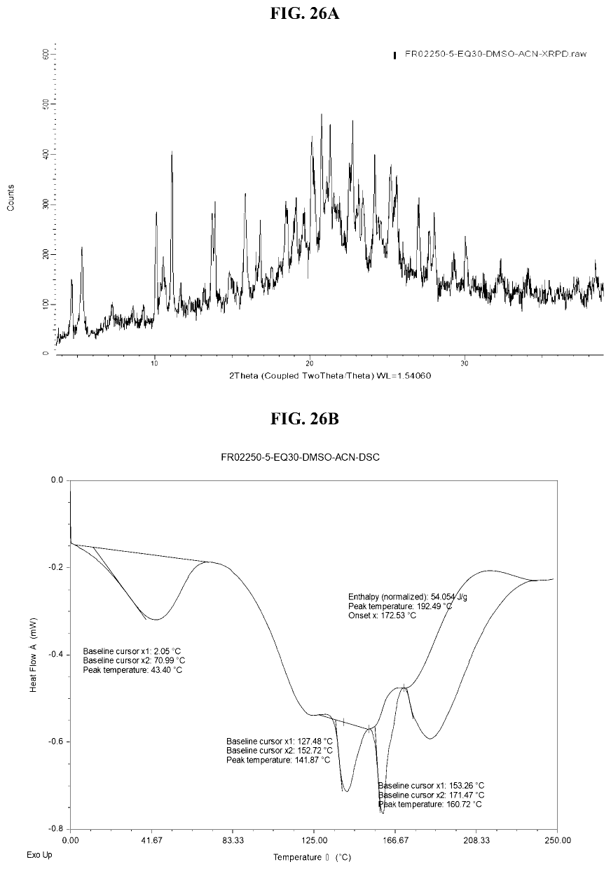

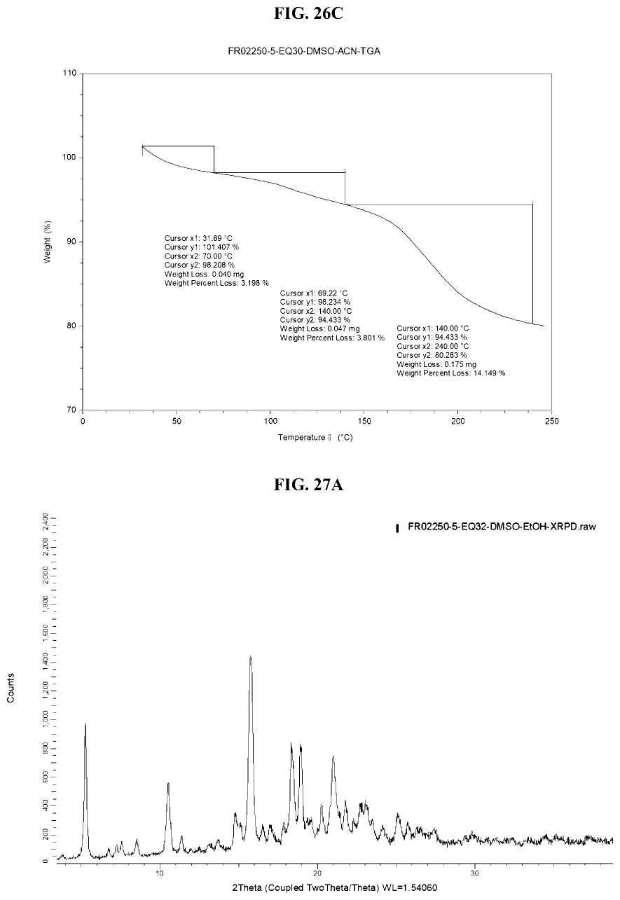

In embodiments, crystalline Form F of Compound I phosphate salt exhibits an XRPD pattern that is substantially similar to FIG. 26A.

TABLE 26
Form F of Compound I phosphate salt

| | | d | Net | Gross | Rel. |
|---|---|---|---|---|---|
| Index | Angle | Value | Intensity | Intensity | Intensity |
| 1 | 4.661° | 18.94433 Å | 63.6857 | 99.3425 | 30.6% |
| 2 | 5.309° | 16.63152 Å | 92.6677 | 132.047 | 44.6% |
| 3 | 7.261° | 12.16499 Å | 30.4462 | 84.0589 | 14.6% |
| 4 | 9.265° | 9.53773 Å | 24.3961 | 88.3072 | 11.7% |
| 5 | 10.104° | 8.74726 Å | 146.721 | 216.631 | 70.6% |
| 6 | 10.539° | 8.38766 Å | 70.9819 | 146.569 | 34.1% |
| 7 | 11.112° | 7.95632 Å | 207.879 | 289.689 | 100.0% |
| 8 | 11.672° | 7.57593 Å | 40.2170 | 126.716 | 19.3% |
| 9 | 13.239° | 6.68250 Å | 33.9395 | 128.245 | 16.3% |
| 10 | 13.783° | 6.41984 Å | 51.5832 | 149.605 | 24.8% |
| 11 | 14.890° | 5.94473 Å | 35.4719 | 138.570 | 17.1% |
| 12 | 14.858° | 5.95746 Å | 35.9789 | 138.958 | 17.3% |
| 13 | 15.839° | 5.59089 Å | 140.517 | 252.391 | 67.6% |
| 14 | 16.531° | 5.35823 Å | 34.5189 | 160.032 | 16.6% |
| 15 | 16.807° | 5.27071 Å | 112.365 | 243.216 | 54.1% |
| 16 | 18.488° | 4.79529 Å | 68.6337 | 229.852 | 33.0% |
| 17 | 19.113° | 4.63976 Å | 89.9564 | 260.240 | 43.3% |
| 18 | 19.608° | 4.52385 Å | 61.5076 | 237.745 | 29.6% |
| 19 | 20.194° | 4.39384 Å | 104.276 | 286.178 | 50.2% |
| 20 | 20.767° | 4.27379 Å | 203.152 | 389.140 | 97.7% |
| 21 | 21.320° | 4.16421 Å | 207.362 | 395.921 | 99.8% |
| 22 | 21.924° | 4.05083 Å | 93.6415 | 283.479 | 45.0% |
| 23 | 22.565° | 3.93726 Å | 128.088 | 317.534 | 61.6% |
| 24 | 22.705° | 3.91327 Å | 116.696 | 305.816 | 56.1% |
| 25 | 23.152° | 3.83867 Å | 123.791 | 311.294 | 59.5% |
| 26 | 23.429° | 3.79393 Å | 99.8487 | 285.911 | 48.0% |
| 27 | 24.196° | 3.67544 Å | 170.163 | 351.957 | 81.9% |
| 28 | 24.488° | 3.63223 Å | 41.2930 | 221.155 | 19.9% |
| 29 | 25.228° | 3.52736 Å | 125.264 | 298.558 | 60.3% |
| 30 | 25.595° | 3.47755 Å | 145.339 | 314.480 | 69.9% |
| 31 | 27.041° | 3.29473 Å | 98.4814 | 250.302 | 47.4% |
| 32 | 27.728° | 3.21468 Å | 77.1404 | 219.506 | 37.1% |
| 33 | 28.046° | 3.17894 Å | 102.471 | 239.754 | 49.3% |
| 34 | 29.305° | 3.04517 Å | 64.7272 | 196.111 | 31.1% |
| 35 | 30.065° | 2.96992 Å | 65.0721 | 194.519 | 31.3% |
| 36 | 32.297° | 2.76956 Å | 61.4644 | 180.526 | 29.6% |
| 37 | 34.127° | 2.62517 Å | 40.1911 | 155.132 | 19.3% |
| 38 | 35.449° | 2.53018 Å | 30.9852 | 144.262 | 14.9% |
| 39 | 38.484° | 2.33739 Å | 43.6708 | 159.334 | 21.0% |
| 40 | 39.751° | 2.26575 Å | 22.7194 | 140.363 | 10.9% |

In embodiments, the crystalline Form F of Compound I phosphate salt exhibits a DSC thermogram comprising an endotherm peak at about 43° C. and/or an endotherm peak at about 142° C. and/or an endotherm peak at about 161° C. and/or an endotherm peak at about 192.5° C. with the error of margin of about ±2.5; about ±2.0; about ±1.5; about ±1.0; about ±0.5; or less. In embodiments, the crystalline Form F of Compound I phosphate salt exhibits a DSC thermogram comprising an endotherm peak with an onset at about 172.5° C. with the error of margin of about ±2.5; about ±2.0; about ±1.5; about ±1.0; about ±0.5; or less.

In some embodiments, the crystalline Form F of Compound I phosphate salt exhibits a DSC thermogram that is substantially similar to FIG. 26B.

In embodiments, the crystalline Form F of Compound I phosphate salt exhibits a TGA thermogram substantially similar to FIG. 26C. In embodiments, the crystalline Form F of Compound I phosphate salt exhibits a weight percent loss of about 3.2% between about 32° C. to about 70° C. by a thermogravimetric analysis (TGA). In embodiments, the crystalline Form F of Compound I phosphate salt exhibits a weight percent loss of about 3.8% between about 69° C. to about 140° C. by a thermogravimetric analysis (TGA). In embodiments, the crystalline Form F of Compound I phosphate salt exhibits a weight percent loss of about 14.2% between about 140° C. to about 240° C. by a thermogravimetric analysis (TGA).

### Compound I Phosphate Salt Form G

In embodiments, the present disclosure relates to a crystalline form of Compound I phosphate salt, which is Form G.

In embodiments of the present disclosure, crystalline Form G of Compound I phosphate salt is a solvate.

In embodiments of the present disclosure, crystalline Form G of Compound I phosphate salt is a DMSO-water solvate.

In embodiments, the crystalline form of Compound I phosphate salt may comprise of over about 99.9%, about 99.8%, about 99.7%, about 99.6%, about 99.5%, about 99.4%, about 99.3%, about 99.2%, about 99.1%, or about 99.0% of Form G of Compound I phosphate salt. In another embodiment, the crystalline form of Compound I phosphate salt may comprise over about 99%, 98%, 97%, 96%, 95%, 94%, 93%, 92%, 91%, or 90% of Form G of Compound I phosphate salt. In some embodiments, the crystalline form of Compound I phosphate salt may comprise over about 90%, 85%, 80%, 75%, 70%, 65%, 60%, 55%, 50%, 45%, or 40% of Form G of Compound I phosphate salt.

In embodiments, crystalline Form G of Compound I phosphate salt exhibits an XRPD pattern comprising peaks at about 5.28, 15.77, and 18.95 degrees two-theta with the margin of error of about ±0.5; about ±0.4; about ±0.3; about ±0.2; about ±0.1; about ±0.05; or less. In embodiments, crystalline Form G of Compound I phosphate salt exhibits an XRPD pattern comprising peaks at about 5.28, 15.77, 18.39 18.95, and 21.00 degrees two-theta with the margin of error of about ±0.5; about ±0.4; about ±0.3; about ±0.2; about ±0.1; about ±0.05; or less. In embodiments, crystalline Form G of Compound I phosphate salt exhibits an XRPD pattern which further comprises at least two peaks selected from about 10.54, 14.81, 15.03, 20.27, 21.79, 22.76, 23.06, and 25.10 degrees two-theta with the margin of error of about ±0.5; about ±0.4; about ±0.3; about ±0.2; about ±0.1; about ±0.05; or less. In embodiments, crystalline Form G of Compound I phosphate salt exhibits an XRPD pattern which further comprises at least three peaks selected from about 10.54, 14.81, 15.03, 20.27, 21.79, 22.76, 23.06, and 25.10 degrees two-theta with the margin of error of about ±0.5; about ±0.4; about ±0.3; about ±0.2; about ±0.1; about ±0.05; or less. In embodiments, crystalline Form G of Compound I phosphate salt exhibits an XRPD pattern which further comprises at least four peaks selected from about 10.54, 14.81, 15.03, 20.27, 21.79, 22.76, 23.06, and 25.10 degrees two-theta with the margin of error of about ±0.5; about ±0.4; about ±0.3; about ±0.2; about ±0.1; about ±0.05; or less. In embodiments, crystalline Form G of Compound I phosphate salt exhibits an XRPD pattern which further comprises at least five peaks selected from about 10.54, 14.81, 15.03, 20.27, 21.79, 22.76, 23.06, and 25.10 degrees two-theta with the margin of error of about ±0.5; about ±0.4; about ±0.3; about ±0.2; about ±0.1; about ±0.05; or less. In embodiments, crystalline Form G of Compound I phosphate salt exhibits an XRPD pattern which further comprises at least six peaks selected from about 10.54, 14.81, 15.03, 20.27, 21.79, 22.76, 23.06, and 25.10 degrees two-theta with the margin of error of about ±0.5; about ±0.4; about ±0.3; about ±0.2; about ±0.1; about ±0.05; or less. In embodiments, crystalline Form G of Compound I phosphate salt exhibits an XRPD pattern which further comprises at least seven peaks selected from about 10.54, 14.81, 15.03, 20.27, 21.79, 22.76, 23.06, and 25.10 degrees two-theta with the margin of error of about ±0.5; about ±0.4; about ±0.3; about ±0.2; about ±0.1; about ±0.05; or less. In embodiments, crystalline Form G of Compound I phosphate salt exhibits an XRPD pattern which further comprises peaks at about 10.54, 14.81, 15.03, 20.27, 21.79, 22.76, 23.06, and 25.10 degrees two-theta with the margin of error of about ±0.5; about ±0.4; about ±0.3; about ±0.2; about ±0.1; about ±0.05; or less.

In embodiments, crystalline Form G of Compound I phosphate salt exhibits an XRPD pattern comprising peaks at about 5.28±0.2, 15.77±0.2, and 18.95±0.2 degrees two-theta.

In embodiments, crystalline Form G of Compound I phosphate salt exhibits an XRPD pattern comprising peaks at about 5.28±0.2, 15.77±0.2, 18.39±0.2 18.95±0.2, and 21.00±0.2 degrees two-theta.

In embodiments, crystalline Form G of Compound I phosphate salt exhibits an XRPD pattern which further comprises at least two peaks selected from about 10.54±0.2, 14.81±0.2, 15.03±0.2, 20.27±0.2, 21.79±0.2, 22.76±0.2, 23.06±0.2, and 25.10±0.2 degrees two-theta.

In some embodiments, the crystalline form of Compound I phosphate salt exhibits an XRPD pattern comprising 1 peak, 2 peaks, 3 peaks, 4 peaks, 5 peaks, 6 peaks, 7 peaks, 8 peaks, 9 peaks, or 10 peaks ±0.2 degrees two-theta of Table 27.

In embodiments, crystalline Form G of Compound I phosphate salt exhibits an XRPD pattern comprising peaks shown in Table 27, below.

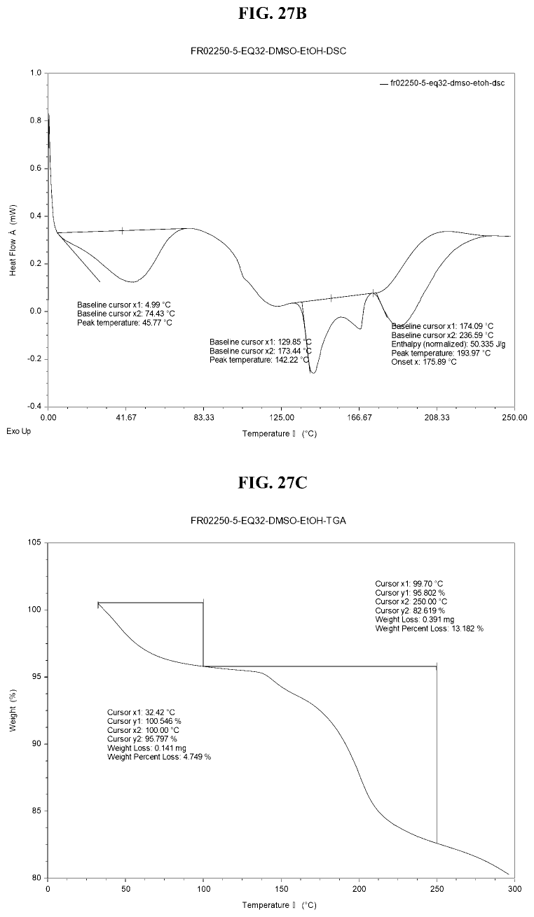

In embodiments, crystalline Form G of Compound I phosphate salt exhibits an XRPD pattern that is substantially similar to FIG. 27A.

TABLE 27
Form G of Compound I phosphate salt

| | | d | Net | Gross | Rel. |
|---|---|---|---|---|---|
| Index | Angle | Value | Intensity | Intensity | Intensity |
| 1 | 5.279° | 16.72661 Å | 577.626 | 618.712 | 66.4% |
| 2 | 6.749° | 13.08619 Å | 35.9764 | 83.7269 | 4.1% |
| 3 | 7.257° | 12.17160 Å | 50.4419 | 102.862 | 5.8% |
| 4 | 7.578° | 11.65630 Å | 59.5657 | 113.928 | 6.8% |
| 5 | 8.531° | 10.35646 Å | 82.0500 | 137.558 | 9.4% |
| 6 | 10.539° | 8.38708 Å | 320.337 | 393.495 | 36.8% |
| 7 | 11.370° | 7.77586 Å | 79.8722 | 156.433 | 9.2% |
| 8 | 11.989° | 7.37603 Å | 22.5333 | 100.669 | 2.6% |
| 9 | 13.123° | 6.74102 Å | 31.6749 | 120.175 | 3.6% |
| 10 | 13.722° | 6.44833 Å | 28.7563 | 127.663 | 3.3% |
| 11 | 14.813° | 5.97547 Å | 156.966 | 276.700 | 18.1% |
| 12 | 15.030° | 5.88975 Å | 99.9309 | 223.947 | 11.5% |
| 13 | 15.770° | 5.61505 Å | 869.593 | 1005.53 | 100.0% |
| 14 | 16.559° | 5.34915 Å | 77.5827 | 221.651 | 8.9% |
| 15 | 17.044° | 5.19796 Å | 77.0740 | 223.789 | 8.9% |
| 16 | 17.876° | 4.95804 Å | 96.5704 | 249.559 | 11.1% |
| 17 | 18.387° | 4.82138 Å | 380.364 | 545.479 | 43.7% |
| 18 | 18.946° | 4.68043 Å | 402.313 | 578.418 | 46.3% |
| 19 | 19.534° | 4.54066 Å | 60.6877 | 245.802 | 7.0% |
| 20 | 20.273° | 4.37690 Å | 141.850 | 334.538 | 16.3% |
| 21 | 21.000° | 4.22697 Å | 373.318 | 569.415 | 42.9% |
| 22 | 21.785° | 4.07635 Å | 180.450 | 375.712 | 20.8% |
| 23 | 22.382° | 3.96896 Å | 65.8447 | 257.338 | 7.6% |
| 24 | 22.761° | 3.90375 Å | 119.880 | 307.574 | 13.8% |
| 25 | 23.058° | 3.85417 Å | 191.991 | 375.947 | 22.1% |
| 26 | 23.496° | 3.78330 Å | 85.1107 | 262.328 | 9.8% |
| 27 | 24.141° | 3.68369 Å | 67.2173 | 231.858 | 7.7% |
| 28 | 25.104° | 3.54451 Å | 129.774 | 286.502 | 14.9% |
| 29 | 25.711° | 3.46212 Å | 81.4945 | 240.720 | 9.4% |
| 30 | 27.403° | 3.25207 Å | 58.3697 | 209.763 | 6.7% |
| 31 | 29.815° | 2.99426 Å | 62.2426 | 206.144 | 7.2% |
| 32 | 35.158° | 2.55046 Å | 46.7253 | 193.175 | 5.4% |

In embodiments, the crystalline Form G of Compound I phosphate salt exhibits a DSC thermogram comprising an endotherm peak at about 46° C. and/or an endotherm peak at about 142° C. and/or an endotherm peak at about 194° C. with the error of margin of about ±2.5; about ±2.0; about ±1.5; about ±1.0; about ±0.5; or less. In embodiments, the crystalline Form G of Compound I phosphate salt exhibits a DSC thermogram comprising an endotherm peak with an onset at about 176° C. with the error of margin of about ±2.5; about ±2.0; about ±1.5; about ±1.0; about ±0.5; or less.

In some embodiments, the crystalline Form G of Compound I phosphate salt exhibits a DSC thermogram that is substantially similar to FIG. 27B.

In embodiments, the crystalline Form G of Compound I phosphate salt exhibits a TGA thermogram substantially similar to FIG. 27C. In embodiments, the crystalline Form G of Compound I phosphate salt exhibits a weight percent loss of about 4.8% between about 32° C. to about 100° C. by a thermogravimetric analysis (TGA). In embodiments, the crystalline Form G of Compound I phosphate salt exhibits a weight percent loss of about 13.2% between about 100° C. to about 250° C. by a thermogravimetric analysis (TGA).

### Compound I Phosphate Salt Form I

In embodiments, the present disclosure relates to a crystalline form of Compound I phosphate salt, which is Form I.

In embodiments of the present disclosure, crystalline Form I of Compound I phosphate salt is a solvate.

In embodiments of the present disclosure, crystalline Form I of Compound I phosphate salt is a 2,2,2-trifluoroethanol (TFE) solvate.

In embodiments, the crystalline form of Compound I phosphate salt may comprise of over about 99.9%, about 99.8%, about 99.7%, about 99.6%, about 99.5%, about 99.4%, about 99.3%, about 99.2%, about 99.1%, or about 99.0% of Form I of Compound I phosphate salt. In another embodiment, the crystalline form of Compound I phosphate salt may comprise over about 99%, 98%, 97%, 96%, 95%, 94%, 93%, 92%, 91%, or 90% of Form I of Compound I phosphate salt. In some embodiments, the crystalline form of Compound I phosphate salt may comprise over about 90%, 85%, 80%, 75%, 70%, 65%, 60%, 55%, 50%, 45%, or 40% of Form I of Compound I phosphate salt.

In embodiments, crystalline Form I of Compound I phosphate salt exhibits an XRPD pattern comprising peaks at about 8.56, 9.63, and 20.48 degrees two-theta with the margin of error of about ±0.5; about ±0.4; about ±0.3; about ±0.2; about ±0.1; about ±0.05; or less. In embodiments, crystalline Form I of Compound I phosphate salt exhibits an XRPD pattern comprising peaks at about 8.56, 9.63, 17.91, 20.48, and 23.87 degrees two-theta with the margin of error of about ±0.5; about ±0.4; about ±0.3; about ±0.2; about ±0.1; about ±0.05; or less. In embodiments, crystalline Form I of Compound I phosphate salt exhibits an XRPD pattern which further comprises at least two peaks selected from about 14.85, 19.61, 21.10, 21.60, 22.68, 23.31, 26.98 and 29.87 degrees two-theta with the margin of error of about ±0.5; about ±0.4; about ±0.3; about ±0.2; about ±0.1; about ±0.05; or less. In embodiments, crystalline Form I of Compound I phosphate salt exhibits an XRPD pattern which further comprises at least three peaks selected from about 14.85, 19.61, 21.10, 21.60, 22.68, 23.31, 26.98 and 29.87 degrees two-theta with the margin of error of about ±0.5; about ±0.4; about ±0.3; about ±0.2; about ±0.1; about ±0.05; or less. In embodiments, crystalline Form I of Compound I phosphate salt exhibits an XRPD pattern which further comprises at least four peaks selected from about 14.85, 19.61, 21.10, 21.60, 22.68, 23.31, 26.98 and 29.87 degrees two-theta with the margin of error of about ±0.5; about ±0.4; about ±0.3; about ±0.2; about ±0.1; about ±0.05; or less. In embodiments, crystalline Form I of Compound I phosphate salt exhibits an XRPD pattern which further comprises at least five peaks selected from about 14.85, 19.61, 21.10, 21.60, 22.68, 23.31, 26.98 and 29.87 degrees two-theta with the margin of error of about ±0.5; about ±0.4; about ±0.3; about ±0.2; about ±0.1; about ±0.05; or less. In embodiments, crystalline Form I of Compound I phosphate salt exhibits an XRPD pattern which further comprises at least six peaks selected from about 14.85, 19.61, 21.10, 21.60, 22.68, 23.31, 26.98 and 29.87 degrees two-theta with the margin of error of about ±0.5; about ±0.4; about ±0.3; about ±0.2; about ±0.1; about ±0.05; or less. In embodiments, crystalline Form I of Compound I phosphate salt exhibits an XRPD pattern which further comprises at least seven peaks selected from about 14.85, 19.61, 21.10, 21.60, 22.68, 23.31, 26.98 and 29.87 degrees two-theta with the margin of error of about ±0.5; about ±0.4; about ±0.3; about ±0.2; about ±0.1; about ±0.05; or less. In embodiments, crystalline Form I of Compound I phosphate salt exhibits an XRPD pattern which further comprises peaks at about 14.85, 19.61, 21.10, 21.60, 22.68, 23.31, 26.98 and 29.87 degrees two-theta with the margin of error of about ±0.5; about ±0.4; about ±0.3; about ±0.2; about ±0.1; about ±0.05; or less.

In embodiments, crystalline Form I of Compound I phosphate salt exhibits an XRPD pattern comprising peaks at about 8.56±0.2, 9.63±0.2, and 20.48±0.2 degrees two-theta.

In embodiments, crystalline Form I of Compound I phosphate salt exhibits an XRPD pattern comprising peaks at about 8.56±0.2, 9.63±0.2, 17.91±0.2, 20.48±0.2, and 23.87±0.2 degrees two-theta.

In embodiments, crystalline Form I of Compound I phosphate salt exhibits an XRPD pattern which further comprises at least two peaks selected from about 14.85±0.2, 19.61±0.2, 21.10±0.2, 21.60±0.2, 22.68±0.2, 23.31±0.2, 26.98±0.2 and 29.87±0.2 degrees two-theta.

In some embodiments, the crystalline form of Compound I phosphate salt exhibits an XRPD pattern comprising 1 peak, 2 peaks, 3 peaks, 4 peaks, 5 peaks, 6 peaks, 7 peaks, 8 peaks, 9 peaks, or 10 peaks ±0.2 degrees two-theta of Table 28.

In embodiments, crystalline Form I of Compound I phosphate salt exhibits an XRPD pattern comprising peaks shown in Table 28, below.

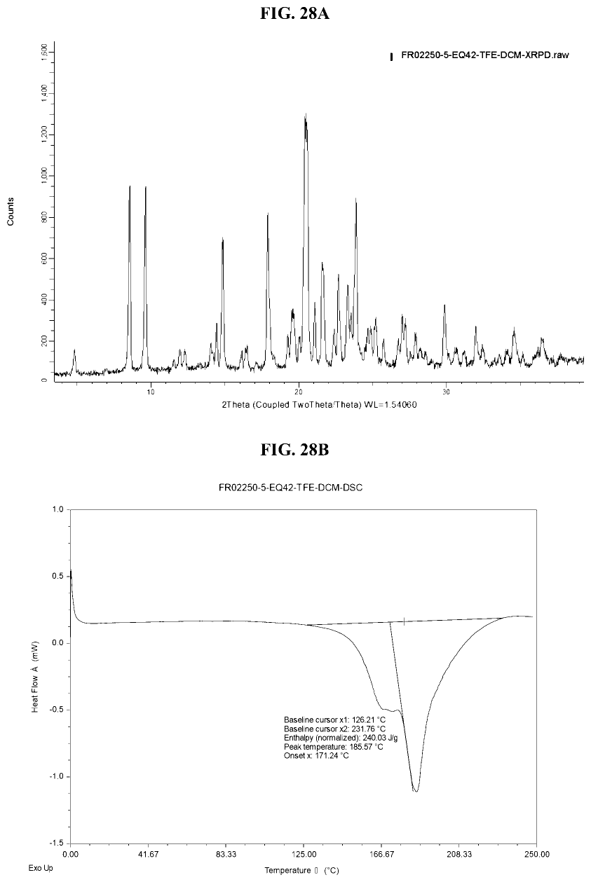

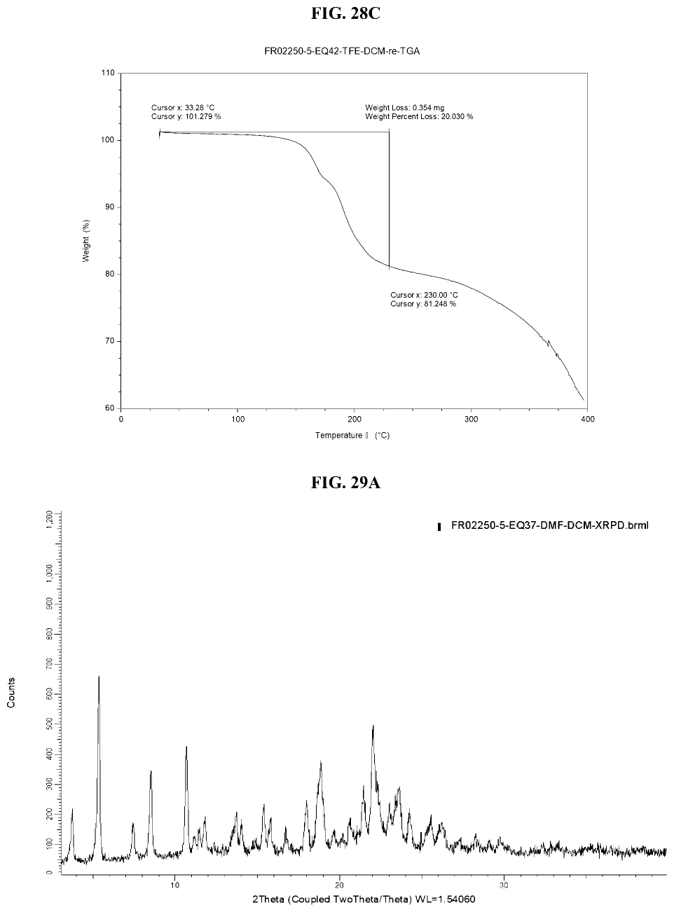

In embodiments, crystalline Form I of Compound I phosphate salt exhibits an XRPD pattern that is substantially similar to FIG. 28A.

TABLE 28
Form I of Compound I phosphate salt

| | | d | Net | Gross | Rel. |
|---|---|---|---|---|---|
| Index | Angle | Value | Intensity | Intensity | Intensity |
| 1 | 4.831° | 18.27546 Å | 71.4247 | 108.226 | 8.8% |
| 2 | 7.017° | 12.58731 Å | 15.1489 | 55.9874 | 1.9% |
| 3 | 8.564° | 10.31677 Å | 601.162 | 658.856 | 74.0% |
| 4 | 9.634° | 9.17272 Å | 601.043 | 660.871 | 74.0% |
| 5 | 11.571° | 7.64120 Å | 30.7136 | 89.5619 | 3.8% |
| 6 | 11.975° | 7.38433 Å | 63.5482 | 126.692 | 7.8% |
| 7 | 12.292° | 7.19466 Å | 69.0023 | 134.689 | 8.5% |
| 8 | 14.053° | 6.29702 Å | 79.5250 | 154.345 | 9.8% |
| 9 | 14.440° | 6.12892 Å | 163.552 | 238.134 | 20.1% |
| 10 | 14.851° | 5.96030 Å | 379.306 | 452.449 | 46.7% |
| 11 | 16.142° | 5.48652 Å | 56.1841 | 124.208 | 6.9% |
| 12 | 16.470° | 5.37784 Å | 64.8890 | 132.951 | 8.0% |
| 13 | 17.910° | 4.94869 Å | 532.155 | 606.984 | 65.5% |
| 14 | 19.273° | 4.60162 Å | 111.207 | 190.784 | 13.7% |
| 15 | 19.611° | 4.52314 Å | 204.639 | 287.920 | 25.2% |
| 16 | 20.041° | 4.42694 Å | 86.5826 | 173.392 | 10.7% |
| 17 | 20.484° | 4.33219 Å | 811.969 | 901.011 | 100.0% |
| 18 | 21.100° | 4.20717 Å | 184.719 | 274.512 | 22.7% |
| 19 | 21.604° | 4.11020 Å | 313.606 | 401.970 | 38.6% |
| 20 | 22.389° | 3.96776 Å | 107.942 | 196.250 | 13.3% |
| 21 | 22.677° | 3.91798 Å | 297.111 | 388.422 | 36.6% |
| 22 | 23.308° | 3.81342 Å | 260.006 | 355.792 | 32.0% |
| 23 | 23.872° | 3.72452 Å | 545.103 | 642.459 | 67.1% |
| 24 | 24.673° | 3.60545 Å | 124.060 | 219.694 | 15.3% |
| 25 | 24.855° | 3.57946 Å | 117.565 | 212.157 | 14.5% |
| 26 | 25.199° | 3.53134 Å | 160.650 | 252.627 | 19.8% |
| 27 | 25.732° | 3.45941 Å | 78.5017 | 164.736 | 9.7% |
| 28 | 26.731° | 3.33233 Å | 88.2098 | 173.099 | 10.9% |
| 29 | 26.978° | 3.30236 Å | 183.650 | 269.515 | 22.6% |
| 30 | 27.212° | 3.27442 Å | 152.527 | 238.907 | 18.8% |
| 31 | 27.903° | 3.19493 Å | 113.073 | 198.663 | 13.9% |
| 32 | 28.197° | 3.16231 Å | 49.3120 | 133.522 | 6.1% |
| 33 | 28.586° | 3.12014 Å | 44.3373 | 125.757 | 5.5% |
| 34 | 29.866° | 2.98921 Å | 222.129 | 304.972 | 27.4% |
| 35 | 30.643° | 2.91517 Å | 50.7813 | 138.569 | 6.3% |
| 36 | 31.214° | 2.86319 Å | 49.4302 | 138.070 | 6.1% |
| 37 | 31.967° | 2.79739 Å | 147.775 | 233.938 | 18.2% |
| 38 | 32.421° | 2.75931 Å | 68.9183 | 151.611 | 8.5% |
| 39 | 33.560° | 2.66822 Å | 40.5136 | 122.943 | 5.0% |
| 40 | 34.070° | 2.62944 Å | 35.8663 | 120.159 | 4.4% |
| 41 | 34.574° | 2.59223 Å | 113.253 | 197.542 | 13.9% |
| 42 | 35.154° | 2.55078 Å | 55.7794 | 137.795 | 6.9% |
| 43 | 36.439° | 2.46373 Å | 87.4696 | 171.235 | 10.8% |
| 44 | 37.742° | 2.38162 Å | 24.2149 | 108.892 | 3.0% |

In embodiments, the crystalline Form I of Compound I phosphate salt exhibits a DSC thermogram comprising a broad desolvation peak from about 126.2° C. with the error of margin of about ±2.5; about ±2.0; about ±1.5; about ±1.0; about ±0.5; or less. In embodiments, the crystalline Form I of Compound I phosphate salt exhibits a DSC thermogram comprising an endotherm peak with an onset at about 171° C. with the error of margin of about ±2.5; about ±2.0; about ±1.5; about ±1.0; about ±0.5; or less. In embodiments, the crystalline Form I of Compound I phosphate salt exhibits a DSC thermogram comprising an endotherm peak at about 185.6° C. with the error of margin of about ±2.5; about ±2.0; about ±1.5; about ±1.0; about ±0.5; or less

In some embodiments, the crystalline Form I of Compound I phosphate salt exhibits a DSC thermogram that is substantially similar to FIG. 28B.

In embodiments, the crystalline Form I of Compound I phosphate salt exhibits a TGA thermogram substantially similar to FIG. 28C. In embodiments, the crystalline Form I of Compound I phosphate salt exhibits a weight percent loss of about 20% between about 33° C. to about 230° C. by a thermogravimetric analysis (TGA).

### Compound I Phosphate Salt Form H1

In embodiments, the present disclosure relates to a crystalline form of Compound I phosphate salt, which is Form H1.

In embodiments of the present disclosure, crystalline Form H1 of Compound I phosphate salt is a solvate.

In embodiments of the present disclosure, crystalline Form H1 of Compound I phosphate salt is a dimethylformamide (DMF)-water hetero solvate.

In embodiments, the crystalline form of Compound I phosphate salt may comprise of over about 99.9%, about 99.8%, about 99.7%, about 99.6%, about 99.5%, about 99.4%, about 99.3%, about 99.2%, about 99.1%, or about 99.0% of Form H1 of Compound I phosphate salt. In another embodiment, the crystalline form of Compound I phosphate salt may comprise over about 99%, 98%, 97%, 96%, 95%, 94%, 93%, 92%, 91%, or 90% of Form H1 of Compound I phosphate salt. In some embodiments, the crystalline form of Compound I phosphate salt may comprise over about 90%, 85%, 80%, 75%, 70%, 65%, 60%, 55%, 50%, 45%, or 40% of Form H1 of Compound I phosphate salt.

In embodiments, crystalline Form H1 of Compound I phosphate salt exhibits an XRPD pattern comprising peaks at about 5.37, 10.69, and 22.05 degrees two-theta with the margin of error of about ±0.5; about ±0.4; about ±0.3; about ±0.2; about ±0.1; about ±0.05; or less. In embodiments, crystalline Form H1 of Compound I phosphate salt exhibits an XRPD pattern comprising peaks at about 5.37, 8.53, 10.69, 18.86, and 22.05 degrees two-theta with the margin of error of about ±0.5; about ±0.4; about ±0.3; about ±0.2; about ±0.1; about ±0.05; or less. In embodiments, crystalline Form H1 of Compound I phosphate salt exhibits an XRPD pattern which further comprises at least two peaks selected from about 3.74, 15.39, 17.99, 18.80, 21.46, 22.29, 23.47, and 23.61 degrees two-theta with the margin of error of about ±0.5; about ±0.4; about ±0.3; about ±0.2; about ±0.1; about ±0.05; or less. In embodiments, crystalline Form H1 of Compound I phosphate salt exhibits an XRPD pattern which further comprises at least three peaks selected from about 3.74, 15.39, 17.99, 18.80, 21.46, 22.29, 23.47, and 23.61 degrees two-theta with the margin of error of about ±0.5; about ±0.4; about ±0.3; about ±0.2; about ±0.1; about ±0.05; or less. In embodiments, crystalline Form H1 of Compound I phosphate salt exhibits an XRPD pattern which further comprises at least four peaks selected from about 3.74, 15.39, 17.99, 18.80, 21.46, 22.29, 23.47, and 23.61 degrees two-theta with the margin of error of about ±0.5; about ±0.4; about ±0.3; about ±0.2; about ±0.1; about ±0.05; or less. In embodiments, crystalline Form H1 of Compound I phosphate salt exhibits an XRPD pattern which further comprises at least five peaks selected from about 3.74, 15.39, 17.99, 18.80, 21.46, 22.29, 23.47, and 23.61 degrees two-theta with the margin of error of about ±0.5; about ±0.4; about ±0.3; about ±0.2; about ±0.1; about ±0.05; or less. In embodiments, crystalline Form H1 of Compound I phosphate salt exhibits an XRPD pattern which further comprises at least six peaks selected from about 3.74, 15.39, 17.99, 18.80, 21.46, 22.29, 23.47, and 23.61 degrees two-theta with the margin of error of about ±0.5; about ±0.4; about ±0.3; about ±0.2; about ±0.1; about ±0.05; or less. In embodiments, crystalline Form H1 of Compound I phosphate salt exhibits an XRPD pattern which further comprises at least seven peaks selected from about 3.74, 15.39, 17.99, 18.80, 21.46, 22.29, 23.47, and 23.61 degrees two-theta with the margin of error of about ±0.5; about ±0.4; about ±0.3; about ±0.2; about ±0.1; about ±0.05; or less. In embodiments, crystalline Form H1 of Compound I phosphate salt exhibits an XRPD pattern which further comprises peaks at about 3.74, 15.39, 17.99, 18.80, 21.46, 22.29, 23.47, and 23.61 degrees two-theta with the margin of error of about ±0.5; about ±0.4; about ±0.3; about ±0.2; about ±0.1; about ±0.05; or less.

In embodiments, crystalline Form H1 of Compound I phosphate salt exhibits an XRPD pattern comprising peaks at about 5.37±0.2, 10.69±0.2, and 22.05±0.2 degrees two-theta.

In embodiments, crystalline Form H1 of Compound I phosphate salt exhibits an XRPD pattern comprising peaks at about 5.37±0.2, 8.53±0.2, 10.69±0.2, 18.86±0.2, and 22.05±0.2 degrees two-theta.

In embodiments, crystalline Form H1 of Compound I phosphate salt exhibits an XRPD pattern which further comprises at least two peaks selected from about 3.74±0.2, 15.39±0.2, 17.99±0.2, 18.80±0.2, 21.46±0.2, 22.29±0.2, 23.47±0.2, and 23.61±0.2 degrees two-theta.

In some embodiments, the crystalline form of Compound I phosphate salt exhibits an XRPD pattern comprising 1 peak, 2 peaks, 3 peaks, 4 peaks, 5 peaks, 6 peaks, 7 peaks, 8 peaks, 9 peaks, or 10 peaks ±0.2 degrees two-theta of Table 29.

In embodiments, crystalline Form H1 of Compound I phosphate salt exhibits an XRPD pattern comprising peaks shown in Table 29, below.

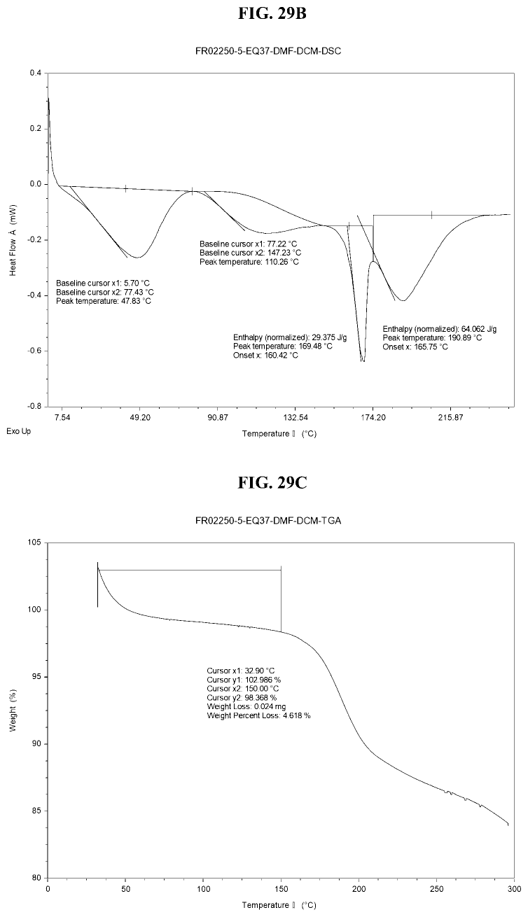

In embodiments, crystalline Form H1 of Compound I phosphate salt exhibits an XRPD pattern that is substantially similar to FIG. 29A.

TABLE 29
Form H1 of Compound I phosphate salt

| | | d | Net | Gross | Rel. |
|---|---|---|---|---|---|
| Index | Angle | Value | Intensity | Intensity | Intensity |
| 1 | 3.743° | 23.58789 Å | 93.1697 | 136.296 | 24.6% |
| 2 | 5.368° | 16.45104 Å | 378.318 | 426.140 | 100.0% |
| 3 | 7.444° | 11.86630 Å | 78.1088 | 133.165 | 20.6% |
| 4 | 8.532° | 10.35521 Å | 191.848 | 250.752 | 50.7% |
| 5 | 10.694° | 8.26619 Å | 239.962 | 307.700 | 63.4% |
| 6 | 11.187° | 7.90319 Å | 35.8924 | 106.351 | 9.5% |
| 7 | 11.479° | 7.70285 Å | 47.5676 | 119.047 | 12.6% |
| 8 | 11.791° | 7.49956 Å | 60.4756 | 132.565 | 16.0% |
| 9 | 12.352° | 7.16031 Å | 21.6246 | 93.5508 | 5.7% |
| 10 | 13.724° | 6.44712 Å | 88.8508 | 164.011 | 23.5% |
| 11 | 14.037° | 6.30431 Å | 68.3052 | 144.794 | 18.1% |
| 12 | 15.392° | 5.75213 Å | 105.238 | 181.690 | 27.8% |
| 13 | 15.795° | 5.60608 Å | 71.5711 | 146.190 | 18.9% |
| 14 | 16.746° | 5.28996 Å | 29.2173 | 104.404 | 7.7% |
| 15 | 17.994° | 4.92563 Å | 119.091 | 205.446 | 31.5% |
| 16 | 18.798° | 4.71692 Å | 154.177 | 247.466 | 40.8% |
| 17 | 18.859° | 4.70169 Å | 201.896 | 295.578 | 53.4% |
| 18 | 19.633° | 4.51796 Å | 30.4316 | 129.691 | 8.0% |
| 19 | 20.179° | 4.39708 Å | 24.6052 | 129.759 | 6.5% |
| 20 | 20.618° | 4.30447 Å | 30.3211 | 139.131 | 8.0% |
| 21 | 21.458° | 4.13768 Å | 131.173 | 244.287 | 34.7% |
| 22 | 22.048° | 4.02830 Å | 245.140 | 359.105 | 64.8% |
| 23 | 22.290° | 3.98516 Å | 121.069 | 234.868 | 32.0% |
| 24 | 23.035° | 3.85787 Å | 97.3790 | 208.773 | 25.7% |
| 25 | 23.472° | 3.78708 Å | 102.901 | 211.558 | 27.2% |
| 26 | 23.613° | 3.76471 Å | 112.052 | 219.612 | 29.6% |
| 27 | 24.253° | 3.66684 Å | 78.6684 | 179.989 | 20.8% |
| 28 | 25.535° | 3.48553 Å | 52.9779 | 145.182 | 14.0% |
| 29 | 26.229° | 3.39496 Å | 68.5547 | 158.110 | 18.1% |
| 30 | 28.247° | 3.15675 Å | 38.9218 | 122.401 | 10.3% |
| 31 | 29.107° | 3.06544 Å | 22.3529 | 104.603 | 5.9% |

In embodiments, the crystalline Form H1 of Compound I phosphate salt exhibits a DSC thermogram comprising an endotherm peak at about 48° C. with the error of margin of about ±2.5; about ±2.0; about ±1.5; about ±1.0; about ±0.5; or less. In embodiments, the crystalline Form H1 of Compound I phosphate salt exhibits a DSC thermogram comprising an endotherm peak at about 110° C. with the error of margin of about ±2.5; about ±2.0; about ±1.5; about ±1.0; about ±0.5; or less. In embodiments, the crystalline Form H1 of Compound I phosphate salt exhibits a DSC thermogram comprising an endotherm peak with an onset at about 160° C. with the error of margin of about ±2.5; about ±2.0; about ±1.5; about ±1.0; about ±0.5; or less. In embodiments, the crystalline Form H1 of Compound I phosphate salt exhibits a DSC thermogram comprising an endotherm peak at about 190.9° C. with the error of margin of about ±2.5; about ±2.0; about ±1.5; about ±1.0; about ±0.5; or less

In some embodiments, the crystalline Form H1 of Compound I phosphate salt exhibits a DSC thermogram that is substantially similar to FIG. 29B.

In embodiments, the crystalline Form H1 of Compound I phosphate salt exhibits a TGA thermogram substantially similar to FIG. 29C. In embodiments, the crystalline Form H1 of Compound I phosphate salt exhibits a weight percent loss of about 4.6% between about 33° C. to about 150° C. by a thermogravimetric analysis (TGA).

### Compound I Phosphate Salt Form H3

In embodiments, the present disclosure relates to a crystalline form of Compound I phosphate salt, which is Form H3.

In embodiments of the present disclosure, crystalline Form H3 of Compound I phosphate salt is a solvate.

In embodiments of the present disclosure, crystalline Form H3 of Compound I phosphate salt is an acetone solvate.

In embodiments, the crystalline form of Compound I phosphate salt may comprise of over about 99.9%, about 99.8%, about 99.7%, about 99.6%, about 99.5%, about 99.4%, about 99.3%, about 99.2%, about 99.1%, or about 99.0% of Form H3 of Compound I phosphate salt. In another embodiment, the crystalline form of Compound I phosphate salt may comprise over about 99%, 98%, 97%, 96%, 95%, 94%, 93%, 92%, 91%, or 90% of Form H3 of Compound I phosphate salt. In some embodiments, the crystalline form of Compound I phosphate salt may comprise over about 90%, 85%, 80%, 75%, 70%, 65%, 60%, 55%, 50%, 45%, or 40% of Form H3 of Compound I phosphate salt.

In embodiments, crystalline Form H3 of Compound I phosphate salt exhibits an XRPD pattern comprising peaks at about 10.67, 18.77, and 22.04 degrees two-theta with the margin of error of about ±0.5; about ±0.4; about ±0.3; about ±0.2; about ±0.1; about ±0.05; or less. In embodiments, crystalline Form H3 of Compound I phosphate salt exhibits an XRPD pattern comprising peaks at about 10.67, 17.95, 18.77, 22.04, and 23.64 degrees two-theta with the margin of error of about ±0.5; about ±0.4; about ±0.3; about ±0.2; about ±0.1; about ±0.05; or less. In embodiments, crystalline Form H3 of Compound I phosphate salt exhibits an XRPD which further comprises at least two peaks selected from about 5.35±0.2, 7.46±0.2, 8.50±0.2, 11.51±0.2, 13.63±0.2, 15.36±0.2, 21.35±0.2, and 25.55±0.2 degrees two-theta with the margin of error of about ±0.5; about ±0.4; about ±0.3; about ±0.2; about ±0.1; about ±0.05; or less. In embodiments, crystalline Form H3 of Compound I phosphate salt exhibits an XRPD which further comprises at least three peaks selected from about 5.35±0.2, 7.46±0.2, 8.50±0.2, 11.51±0.2, 13.63±0.2, 15.36±0.2, 21.35±0.2, and 25.55±0.2 degrees two-theta with the margin of error of about ±0.5; about ±0.4; about ±0.3; about ±0.2; about ±0.1; about ±0.05; or less. In embodiments, crystalline Form H3 of Compound I phosphate salt exhibits an XRPD which further comprises at least four peaks selected from about 5.35±0.2, 7.46±0.2, 8.50±0.2, 11.51±0.2, 13.63±0.2, 15.36±0.2, 21.35±0.2, and 25.55±0.2 degrees two-theta with the margin of error of about ±0.5; about ±0.4; about ±0.3; about ±0.2; about ±0.1; about ±0.05; or less. In embodiments, crystalline Form H3 of Compound I phosphate salt exhibits an XRPD which further comprises at least five peaks selected from about 5.35±0.2, 7.46±0.2, 8.50±0.2, 11.51±0.2, 13.63±0.2, 15.36±0.2, 21.35±0.2, and 25.55±0.2 degrees two-theta with the margin of error of about ±0.5; about ±0.4; about ±0.3; about ±0.2; about ±0.1; about ±0.05; or less. In embodiments, crystalline Form H3 of Compound I phosphate salt exhibits an XRPD which further comprises at least six peaks selected from about 5.35±0.2, 7.46±0.2, 8.50±0.2, 11.51±0.2, 13.63±0.2, 15.36±0.2, 21.35±0.2, and 25.55±0.2 degrees two-theta with the margin of error of about ±0.5; about ±0.4; about ±0.3; about ±0.2; about ±0.1; about ±0.05; or less. In embodiments, crystalline Form H3 of Compound I phosphate salt exhibits an XRPD which further comprises at least seven peaks selected from about 5.35±0.2, 7.46±0.2, 8.50±0.2, 11.51±0.2, 13.63±0.2, 15.36±0.2, 21.35±0.2, and 25.55±0.2 degrees two-theta with the margin of error of about ±0.5; about ±0.4; about ±0.3; about ±0.2; about ±0.1; about ±0.05; or less. In embodiments, crystalline Form H3 of Compound I phosphate salt exhibits an XRPD which further comprises peaks at about 5.35±0.2, 7.46±0.2, 8.50±0.2, 11.51±0.2, 13.63±0.2, 15.36±0.2, 21.35±0.2, and 25.55±0.2 degrees two-theta with the margin of error of about ±0.5; about ±0.4; about ±0.3; about ±0.2; about ±0.1; about ±0.05; or less.

In embodiments, crystalline Form H3 of Compound I phosphate salt exhibits an XRPD pattern comprising peaks at about 10.67±0.2, 18.77±0.2, and 22.04±0.2 degrees two-theta.

In embodiments, crystalline Form H3 of Compound I phosphate salt exhibits an XRPD pattern comprising peaks at about 10.67±0.2, 17.95±0.2, 18.77±0.2, 22.04±0.2, and 23.64±0.2 degrees two-theta.

In embodiments, crystalline Form H3 of Compound I phosphate salt exhibits an XRPD which further comprises at least two peaks selected from about 5.35±0.2, 7.46±0.2, 8.50±0.2, 11.51±0.2, 13.63±0.2, 15.36±0.2, 21.35±0.2, and 25.55±0.2 degrees two-theta.

In some embodiments, the crystalline form of Compound I phosphate salt exhibits an XRPD pattern comprising 1 peak, 2 peaks, 3 peaks, 4 peaks, 5 peaks, 6 peaks, 7 peaks, 8 peaks, 9 peaks, or 10 peaks ±0.2 degrees two-theta of Table 30.

In embodiments, crystalline Form H3 of Compound I phosphate salt exhibits an XRPD pattern comprising peaks shown in Table 30, below.

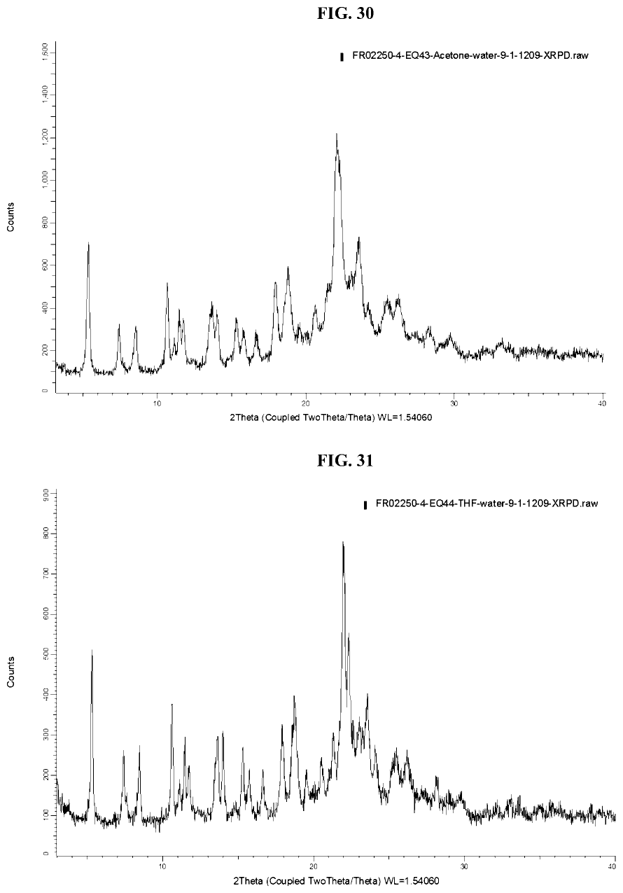

In embodiments, crystalline Form H3 of Compound I phosphate salt exhibits an XRPD pattern that is substantially similar to FIG. 30.

TABLE 30
Form H3 of Compound I phosphate salt

| | | d | Net | Gross | Rel. |
|---|---|---|---|---|---|
| Index | Angle | Value | Intensity | Intensity | Intensity |
| 1 | 5.348° | 16.51079 Å | 150.818 | 225.426 | 38.4% |
| 2 | 7.456° | 11.84726 Å | 102.884 | 178.123 | 26.2% |
| 3 | 8.496° | 10.39965 Å | 90.9699 | 169.621 | 23.1% |
| 4 | 10.669° | 8.28576 Å | 202.128 | 292.105 | 51.4% |
| 5 | 11.145° | 7.93292 Å | 57.8289 | 151.256 | 14.7% |
| 6 | 11.508° | 7.68301 Å | 116.485 | 211.702 | 29.6% |
| 7 | 11.776° | 7.50905 Å | 75.0187 | 171.085 | 19.1% |
| 8 | 13.629° | 6.49210 Å | 135.730 | 234.560 | 34.5% |
| 9 | 14.033° | 6.30583 Å | 109.136 | 207.207 | 27.8% |
| 10 | 14.869° | 5.95304 Å | 20.5657 | 119.651 | 5.2% |
| 11 | 15.363° | 5.76289 Å | 129.072 | 231.310 | 32.8% |
| 12 | 15.786° | 5.60941 Å | 69.0019 | 172.868 | 17.5% |
| 13 | 16.687° | 5.30838 Å | 78.5388 | 186.165 | 20.0% |
| 14 | 17.952° | 4.93728 Å | 157.254 | 280.766 | 40.0% |
| 15 | 18.770° | 4.72380 Å | 217.021 | 355.314 | 55.2% |
| 16 | 19.547° | 4.53768 Å | 41.7352 | 190.634 | 10.6% |
| 17 | 20.579° | 4.31246 Å | 65.8508 | 225.098 | 16.7% |
| 18 | 21.354° | 4.15763 Å | 109.332 | 275.428 | 27.8% |
| 19 | 22.038° | 4.03017 Å | 393.221 | 562.595 | 100.0% |
| 20 | 22.358° | 3.97325 Å | 279.642 | 449.662 | 71.1% |
| 21 | 23.635° | 3.76130 Å | 181.519 | 348.470 | 46.2% |
| 22 | 24.145° | 3.68304 Å | 80.9028 | 244.110 | 20.6% |
| 23 | 25.288° | 3.51905 Å | 63.5143 | 220.284 | 16.2% |
| 24 | 25.546° | 3.48413 Å | 98.4706 | 255.151 | 25.0% |
| 25 | 26.245° | 3.39285 Å | 75.2938 | 229.878 | 19.1% |
| 26 | 28.219° | 3.15982 Å | 64.6443 | 198.701 | 16.4% |
| 27 | 29.040° | 3.07237 Å | 47.2049 | 169.499 | 12.0% |
| 28 | 29.722° | 3.00345 Å | 33.2063 | 145.712 | 8.4% |
| 29 | 32.268° | 2.77204 Å | 37.1622 | 144.951 | 9.5% |
| 30 | 33.110° | 2.70345 Å | 35.8289 | 141.933 | 9.1% |
| 31 | 35.121° | 2.55310 Å | 24.1336 | 128.110 | 6.1% |

### Compound I Phosphate Salt Form H4

In embodiments, the present disclosure relates to a crystalline form of Compound I phosphate salt, which is Form H4.

In embodiments of the present disclosure, crystalline Form H4 of Compound I phosphate salt is a solvate.

In embodiments of the present disclosure, crystalline Form H4 of Compound I phosphate salt is an THF solvate.

In embodiments, the crystalline form of Compound I phosphate salt may comprise of over about 99.9%, about 99.8%, about 99.7%, about 99.6%, about 99.5%, about 99.4%, about 99.3%, about 99.2%, about 99.1%, or about 99.0% of Form H4 of Compound I phosphate salt. In another embodiment, the crystalline form of Compound I phosphate salt may comprise over about 99%, 98%, 97%, 96%, 95%, 94%, 93%, 92%, 91%, or 90% of Form H4 of Compound I phosphate salt. In some embodiments, the crystalline form of Compound I phosphate salt may comprise over about 90%, 85%, 80%, 75%, 70%, 65%, 60%, 55%, 50%, 45%, or 40% of Form H4 of Compound I phosphate salt.

In embodiments, crystalline Form H4 of Compound I phosphate salt exhibits an XRPD pattern comprising peaks at about 5.32, 22.00, and 22.34 degrees two-theta with the margin of error of about ±0.5; about ±0.4; about ±0.3; about ±0.2; about ±0.1; about ±0.05; or less. In embodiments, crystalline Form H4 of Compound I phosphate salt exhibits an XRPD pattern comprising peaks at about 5.32, 10.63, 18.74, 22.00, and 22.34 degrees two-theta with the margin of error of about ±0.5; about ±0.4; about ±0.3; about ±0.2; about ±0.1; about ±0.05; or less. In embodiments, crystalline Form H4 of Compound I phosphate salt exhibits an XRPD pattern which further comprises at least two peaks selected from about 7.41, 8.47, 11.48, 11.75, 13.62, 14.01, 17.90, 23.05, and 23.54 degrees two-theta with the margin of error of about ±0.5; about ±0.4; about ±0.3; about ±0.2; about ±0.1; about ±0.05; or less. In embodiments, crystalline Form H4 of Compound I phosphate salt exhibits an XRPD pattern which further comprises at least three peaks selected from about 7.41, 8.47, 11.48, 11.75, 13.62, 14.01, 17.90, 23.05, and 23.54 degrees two-theta with the margin of error of about ±0.5; about ±0.4; about ±0.3; about ±0.2; about ±0.1; about ±0.05; or less. In embodiments, crystalline Form H4 of Compound I phosphate salt exhibits an XRPD pattern which further comprises at least four peaks selected from about 7.41, 8.47, 11.48, 11.75, 13.62, 14.01, 17.90, 23.05, and 23.54 degrees two-theta with the margin of error of about ±0.5; about ±0.4; about ±0.3; about ±0.2; about ±0.1; about ±0.05; or less. In embodiments, crystalline Form H4 of Compound I phosphate salt exhibits an XRPD pattern which further comprises at least five peaks selected from about 7.41, 8.47, 11.48, 11.75, 13.62, 14.01, 17.90, 23.05, and 23.54 degrees two-theta with the margin of error of about ±0.5; about ±0.4; about ±0.3; about ±0.2; about ±0.1; about ±0.05; or less. In embodiments, crystalline Form H4 of Compound I phosphate salt exhibits an XRPD pattern which further comprises at least six peaks selected from about 7.41, 8.47, 11.48, 11.75, 13.62, 14.01, 17.90, 23.05, and 23.54 degrees two-theta with the margin of error of about ±0.5; about ±0.4; about ±0.3; about ±0.2; about ±0.1; about ±0.05; or less. In embodiments, crystalline Form H4 of Compound I phosphate salt exhibits an XRPD pattern which further comprises at least seven peaks selected from about 7.41, 8.47, 11.48, 11.75, 13.62, 14.01, 17.90, 23.05, and 23.54 degrees two-theta with the margin of error of about ±0.5; about ±0.4; about ±0.3; about ±0.2; about ±0.1; about ±0.05; or less. In embodiments, crystalline Form H4 of Compound I phosphate salt exhibits an XRPD pattern which further comprises peaks at about 7.41, 8.47, 11.48, 11.75, 13.62, 14.01, 17.90, 23.05, and 23.54 degrees two-theta with the margin of error of about ±0.5; about ±0.4; about ±0.3; about ±0.2; about ±0.1; about ±0.05; or less.

In embodiments, crystalline Form H4 of Compound I phosphate salt exhibits an XRPD pattern comprising peaks at about 5.32±0.2, 22.00±0.2, and 22.34±0.2 degrees two-theta.

In embodiments, crystalline Form H4 of Compound I phosphate salt exhibits an XRPD pattern comprising peaks at about 5.32±0.2, 10.63±0.2, 18.74±0.2, 22.00±0.2, and 22.34±0.2 degrees two-theta.

In embodiments, crystalline Form H4 of Compound I phosphate salt exhibits an XRPD pattern which further comprises at least two peaks selected from about 7.41±0.2, 8.47±0.2, 11.48±0.2, 11.75±0.2, 13.62±0.2, 14.01±0.2, 17.90±0.2, 23.05±0.2, and 23.54±0.2 degrees two-theta with the margin of error of about ±0.5; about ±0.4; about ±0.3; about ±0.2; about ±0.1; about ±0.05; or less.

In some embodiments, the crystalline form of Compound I phosphate salt exhibits an XRPD pattern comprising 1 peak, 2 peaks, 3 peaks, 4 peaks, 5 peaks, 6 peaks, 7 peaks, 8 peaks, 9 peaks, or 10 peaks ±0.2 degrees two-theta of Table 31.

In embodiments, crystalline Form H4 of Compound I phosphate salt exhibits an XRPD pattern comprising peaks shown in Table 31, below.

In embodiments, crystalline Form H4 of Compound I phosphate salt exhibits an XRPD pattern that is substantially similar to FIG. 31.

TABLE 31
Form H4 of Compound I phosphate salt

| | | d | Net | Gross | Rel. |
|---|---|---|---|---|---|
| Index | Angle | Value | Intensity | Intensity | Intensity |
| 1 | 5.324° | 16.58593 Å | 258.893 | 348.050 | 64.3% |
| 2 | 7.414° | 11.91343 Å | 120.530 | 204.880 | 29.9% |
| 3 | 8.468° | 10.43374 Å | 118.154 | 199.719 | 29.4% |
| 4 | 10.627° | 8.31783 Å | 192.722 | 277.551 | 47.9% |
| 5 | 11.126° | 7.94585 Å | 70.3903 | 159.519 | 17.5% |
| 6 | 11.475° | 7.70501 Å | 121.671 | 212.983 | 30.2% |
| 7 | 11.747° | 7.52718 Å | 67.4131 | 159.953 | 16.7% |
| 8 | 13.622° | 6.49508 Å | 128.773 | 223.801 | 32.0% |
| 9 | 14.008° | 6.31690 Å | 150.691 | 245.518 | 37.4% |
| 10 | 15.321° | 5.77863 Å | 108.204 | 210.600 | 26.9% |
| 11 | 15.754° | 5.62076 Å | 79.1854 | 183.152 | 19.7% |
| 12 | 16.640° | 5.32332 Å | 77.1263 | 182.115 | 19.2% |
| 13 | 17.900° | 4.95128 Å | 127.297 | 242.927 | 31.6% |
| 14 | 18.742° | 4.73080 Å | 189.607 | 319.089 | 47.1% |
| 15 | 19.487° | 4.55162 Å | 53.0096 | 192.503 | 13.2% |
| 16 | 20.511° | 4.32663 Å | 57.6485 | 210.179 | 14.3% |
| 17 | 21.334° | 4.16155 Å | 84.9465 | 243.708 | 21.1% |
| 18 | 22.002° | 4.03661 Å | 402.549 | 563.589 | 100.0% |
| 19 | 22.336° | 3.97696 Å | 259.927 | 421.170 | 64.6% |
| 20 | 23.046° | 3.85614 Å | 134.933 | 294.538 | 33.5% |
| 21 | 23.541° | 3.77608 Å | 166.255 | 323.049 | 41.3% |
| 22 | 24.100° | 3.68986 Å | 79.4614 | 231.446 | 19.7% |
| 23 | 25.375° | 3.50725 Å | 70.1630 | 214.095 | 17.4% |
| 24 | 25.520° | 3.48764 Å | 85.4600 | 229.742 | 21.2% |
| 25 | 26.204° | 3.39812 Å | 77.3229 | 221.671 | 19.2% |
| 26 | 28.163° | 3.16601 Å | 32.6293 | 162.698 | 8.1% |
| 27 | 33.042° | 2.70880 Å | 23.8317 | 123.907 | 5.9% |
| 28 | 38.383° | 2.34326 Å | 25.1729 | 120.624 | 6.3% |

### Compound I Phosphate Salt Form H5

In embodiments, the present disclosure relates to a crystalline form of Compound I phosphate salt, which is Form H5.

In embodiments of the present disclosure, crystalline Form H5 of Compound I phosphate salt is a solvate.

In embodiments of the present disclosure, crystalline Form H5 of Compound I phosphate salt is an DMSO solvate.

In embodiments, the crystalline form of Compound I phosphate salt may comprise of over about 99.9%, about 99.8%, about 99.7%, about 99.6%, about 99.5%, about 99.4%, about 99.3%, about 99.2%, about 99.1%, or about 99.0% of Form H5 of Compound I phosphate salt. In another embodiment, the crystalline form of Compound I phosphate salt may comprise over about 99%, 98%, 97%, 96%, 95%, 94%, 93%, 92%, 91%, or 90% of Form H5 of Compound I phosphate salt. In some embodiments, the crystalline form of Compound I phosphate salt may comprise over about 90%, 85%, 80%, 75%, 70%, 65%, 60%, 55%, 50%, 45%, or 40% of Form H5 of Compound I phosphate salt.

In embodiments, crystalline Form H5 of Compound I phosphate salt exhibits an XRPD pattern comprising peaks at about 15.55, 18.84, and 21.66 degrees two-theta with the margin of error of about ±0.5; about ±0.4; about ±0.3; about ±0.2; about ±0.1; about ±0.05; or less. In embodiments, crystalline Form H5 of Compound I phosphate salt exhibits an XRPD pattern comprising peaks at about 5.33, 15.55, 18.84, 21.30, and 21.66 degrees two-theta with the margin of error of about ±0.5; about ±0.4; about ±0.3; about ±0.2; about ±0.1; about ±0.05; or less. In embodiments, crystalline Form H5 of Compound I phosphate salt exhibits an XRPD pattern which further comprises at least two peaks selected from about 7.54, 10.63, 11.32, 13.54, 17.98, 20.90, 22.52, and 23.36 degrees two-theta with the margin of error of about ±0.5; about ±0.4; about ±0.3; about ±0.2; about ±0.1; about ±0.05; or less. In embodiments, crystalline Form H5 of Compound I phosphate salt exhibits an XRPD pattern which further comprises at least three peaks selected from about 7.54, 10.63, 11.32, 13.54, 17.98, 20.90, 22.52, and 23.36 degrees two-theta with the margin of error of about ±0.5; about ±0.4; about ±0.3; about ±0.2; about ±0.1; about ±0.05; or less. In embodiments, crystalline Form H5 of Compound I phosphate salt exhibits an XRPD pattern which further comprises at least four peaks selected from about 7.54, 10.63, 11.32, 13.54, 17.98, 20.90, 22.52, and 23.36 degrees two-theta with the margin of error of about ±0.5; about ±0.4; about ±0.3; about ±0.2; about ±0.1; about ±0.05; or less. In embodiments, crystalline Form H5 of Compound I phosphate salt exhibits an XRPD pattern which further comprises at least five peaks selected from about 7.54, 10.63, 11.32, 13.54, 17.98, 20.90, 22.52, and 23.36 degrees two-theta with the margin of error of about ±0.5; about ±0.4; about ±0.3; about ±0.2; about ±0.1; about ±0.05; or less. In embodiments, crystalline Form H5 of Compound I phosphate salt exhibits an XRPD pattern which further comprises at least six peaks selected from about 7.54, 10.63, 11.32, 13.54, 17.98, 20.90, 22.52, and 23.36 degrees two-theta with the margin of error of about ±0.5; about ±0.4; about ±0.3; about ±0.2; about ±0.1; about ±0.05; or less. In embodiments, crystalline Form H5 of Compound I phosphate salt exhibits an XRPD pattern which further comprises at least seven peaks selected from about 7.54, 10.63, 11.32, 13.54, 17.98, 20.90, 22.52, and 23.36 degrees two-theta with the margin of error of about ±0.5; about ±0.4; about ±0.3; about ±0.2; about ±0.1; about ±0.05; or less. In embodiments, crystalline Form H5 of Compound I phosphate salt exhibits an XRPD pattern which further comprises peaks at about 7.54, 10.63, 11.32, 13.54, 17.98, 20.90, 22.52, and 23.36 degrees two-theta with the margin of error of about ±0.5; about ±0.4; about ±0.3; about ±0.2; about ±0.1; about ±0.05; or less.

In embodiments, crystalline Form H5 of Compound I phosphate salt exhibits an XRPD pattern comprising peaks at about 15.55±0.2, 18.84±0.2, and 21.66±0.2 degrees two-theta.

In embodiments, crystalline Form H5 of Compound I phosphate salt exhibits an XRPD pattern comprising peaks at about 5.33±0.2, 15.55±0.2, 18.84±0.2, 21.30±0.2, and 21.66±0.2 degrees two-theta.

In embodiments, crystalline Form H5 of Compound I phosphate salt exhibits an XRPD pattern which further comprises at least two peaks selected from about 7.54±0.2, 10.63±0.2, 11.32±0.2, 13.54±0.2, 17.98±0.2, 20.90±0.2, 22.52±0.2, and 23.36±0.2 degrees two-theta

In some embodiments, the crystalline form of Compound I phosphate salt exhibits an XRPD pattern comprising 1 peak, 2 peaks, 3 peaks, 4 peaks, 5 peaks, 6 peaks, 7 peaks, 8 peaks, 9 peaks, or 10 peaks ±0.2 degrees two-theta of Table 32.

In embodiments, crystalline Form H5 of Compound I phosphate salt exhibits an XRPD pattern comprising peaks shown in Table 32, below.

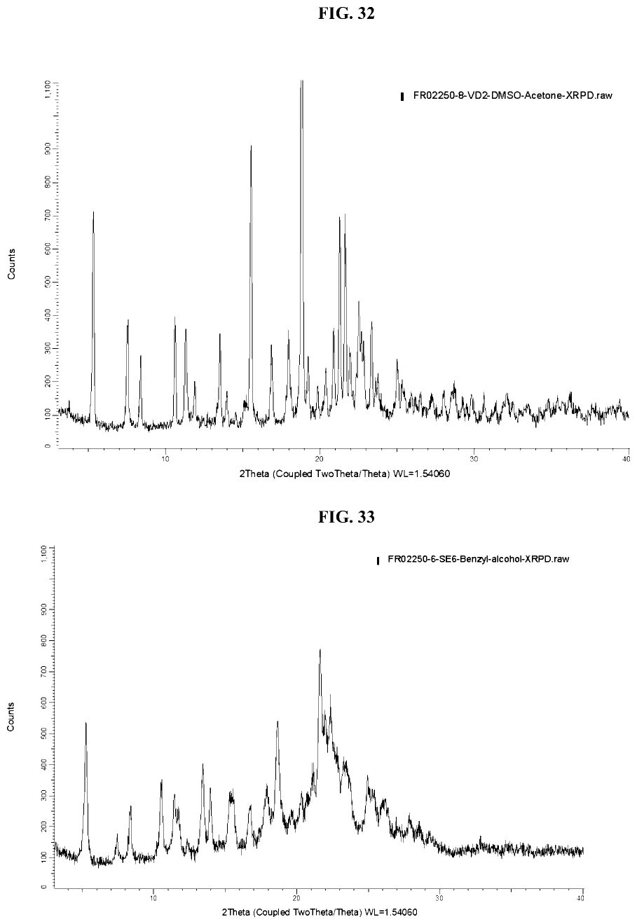

In embodiments, crystalline Form H5 of Compound I phosphate salt exhibits an XRPD pattern that is substantially similar to FIG. 32.

TABLE 32
Form H5 of Compound I phosphate salt

| | | d | Net | Gross | Rel. |
|---|---|---|---|---|---|
| Index | Angle | Value | Intensity | Intensity | Intensity |
| 1 | 5.331° | 16.56351 Å | 396.024 | 466.392 | 36.1% |
| 2 | 7.540° | 11.71541 Å | 205.826 | 268.968 | 18.8% |
| 3 | 8.393° | 10.52622 Å | 143.669 | 207.773 | 13.1% |
| 4 | 10.628° | 8.31758 Å | 219.015 | 289.189 | 20.0% |
| 5 | 11.316° | 7.81346 Å | 193.771 | 266.383 | 17.7% |
| 6 | 11.908° | 7.42605 Å | 87.8285 | 159.425 | 8.0% |
| 7 | 13.537° | 6.53594 Å | 193.757 | 269.101 | 17.7% |
| 8 | 13.989° | 6.32581 Å | 58.0518 | 131.997 | 5.3% |
| 9 | 14.561° | 6.07826 Å | 29.9231 | 100.786 | 2.7% |
| 10 | 15.250° | 5.80537 Å | 64.6203 | 138.826 | 5.9% |
| 11 | 15.553° | 5.69283 Å | 593.241 | 669.233 | 54.1% |
| 12 | 16.877° | 5.24900 Å | 162.488 | 243.571 | 14.8% |
| 13 | 17.984° | 4.92850 Å | 180.887 | 271.570 | 16.5% |
| 14 | 18.840° | 4.70647 Å | 1096.07 | 1196.82 | 100.0% |
| 15 | 19.249° | 4.60722 Å | 115.909 | 219.339 | 10.6% |
| 16 | 19.867° | 4.46533 Å | 60.2427 | 165.330 | 5.5% |
| 17 | 20.395° | 4.35099 Å | 98.5757 | 202.992 | 9.0% |
| 18 | 20.900° | 4.24699 Å | 195.180 | 303.434 | 17.8% |
| 19 | 21.300° | 4.16802 Å | 441.817 | 554.376 | 40.3% |
| 20 | 21.655° | 4.10062 Å | 445.324 | 560.591 | 40.6% |
| 21 | 21.958° | 4.04459 Å | 111.135 | 227.901 | 10.1% |
| 22 | 22.523° | 3.94441 Å | 255.344 | 372.881 | 23.3% |
| 23 | 22.839° | 3.89054 Å | 158.291 | 275.112 | 14.4% |
| 24 | 23.358° | 3.80532 Å | 163.369 | 277.236 | 14.9% |
| 25 | 23.707° | 3.75012 Å | 43.0783 | 153.717 | 3.9% |
| 26 | 25.025° | 3.55546 Å | 113.412 | 219.927 | 10.3% |
| 27 | 25.305° | 3.51675 Å | 61.7724 | 170.236 | 5.6% |
| 28 | 25.955° | 3.43018 Å | 42.2343 | 152.737 | 3.9% |
| 29 | 26.200° | 3.39862 Å | 22.7881 | 133.158 | 2.1% |
| 30 | 26.517° | 3.35875 Å | 39.1121 | 148.578 | 3.6% |
| 31 | 27.263° | 3.26841 Å | 21.1169 | 125.185 | 1.9% |
| 32 | 28.039° | 3.17979 Å | 40.4731 | 140.414 | 3.7% |
| 33 | 28.728° | 3.10505 Å | 52.4900 | 152.212 | 4.8% |
| 34 | 29.256° | 3.05017 Å | 22.6510 | 119.968 | 2.1% |
| 35 | 29.836° | 2.99217 Å | 53.1440 | 145.174 | 4.8% |
| 36 | 30.662° | 2.91344 Å | 44.0493 | 132.598 | 4.0% |
| 37 | 31.393° | 2.84723 Å | 28.7417 | 116.363 | 2.6% |
| 38 | 32.118° | 2.78458 Å | 58.5001 | 146.193 | 5.3% |
| 39 | 32.539° | 2.74955 Å | 31.5504 | 118.645 | 2.9% |
| 40 | 33.471° | 2.67506 Å | 34.6310 | 115.569 | 3.2% |
| 41 | 34.804° | 2.57563 Å | 52.5968 | 137.907 | 4.8% |
| 42 | 35.405° | 2.53327 Å | 50.4187 | 141.070 | 4.6% |
| 43 | 35.737° | 2.51051 Å | 36.5665 | 128.893 | 3.3% |
| 44 | 36.217° | 2.47830 Å | 41.5414 | 134.690 | 3.8% |
| 45 | 39.433° | 2.28325 Å | 31.5968 | 124.082 | 2.9% |

### Compound I Phosphate Salt Form H6

In embodiments, the present disclosure relates to a crystalline form of Compound I phosphate salt, which is Form H6.

In embodiments of the present disclosure, crystalline Form H6 of Compound I phosphate salt is a solvate.

In embodiments of the present disclosure, crystalline Form H6 of Compound I phosphate salt is an benzyl alcohol solvate.

In embodiments, the crystalline form of Compound I phosphate salt may comprise of over about 99.9%, about 99.8%, about 99.7%, about 99.6%, about 99.5%, about 99.4%, about 99.3%, about 99.2%, about 99.1%, or about 99.0% of Form H6 of Compound I phosphate salt. In another embodiment, the crystalline form of Compound I phosphate salt may comprise over about 99%, 98%, 97%, 96%, 95%, 94%, 93%, 92%, 91%, or 90% of Form H6 of Compound I phosphate salt. In some embodiments, the crystalline form of Compound I phosphate salt may comprise over about 90%, 85%, 80%, 75%, 70%, 65%, 60%, 55%, 50%, 45%, or 40% of Form H6 of Compound I phosphate salt.

In embodiments, crystalline Form H6 of Compound I phosphate salt exhibits an XRPD pattern comprising peaks at about 5.27, 21.64, and 22.35 degrees two-theta with the margin of error of about ±0.5; about ±0.4; about ±0.3; about ±0.2; about ±0.1; about ±0.05; or less. In embodiments, crystalline Form H6 of Compound I phosphate salt exhibits an XRPD pattern comprising peaks at about 5.27, 18.67, 21.64, 21.99, and 22.35 degrees two-theta with the margin of error of about ±0.5; about ±0.4; about ±0.3; about ±0.2; about ±0.1; about ±0.05; or less. In embodiments, crystalline Form H6 of Compound I phosphate salt exhibits an XRPD pattern which further comprises at least two peaks selected from about 8.38, 10.55, 11.47, 11.71, 13.44, 13.96, 15.45, and 23.36 degrees two-theta with the margin of error of about ±0.5; about ±0.4; about ±0.3; about ±0.2; about ±0.1; about ±0.05; or less. In embodiments, crystalline Form H6 of Compound I phosphate salt exhibits an XRPD pattern which further comprises at least three peaks selected from about 8.38, 10.55, 11.47, 11.71, 13.44, 13.96, 15.45, and 23.36 degrees two-theta with the margin of error of about ±0.5; about ±0.4; about ±0.3; about ±0.2; about ±0.1; about ±0.05; or less. In embodiments, crystalline Form H6 of Compound I phosphate salt exhibits an XRPD pattern which further comprises at least four peaks selected from about 8.38, 10.55, 11.47, 11.71, 13.44, 13.96, 15.45, and 23.36 degrees two-theta with the margin of error of about ±0.5; about ±0.4; about ±0.3; about ±0.2; about ±0.1; about ±0.05; or less. In embodiments, crystalline Form H6 of Compound I phosphate salt exhibits an XRPD pattern which further comprises at least five peaks selected from about 8.38, 10.55, 11.47, 11.71, 13.44, 13.96, 15.45, and 23.36 degrees two-theta with the margin of error of about ±0.5; about ±0.4; about ±0.3; about ±0.2; about ±0.1; about ±0.05; or less. In embodiments, crystalline Form H6 of Compound I phosphate salt exhibits an XRPD pattern which further comprises at least six peaks selected from about 8.38, 10.55, 11.47, 11.71, 13.44, 13.96, 15.45, and 23.36 degrees two-theta with the margin of error of about ±0.5; about ±0.4; about ±0.3; about ±0.2; about ±0.1; about ±0.05; or less. In embodiments, crystalline Form H6 of Compound I phosphate salt exhibits an XRPD pattern which further comprises peaks at about 8.38, 10.55, 11.47, 11.71, 13.44, 13.96, 15.45, and 23.36 degrees two-theta with the margin of error of about ±0.5; about ±0.4; about ±0.3; about ±0.2; about ±0.1; about ±0.05; or less.

In embodiments, crystalline Form H6 of Compound I phosphate salt exhibits an XRPD pattern comprising peaks at about 5.27±0.2, 21.64±0.2, and 22.35±0.2 degrees two-theta.

In embodiments, crystalline Form H6 of Compound I phosphate salt exhibits an XRPD pattern comprising peaks at about 8.38±0.2, 10.55±0.2, 11.47±0.2, 11.71±0.2, 13.44±0.2, 13.96±0.2, 15.45±0.2, and 23.36±0.2 degrees two-theta.

In embodiments, crystalline Form H6 of Compound I phosphate salt exhibits an XRPD pattern comprising peaks at about 5.27±0.2, 18.67±0.2, 21.64±0.2, 21.99±0.2, and 22.35±0.2 degrees two-theta.

In embodiments, crystalline Form H6 of Compound I phosphate salt exhibits an XRPD pattern which comprises at least two peaks selected from about 8.38±0.2, 10.55±0.2, 11.47±0.2, 11.71±0.2, 13.44±0.2, 13.96±0.2, 15.45±0.2, and 23.36±0.2 degrees two-theta.

In some embodiments, the crystalline form of Compound I phosphate salt exhibits an XRPD pattern comprising 1 peak, 2 peaks, 3 peaks, 4 peaks, 5 peaks, 6 peaks, 7 peaks, 8 peaks, 9 peaks, or 10 peaks ±0.2 degrees two-theta of Table 33.

In embodiments, crystalline Form H6 of Compound I phosphate salt exhibits an XRPD pattern comprising peaks shown in Table 33, below.

In embodiments, crystalline Form H6 of Compound I phosphate salt exhibits an XRPD pattern that is substantially similar to FIG. 33.

TABLE 33
Form H6 of Compound I phosphate salt

| | | d | Net | Gross | Rel. |
|---|---|---|---|---|---|
| Index | Angle | Value | Intensity | Intensity | Intensity |
| 1 | 5.272° | 16.75008 Å | 276.510 | 367.309 | 72.9% |
| 2 | 7.451° | 11.85440 Å | 55.0806 | 144.910 | 14.5% |
| 3 | 8.382° | 10.54038 Å | 114.233 | 210.557 | 30.1% |
| 4 | 10.547° | 8.38105 Å | 134.223 | 239.582 | 35.4% |
| 5 | 11.465° | 7.71161 Å | 132.257 | 243.831 | 34.9% |
| 6 | 11.713° | 7.54924 Å | 104.767 | 217.212 | 27.6% |
| 7 | 12.361° | 7.15498 Å | 22.4485 | 137.257 | 5.9% |
| 8 | 13.435° | 6.58545 Å | 204.811 | 327.561 | 54.0% |
| 9 | 13.959° | 6.33940 Å | 133.466 | 260.019 | 35.2% |
| 10 | 15.453° | 5.72957 Å | 124.116 | 256.071 | 32.7% |
| 11 | 16.727° | 5.29587 Å | 71.6346 | 214.693 | 18.9% |
| 12 | 17.917° | 4.94671 Å | 91.4590 | 260.211 | 24.1% |
| 13 | 18.665° | 4.75008 Å | 237.774 | 420.963 | 62.7% |
| 14 | 19.621° | 4.52073 Å | 45.9436 | 243.050 | 12.1% |
| 15 | 20.343° | 4.36189 Å | 67.4076 | 271.650 | 17.8% |
| 16 | 21.142° | 4.19891 Å | 54.8722 | 263.632 | 14.5% |
| 17 | 21.642° | 4.10299 Å | 379.073 | 588.856 | 100.0% |
| 18 | 21.986° | 4.03958 Å | 262.731 | 472.407 | 69.3% |
| 19 | 22.348° | 3.97499 Å | 309.659 | 518.515 | 81.7% |
| 20 | 23.357° | 3.80552 Å | 98.5175 | 301.238 | 26.0% |
| 21 | 24.998° | 3.55920 Å | 62.6388 | 254.217 | 16.5% |
| 22 | 25.342° | 3.51172 Å | 59.7223 | 250.095 | 15.8% |
| 23 | 27.874° | 3.19825 Å | 63.1729 | 226.270 | 16.7% |
| 24 | 29.281° | 3.04766 Å | 26.7358 | 164.699 | 7.1% |

A Summary of Crystal Forms of Compound I phosphate salt are shown in Table 34, below.

TABLE 34
Characterization Summary of Crystal
Forms of Phosphate Salt of Compound I

| Crystalline Form | Speculated Form |
|---|---|
| Form A* | Hydrate |
| Form B | Anhydrate |
| Form C | Hydrate |
| Form J | Hydrate |
| Form K | Hydrate |
| Form H2 | Hydrate |
| Form E | Solvate |
| Form F | Solvate |
| Form G | Solvate |
| Form I | Solvate |
| Form H1 | Solvate |
| Form H3 | Solvate |
| Form H4 | Solvate |
| Form H5 | Solvate |
| Form H6 | Solvate |

### Pharmaceutical Compositions and Formulations

In embodiments the present disclosure provides a pharmaceutical composition comprising a therapeutically effective amount of a crystalline form of Compound I, or a pharmaceutically acceptable salt thereof, solvate thereof, or salt solvate thereof, as disclosed herein, as the active ingredient, combined with a pharmaceutically acceptable excipient or carrier. In embodiments, the present disclosure provides a pharmaceutical composition comprising a therapeutically effective amount of a crystalline form of Compound I, or a pharmaceutically acceptable salt thereof, solvate thereof, or salt solvate thereof, e.g., Form A, Form A*, Form A**, Form B*, Form A-1, Form B-1, Form A-2, Form B-2, Form A-3, Form B-3, Form C-3, Form A-4, Form A-5, Form A-6, Form A-7, Form B-7, Form A-8, Form B-8, Form A-9, Form B, Form C, Form J, Form K, Form H2, Form E, Form F, Form G, Form I, Form H1, Form H3, Form H4, Form H5, or Form H6. In embodiments, the present disclosure provides a pharmaceutical composition comprising a therapeutically effective amount of a crystalline Form A* of Compound I and a pharmaceutically acceptable excipient or carrier. The excipients are added to the formulation for a variety of purposes.

In one embodiment of the present disclosure, the pharmaceutical composition comprises a Compound I or a pharmaceutically acceptable salt thereof, solvate thereof, or salt solvate thereof as a mixture of different forms.

In embodiments, the pharmaceutical composition comprises a crystalline form of Compound I or a pharmaceutically acceptable salt thereof, solvate thereof, or salt solvate thereof (e.g., Form A, Form A*, Form A**, Form B*, Form A-1, Form B-1, Form A-2, Form B-2, Form A-3, Form B-3, Form C-3, Form A-4, Form A-5, Form A-6, Form A-7, Form B-7, Form A-8, Form B-8, Form A-9, Form B, Form C, Form J, Form K, Form H2, Form E, Form F, Form G, Form I, Form H1, Form H3, Form H4, Form H5, or Form H6) in about 99.9%, about 99.8%, about 99.7%, about 99.6%, about 99.5%, about 99.4%, about 99.3%, about 99.2%, about 99.1%, or about 99.0% of the total amount of Compound I or a pharmaceutically acceptable salt thereof, solvate thereof, or salt solvate thereof. In embodiments, the pharmaceutical composition comprises a crystalline form of Compound I or a pharmaceutically acceptable salt thereof, solvate thereof, or salt solvate thereof (e.g., Form A, Form A*, Form A**, Form B*, Form A-1, Form B-1, Form A-2, Form B-2, Form A-3, Form B-3, Form C-3, Form A-4, Form A-5, Form A-7, Form A-6, Form B-7, Form A-8, Form B-8, Form A-9, Form B, Form C, Form J, Form K, Form H2, Form E, Form F, Form G, Form I, Form H1, Form H3, Form H4, Form H5, or Form H6) in about 99%, 98%, 97%, 96%, 95%, 94%, 93%, 92%, 91%, or 90% of the total amount of Compound I or a pharmaceutically acceptable salt thereof, solvate thereof, or salt solvate thereof. In embodiments, the pharmaceutical composition comprises a crystalline form of Compound I or a pharmaceutically acceptable salt thereof, solvate thereof, or salt solvate thereof (e.g., Form A, Form A+, Form A**, Form B*, Form A-1, Form B-1, Form A-2, Form B-2, Form A-3, Form B-3, Form C-3, Form A-4, Form A-5, Form A-6, Form A-7, Form B-7, Form A-8, Form B-8, Form A-9, Form B, Form C, Form J, Form K, Form H2, Form E, Form F, Form G, Form I, Form H1, Form H3, Form H4, Form H5, or Form H6) in about 90%, 85%, 80%, 75%, 70%, 65%, 60%, 55%, 50%, 45%, 40%, 35%, 30%, 25%, or 20% of the total amount of Compound I or a pharmaceutically acceptable salt thereof, solvate thereof, or salt solvate thereof. In one embodiment, the pharmaceutical composition comprises a crystalline form of Compound I or a pharmaceutically acceptable salt thereof, solvate thereof, or salt solvate thereof (e.g., Form A, Form A*, Form A**, Form B*, Form A-1, Form B-1, Form A-2, Form B-2, Form A-3, Form B-3, Form C-3, Form A-4, Form A-5, Form A-6, Form A-7, Form B-7, Form A-8, Form B-8, Form A-9, Form B, Form C, Form J, Form K, Form H2, Form E, Form F, Form G, Form I, Form H1, Form H3, Form H4, Form H5, or Form H6) in about 0.1%, 0.2%, 0.3%, 0.4%, 0.5%, 0.6%, 0.7%, 0.8%, 0.9%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 11%, 12%, 13%, 14%, 15%, 16%, 17%, 18%, 18%, or 20% of the total amount of Compound I or a pharmaceutically acceptable salt, solvate thereof, or salt solvate thereof.

In embodiments, the Compound I can be present in the pharmaceutical composition as a pharmaceutically acceptable salt. In embodiments, the Compound I can be present in the pharmaceutical composition as a pharmaceutical solvate. In embodiments, the Compound I can be present in the pharmaceutical composition as a pharmaceutical salt solvate. In embodiments, the Compound I can be present in the pharmaceutical composition as a crystalline form that is an anhydrous free base of Compound I. In embodiments, the Compound I can be present in the pharmaceutical composition a crystalline form of a pharmaceutically acceptable salt that is anhydrous.

In embodiments, a pharmaceutical composition, as described herein, further comprises one or more additional therapeutically active agents. In embodiments, one or more additional therapeutically active agents are selected from therapeutics useful for treating cancer, neurodegenerative disease, autoimmune disorder and aging.

In a further embodiment of the present disclosure, a pharmaceutical composition comprising one or more solid forms of Compound I or a pharmaceutically acceptable salt thereof, solvate thereof, or salt solvate thereof (e.g., a crystalline form such as Form A, Form A*, Form A**, Form B*, Form A-1, Form B-1, Form A-2, Form B-2, Form A-3, Form B-3, Form C-3, Form A-4, Form A-5, Form A-6, Form A-7, Form B-7, Form A-8, Form B-8, Form A-9, Form B, Form C, Form J, Form K, Form H2, Form E, Form F, Form G, Form I, Form H1, Form H3, Form H4, Form H5, or Form H6), and a pharmaceutically acceptable excipient or adjuvant is provided. The pharmaceutically acceptable excipients and adjuvants are added to the composition or formulation for a variety of purposes. In another embodiment, a pharmaceutical composition comprising one or more solid forms of Compound I, or a pharmaceutically acceptable salt thereof, solvate thereof, or salt solvate thereof, further comprises a pharmaceutically acceptable carrier. In one embodiment, a pharmaceutically acceptable carrier includes a pharmaceutically acceptable excipient, binder, and/or diluent. In one embodiment, suitable pharmaceutically acceptable excipients include, but are not limited to, water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, the pharmaceutical compositions of the present disclosure may additionally contain other adjunct components conventionally found in pharmaceutical compositions, at their art-established usage levels. Thus, for example, the pharmaceutical compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the oligonucleotide(s) of the formulation.

For the purposes of this disclosure, the solid forms of Compound I of the present disclosure can be formulated for administration by a variety of means including orally, parenterally, by inhalation spray, topically, or rectally in formulations containing pharmaceutically acceptable carriers, adjuvants and vehicles. The term parenteral as used here includes subcutaneous, intravenous, intramuscular, and intraarterial injections with a variety of infusion techniques. Intraarterial and intravenous injection as used herein includes administration through catheters.

The solid forms of Compound I disclosed herein can be formulated in accordance with the routine procedures adapted for desired administration route. Accordingly, the solid forms of Compound I disclosed herein can take such forms as suspensions, solutions or emulsions in oily or aqueous vehicles, and can contain formulatory agents such as suspending, stabilizing and/or dispersing agents. The solid forms of Compound I disclosed herein can also be formulated as a preparation for implantation or injection. Thus, for example, the solid forms of Compound I can be formulated with suitable polymeric or hydrophobic materials (e.g., as an emulsion in an acceptable oil) or ion exchange resins, or as sparingly soluble derivatives (e.g., as a sparingly soluble salt). Alternatively, the active ingredient can be in powder form for constitution with a suitable vehicle, e.g., sterile pyrogen-free water, before use. Suitable formulations for each of these methods of administration can be found, for example, in Remington: The Science and Practice of Pharmacy, A. Gennaro, ed., 20th edition, Lippincott, Williams & Wilkins, Philadelphia, PA.

In certain embodiments, a pharmaceutical composition of the present disclosure is prepared using known techniques, including, but not limited to mixing, dissolving, granulating, dragee-making, levigating, emulsifying, encapsulating, entrapping or tableting processes.

In embodiments embodiment, the present disclosure provides a pharmaceutical composition comprising a compound of formula (I-A), (I-B), (I-C), or a pharmaceutically acceptable salt thereof, solvate thereof, or salt solvate thereof, as disclosed herein, combined with a pharmaceutically acceptable carrier. In one embodiment, suitable pharmaceutically acceptable carriers include, but are not limited to, inert solid fillers or diluents and sterile aqueous or organic solutions. Pharmaceutically acceptable carriers are well known to those skilled in the art and include, but are not limited to, from about 0.01 to about 0.1 M and preferably 0.05M phosphate buffer or 0.8% saline. Such pharmaceutically acceptable carriers can be aqueous or non-aqueous solutions, suspensions and emulsions. Examples of non-aqueous solvents suitable for use in the present application include, but are not limited to, propylene glycol, polyethylene glycol, vegetable oils such as olive oil, and injectable organic esters such as ethyl oleate.

Aqueous carriers suitable for use in the present application include, but are not limited to, water, ethanol, alcoholic/aqueous solutions, glycerol, emulsions or suspensions, including saline and buffered media. Oral carriers can be elixirs, syrups, capsules, tablets and the like.

Liquid carriers suitable for use in the present application can be used in preparing solutions, suspensions, emulsions, syrups, elixirs and pressurized compounds. The active ingredient can be dissolved or suspended in a pharmaceutically acceptable liquid carrier such as water, an organic solvent, a mixture of both or pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators.

Liquid carriers suitable for use in the present application include, but are not limited to, water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the carrier can also include an oily ester such as ethyl oleate and isopropyl myristate. Sterile liquid carriers are useful in sterile liquid form comprising solid forms of Compound I for parenteral administration. The liquid carrier for pressurized compounds disclosed herein can be halogenated hydrocarbon or other pharmaceutically acceptable propellent.

Solid carriers suitable for use in the present application include, but are not limited to, inert substances such as lactose, starch, glucose, methyl-cellulose, magnesium stearate, dicalcium phosphate, mannitol and the like. A solid carrier can further include one or more substances acting as flavoring agents, lubricants, solubilizers, suspending agents, fillers, glidants, compression aids, binders or tablet-disintegrating agents; it can also be an encapsulating material. In powders, the carrier can be a finely divided solid which is in admixture with the finely divided active compound. In tablets, the active compound is mixed with a carrier having the necessary compression properties in suitable proportions and compacted in the shape and size desired. The powders and tablets preferably contain up to 99% of the active compound. Suitable solid carriers include, for example, calcium phosphate, magnesium stearate, talc, sugars, lactose, dextrin, starch, gelatin, cellulose, polyvinylpyrrolidine, low melting waxes and ion exchange resins. A tablet may be made by compression or molding, optionally with one or more accessory ingredients. Compressed tablets may be prepared by compressing in a suitable machine the active ingredient in a free flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose) surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein using, for example, hydroxypropyl methylcellulose in varying proportions to provide the desired release profile. Tablets may optionally be provided with an enteric coating, to provide release in parts of the gut other than the stomach.

Parenteral carriers suitable for use in the present application include, but are not limited to, sodium chloride solution, Ringer's dextrose, dextrose and sodium chloride, lactated Ringer's and fixed oils. Intravenous carriers include fluid and nutrient replenishers, electrolyte replenishers such as those based on Ringer's dextrose and the like. Preservatives and other additives can also be present, such as, for example, antimicrobials, antioxidants, chelating agents, inert gases and the like.

Carriers suitable for use in the present application can be mixed as needed with disintegrants, diluents, granulating agents, lubricants, binders and the like using conventional techniques known in the art. The carriers can also be sterilized using methods that do not deleteriously react with the compounds, as is generally known in the art.

Diluents may be added to the formulations of the present invention. Diluents increase the bulk of a solid pharmaceutical composition and/or combination and may make a pharmaceutical dosage form containing the composition and/or combination easier for the patient and care giver to handle. Diluents for solid compositions and/or combinations include, for example, microcrystalline cellulose (e.g., AVICEL), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., EUDRAGIT®), potassium chloride, powdered cellulose, sodium chloride, sorbitol, and talc.

Additional embodiments relate to the pharmaceutical formulations wherein the formulation is selected from the group consisting of a solid, powder, liquid and a gel. In certain embodiments, a pharmaceutical composition of the present invention is a solid (e.g., a powder, tablet, a capsule, granulates, and/or aggregates). In certain of such embodiments, a solid pharmaceutical composition comprising one or more ingredients known in the art, including, but not limited to, starches, sugars, diluents, granulating agents, lubricants, binders, and disintegrating agents.

Solid pharmaceutical compositions that are compacted into a dosage form, such as a tablet, may include excipients whose functions include helping to bind the active ingredient and other excipients together after compression. Binders for solid pharmaceutical compositions and/or combinations include acacia, alginic acid, carbomer (e.g., carbopol), carboxymethylcellulose sodium, dextrin, ethyl cellulose, gelatin, guar gum, gum tragacanth, hydrogenated vegetable oil, hydroxyethyl cellulose, hydroxypropyl cellulose (e.g., KLUCEL), hydroxypropyl methyl cellulose (e.g., METHOCEL), liquid glucose, magnesium aluminum silicate, maltodextrin, methylcellulose, polymethacrylates, povidone (e.g., KOLLIDON, PLASDONE), pregelatinized starch, sodium alginate, and starch.

The dissolution rate of a compacted solid pharmaceutical composition in the patient's stomach may be increased by the addition of a disintegrant to the composition and/or combination. Disintegrants include alginic acid, carboxymethylcellulose calcium, carboxymethylcellulose sodium (e.g., AC-DI-SOL and PRIMELLOSE), colloidal silicon dioxide, croscarmellose sodium, crospovidone (e.g., KOLLIDON and POLYPLASDONE), guar gum, magnesium aluminum silicate, methyl cellulose, microcrystalline cellulose, polacrilin potassium, powdered cellulose, pregelatinized starch, sodium alginate, sodium starch glycolate (e.g., EXPLOTAB), potato starch, and starch.

Glidants can be added to improve the flowability of a non-compacted solid composition and/or combination and to improve the accuracy of dosing. Excipients that may function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition and/or combination to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that may be included in the composition and/or combination of the present invention include maltol, vanillin, ethyl vanillin, menthol, citric acid, fumaric acid, ethyl maltol, and tartaric acid.

Solid and liquid compositions may also be dyed using any pharmaceutically acceptable colorant to improve their appearance and/or facilitate patient identification of the product and unit dosage level.

In certain embodiments, a pharmaceutical composition of the present invention is a liquid (e.g., a suspension, elixir and/or solution). In certain of such embodiments, a liquid pharmaceutical composition is prepared using ingredients known in the art, including, but not limited to, water, glycols, oils, alcohols, flavoring agents, preservatives, and coloring agents.

Liquid pharmaceutical compositions can be prepared using one or more solid forms of Compound I, or a pharmaceutically acceptable salt, solvate thereof, or salt solvate thereof, and any other solid excipients where the components are dissolved or suspended in a liquid carrier such as water, vegetable oil, alcohol, polyethylene glycol, propylene glycol, or glycerin.

For example, formulations for parenteral administration can contain as common excipients sterile water or saline, polyalkylene glycols such as polyethylene glycol, oils of vegetable origin, hydrogenated naphthalenes and the like. In particular, biocompatible, biodegradable lactide polymer, lactide/glycolide copolymer, or polyoxyethylene-polyoxypropylene copolymers can be useful excipients to control the release of active compounds. Other potentially useful parenteral delivery systems include ethylene-vinyl acetate copolymer particles, osmotic pumps, implantable infusion systems, and liposomes. Formulations for inhalation administration contain as excipients, for example, lactose, or can be aqueous solutions containing, for example, polyoxyethylene-9-auryl ether, glycocholate and deoxycholate, or oily solutions for administration in the form of nasal drops, or as a gel to be applied intranasally. Formulations for parenteral administration can also include glycocholate for buccal administration, methoxysalicylate for rectal administration, or citric acid for vaginal administration.

Liquid pharmaceutical compositions can contain emulsifying agents to disperse uniformly throughout the composition and/or combination an active ingredient or other excipient that is not soluble in the liquid carrier. Emulsifying agents that may be useful in liquid compositions and/or combinations of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions can also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, and xanthan gum.

Sweetening agents such as aspartame, lactose, sorbitol, saccharin, sodium saccharin, sucrose, aspartame, fructose, mannitol, and invert sugar may be added to improve the taste.

Preservatives and chelating agents such as alcohol, sodium benzoate, butylated hydroxyl toluene, butylated hydroxyanisole, and ethylenediamine tetraacetic acid may be added at levels safe for ingestion to improve storage stability.

A liquid composition can also contain a buffer such as gluconic acid, lactic acid, citric acid or acetic acid, sodium gluconate, sodium lactate, sodium citrate, or sodium acetate. Selection of excipients and the amounts used may be readily determined by the formulation scientist based upon experience and consideration of standard procedures and reference works in the field.

In one embodiment, a pharmaceutical composition is prepared for administration by injection (e.g., intravenous, subcutaneous, intramuscular, etc.). In certain of such embodiments, a pharmaceutical composition comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes. Aqueous injection suspensions may contain substances that increase the viscosity of the suspension, such as sodium carboxymethyl cellulose, sorbitol, or dextran. Optionally, such suspensions may also contain suitable stabilizers or agents that increase the solubility of the pharmaceutical agents to allow for the preparation of highly concentrated solutions.

The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables. Formulations for intravenous administration can comprise solutions in sterile isotonic aqueous buffer. Where necessary, the formulations can also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachet indicating the quantity of active agent. Where the solid form of Compound I is to be administered by infusion, it can be dispensed in a formulation with an infusion bottle containing sterile pharmaceutical grade water, saline or dextrose/water. Where the solid form of Compound I is administered by injection, an ampule of sterile water for injection or saline can be provided so that the ingredients can be mixed prior to administration.

Suitable formulations further include aqueous and non-aqueous sterile injection solutions that can contain antioxidants, buffers, bacteriostats, bactericidal antibiotics and solutes that render the formulation isotonic with the bodily fluids of the intended recipient; and aqueous and non-aqueous sterile suspensions, which can include suspending agents and thickening agents.

In certain embodiments, a pharmaceutical composition of the present invention is formulated as a depot preparation. Certain such depot preparations are typically longer acting than non-depot preparations. In certain embodiments, such preparations are administered by implantation (for example subcutaneously or intramuscularly) or by intramuscular injection. In certain embodiments, depot preparations are prepared using suitable polymeric or hydrophobic materials (for example an emulsion in an acceptable oil) or ion exchange resins, or as sparingly soluble derivatives, for example, as a sparingly soluble salt.

In certain embodiments, a pharmaceutical composition of the present invention comprises a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethylsulfoxide are used.

In certain embodiments, a pharmaceutical composition of the present invention comprises a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80 and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, a pharmaceutical composition of the present invention comprises a sustained-release system. A non-limiting example of such a sustained-release system is a semi-permeable matrix of solid hydrophobic polymers. In certain embodiments, sustained-release systems may, depending on their chemical nature, release pharmaceutical agents over a period of hours, days, weeks or months.

Appropriate pharmaceutical compositions of the present disclosure can be determined according to any clinically-acceptable route of administration of the composition to the subject. The manner in which the composition is administered is dependent, in part, upon the cause and/or location. One skilled in the art will recognize the advantages of certain routes of administration. The method includes administering an effective amount of the therapeutically active agent or one or more solid forms of Compound I (or composition comprising the therapeutic agent or Compound I) to achieve a desired biological response, e.g., an amount effective to alleviate, ameliorate, or prevent, in whole or in part, a symptom of a condition to be treated, e.g., oncology and neurology disorders. In various aspects, the route of administration is systemic, e.g., oral or by injection. The therapeutic agents or Compound I, or pharmaceutically acceptable salts or derivatives thereof, are administered orally, nasally, transdermally, pulmonary, inhalationally, buccally, sublingually, intraperintoneally, subcutaneously, intramuscularly, intravenously, rectally, intrapleurally, intrathecally, intraportally, and parenterally. Alternatively or in addition, the route of administration is local, e.g., topical, intra-tumor and peri-tumor. In some embodiments, the solid form of Compound I is administered orally.

In certain embodiments, a pharmaceutical composition of the present disclosure is prepared for oral administration. In certain of such embodiments, a pharmaceutical composition is formulated by combining one or more agents and pharmaceutically acceptable carriers. Certain of such carriers enable pharmaceutical compositions to be formulated as tablets, pills, dragees, capsules, liquids, gels, syrups, slurries, suspensions and the like, for oral ingestion by a subject. Suitable excipients include, but are not limited to, fillers, such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). In certain embodiments, such a mixture is optionally ground and auxiliaries are optionally added. In certain embodiments, pharmaceutical compositions are formed to obtain tablets or dragee cores. In certain embodiments, disintegrating agents (e.g., cross-linked polyvinyl pyrrolidone, agar, or alginic acid or a salt thereof, such as sodium alginate) are added.

In certain embodiments, dragee cores are provided with coatings. In certain such embodiments, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to tablets or dragee coatings.

In certain embodiments, pharmaceutical compositions for oral administration are push-fit capsules made of gelatin. Certain of such push-fit capsules comprise one or more pharmaceutical agents of the present invention in admixture with one or more filler such as lactose, binders such as starches, and/or lubricants such as talc or magnesium stearate and, optionally, stabilizers. In certain embodiments, pharmaceutical compositions for oral administration are soft, sealed capsules made of gelatin and a plasticizer, such as glycerol or sorbitol. In certain soft capsules, one or more pharmaceutical agents of the present invention are be dissolved or suspended in suitable liquids, such as fatty oils, liquid paraffin, or liquid polyethylene glycols. In addition, stabilizers may be added.

In certain embodiments, pharmaceutical compositions are prepared for buccal administration. Certain of such pharmaceutical compositions are tablets or lozenges formulated in conventional manner.

In certain embodiments, a pharmaceutical composition is prepared for transmucosal administration. In certain of such embodiments, penetrants appropriate to the barrier to be permeated are used in the formulation. Such penetrants are generally known in the art.

In certain embodiments, a pharmaceutical composition is prepared for administration by inhalation. Certain of such pharmaceutical compositions for inhalation are prepared in the form of an aerosol spray in a pressurized pack or a nebulizer. Certain of such pharmaceutical compositions comprise a propellant, e.g., dichlorodifluoromethane, trichlorofluoromethane, dichlorotetrafluoroethane, carbon dioxide or other suitable gas. In certain embodiments using a pressurized aerosol, the dosage unit may be determined with a valve that delivers a metered amount. In certain embodiments, capsules and cartridges for use in an inhaler or insufflator may be formulated. Certain of such formulations comprise a powder mixture of a pharmaceutical agent of the invention and a suitable powder base such as lactose or starch.

In other embodiments the solid forms of Compound I of the present disclosure are administered by the intravenous route. In further embodiments, the parenteral administration may be provided in a bolus or by infusion.

In certain embodiments, a pharmaceutical composition is prepared for rectal administration, such as a suppository or retention enema. Certain of such pharmaceutical compositions comprise known ingredients, such as cocoa butter and/or other glycerides.

In certain embodiments, a pharmaceutical composition is prepared for topical administration. Certain of such pharmaceutical compositions comprise bland moisturizing bases, such as ointments or creams. Exemplary suitable ointment bases include, but are not limited to, petrolatum, petrolatum plus volatile silicones, and lanolin and water in oil emulsions. Exemplary suitable cream bases include, but are not limited to, cold cream and hydrophilic ointment.

In certain embodiments, the therapeutically effective amount is sufficient to prevent, alleviate or ameliorate symptoms of a disease or to prolong the survival of the subject being treated. Determination of a therapeutically effective amount is well within the capability of those skilled in the art.

In certain embodiments, one or more solid forms of Compound I, or a pharmaceutically acceptable salt thereof, solvate thereof, or salt solvate thereof, are formulated as a prodrug. In certain embodiments, upon in vivo administration, a prodrug is chemically converted to the biologically, pharmaceutically or therapeutically more active form.

The concentration of a disclosed solid forms of Compound I in a pharmaceutically acceptable mixture will vary depending on several factors, including the dosage of the solid forms of Compound I to be administered, the pharmacokinetic characteristics of the solid form(s) employed, and the route of administration. The agent may be administered in a single dose or in repeat doses. The dosage regimen utilizing the solid forms of Compound I of the present invention is selected in accordance with a variety of factors including type, species, age, weight, sex and medical condition of the patient; the severity of the condition to be treated; the route of administration; and the particular solid forms or salt thereof employed. Treatments may be administered daily or more frequently depending upon a number of factors, including the overall health of a patient, and the formulation and route of administration of the selected form(s).

The solid forms of Compound I, or a pharmaceutically acceptable salt thereof, solvate thereof, or salt solvate thereof, or pharmaceutical compositions of the present disclosure may be manufactured and/or administered in single or multiple unit dose forms.

### Therapeutic Use

The crystalline forms and the pharmaceutical compositions of the present disclosure find use in any number of methods. For example, in some embodiments the crystalline forms and the pharmaceutical compositions are useful in methods for modulating a phosphoinositide 3-kinase (PI3K). In embodiments, modulating phosphoinositide 3-kinase (PI3K) activity is in a mammalian cell. In embodiments, modulating phosphoinositide 3-kinase (PI3K) can be in a subject in need thereof (e.g., a mammalian subject) and for treatment of a condition or disease described herein, including diseases or conditions wherein irreversible inhibition of PI3K provides therapeutic benefit to a subject having the disease or condition.

In one embodiment, the modulating PI3K is binding to PI3K. In other embodiments, the modulating PI3K is inhibiting PI3K, including irreversibly inhibiting the activity of PI3K. In embodiments, the inhibiting PI3K is inhibiting PI3Kα, including irreversibly inhibiting the activity of PI3Kα, for example by forming a covalent bond with a cysteine residue on PI3Kα.

In embodiments, modulating phosphoinositide 3-kinase (PI3K) activity is for treatment of diseases or conditions wherein irreversible inhibition of PI3K provides therapeutic benefit to a subject having the disease or condition. In embodiments, modulating phosphoinositide 3-kinase (PI3K) activity is for treatment of at least one indication selected from the group consisting of cancer, neurodegenerative diseases, autoimmune diseases and aging. In embodiments, modulating phosphoinositide 3-kinase (PI3K) activity is for treatment of at least one indication selected from the group consisting of ampullary cancer, anal cancer, bladder cancer, brain cancer, breast cancer, breast cancers, cervical cancer, colon cancer, colorectal cancer, endometrial cancer, esophageal cancer, gastric cancer, head and neck cancer, hematologic cancer, lung cancer, liver cancer, ovary cancer, pancreatic cancer, penile cancer, prostate cancer, renal cancer, salivary gland cancer, skin cancer, vaginal cancer, and urothelial cancer. In embodiments, modulating phosphoinositide 3-kinase (PI3K) activity is for treatment of cancer with a mutation in the PIK3CA gene.

In embodiments of the present disclosure, a method of treating cancer, neurodegenerative diseases, autoimmune diseases or aging is provided.

In embodiments of the present disclosure, a method of treating a condition associated with cell proliferation in a patient in need thereof is provided. In one embodiment, the present invention provides a method of treating cancer or tumors e.g., a solid tumor. In embodiments, the present disclosure provides a method of treating cancer with a mutation in the PIK3CA gene. In another embodiment, the present disclosure provides a method of treating ampullary cancer, anal cancer, bladder cancer, brain cancer, breast cancer, breast cancers, cervical cancer, colon cancer, colorectal cancer, endometrial cancer, esophageal cancer, gastric cancer, head and neck cancer, hematologic cancer, lung cancer, liver cancer, ovary cancer, pancreatic cancer, penile cancer, prostate cancer, renal cancer, salivary gland cancer, skin cancer, vaginal cancer, or urothelial cancer.

In embodiments of the present disclosure, a method of reducing, inhibiting, or ameliorating cell proliferation in a patient in need thereof is provided. In embodiments, the reducing, inhibiting, or ameliorating in the method disclosed herein, is in vivo. In another embodiment, the reducing, inhibiting, or ameliorating is in vitro. In embodiments, the cells in the method disclosed herein, are a cancer cells. In embodiments, the cancer cells are a prostate cancer cells.

In embodiments, the condition or disease associated with cell proliferation is cancer. In embodiments, the cancer has a mutation in the PIK3CA gene. In embodiments of the methods disclosed herein, the cancer is selected from the group consisting of: ampullary cancer, anal cancer, bladder cancer, brain cancer, breast cancer, breast cancers, cervical cancer, colon cancer, colorectal cancer, endometrial cancer, esophageal cancer, gastric cancer, head and neck cancer, hematologic cancer, lung cancer, liver cancer, ovary cancer, pancreatic cancer, penile cancer, prostate cancer, renal cancer, salivary gland cancer, skin cancer, vaginal cancer, and urothelial cancer.

In embodiments of the present disclosure, a method for reducing or preventing tumor growth, comprising contacting tumor cells with a compound or pharmaceutical composition as disclosed herein. In one embodiment, reducing or preventing tumor growth includes reduction in tumor volume. In one embodiment, reducing or preventing tumor growth includes complete elimination of tumors. In one embodiment, reducing or preventing tumor growth includes stopping or halting the existing tumor to grow. In one embodiment, reducing or preventing tumor growth includes reduction in the rate of tumor growth.

### EXAMPLES

The disclosure now being generally described, it will be more readily understood by reference to the following examples which are included merely for purposes of illustration of certain aspects and embodiments of the present invention and are not intended to limit the invention.

The examples below are intended to be exemplary and efforts have been made to ensure accuracy with respect to numbers used (for example, amounts, temperature, etc.), but some experimental errors and deviations should be accounted for within the knowledge of a person skilled in the art. Unless indicated otherwise, temperature is in degrees Centigrade. Reagents were purchased from commercial suppliers such as Sigma-Aldrich, Alfa Aesar, or TCI, and were used without further purification unless otherwise indicated.

### General Procedures—Analytical Methods

X-ray Powder Diffractometer (XRPD)

| | |
|---|---|
| Instrument | Bruker D8 Advance |

Method 1 (About 10 min)

| | |
|---|---|
| Detector | LYNXEYE_XE_T(1D mode) |
| Open angle | 2.94° |
| Radiation | Cu/K-Alpha1 (λ = 1.5406 Å) |
| X-ray generator power | 40 kV, 40 mA |
| Primary beam path slits | Twin_Primary motorized slit 10.0 mm by sample length; |
| | SollerMount axial soller 2.5° |
| Secondary beam path slits | Detector OpticsMount soller slit 2.5°; Twin_Secondary |
| | motorized slit 5.2 mm |
| Scan mode | Continuous scan |
| Scan type | Locked coupled |
| Step size | 0.02° |
| Time per step | 0.3 second per step |
| Scan range | 2° to 40° |
| Sample rotation speed | 15 rpm |
| Sample holder | Monocrystalline silicon, with cavity |

Differential Scanning Calorimetric (DSC)

| | |
|---|---|
| Instrument | TA Discovery 2500 |

Method

| | |
|---|---|
| Sample pan | Tzero pan and Tzero hermetic lid with a pin hole of |
| | 0.7 mm in diameter |
| Temperature range | 0 to 250° C. |
| Heating rate | 10° C./min |
| Nitrogen flow | 50 mL/min |
| Sample mass | About 1-2 mg |

Thermal Gravimetric Analysis (TGA)

| | |
|---|---|
| Instrument | Discovery 5500 or Q5000 |
| Sample pan | Aluminum, open |
| Start temperature | Ambient condition (below 35° C.) |
| Final temperature | 300° C. |
| Heating rate | 10° C./min |
| Nitrogen flow | Balance 10 mL/min; sample chamber 25 mL/min |
| Sample mass | About 2-10 mg |

High-throughput Dynamic Vapor Sorption (High-throughput DVS)
Method

| | |
|---|---|
| Instrument | SPSadv-1μ |
| Total gas flow | 4000 ml/min |
| Oven temperature | 25° C. |
| Solvent | Water |
| Method | Cycle: 40-95-0-95-40% RH |
| | Stage Step: 10% |
| | dm/dt = 0.002%/min |
| | Equilibrium: 240 min for each step |
| Sample mass | About 10-100 mg |

Karl Fischer (KF)

| | |
|---|---|
| Instrument | Mettler Toledo Coulometric KF Titrator C30 |
| Method | Coulometric |
| Sample mass | About 3-10 mg |

Nuclear Magnetic Resonance (NMR)

| | |
|---|---|
| Instrument | Bruker Avance-AV 400M (for ¹H-NMR) |
| Frequency | 400 MHz |
| Probe | 5 mm PABBO BB/19F-1H/D Z-GRD Z108618/0406 |
| | (for ¹H-NMR) |
| Number of scan | 8 |
| Temperature | 297.6K |
| Relaxation delay | 1 second |

Ion Chromatography (IC)

| | |
|---|---|
| Instrument | Metrohm 940 professional IC |
| Sample center | 889 IC |
| Detector | Conductivity detector |
| Eluent (anion) | 3.2 mmol/L Na₂CO₃ + 1.0 mmol/L NaHCO₃ |
| Suppressor solutions | 0.5% H₂SO₄ |
| Column: | Anion A SUPP 5-150 |
| Column temperature: | 30° C. |
| Flow rate: | 0.7 mL/min (anion) |
| Diluent: | Acetonitrile/H₂O = 1:1 (v:v) |
| Injection volume: | 20 μL |

High Performance Liquid Chromatograph (HPLC)

| Instrument | Agilent 1260 infinityll Binary Pump | | |
|---|---|---|---|
| HPLC method | Wave length: 254 nm | | |
| | Column: ZORBAX Eclipse XDB-C18, 4.6 mm*150 mm 5- | | |
| | Micron or Zorbax SB-C18, 3.5-Micron, 4.6*150 mm | | |
| | Detector: DAD | | |
| | Column temperature: 40° C. | | |
| | Flow rate: 1.2 mL/min | | |
| | Mobile phase A: 0.1% TFA in Water | | |
| | Mobile phase B: Acetonitrile | | |
| | Diluent: Acetonitrile/H₂O = 1:1 (v:v) | | |
| | Injection volume: 5 μL | | |
| | Gradient: | | |
| | Time | Mobile Phase | Mobile Phase |
| | (min) | A (%) | B (%) |
| | 0 | 95 | 5 |
| | 9 | 5 | 95 |
| | 13 | 5 | 95 |
| | 13.1 | 95 | 5 |
| | 17 | 95 | 5 |

### Example 1: Synthesis and Characterization of Crystalline form of methyl (5-(6-((4-(acryloylglycyl) piperazin-1-yl)methyl)-4-morpholinopyrrolo[2,1-f][1,2,4]triazin-2-yl)-4-(trifluoromethyl)pyridin-2-yl) carbamate (Compound I Form A)

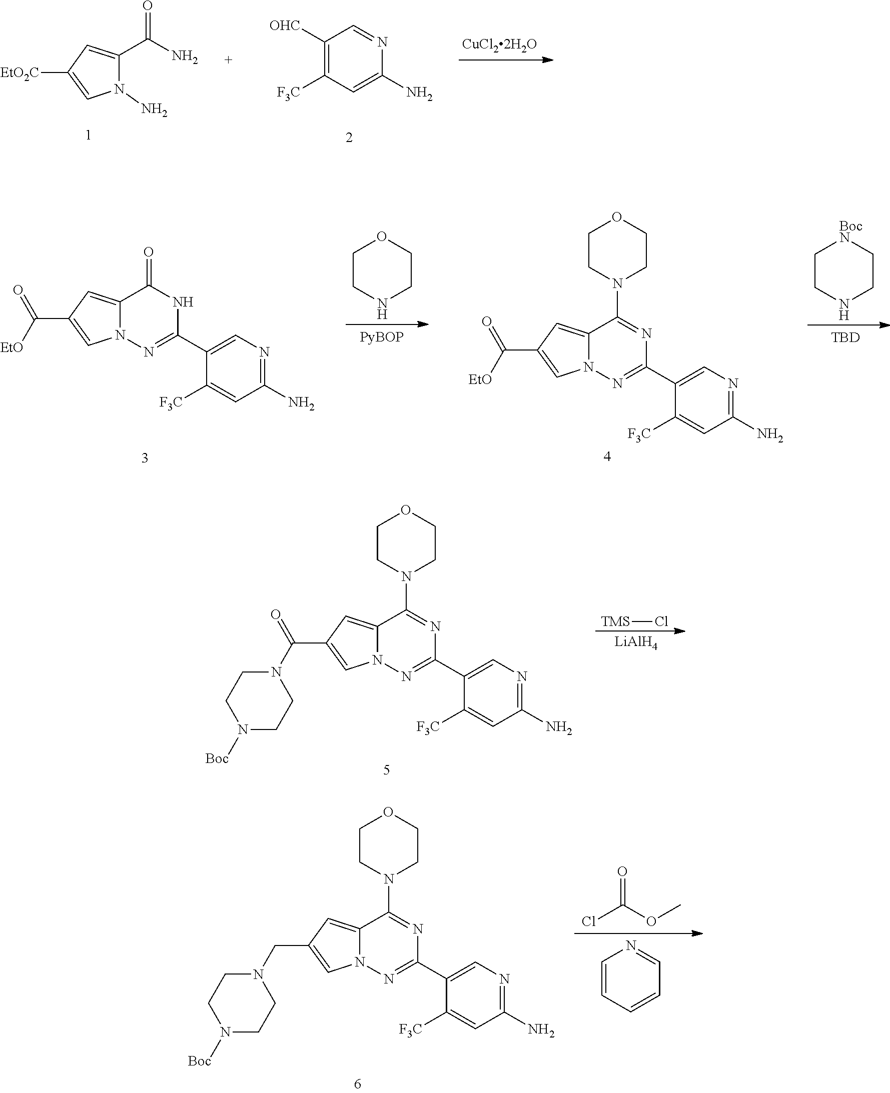

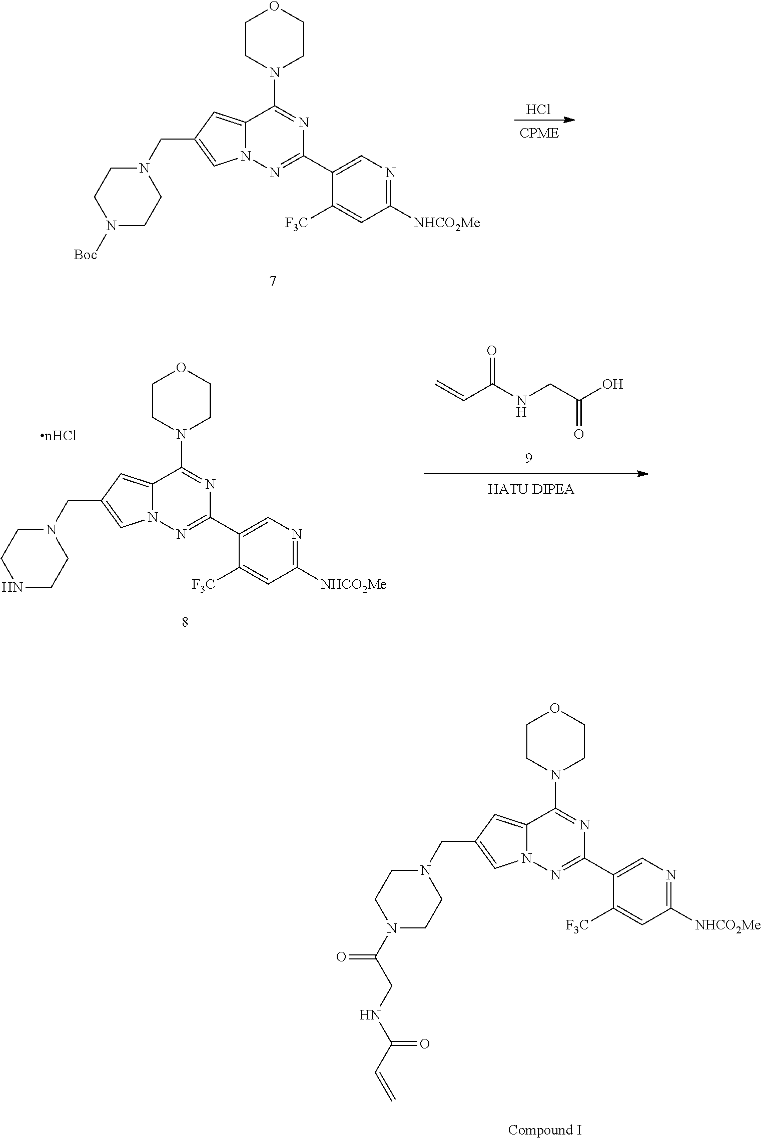

### Synthesis of Ethyl 1-amino-5-carbamoyl-1H-pyrrole-3-carboxylate (1)

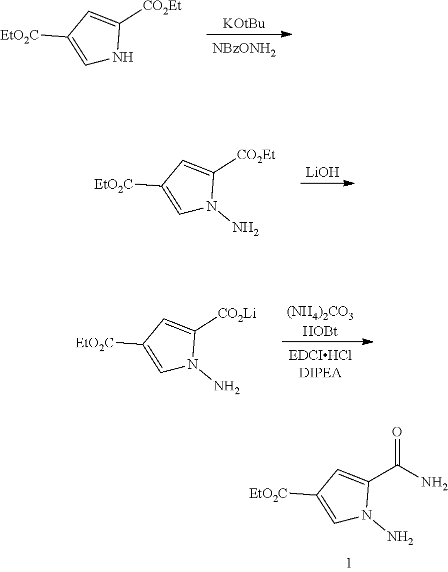

Diethyl 1-amino-1H-pyrrole-2,4-dicarboxylate. A 20 L reaction vessel was loaded with diethyl 1H-pyrrole-2,4-dicarboxylate (1002 g, 1 equiv, 4.507 mol) and NMP (7.8 L). Then potassium tert-butoxide (557.3 g, 1.1 equiv, 4.967 mol) was added, the mixture became pink, and the temperature reached 37° C. The mixture was stirred until all potassium tert-butoxide had dissolved and it was allowed to cool down to 21° C. Subsequently, O-(4-nitrobenzoyl) hydroxylamine (839.6 g, 1.02 equiv, 4.610 mol) was added portion-wise (exothermic, reached 36° C.). Reaction mixture turns into a dark purple suspension. The reaction mixture was stirred at 45° C. overnight (T slowly drops, mixture turns orange). A sample was taken after 18 h and diluted with ACN/water for HPLC analysis. Then, a solution of sodium dithionite (478.6 g, 0.6 equiv, 2.749 mol) in water (2.5 L) was slowly added keeping the temperature below 30°. The reaction mixture was transferred into a 50 L separation funnel. Toluene (15 L) and water (5 L) were added, and the phases were separated. The aqueous phase was extracted with toluene (3×1 L). The combined org phases were washed with water (5×1 L), sat. sodium bicarbonate (5×1 L) and brine (1 L), dried over sodium sulfate, filtered and concentrated to give diethyl 1-amino-1H-pyrrole-2,4-dicarboxylate (1077 g, 3.90 mol, 86%) as an orange oil (81% QNMR purity), which solidified upon standing. 1H NMR (299 MHz, DMSO-d6) δ 7.52 (d, J=2.1 Hz, 1H), 7.03 (d, J=2.1 Hz, 1H), 6.51 (d, J=1.8 Hz, 2H), 4.34-4.06 (m, 4H), 1.38-1.14 (m, 6H). LCMS (ESI): found 227.0 [M+H]⁺ (calculated 227.1 [M+H]⁺).

Lithium 1-amino-4-(ethoxycarbonyl)-1H-pyrrole-2-carboxylate. A 10 L reaction vessel was charged with diethyl 1-amino-1H-pyrrole-2,4-dicarboxylate (1077 g, 1 equiv, 3.90 mol), ethanol (3.8 L) and water (1.9 L). Lithium hydroxide monohydrate (163.9 g, 56 wt %, 0.99 equiv, 3.831 mol) was added and the mixture was stirred at 60° C. The conversion was monitored by LCMS analysis. The reaction was stopped after 6 hours and cooled down to RT. The mixture was diluted with 2 L toluene and layers were separated. The aqueous phase was washed with toluene (3×750 ml) and concentrated at the rotary evaporator at 60° C. The resulting solids was suspended in TBME (2 L), filtered and washed with TBME (1 L). Lithium 1-amino-4-(ethoxycarbonyl)-1H-pyrrole-2-carboxylate (853.1 g, 3.50 mol, 90%) was obtained as a pale-yellow solid (83% QNMR purity). 1H NMR (400 MHz, DMSO-D6) δ 7.37 (s, 2H), 7.09 (d, J=2.2 Hz, 1H), 6.63 (d, J=2.2 Hz, 1H), 4.12 (q, J=7.1 Hz, 2H), 1.22 (t, J=7.1 Hz, 3H). LCMS (ESI): found 199.0 [M−Li+2H]⁺ (calculated 199.1 [M−Li+2H]⁺).

Ethyl 1-amino-5-carbamoyl-1H-pyrrole-3-carboxylate (1). To a suspension of lithium 1-amino-4-(ethoxycarbonyl)-1H-pyrrole-2-carboxylate (835.8 g, 1 equiv, 3.399 mmol) in a mixture of DMF (3.4 L)/MeTHF (8.5 mL) was added ammonium carbonate (2939 g, 9.00 equiv, 30.59 mol) followed by HOBt (1041 g, 2.0 equiv, 6.797 mol), EDCl HCl (1303 g, 2.0 equiv, 6.797 mol) and DIPEA (2.96 L, 5 equiv, 16.99 mol). Adding the reagents causes an endotherm. The flask was stirred at room temperature (suspension) for 3 days. The conversion was monitored by LCMS analysis. The solids were filtered off and washed with MeTHF (2 L) and the filtrate was concentrated at 60° C. The crude material (2 kg) was redissolved in MeTHF (10 L) and washed with a saturated solution of sodium bicarbonate (2×2 L). Three phases were formed. The top phase (product fraction) was separated, dried over sodium sulfate, filtered and concentrated to dryness. The resulting crude material (908 g) was recrystallized from EtOH (1.8 L). The solids were filtered, washed with EtOH (200 mL) and dried to give the first crop of ethyl 1-amino-5-carbamoyl-1H-pyrrole-3-carboxylate (67 g) as a white solid. The mother liquor was concentrated (794 g) and purified by column chromatography (2 kg silica, gradient: DCM to 5% MeOH). The purified material (505 g) was recrystallized from EtOH (750 mL) and washed with EtOH (100 mL) to give a second crop of ethyl 1-amino-5-carbamoyl-1H-pyrrole-3-carboxylate (68 g). The concentrated mother liquor (435 g) was purified by column chromatography once more (6 kg silica, gradient: DCM to 5% MeOH). Recrystallizing the product fractions resulted into an additional crop 61 g of ethyl 1-amino-5-carbamoyl-1H-pyrrole-3-carboxylate. All crops merged gave ethyl 1-amino-5-carbamoyl-1H-pyrrole-3-carboxylate (1) (195.7 g, 992.4 mmol, 29%) as a white solid. ¹H NMR (299 MHz, DMSO-d6) δ 7.96 (s, 1H), 7.36 (d, J=2.0 Hz, 1H), 7.31 (s, 1H), 7.13 (d, J=2.1 Hz, 1H), 6.86 (d, J=1.8 Hz, 2H), 4.18 (q, J=7.1 Hz, 2H), 1.25 (t, J=7.1 Hz, 3H). LCMS (ESI): found 198.0 [M+H]⁺ (calculated 198.1 [M+H]⁺).

### Synthesis of 6-Amino-4-(trifluoromethyl) nicotinaldehyde

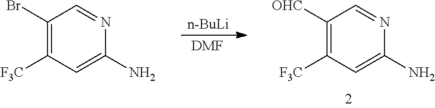

6-Amino-4-(trifluoromethyl) nicotinaldehyde (2). A 5 L three-necked flask, equipped with a thermometer and under nitrogen, was charged with 5-bromo-4-(trifluoromethyl)-2-pyridylamine (200.0 g, 1 equiv, 829.8 mmol) and dry THF (2000 mL). The solution was cooled down to −70° C. A 2.5 M solution of n-butyllithium in hexane (995.8 mL, 3.00 equiv, 2.49 mol) was added dropwise over 90 min, keeping the temperature below −60° C. The mixture was stirred at −70° C. for 15 min. Then, DMF (160.6 mL, 2.50 equiv, 2.074 mol) was added dropwise over 45 min, keeping the temperature below −60° C. The mixture was stirred at −70° C. for 30 min. The mixture was warmed-up to −40° C. and carefully quenched with water (74.8 mL, 5 equiv, 4.149 mol). The resulting solution was left to warm up to room temperature and stirred overnight. The mixture was further diluted with water/ethyl acetate (1 L/1 L) and transferred to a separating funnel. The organic layer was washed with water (5×300 mL). The combined aqueous phases were extracted with ethyl acetate (3×300 mL). The combined organic phases were washed with brine (300 mL), dried over sodium sulfate, filtered and concentrated until a small volume of solvent is left and a solid is formed. The mixture was cooled down to rt and diluted with heptane (300 mL). The mixture was filtered, the solid washed with heptane and dried to give 6-amino-4-(trifluoromethyl) nicotinaldehyde (2) (51.5 g, 271 mmol, 33%) as an orange solid.

¹H NMR (299 MHz, DMSO-d6) δ 9.80 (q, J=1.7 Hz, 1H), 8.63 (d, J=2.1 Hz, 1H), 7.73 (s, 2H), 6.84 (d, J=2.0 Hz, 1H). LCMS (ESI): found 191.0 [M+H]⁺ (calculated 191.0 [M+H]⁺).

Step 1: Synthesis of Ethyl 2-(6-amino-4-(trifluoromethyl)pyridin-3-yl)-4-oxo-3,4-dihydropyrrolo[2,1-f][1,2,4]triazine-6-carboxylate (3).

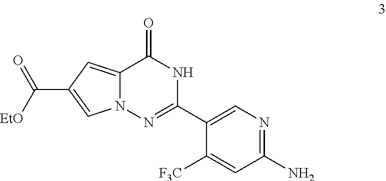 [SMILES: CCOC(=O)c1cc2c(=O)[nH]c(-c3cnc(N)cc3C(F)(F)F)nn2c1]

A 3 L 3-neck flask was equipped with a condenser, temperature probe and a mechanical stirrer and charged with a solution of ethyl 1-amino-5-carbamoyl-1H-pyrrole-3-carboxylate (1) (195.7 g, 1.00 equiv, 962.7 mmol) and 6-amino-4-(trifluoromethyl) nicotinaldehyde (2) (192.7 g, 1.00 equiv, 962.7 mmol) in DMSO (1.9 L). Then cupric chloride dihydrate (213.4 g, 1.30 equiv, 633.4 mmol) was added and the mixture was stirred at 100° C. for 18 hours. The mixture was cooled down to room temperature and poured onto ice water (10 L) causing the precipitation of the product. The suspension was stirred for 30 min before filtering (Buchner filter). The pale brown filter cake was washed with water (3×1 L) and TBME (3×1 L). The solids were stripped with toluene (3×1 L) on a rotary evaporator to remove residues of water. Ethyl 2-(6-amino-4-(trifluoromethyl)pyridin-3-yl)-4-oxo-3,4-dihydropyrrolo[2,1-f][1,2,4]triazine-6-carboxylate (3) (340.6 g, 760.0 mmol, 79%) was obtained as a solid. ¹H NMR (299 MHZ, DMSO-d6) δ 12.20 (s, 1H), 8.08 (d, J=1.8 Hz, 1H), 7.23 (d, J=1.8 Hz, 1H), 7.05 (s, 1H), 6.87 (s, 1H), 4.26 (q, J=6.9 Hz, 2H), 1.29 (t, J=7.1, 3H). LCMS (ESI): found 368.0 [M+H]⁺ (calculated 368.1 [M+H]⁺); 366.0 [M−H]⁻ (calculated 366.1 [M−H]⁻).

### Step 2: Synthesis of Ethyl 2-(6-amino-4-(trifluoromethyl)pyridin-3-yl)-4-morpholinopyrrolo[2,1-f][1,2,4]triazine-6-carboxylate (4)

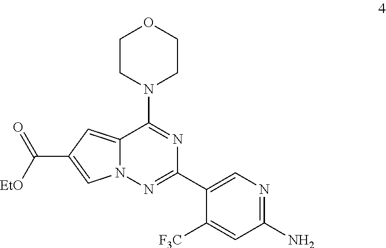 [SMILES: CCOC(=O)c1cc2c(N3CCOCC3)nc(-c3cnc(N)cc3C(F)(F)F)nn2c1]

A 5 L three-necked flask, under nitrogen and equipped with a thermometer, was charged with ethyl 2-(6-amino-4-(trifluoromethyl)pyridin-3-yl)-4-oxo-3,4-dihydropyrrolo[2,1-f][1,2,4]triazine-6-carboxylate (3) (335.6 g, 1 equiv, 749.2 mmol), DMF (2.5 L) and morpholine (400 mL, 6.2 equiv, 2.580 mol). Then PyBOP (606.9 g, 1.55 equiv, 1.166 mol) was added in several portions keeping the temperature around 15° C. (exothermic). The mixture was stirred at room temperature for 48 hours. The reaction mixture was transferred into a 20 L vessel and water (12.5 L) was slowly added. The suspension was filtered over a Buchner filter, washed with water (3×5 L) and TBME (2×5 L). The remaining clay like substance was re-dissolved in ethyl acetate at reflux. The hot suspension was filtered over a glass filter with Celite to remove the copper and the filtrate was concentrated. The remaining solid was suspended in ethyl acetate (1 L) at 60° C. The suspension was centrifuged at 3000 rpm. The resulting solution was decanted and concentrated. The residue was treated with warm ethyl acetate three more times. All organic phases were concentrated, and the obtained solids were filtered, washed with TBME, and dried under vacuum at 60° C. The solids were stripped with toluene (2×1 L) to obtain Ethyl 2-(6-amino-4-(trifluoromethyl)pyridin-3-yl)-4-morpholinopyrrolo[2,1-f][1,2,4]triazine-6-carboxylate (4) (206.5 g, 473.2 mmol, 63%) as a pale-yellow solid with a QNMR purity of 91%. 1H NMR (300 MHz, cdcl₃) δ 8.56 (s, 1H), 8.09 (d, J=1.6 Hz, 1H), 7.19 (d, J=1.6 Hz, 1H), 6.81 (s, 1H), 4.88 (s, 2H), 4.37 (q, J=7.1 Hz, 2H), 4.08 (t, J=4.8 Hz, 4H), 3.84 (t, J=4.8 Hz, 4H), 1.39 (t, J=7.1 Hz, 3H). LCMS (ESI): found 437.2 [M+H]⁺ (calculated 437.2 [M+H]⁺).

### Step 3: Synthesis of tert-Butyl 4-(2-(6-amino-4-(trifluoromethyl)pyridin-3-yl)-4-morpholinopyrrolo[2,1-f][1,2,4]triazine-6-carbonyl) piperazine-1-carboxylate (5)

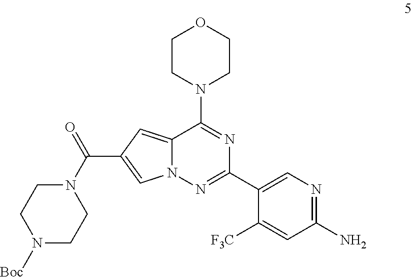 [SMILES: CC(C)(C)OC(=O)N1CCN(C(=O)c2cc3c(N4CCOCC4)nc(-c4cnc(N)cc4C(F)(F)F)nn3c2)CC1]

A 3 L three-necked flask under nitrogen was charged with ethyl 2-(6-amino-4-(trifluoromethyl)pyridin-3-yl)-4-morpholinopyrrolo[2,1-f][1,2,4]triazine-6-carboxylate (4) (206.5 g, 91 wt %, 1 equiv, 473.2 mmol) and THF (2 L). Then tert-butyl piperazine-1-carboxylate (440.7 g, 5.0 equiv, 2.366 mol) and TBD (65.87 g, 1.0 equiv, 473.2 mmol) were added and the solution was stirred at 65° C. for 2 days. The conversion was monitored by LCMS and proton NMR analysis. The reaction mixture was concentrated at 50° C. to remove the majority of THF. The material was dissolved in ethyl acetate (1 L) and washed with a 1M solution of potassium bisulfate (2×500 mL), water (3×500 ml, diluted with brine to enhance separation) and brine (250 mL). The organic phase was dried over sodium sulfate, filtered and concentrated at 50° C. (foaming brown oil). The brown foam was stripped with toluene (1.5 L) to remove traces of ethyl acetate and tert-butyl 4-(2-(6-amino-4-(trifluoromethyl)pyridin-3-yl)-4-morpholinopyrrolo[2,1-f][1,2,4]triazine-6-carbonyl) piperazine-1-carboxylate (5) (382.0 g, 464 mmol, 98%) was obtained as a brown foam with a QNMR purity of 70%. 1H NMR (300 MHZ, cdcl₃) δ 8.52 (s, 1H), 7.77 (d, J=1.6 Hz, 1H), 6.98 (d, J=1.7 Hz, 1H), 6.80 (s, 1H), 4.06 (t, J=4.9 Hz, 4H), 3.82 (t, J=4.9 Hz, 4H), 3.77-3.66 (m, overlaps with a THF signal, but subtraction of another THF signal gives 4H), 3.48 (t, J=5.0 Hz, 4H), 1.47 (m, 27H). LCMS (ESI): found 577.2 [M+H]⁺ (calculated 577.3 [M+H]⁺).

### Step 4: tert-Butyl 4-((2-(6-amino-4-(trifluoromethyl)pyridin-3-yl)-4-morpholinopyrrolo[2,1-f][1,2,4]triazin-6-yl)methyl) piperazine-1-carboxylate (6)

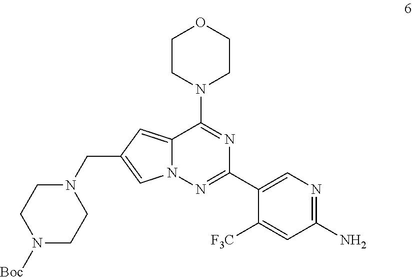 [SMILES: CC(C)(C)OC(=O)N1CCN(Cc2cc3c(N4CCOCC4)nc(-c4cnc(N)cc4C(F)(F)F)nn3c2)CC1]

A 5 L three-necked flask was charged with tert-butyl 4-(2-(6-amino-4-(trifluoromethyl)pyridin-3-yl)-4-morpholinopyrrolo[2,1-f][1,2,4]triazine-6-carbonyl) piperazine-1-carboxylate (5) (271.0 g, 1 equiv, 470.0 mmol) and dry THF (2 L) under nitrogen. The resulting solution was cooled to 0° C. and a solution of TMS-CI (107.0 n mL, 1.80 equiv, 846.0 mmol) in dry THF (100 mL) was added (mixture forms a brown suspension). The mixture was cooled further to −20° C. and a 2.4 M LiAlH₄ in THF (294.0 mL, 1.50 equiv, 705.0 mmol) was added over 80 min. The reaction mixture was stirred at −20° C. for 60 minutes. A 2M solution of Rochelle salt (1 L) was slowly added (very exothermic in the beginning, gas evolution) between-20° C. and −10° C. (the mixture become very thick, a solid is formed after 100 ml addition. The solid slowly dissolves at −8° C. and becomes easier to stir again). The mixture was allowed to warm-up to RT overnight. The organic layer was collected, the aq. layer was extracted with ethyl acetate. The combined organic layers were washed with brine, dried over sodium sulfate, filtered and concentrated. The residue was purified by column chromatography (6 kg silica; eluent: DCM/3.5 M NH₃ in MeOH) to give tert-butyl 4-((2-(6-amino-4-(trifluoromethyl)pyridin-3-yl)-4-morpholinopyrrolo[2,1-f][1,2,4]triazin-6-yl)methyl) piperazine-1-carboxylate (6) (172.2 g, 306.1 mmol, 65%) as an off-white fluffy solid. 1H NMR (299 MHZ, cdcl₃) δ 8.53 (s, 1H), 7.58 (d, J=1.5 Hz, 1H), 6.80 (s, 1H), 6.67 (d, J=1.6 Hz, 1H), 4.82 (s, 2H), 4.14-3.95 (m, 4H), 3.82 (t, J=4.8 Hz, 4H), 3.58 (s, 2H), 3.44 (t, J=5.3 Hz, 4H), 2.43 (t, J=5.1 Hz, 4H), 1.45 (d, J=1.4 Hz, 9H). LCMS (ESI): found 563.3 [M+H]⁺ (calculated 563.3 [M+H]⁺); 561.2 [M−H]⁻ (calculated 561.3 [M−H]⁻).

### Step 5: tert-Butyl 4-((2-(6-((methoxycarbonyl)amino)-4-(trifluoromethyl)pyridin-3-yl)-4-morpholinopyrrolo[2,1-f][1,2,4]triazin-6-yl)methyl) piperazine-1-carboxylate (7)

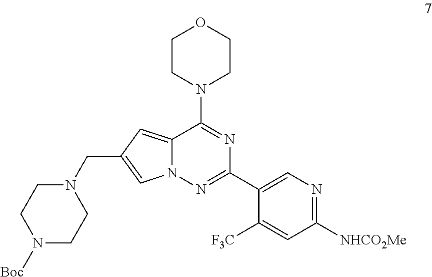

A 3 L three-necked flask was charged with tert-butyl 4-((2-(6-amino-4-(trifluoromethyl)pyridin-3-yl)-4-morpholinopyrrolo[2,1-f][1,2,4]triazin-6-yl)methyl) piperazine-1-carboxylate (6) (172.2 g, 1 Eq, 306.1 mmol), DCM (1.4 L) and pyridine (74 mL, 3.0 Eq, 918.2 mmol) and the solution was cooled down to 0° C. Methyl carbonochloridate (26.1 mL, 1.10 Eq, 336.7 mmol) was added to the reaction mixture over 30 min. The mixture was allowed to slowly warm-up to room temperature and stirred overnight. HPLC analysis showed about 90% conversion. Additional methyl carbonochloridate (2.4 mL, 0.10 Eq, 30.6 mmol) was added dropwise over 5 min and the mixture stirred at room temperature. After 4 h, HPLC analysis showed full conversion reach full conversion. The reaction mixture was poured into cold water (500 ml) and transferred to a separating funnel. The organic layer was collected, washed with water (3×500 ml) and brine, dried over sodium sulfate, filtered and concentrated. The residue was stripped with toluene to give tert-butyl 4-((2-(6-((methoxycarbonyl)amino)-4-(trifluoromethyl)pyridin-3-yl)-4-morpholinopyrrolo[2,1-f][1,2,4]triazin-6-yl)methyl) piperazine-1-carboxylate (7) (174.1 g, 280.5 mmol, 92% yield) as a solid. 1H NMR (299 MHZ, cdcl₃) δ 8.87 (s, 1H), 8.74 (s, 1H), 8.43 (s, 1H), 7.59 (d, J=1.5 Hz, 1H), 6.69 (d, J=1.6 Hz, 1H), 4.05 (t, J=4.8 Hz, 4H), 3.84 (d, J=7.1 Hz, 7H), 3.59 (s, 2H), 3.45 (t, J=5.1 Hz, 4H), 2.44 (t, J=4.9 Hz, 4H), 1.45 (s, 9H). LCMS (ESI): found 621.3 [M+H]⁺ (calculated 621.8 [M+H]⁺); 619.2 [M−H]⁻ (calculated 619.3 [M−H]⁻).

### Step 6: Methyl (5-(4-morpholino-6-(piperazin-1-ylmethyl) pyrrolo[2,1-f][1,2,4]triazin-2-yl)-4-(trifluoromethyl)pyridin-2-yl) carbamate hydrochloride (8)

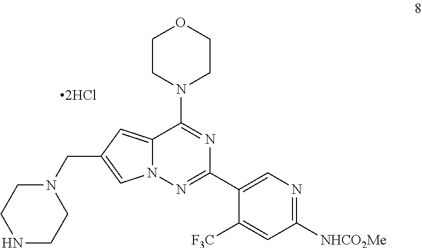

A flask was charged, under nitrogen, with tert-butyl 4-((2-(6-((methoxycarbonyl)amino)-4-(trifluoromethyl)pyridin-3-yl)-4-morpholinopyrrolo[2,1-f][1,2,4]triazin-6-yl)methyl) piperazine-1-carboxylate (7) (172.1 g, 1 Eq, 277.3 mmol) and CPME (1.0 L) (white suspension) and the mixture was cooled down to 6° C. A solution of HCl in CPME (1.5 L, 3.0 molar, 16.2 Eq, 4.50 mol) was added over 30 min (only slightly exothermic) and the mixture stirred at room temperature (yellow precipitate immediately formed) overnight. HPLC analysis showed full conversion. The mixture was filtered, the solid washed with 250 ml CPME and 500 ml TBME. The solid was transferred to a flask, but because it was very sticky (probably hygroscopic) MeOH was used to transfer the solid. The mixture was concentrated at the rotary evaporator and the solid dried to give 198 g of crude product. The material was stripped with toluene (2×1 L) before recrystallizing. The crude material (191 g) was recrystallized from MeOH (300 mL) The solid was collected by filtration, washed with cold MeOH (50 mL) and dried to give methyl (5-(4-morpholino-6-(piperazin-1-ylmethyl) pyrrolo[2,1-f][1,2,4]triazin-2-yl)-4-(trifluoromethyl)pyridin-2-yl) carbamate dihydrochloride (8) (88.8 g, 150 mmol, 54% yield) as a solid. ¹H NMR (400 MHZ, DMSO-D6) δ 12.13 (s, 1H), 10.92 (s, 1H), 9.59 (s, 2H), 8.73 (s, 1H), 8.31 (s, 1H), 8.07 (s, 1H), 7.38 (s, 1H), 4.41 (s, 2H), 4.02 (t, J=4.9 Hz, 4H), 3.80-3.69 (m, 8H), 3.69-3.33 (m, 13H), 3.16 (s, 3H). LCMS (ESI): found 521.2 [M+H]⁺ (calculated 521.2 [M+H]⁺).

### Step 7: Methyl (5-(6-((4-(acryloylglycyl) piperazin-1-yl)methyl)-4-morpholinopyrrolo[2,1-f][1,2,4]triazin-2-yl)-4-(trifluoromethyl)pyridin-2-yl) carbamate (Compound 1)

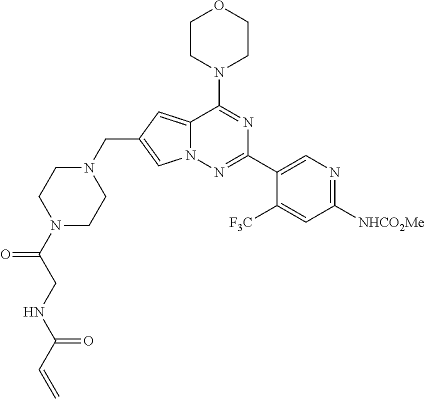

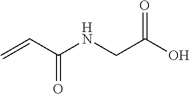

Acryloyl glycine. A 2 L RBF was charged with glycine (200 g, 1.0 Eq, 2.66 mol), deionized water (88 mL) and sodium hydroxide (710 mL, 30% Wt, 2.0 Acryloyl glycine Eq, 5.32 mol). The mixture was cooled to −5° C. before acryloyl chloride (225 mL, 96% Wt, 1.0 Eq, 2.66 mol) was added while maintaining the temperature around 0° C. The mixture was stirred at 0° C. for 1 h. After 1 h the mixture was acidified with HCl (310 mL, 12 molar, 1.4 Eq, 3.72 mol) until pH 2. The mixture was saturated with sodium sulphate (+100 g) and diluted with warm MeTHF (300 mL). Phases were separated, and the aqueous phase was extracted with MeTHF (3×250 mL). The combined organic layers were washed with brine (150 mL), dried over sodium sulphate and concentrated. At a volume of roughly 500 mL, a solvent swap to ethyl acetate (added 500 mL) was performed. After removing 500 mL ethyl acetate, the solvent switch was repeated twice more. The resulting slurry was agitated at room temperature for 1 h before the solids were collected and rinsed with ethyl acetate (2×60 mL), dried under vacuum, and dried further on a rotary evaporator to give Acryloyl glycine (76.00 g, 588.6 mmol, 22% yield) as white solid. ¹H NMR (299 MHZ, DMSO-d6) δ 12.58 (s, 1H), 8.42 (t, J=6.0 Hz, 1H), 6.30 (ddd, J=17.1, 10.1, 0.8 Hz, 1H), 6.10 (ddd, J=17.2, 2.3, 0.8 Hz, 1H), 5.62 (ddd, J=10.1, 2.3, 0.8 Hz, 1H), 3.84 (dd, J=6.0, 0.8 Hz, 2H). LCMS (ESI): found 130.1 [M+H]⁺ (calculated 130.1 [M+H]⁺).

To a suspension of methyl (5-(4-morpholino-6-(piperazin-1-ylmethyl) pyrrolo[2,1-f][1,2,4]triazin-2-yl)-4-(trifluoromethyl)pyridin-2-yl) carbamate dihydrochloride (8) (73.2 g, 1 Eq, 123 mmol) in THF (750 mL) was added DIPEA (100 mL, 4.7 Eq, 416 mmol) and the mixture slowly became a solution and then turbid again. Acryloyl glycine (23.9 g, 1.5 Eq, 185 mmol) was added followed by HATU (93.8 g, 2 Eq, 247 mmol) and the mixture (yellow) was stirred at room temperature. After 20 min the mixture became a black suspension. After 1 h the reaction was finished and diluted with 600 mL of ethyl acetate and quenched with 600 mL of a sat. solution of sodium bicarbonate. The organic phase was separated and washed with a sat. solution of sodium bicarbonate (600 mL) and brine (2×400 mL). The organic phase was dried over sodium sulphate, filtered, and concentrated to give the 147.2 g of crude material as a red sticky oil. The crude material was dissolved in NMP (600 mL) at room temperature and water (1.5 L) was added dropwise. The mixture was stirred for 1 h at room temperature and then filtered off. The residue was extensively washed with water and TBME to remove most of the NMP. The obtained solids were dissolved in DMSO (470 mL) and precipitated by adding water (900 mL). Once again, the residue was extensively washed with water and TBME to remove most of the NMP and DMSO. After drying the material in a circulation oven at 40° C. overnight, methyl (5-(6-((4-(acryloylglycyl) piperazin-1-yl)methyl)-4-morpholinopyrrolo[2,1-f][1,2,4]triazin-2-yl)-4-(trifluoromethyl)pyridin-2-yl) carbamate (Compound I) (57.7 g, 91.4 mmol, 74% yield) was obtained as a solid. ¹H NMR (400 MHZ, DMSO-D6) δ 10.89 (s, 1H), 8.72 (s, 1H), 8.30 (s, 1H), 8.20 (t, J=5.5 Hz, 1H), 7.75 (d, J=1.5 Hz, 1H), 6.97 (d, J=1.6 Hz, 1H), 6.37 (dd, J=17.1, 10.2 Hz, 1H), 6.09 (dd, J=17.1, 2.2 Hz, 1H), 5.59 (dd, J=10.2, 2.2 Hz, 1H), 4.02 (d, J=5.5 Hz, 2H), 3.98 (t, J=4.9 Hz, 4H), 3.74 (d, J=4.4 Hz, 7H), 3.57 (s, 2H), 3.52-3.37 (m, 4H), 2.39 (dt, J=17.8, 4.9 Hz, 4H). LCMS (ESI): found 632.4 [M+H]⁺ (calculated 632.3 [M+H]⁺); 630.2 [M−H]⁻ (calculated 630.2 [M−H]⁻).

### Compound I Free Base Form A

Chemical purity was 97.1% by HPLC [area %].

X-ray powder diffraction (XRPD) analysis was conducted on the solid crystals of Crystalline Form A, of Compound I. The XRPD is shown in FIG. 1A. Characteristic peaks include on or more of the peaks shown in Table 1. Form A was determined successfully in this study, and the results indicate that it is an anhydrate.

DSC and TGA thermograms were also obtained for Form A of Compound I as shown in FIG. 1B and FIG. 1C, respectively. DSC shows a small endothermic peak from about 12° C., which could correspond to evaporation of free water and a decomposition endothermic peak at Tₒₙₛₑₜ of 199.2° C. It decomposes upon melting. TGA shows about 0.6% weight loss at about 160° C. ¹H-NMR shows no detectable residual solvent.

A DVS (dynamic vapor sorption) analysis at 25° C. was carried out on Form A of Compound I as shown in FIG. 1D. The Form A of Compound I is slightly hygroscopic with about 1.4% water uptake from 40% to 95% RH. After the DVS test, obtained sample was still the free base Pattern A.

### Example 2: Preparation and Characterization of Salt Polymorphs of Compound I

Experiments were carried out as follows.

Conditions A About 30 mg of the free base Form A of Compound I and 1 equiv. of acid was added into DCM in a 2 mL glass vial. Obtained mixtures were stirred at 25° C. for at least 72 hours.

Conditions B/C About 30 mg of the free base Form A of Compound I and 1 equiv. of acid was added into MeOH or acetone in a 2 mL glass vial. Obtained mixtures was stirred at 50° C. for 2 hours and then at 25° C. for at least 72 hours.

Obtained suspensions were filtered through a 0.45 μm nylon membrane filter by centrifugation at 14,000 rpm. After being dried at 50° C. under vacuum for 2 h, solids were analyzed by XRPD. See e.g., FIGS. 5A, 6A, 7A, 8A, 9A, 10A, 11A, 12A, 13A, 14A, 15A, 16A, 17, 18A, and 19A.

The results are summarized in Table A below.

TABLE A
Preparation of salts of Compound I

| | Counter | A | B | C |
|---|---|---|---|---|
| Exp. ID | ions | DCM | MeOH | Acetone |
| 1 | Free base only | Form A of Compound I | Form A of Compound I | Form A of Compound I |
| 2 | HCl (1.0 equiv.) | Form A of Compound I | Form A-1 of | Form B-1 of |
| | | | Compound I HCl salt | Compound I HCl salt |
| 3 | Sulfuric acid | Free base Pattern A | Form A-2 of | Form B-2 of |
| | (1.0 equiv.) | | Compound I sulfuric | Compound I sulfuric |
| | | | acid salt | acid salt |
| 4 | Phosphoric acid | Free base Pattern A | Form A* of Compound | Free base Pattern A |
| | (1.0 equiv.) | | I phosphate salt | |
| 5 | Methanesulfonic | Form A-3 of Compound | Form B-3 of Compound | Form C-3 of Compound |
| | acid (1.0 equiv.) | I mesylate salt | I mesylate salt | I mesylate salt |
| 6 | Benzenesulfonic | Form A** of | Form A** of | Form A** of |
| | acid (1.0 equiv.) | Compound I | Compound I | Compound I |
| | | benzenesulfonate salt | benzenesulfonate salt | benzenesulfonate salt |
| 7 | p- | Form A-4 of | Form A-4 of | Form A-4 of |
| | Toluenesulfonic | Compound I tosylate | Compound I tosylate | Compound I tosylate |
| | acid (1.0 equiv.) | salt | salt | salt |
| 8 | Fumaric acid | Form A-5 of Compound | Free base Pattern A | Free base Pattern A |
| | (1.0 equiv.) | I fumarate salt | | |
| 9 | Maleic acid | Form A-6 of Compound | Form B* of Compound | Form A-6 of Compound |
| | (1.0 equiv.) | I maleate salt | I maleate salt | I maleate salt |
| 10 | L-Malic acid | Sticky sample | Free base Pattern A | Sticky sample |
| | (1.0 equiv.) | | | |
| 11 | L-Tartaric acid | Form A-7 of Compound | Form A-7 of Compound | Form B-7 of Compound |
| | (1.0 equiv.) | I L-tartrate salt | I L-tartrate salt | I L-tartrate salt |
| | | low crystallinity | low crystallinity | low crystallinity |
| 12 | Hippuric acid | Free base Pattern A + | Free base Pattern A | Free base Pattern A + |
| | (1.0 equiv.) | hippuric acid | | hippuric acid |
| 13 | Citric acid | Free base Pattern A | Form A-8 of | Form B-8 of Compound |
| | (1.0 equiv.) | | Compound I citrate salt | I citrate salt |
| 14 | Succinic acid | Form A-9 of Compound | Free base Pattern A | Free base Pattern A |
| | (1.0 equiv.) | I succinate salt, low | | |
| | | crystallinity | | |
| 15 | Adipic acid | Free base Pattern A + | Free base Pattern A | Free base Pattern A |
| | (1.0 equiv.) | adipic acid | | |
| 16 | L-Aspartic acid | Free base Pattern A | Free base Pattern A | Free base Pattern A |
| | (1.0 equiv.) | | | |

### Example 3: Preparation and Characterization of Crystalline form of Methyl (5-(6-((4-(acryloylglycyl) piperazin-1-yl)methyl)-4-morpholinopyrrolo[2,1-f][1,2,4]triazin-2-yl)-4-(trifluoromethyl)pyridin-2-yl) carbamate phosphate salt (Compound I Phosphate Salt Form A*)

3-1: Crystallization from MeOH/Water System

Crystalline Form A* of Compound I phosphate salt was prepared using the following procedure:

500 mg of the free base Form A of Compound I was weighed into a 20 mL glass vial. 6 mL of MeOH and 40 μL of water were added into the vial under stirring at 50° C. Next, ˜1.05 equivalent phosphoric acid was added into the resultant suspension. About 2 mg of the Compound I Phosphate salt Form A* seeds was then added to the suspension. The suspension was stirred at 50° C. for about 2 hours then cooled to 25° C. and kept stirring at 25° C. for about 5 days. Next, 500 mg of the free base Form A of Compound I was weighed into a 20 mL glass vial and added into above-mentioned suspension and another 4 mL of MeOH and 40 μL of water were added into the vial. The resultant suspension was stirred at 50° C. and ˜1.05 equivalent phosphoric acid was added into the suspension and the resultant suspension was stirred at 50° C. for about 2 hours then cooled to 25° C. and kept stirring at 25° C. for about 1 day. Solids were collected by suction filtration and then dried at 50° C. under vacuum for about 2 hours, to obtain 1.1 g of crystalline Form A* of Compound I Phosphate salt in 95% yield. The chemical purity was 97.4% by HPLC [area %].

X-ray powder diffraction (XRPD) analysis was conducted on the solid crystals of Crystalline Form A* of Compound I phosphate salt. The XRPD is shown in FIG. 2A. Characteristic peaks include on or more of the peaks shown in Table 2. Form A was determined successfully in this study, and the results indicate that it is a hydrate. Karl Fischer (KF) shows Crystalline Form A* of Compound I phosphate salt contains about 4.5% water by weight, equivalent to 1.9 water molecule.

DSC and TGA thermograms were also obtained for Form A* of Compound I phosphate salt as shown in FIG. 2B and FIG. 2C, respectively. DSC shows a dehydration peak from about 9° C. and a decomposition endothermic peak at Tₒₙₛₑₜ of 188.7° C. It decomposes upon melting. TGA shows about 3.7% weight loss at about 170° C. Ion Chromatography (IC) shows free base: PO₄³⁻ is 1:1.1. ¹H-NMR shows no detectable residual solvent.

A DVS (dynamic vapor sorption) analysis at 25° C. was carried out on Form A* of Compound I phosphate salt as shown in FIG. 2D. Form A* of Compound I phosphate salt is slightly hygroscopic in <90% RH. Then it becomes hygroscopic and shows 3.3% water uptake from 40% RH to 95% RH. Form A* of Compound I phosphate salt dehydrates in <30% RH and it converts to hydrate form in >30% RH. After the DVS test, obtained sample was still the Form A* of Compound I phosphate salt.

Variable humidity XRPD (VH-XRPD) experiments.

About 10 mg of Form A* of Compound I phosphate salt was used as starting material. XRPD analysis was carried out in each specific relative humidity at 25° C. Step: 40% RH (initial)-60% RH (4 h)-80% RH (4 h)-40% RH (4 h)-20% RH (4 h)-0% RH (4 h)-40% RH (4 h).

Variable humidity XRPD (VH-XRPD) experiments

| | Exp. ID | |
|---|---|---|
| | Relative humidity at 25° C. | |
| | (equilibration time) | XRPD |
| | Initial | |
| | Phosphate salt Form A* | |
| 1 | 40% RH (initial) | Form A* of Compound I phosphate salt |
| 2 | 60% RH (4 h) | Form A* of Compound I phosphate salt |
| 3 | 80% RH (4 h) | Form A* of Compound I phosphate salt |
| 4 | 40% RH (4 h) | Form A* of Compound I phosphate salt |
| 5 | 20% RH (4 h) | Form A* of Compound I phosphate salt |
| 6 | <20% RH (4 h) | Form B of Compound I phosphate salt |
| 7 | 40% RH (4 h) | Form A* of Compound I phosphate salt |

According to variable humidity XRPD experiments and DVS isotherms, Form A* of Compound I phosphate salt dehydrated and converted to anhydrate Crystalline Form B of Compound I phosphate salt gradually when RH was <20% RH. The anhydrate Form B of Compound I phosphate salt reverted to hydrate Form A* of Compound I phosphate salt in ≥20% RH.

3-2: Crystallization from EtOH/Water System:

| | | | Purity | |
|---|---|---|---|---|
| Solvent System | Procedure | XRPD | (HPLC) | Yield |
| solvent (12V): | Charge freebase, 12 V solvent | Form A* of Compound | 98.6% | 87% |
| EtOH/Water = 2/1 | mixture and 1.1eq H₃PO₄ | I phosphate salt | | |
| Anti-solvent | Adjust to 65° C. and stir | | | |
| (28 V): | until dissolved | | | |
| EtOH | Adjust to 40° C. | | | |
| Total: 40 V | Charge the 1% w/w seed | | | |
| | Stir at 40° C. for 2 h | | | |
| | Adjust to 35° C. | | | |
| | Dropwise 28 V anti-solvent | | | |
| | over 3 h | | | |
| | Stir at 35° C. for 2 h | | | |
| | Cool to 25° over 1 h | | | |
| | Stir at 25° C., take sample | | | |
| | Filter and wash | | | |
| | Dry solely by vacuum at | | | |
| | 50° C. for 18 h. | | | |
| | Dry by wet N₂ at 25° | | | |
| | C. for 18 h. | | | |

### Humidified Drying Process

In order to avoid the form conversion during drying, a humidified drying process was developed, as follows:

Test 1: humidify drying the wet sample at 25° C. with 30-40% of Relative Humidify (RH) for 43 hours, the EtOH was less than 0.01%, while the KF was about 6.0%, the crystal form was Form A* of Compound I phosphate salt.

Test 2: humidify drying the wet sample at 30° C. with 60-70% RH for 43 hours, the EtOH was less than 0.01%, the KF was about 6.3%, the crystal form was Form A* of Compound I phosphate salt.

Test 3: humidify drying the wet sample at 35° C. with 30-40% of Relative Humidify (RH) for 67 hours, the EtOH was less than 0.01%, while the KF was about 5.7%, the crystal form was Form A* of Compound I phosphate salt.

Test 4: humidify drying the wet sample at 35° C. with 60-70% of Relative Humidify (RH) for 67 hours, the EtOH was less than 0.01%, while the KF was about 6.1%, the crystal form was Form A* of Compound I phosphate salt.

### Example 4: Preparation and Characterization of Crystalline form of Methyl (5-(6-((4-(acryloylglycyl) piperazin-1-yl)methyl)-4-morpholinopyrrolo[2,1-f][1,2,4]triazin-2-yl)-4-(trifluoromethyl)pyridin-2-yl) carbamate benzenesulfonate salt (Compound I benzenesulfonate salt Form A**)

Crystalline Form A** of Compound I benzenesulfonate salt was prepared using the following procedure:

500 mg of the free base Form A of Compound I was weighed into a 20 mL glass vial. 2.5 mL of MeOH was added into the vial and the resulting suspension was stirred at 50° C. Next, ˜1.05 equivalent benzenesulfonic acid was dissolved in 1 mL of MeOH and added into the suspension. Next about 2 mg of the crystalline Form A** of Compound I benzenesulfonate salt seeds was added into above suspension. The resulting suspension was stirred at 50° C. for about 2 hours then cooled to 25° C. and kept stirring at 25° C. for about 5 days. 500 mg of the free base Form A of Compound I was weighed into a 20 mL glass vial and added into above-mentioned suspension. Another 4.5 mL of MeOH were added into the vial and the suspension was stirred at 50° C. Next, ˜1.05 equivalent benzenesulfonic acid was dissolved in 1 mL of MeOH, then this clear solution was added to the suspension. The resulting suspension was stirred at 50° C. for about 2 hours then cooled to 25° C. and kept stirring at 25° C. for about 1 day. The solids were collected by suction filtration and then dried at 50° C. under vacuum for about 2 hours to obtain 1.14 g of Crystalline Form A** of Compound I benzenesulfonate salt in 91% yield. The chemical purity was 97.3% by HPLC [area %].

X-ray powder diffraction (XRPD) analysis was conducted on the solid crystals of Crystalline Form A** of Compound I benzenesulfonate salt. The XRPD is shown in FIG. 3A. Characteristic peaks include on or more of the peaks shown in Table 3. Form A was determined successfully in this study, and the results indicate that it is an anhydrate.

DSC and TGA thermograms were also obtained for Form A** of Compound I benzenesulfonate salt as shown in FIG. 3B and FIG. 3C, respectively. DSC shows that it decomposed (around 250° C.) upon melting. TGA shows about 1.1% weight loss at about 190° C. ¹H-NMR shows free base: benzenesulfonic acid is 1:1. ¹H-NMR shows no detectable residual solvent.

A DVS (dynamic vapor sorption) analysis at 25° C. was carried out on Form A** of Compound I benzenesulfonate salt as shown in FIG. 3D. Form A** of Compound I benzenesulfonate salt is slightly hygroscopic with about 1.4% water uptake from 40% to 95% RH. After the DVS test, obtained sample was still the Form A** of Compound I benzenesulfonate salt.

### Example 5: Preparation and Characterization of Crystalline form of Methyl (5-(6-((4-(acryloylglycyl) piperazin-1-yl)methyl)-4-morpholinopyrrolo[2,1-f][1,2,4]triazin-2-yl)-4-(trifluoromethyl)pyridin-2-yl) carbamate maleate salt (Compound I maleate salt Form B*)

Crystalline Form B* of Compound I maleate salt was prepared using the following procedure:

1.0 g of the free base Form A of Compound I was weighed into a 20 mL glass vial. 11 mL of MeOH and 40 μL of water were added into the vial and the resulting suspension was stirred at 50° C. Maleic acid (1.05 equivalent) was added into the suspension and then about 2 mg of seeds of the crystalline Form B* of Compound I maleate salt was added. The resulting suspension was stirred at 50° C. for about 2 hours then cooled to 25° C. and kept stirring at 25° C. for about 2 days. Solids were collected by suction filtration and then dried at 50° C. under vacuum for about 2 hours to obtain 1.1 g of crystalline Form B* of Compound I maleate salt in 92% yield. The chemical purity was 97.9% by HPLC [area %].

X-ray powder diffraction (XRPD) analysis was conducted on the solid crystals of crystalline Form B* of Compound I maleate salt. The XRPD is shown in FIG. 4A. Characteristic peaks include on or more of the peaks shown in Table 4. Form B* of Compound I maleate salt was determined successfully in this study, and the results indicate that it is a hydrate. KF shows it contains about 3.2% water by weight, equivalent to 1.4 water molecule.

DSC and TGA thermograms were also obtained for Form B* of Compound I maleate salt as shown in FIG. 4B and FIG. 4C. DSC shows a dehydration peak from about 10° C. and a decomposition endothermic peak at Tₒₙₛₑₜ of 166.2° C. It decomposes upon melting. TGA shows about 1.8% weight loss at about 130° C. ¹H-NMR shows free base: maleic acid is 1:1.1. ¹H-NMR also shows no detectable residual solvent.

A DVS (dynamic vapor sorption) analysis at 25° C. was carried out on Form B* of Compound I maleate salt as shown in FIG. 4D. Form B* of Compound I maleate salt shows a nearly monotonic water sorption-desorption behavior depending on RH. This suggests that Form B* of Compound I maleate salt could be a channel hydrate. It shows 2.7% water uptake from 40% to 90% RH. After the DVS test, obtained sample was still Form B* of Compound I maleate salt.

### Example 6: Preparation of Crystalline Form B of Compound I Phosphate Salt

Crystalline Form B of Compound I phosphate salt was prepared by heating Form A* of Compound I phosphate salt to 100° C. and drying under a nitrogen flow, or by placing Form A* of Compound I phosphate salt in 0% RH at 25° C. for 4 hours.

X-ray powder diffraction (XRPD) analysis was conducted on the solid crystals of crystalline Form B of Compound I phosphate salt. The XRPD is shown in FIG. 20. Characteristic peaks include on or more of the peaks shown in Table 20. Form B of Compound I phosphate salt was determined successfully in this study, and the results indicate that it is an anhydrate.

### Example 7: Preparation of Crystalline Form C of Compound I Phosphate Salt

Crystalline Form C of Compound I phosphate salt was prepared from Form A* of Compound I phosphate salt by equilibration in acetone/water (a.w.=0.3*, v:v=97:3) at 25° C. for about 11 days and at 50° C. for about 1 week.

Equilibration at 50° C. for about 1 week:

50 mg of Form A* of Compound I phosphate salt was equilibrated in 0.2˜1 mL of acetone/water (a.w.=0.9, v:v=35:65) at 50° C. for 1 week with a stirring bar on a magnetic stirring plate at a rate of 400 rpm. Obtained suspensions were filtered through a 0.45 μm nylon membrane filter by centrifugation at 14,000 rpm.

X-ray powder diffraction (XRPD) analysis was conducted on the solid crystals of crystalline Form C of Compound I phosphate salt. The XRPD is shown in FIG. 21A. Characteristic peaks include on or more of the peaks shown in Table 21. Form C of Compound I phosphate salt was determined successfully in this study, and the results indicate that it is a hydrate.

DSC and TGA thermograms were also obtained for Form B of Compound I phosphate salt as shown in FIG. 21B and FIG. 21C. DSC shows a dehydration peak at about 6.2° C. and an endothermic peak at Tₒₙₛₑₜ of 161° C. TGA shows about 5.23% weight loss at about 130° C. IC shows that the stoichiometric of free form: phosphate salt is 1:1.1.

Competitive ripening between Form A* of Compound I phosphate salt and Form C of Compound I phosphate salt was carried out in different solvents. Form A* is more stable than Form C. The transformation from Form C to A* is slower in n-PrOH or IPA than MeOH or EtOH.

Competitive ripening between Form A* and Form C of Compound I phosphate salt in different solvent at 50/25° C.

| NO | Input | Procedure | Solvent | Temp. | 0 h | 18 h | 64 h |
|---|---|---|---|---|---|---|---|
| 1 | Form | Prepare the saturated | MeOH/Water = 9/1 | 25° C. | A* + C | A* | B |
| | A*/Form | solution of Form A* of | | | | | |
| 2 | C = 1/1 | Compound I phosphate | EtOH/Water = 9/1 | | A* + C | A* | B |
| | | salt | | | | | |
| 3 | | Charge Form A* of | n-PrOH/Water = 9/1 | | A* + C | A* + C | B |
| | | Compound I phosphate | | | | | |
| 4 | | salt 20 mg and Form C | IPA/Water = 9/1 | | A* + C | A* + C | A* + C |
| 5 | | 20 mg into saturated | MeOH/Water = 9/1 | 50° C. | A* | A* | A* |
| 6 | | solution | EtOH/Water = 9/1 | | A* | A* | A* |
| 7 | | Stir and take sample | n-PrOH/Water = 9/1 | | A* | A* | A* |
| 8 | | for XRPD | IPA/Water = 9/1 | | A* | A* | A* |

### Example 8: Preparation of Crystalline Form J of Compound I Phosphate Salt

Crystalline Form J of Compound I phosphate salt was prepared by fast evaporation of from Form A* of Compound I phosphate salt from MeOH.

About 20 mg of Form A* of Compound I phosphate salt was dissolved in MeOH. The obtained solution was filtered through a 0.45 μm nylon membrane filter by centrifugation at 14,000 rpm. The obtained clear solution was fast evaporated at room temperature (about 20-25° C.) under a dry nitrogen flow.

X-ray powder diffraction (XRPD) analysis was conducted on the solid crystals of crystalline Form J of Compound I phosphate salt. The XRPD is shown in FIG. 22A. Characteristic peaks include on or more of the peaks shown in Table 22. Form J of Compound I phosphate salt was determined successfully in this study, and the results indicate that it is a hydrate.

DSC and TGA thermograms were also obtained for Form J of Compound I phosphate salt as shown in FIG. 22B and FIG. 22C. DSC shows a dehydration peak at about 7.6° C. and broad endothermic peaks from 158° C. and 194° C. It decomposes upon melting. TGA shows about 7.1% weight loss at about 160° C. ¹H-NMR shows no detectable residual solvent. IC shows that the stoichiometric of free form: phosphate salt is 1:1.1.

### Example 9: Preparation of Crystalline Form K of Compound I Phosphate Salt

Crystalline Form K of Compound I phosphate salt was prepared by fast evaporation of from Form A* of Compound I phosphate salt from water, and by slow evaporation from water and MeOH.

For example, 20 mg of Form A* of Compound I phosphate salt was dissolved in water. The obtained solution was filtered through a 0.45 μm nylon membrane filter by centrifugation at 14,000 rpm. The obtained clear solution was slowly evaporated in ambient conditions (about 20-25° C., 40%-70% RH).

X-ray powder diffraction (XRPD) analysis was conducted on the solid crystals of crystalline Form K of Compound I phosphate salt. The XRPD is shown in FIG. 23A. Characteristic peaks include on or more of the peaks shown in Table 23. Form K of Compound I phosphate salt was determined successfully in this study, and the results indicate that it is a hydrate.

DSC and TGA thermograms were also obtained for crystalline Form K of Compound I phosphate salt as shown in FIG. 23B and FIG. 23C. DSC shows a dehydration peak at about 5° C. and a melting onset of 152.6° C. It decomposes upon melting. TGA shows about 5.8% weight loss at about 120° C. ¹H-NMR shows no detectable residual solvent. IC shows that the stoichiometric of free form: phosphate salt is 1:1.1. KF was not conducted as Pattern K is non-reproducible. Pattern A* was obtained instead by using the same preparation procedure.

### Example 10: Preparation of Crystalline Form H2 of Compound I Phosphate Salt

Crystalline Form H2 of Compound I phosphate salt was prepared from Form A* of Compound I phosphate salt (30 mg) by equilibration in MeOH/DCM ((v:v=1:1) at 25° C. for 2 weeks with a stirring bar on a magnetic stirring plate at a rate of 400 rpm. The obtained suspension was filtered through a 0.45 μm nylon membrane filter by centrifugation at 14,000 rpm.

X-ray powder diffraction (XRPD) analysis was conducted on the solid crystals of crystalline Form H2 of Compound I phosphate salt. The XRPD is shown in FIG. 24. Characteristic peaks include on or more of the peaks shown in Table 24. Form H2 of Compound I phosphate salt was determined successfully in this study, and the results indicate that it is a hydrate. ¹H-NMR shows no detectable residual solvent. KF shows 2.5% water by weight (1.0 equivalent by molar ratio).

### Example 11: Preparation of Crystalline Form E of Compound I Phosphate Salt

Crystalline Form E of Compound I phosphate salt was prepared from Form A* of Compound I phosphate salt (30 mg) by equilibration DMSO/water (a.w.=0.9*, v:v=24:76) at 25° C. for 2 weeks with a stirring bar on a magnetic stirring plate at a rate of 400 rpm and was also prepared from Form A* of Compound I phosphate salt (50 mg) by equilibration DMSO/water (a.w.=0.9*, v:v=24:76) at 50° C. for 1 week with a stirring bar on a magnetic stirring plate at a rate of 400 rpm. Obtained suspensions were filtered through a 0.45 μm nylon membrane filter by centrifugation at 14,000 rpm.

X-ray powder diffraction (XRPD) analysis was conducted on the solid crystals of crystalline Form E of Compound I phosphate salt. The XRPD is shown in FIG. 25A. Characteristic peaks include on or more of the peaks shown in Table 25. Form E of Compound I phosphate salt was determined successfully in this study, and the results indicate that it is a DMSO solvate.

DSC and TGA thermograms were also obtained for crystalline Form E of Compound I phosphate salt as shown in FIG. 25B and FIG. 25C. DSC shows 3 endothermic peaks from 55.5° C., 118.0° C. and 159.6° C., respectively. It de-solvates from about 250° C. TGA shows about 0.9% weight loss at about 190° C. ¹H-NMR shows 3.3 equivalents (26.2% by weight) DMSO. IC shows that the stoichiometric of free form: phosphate salt is 1:1.1.

### Example 12: Preparation of Crystalline Form F of Compound I Phosphate Salt

Crystalline Form F of Compound I phosphate salt was prepared from Form A* of Compound I phosphate salt (30 mg) by equilibration in DMSO/ACN (v:v=1:4) at 25° C. for 2 weeks with a stirring bar on a magnetic stirring plate at a rate of 400 rpm; and was also prepared from Form A* of Compound I phosphate salt (50 mg) by equilibration DMSO/ACN (v:v=1:4) at 50° C. for 1 week with a stirring bar on a magnetic stirring plate at a rate of 400 rpm. Obtained suspensions were filtered through a 0.45 μm nylon membrane filter by centrifugation at 14,000 rpm.

X-ray powder diffraction (XRPD) analysis was conducted on the solid crystals of crystalline Form F of Compound I phosphate salt. The XRPD is shown in FIG. 26A. Characteristic peaks include on or more of the peaks shown in Table 26. Form F of Compound I phosphate salt was determined successfully in this study, and the results indicate that it is a DMSO solvate.

DSC and TGA thermograms were also obtained for crystalline Form E of Compound I phosphate salt as shown in FIG. 26B and FIG. 26C. DSC shows multiple desolvation peaks from 2.1° C., 127.5° C., 153.3° C. and 172.5° C., respectively. No melting point was observed after desolvation. TGA shows about 3.2% weight loss at about 70° C., about 3.8% weight loss from 70° C. to 140° C. and about 3.8% from 140° C. to 240° C. ¹H-NMR shows 2.6 equivalents DMSO (21.8% by weight) and 0.3% residual ACN by weight. IC shows that the stoichiometric of free form: phosphate salt is 1:1.0.

### Example 13: Preparation of Crystalline Form G of Compound I Phosphate Salt

Crystalline Form G of Compound I phosphate salt was prepared from Form A* of Compound I phosphate salt (30 mg) by equilibration in DMSO/EtOH (v:v=1:4), DMSO/acetone (v:v=1:4), and DMSO/ethyl acetate (v:v=1:4) at 25° C. for 2 weeks with a stirring bar on a magnetic stirring plate at a rate of 400 rpm. Crystalline Form G of Compound I phosphate salt was also prepared from Form A* of Compound I phosphate salt (50 mg) by equilibration in DMSO/EtOH (v:v=1:4), DMSO/acetone (v:v=1:4), and DMSO/ethyl acetate (v:v=1:4) at 50° C. for 1 week with a stirring bar on a magnetic stirring plate at a rate of 400 rpm. Obtained suspensions were filtered through a 0.45 μm nylon membrane filter by centrifugation at 14,000 rpm.

X-ray powder diffraction (XRPD) analysis was conducted on the solid crystals of crystalline Form G of Compound I phosphate salt. The XRPD is shown in FIG. 27A. Characteristic peaks include on or more of the peaks shown in Table 27. Form G of Compound I phosphate salt was determined successfully in this study, and the results indicate that it is a DMSO-water solvate.

DSC and TGA thermograms were also obtained for crystalline Form G of Compound I phosphate salt as shown in FIG. 27B and FIG. 27C. DSC shows multiple desolvation peaks from 5.0° C., 129.9° C. and 174.1° C., respectively. No melting point was observed after desolvation. TGA shows about 4.7% weight loss at about 100° C., and about 13.2% weight loss from 100° C. to 250° C. ¹H-NMR shows 4.4 equivalents (32.1% by weight) DMSO. IC shows that the stoichiometric of free form: phosphate salt is 1:1.1.

### Example 14: Preparation of Crystalline Form I of Compound I Phosphate Salt

Crystalline Form I of Compound I phosphate salt was prepared from Form A* of Compound I phosphate salt (30 mg) by equilibration in TFE/CAN (v:v=1:4) and TFE/DCM (v:v=1:4) at 25° C. for 2 weeks with a stirring bar on a magnetic stirring plate at a rate of 400 rpm. Crystalline Form I of Compound I phosphate salt was also prepared from Form A* of Compound I phosphate salt (50 mg) by equilibration in TFE/DCM (v:v=1:4) and TFE/water (v:v=1:4) at 50° C. for 1 week with a stirring bar on a magnetic stirring plate at a rate of 400 rpm. Obtained suspensions were filtered through a 0.45 μm nylon membrane filter by centrifugation at 14,000 rpm.

X-ray powder diffraction (XRPD) analysis was conducted on the solid crystals of crystalline Form I of Compound I phosphate salt. The XRPD is shown in FIG. 28A. Characteristic peaks include on or more of the peaks shown in Table 28. Form I of Compound I phosphate salt was determined successfully in this study, and the results indicate that it is a 2,2,2-trifluoroethanol (TFE) solvate.

DSC and TGA thermograms were also obtained for crystalline Form I of Compound I phosphate salt as shown in FIG. 28B and FIG. 28C. DSC shows a broad desolvation peak from 126.2° C. It decomposes upon desolvation. No melting point was observed after desolvation. TGA shows about 20% weight loss at about 230° C. ¹H-NMR shows 1.9 equivalents (20.7% by weight) TFE. IC shows that the stoichiometric of free form: phosphate salt is 1:1.0.

### Example 15: Preparation of Crystalline Form H1 of Compound I Phosphate Salt

Crystalline Form H1 of Compound I phosphate salt was prepared from Form A* of Compound I phosphate salt (30 mg) by equilibration in DMF: ACN (v:v=1:4) and DMF: DCM (v:v=1:4) at 25° C. for 2 weeks with a stirring bar on a magnetic stirring plate at a rate of 400 rpm. Crystalline Form H1 of Compound I phosphate salt was also prepared by equilibration in DMF: ACN (v:v=1:4) and DMF: DCM (v:v=1:4) at 50° C. for 1 week with a stirring bar on a magnetic stirring plate at a rate of 400 rpm. Obtained suspensions were filtered through a 0.45 μm nylon membrane filter by centrifugation at 14,000 rpm.

X-ray powder diffraction (XRPD) analysis was conducted on the solid crystals of crystalline Form H1 of Compound I phosphate salt. The XRPD is shown in FIG. 29A. Characteristic peaks include on or more of the peaks shown in Table 29. Form H1 of Compound I phosphate salt was determined successfully in this study, and the results indicate that it is a DMF-water hetero solvate.

DSC and TGA thermograms were also obtained for crystalline Form H1 of Compound I phosphate salt as shown in FIG. 29B and FIG. 29C. DSC shows dehydration/desolvation endothermic peaks from 5.7° C. and 77.2° C. Then it melts at an onset of 160.4° C. It decomposes upon melting. TGA shows about 4.6% weight loss at about 150° C. ¹H-NMR shows 2.0% DMF by weight (about 0.2 equivalent by molar ratio). KF shows 3.6% water by weight (1.5 equivalents by molar ratio). IC shows that the stoichiometric of free form: phosphate salt is 1:1.0.

### Example 16: Preparation of Crystalline Form H3 of Compound I Phosphate Salt

Crystalline Form H3 of Compound I phosphate salt was prepared from Form A* of Compound I phosphate salt (30 mg) by equilibration in Acetone:water (v:v=9:1) at 25° C. for about 15 days with a stirring bar on a magnetic stirring plate at a rate of 400 rpm. Crystalline Form H3 of Compound I phosphate salt was also prepared by equilibration in Acetone:water (v:v=9:1) at 50° C. for about 15 days with a stirring bar on a magnetic stirring plate at a rate of 400 rpm. Obtained suspensions were filtered through a 0.45 μm nylon membrane filter by centrifugation at 14,000 rpm.

X-ray powder diffraction (XRPD) analysis was conducted on the solid crystals of crystalline Form H3 of Compound I phosphate salt. The XRPD is shown in FIG. 30. Characteristic peaks include on or more of the peaks shown in Table 30. Form H3 of Compound I phosphate salt was determined successfully in this study, and the results indicate that it is an acetone solvate. 1H-NMR shows 0.3 equivalent (2.3% by weight) acetone.

### Example 17: Preparation of Crystalline Form H4 of Compound I Phosphate Salt

Crystalline Form H4 of Compound I phosphate salt was prepared from Form A* of Compound I phosphate salt (30 mg) by equilibration in THF at 25° C. for about 11 days, and by equilibration in THF/water (v:v=9:1) at 25° C. for about 15 days with a stirring bar on a magnetic stirring plate at a rate of 400 rpm. Crystalline Form H4 of Compound I phosphate salt was also prepared by equilibration in THF, THF/water (a.w.=0.3*, v:v=97:3) at 50° C. for about 11 days with a stirring bar on a magnetic stirring plate at a rate of 400 rpm. Obtained suspensions were filtered through a 0.45 μm nylon membrane filter by centrifugation at 14,000 rpm.

X-ray powder diffraction (XRPD) analysis was conducted on the solid crystals of crystalline Form H4 of Compound I phosphate salt. The XRPD is shown in FIG. 31. Characteristic peaks include on or more of the peaks shown in Table 31. Form H4 of Compound I phosphate salt was determined successfully in this study, and the results indicate that it is a THF solvate. ¹H-NMR shows 0.2 equivalent (1.9% by weight) THF.

### Example 18: Preparation of Crystalline Form H5 of Compound I Phosphate Salt

Crystalline Form H5 of Compound I phosphate salt was prepared from Form A* of Compound I phosphate salt by vapor diffusion from DMSO/acetone. Approximately 30 mg of Form A* of Compound I phosphate salt was dissolved in DMSO (0.4 mL) in a 4-8 mL glass vial without lid at ambient temperature (about 20-25° C.). Then the 8 mL lid less vial was placed into a 20-40 mL glass vial. To the 20-40 mL vial was added anti-solvent acetone (1.6 mL). Then the 20-40 mL vial was capped and placed at ambient condition for up to 14 days. Precipitates were collected by centrifugation filtration through a 0.45 μm nylon membrane filter at 14,000 rpm.

X-ray powder diffraction (XRPD) analysis was conducted on the solid crystals of crystalline Form H5 of Compound I phosphate salt. The XRPD is shown in FIG. 32. Characteristic peaks include on or more of the peaks shown in Table 32. Form H5 of Compound I phosphate salt was determined successfully in this study, and the results indicate that it is a DMSO solvate. 1H-NMR shows 2.5 equivalents (21.2% by weight) DMSO.

### Example 19: Preparation of Crystalline Form H6 of Compound I Phosphate Salt

Crystalline Form H6 of Compound I phosphate salt was prepared from Form A* of Compound I phosphate salt by slow evaporation from benzyl alcohol. About 20 mg of Form A* of Compound I phosphate salt was dissolved in benzyl alcohol. The obtained solution was filtered through a 0.45 μm nylon membrane filter by centrifugation at 14,000 rpm and slowly evaporated in ambient conditions (about 20-25° C., 40%-70% RH).

X-ray powder diffraction (XRPD) analysis was conducted on the solid crystals of crystalline Form H6 of Compound I phosphate salt. The XRPD is shown in FIG. 33. Characteristic peaks include on or more of the peaks shown in Table 33. Form H6 of Compound I phosphate salt was determined successfully in this study, and the results indicate that it is a benzyl alcohol solvate. ¹H-NMR shows 0.9 equivalent (11.8% benzyl alcohol by weight) by slow evaporation in benzyl alcohol.

Form H1, Form H2, Form H3, Form H4, Form H5, and Form H6 of Compound I phosphate salt are hetero-solvates with similar XRPD, suggesting they are in iso-crystal structure. Form H2 is their hydrous state.

### Example 20: Solubility Experiments

The solubility of the free base crystalline Form A of Compound I, crystalline Form A* of Compound I phosphate salt, crystalline Form A** of Compound I benzenesulfonate salt, and crystalline Form B* of Compound I maleate salt was evaluated.

20 mg of the free base Form A of Compound I was weighed into a 20 mL glass vial. Amounts of the salts equivalent to 20 mg anhydrous free base were weighed into a glass vial. 10 mL of solubility medium was added. The resulting suspensions/solutions were stirred at 37° C. at 400 rpm and sampled at 2 hours and at 24 hours, respectively. The samples were centrifuged at 37° C. at 14,000 rpm for 5 min. Supernatants were analyzed by HPLC and pH meter for solubility and pH value, respectively. Residual solids (wet cakes) from the 24 hours samples were also characterized by XRPD to determine physical form.

Solubility of the free base crystalline Form A of Compound I and crystalline Form A* of Compound I phosphate salt, crystalline Form A** of Compound I benzenesulfonate salt, and crystalline Form B* of Compound I maleate salt was measured in 7 aqueous pH buffers and bio-relevant fluids including pH 1.2 HCl solution (0.1N), pH 4.5 acetate buffer (50 mM), pH 6.8 phosphate buffer (50 mM), pH 7.4 phosphate buffer (50 mM), pH 2.0 SGF, pH 6.5 FaSSIF-v1, and pH 5.0 FeSSIF-v1, at 37° C. for 2 h and 24 h, respectively. Residual solids after the solubility test were analyzed by XRPD.

The Form A* of Compound I phosphate salt showed highest solubility, followed by Form B* of Compound I maleate salt. Supersaturation of Form A* of Compound I phosphate salt can be maintained for a longer time than the crystalline Form B* of Compound I maleate salt in pH 4.5 buffer. These two salts showed overall higher solubility than Form A of Compound I. Form A** of Compound I benzenesulfonate salt showed overall lower solubility than that of Form A of Compound I.

The results are shown in Table 35, below.

TABLE 35
Solubility results for crystalline Forms A, A*, A**, and B*

| | Solubility at 37° C., equilibration for 2 hours or 24 hours, LOQ: 0.05 μg/mL | | | | | | |
|---|---|---|---|---|---|---|---|
| | | Free base Form A | | | Form A* of | | |
| | | of Compound I | | | Compound I phosphate salt | | |
| | | Solubility | Solubility | | Solubility | Solubility | |
| | Solubility | for 2 h | for 24 h | | for 2 h | for 24 h | |
| Exp. | media | (pH) | (pH) | XRPD | (pH) | (pH) | XRPD |
| ES1 | pH 1.2 | 1.2 | 1.0 | Amorphous | 1.2 | 1.0 | Amorphous |
| | HCl | mg/mL | mg/mL | form with | mg/mL | mg/mL | form with |
| | solution | (1.2) | (1.0) | one extra | (1.2) | (1.0) | one extra |
| | (0.1N) | | | peak | | | peak |
| ES2 | pH 4.5 | 0.2 | 0.3 | No form | >2 | >2 mg/mL | // |
| | acetate | mg/mL | mg/mL | change | mg/mL | mg/mL | |
| | buffer | (4.5) | (4.4) | | (4.4) | (4.4) | |
| | (50 mM) | | | | | | |
| ES3 | pH 6.8 | 2.9 | 5.1 | No form | 8.8 | 6.4 | Dissociation |
| | phosphate | μg/mL | μg/mL | change | μg/mL | μg/mL | to free |
| | buffer | (6.8) | (6.7) | | (6.6) | (6.5) | base |
| | (50 mM) | | | | | | Pattern B |
| ES4 | pH 7.4 | 2.2 | 3.8 | No form | 6.1 | 4.1 | Dissociation |
| | phosphate | μg/mL | μg/mL | change | μg/mL | μg/mL | to free |
| | buffer | (7.5) | (7.4) | | (7.2) | (7.1) | base |
| | (50 mM) | | | | | | Pattern B |
| ES5 | SGF, pH | >2 | >2 | // | >2 | >2 | // |
| | 2.0 | mg/mL | mg/mL | | mg/mL | mg/mL | |
| | | (2.0) | (1.9) | | (1.9) | (1.8) | |
| ES6 | FaSSIF-v1, | 9.4 | 11.5 | No form | 26.8 | 21.1 | Dissociation |
| | pH 6.5 | μg/mL | μg/mL | change | μg/mL | μg/mL | to free |
| | | (6.5) | (6.5) | | (6.2) | (6.1) | base |
| | | | | | | | Pattern B |
| ES7 | FeSSIF-v1, | 0.6 | 0.6 | No form | 1.6 | 1.7 | Amorphous |
| | pH 5.0 | mg/mL | mg/mL | change | mg/mL | mg/ml | form with |
| | | (5.0) | (5.0) | | (5.0) | (4.9) | peaks of |
| | | | | | | | NaCl |

| | Solubility at 37° C., equilibration for 2 hours or 24 hours, LOQ: 0.05 μg/mL | | | | | |
|---|---|---|---|---|---|---|
| | Form A** of Compound I | | | Form B* of | | |
| | benzenesulfonate salt | | | Compound I maleate salt | | |
| | Solubility | Solubility | | | Solubility | |
| | for 2 h | for 24 h | | Solubility | for 24 h | |
| Exp. | (pH) | (pH) | XRPD | for 2 h (pH) | (pH) | XRPD |
| ES1 | 0.3 | 0.4 | No form | 1.4 | 1.5 | Not enough |
| | mg/mL | mg/mL | change | mg/mL | mg/mL | solids |
| | (1.2) | (1.0) | | (1.3) | (1.3) | for XRPD |
| | | | | | | characterization |
| ES2 | 0.2 | 0.3 | No form | >2 | 0.3 | Dissociation |
| | mg/mL | mg/mL | change | mg/mL | mg/mL | to free |
| | (4.5) | (4.4) | | (4.5) | (4.5) | base |
| | | | | | | Pattern B |
| ES3 | 8.5 | 6.9 | Dissociation | 6.6 | 4.7 | Dissociation |
| | μg/mL | μg/mL | to free | μg/mL | μg/mL | to free |
| | (6.7) | (6.6) | base | (6.6) | (6.6) | base |
| | | | Pattern B | | | Pattern B |
| ES4 | 5.7 | 4.1 | Dissociation | 1.5 | 1.4 | Dissociation |
| | μg/mL | μg/mL | to free | μg/mL | μg/mL | to free |
| | (7.3) | (7.3) | base | (7.3) | (7.3) | base |
| | | | Pattern B | | | Pattern B |
| ES5 | 0.2 | 0.3 | No form | >2 | >2 | // |
| | mg/mL | mg/mL | change | mg/mL | mg/mL | |
| | (2.0) | (1.8) | | (2.0) | (2.0) | |
| ES6 | 19.6 | 18.6 | Dissociation | 22.9 | 16.2 | Dissociation |
| | μg/mL | μg/mL | to free | μg/mL | μg/mL | to free |
| | (6.3) | (6.2) | base | (6.1) | (6.1) | base |
| | | | Pattern B | | | Pattern B |
| ES7 | 0.7 | 0.8 | No form | 1.1 | 1.0 | Dissociation |
| | mg/mL | mg/mL | change | mg/mL | mg/mL | to free |
| | (5.0) | (4.9) | | (5.0) | (5.0) | base |
| | | | | | | Pattern B |

“//”: Not carried out because no sufficient solid was obtained

### Example 21: Transfection Protocol and Readout for NanoBRET Screening

Human embryonic kidney 293-H (HEK 293, Gibco 293-H, #11631017) cell lines were maintained in Dulbecco's Modified Eagle Medium, high glucose, pyruvate (DMEM, Gibco, #11995065) supplemented with 10% fetal bovine serum (FBS, Gibco, #10082147) and 1× penicillin-streptomycin (100× solution, Gibco, #15140148) at 37° C. and 5% CO2 in a water-saturated incubator. Cell were trypsinized using 0.05% or 0.25% Trypsin-EDTA solution (Trypsin-EDTA, phenol red, Gibco, #25200056 (0.25%) or #25300054). Opti-MEM media supplemented with 10% fetal bovine serum (Opti-MEM I reduced serum media, no phenol red, Gibco, #11058021) was used for culturing cells overnight for NanoBRET readout experiments.

HEK293 cells were cultivated appropriately prior to assay. The medium from cell flask was removed via aspiration, washed 1× with PBS followed by aspiration, trypsinized, and cells were allowed to dissociate from the flask. Trypsin was neutralized using growth medium and cells were pelleted via centrifugation at 200×g for 5 minutes. The medium was aspirated and the cells were resuspended into a single cell suspension using Opti-MEM I supplemented with 10% FBS. The cell density was adjusted to 2×10⁵/mL in Opti-MEM I supplemented with 10% FBS in a sterile, conical tube. The cells were transfected and aliquoted directly in a 96-well plate for the NanoBRET assay the next day, and therefore, the cells were cultured overnight in Opti-MEM. The cells were also transfected in bulk and dispensed into a 96-well plate to allow cells to adhere to the plate overnight, thereby enabling washout studies.

The lipid: DNA complexes were prepared as follows:

A 10 μg/mL solution of DNA was prepared in Opti-MEM without serum. This solution contains the following ratios of carrier DNA and DNA encoding NanoLuc fused to the biological target. Serial dilution steps may be warranted to accurately dilute the NanoLuc fusion DNA. Added, in order, the following reagents were added to a sterile polystyrene test tube: 1 mL of Opti-MEM without phenol red; 9.0 g/mL of carrier DNA; 1.0 μg/mL of NanoLuc fusion DNA (for some targets, the amount is less). The reagents were mixed thoroughly. 30 μL of FuGENE HD is added into each mL of DNA mixture to form lipid: DNA complex. Care is taken such that FuGENE HD does not touch the plastic side of the tube and pipetted directly into the liquid in the tube. It is mixed by pipetting up and down 5-10 times and incubated at room temperature for 20 minutes to allow complexes to form. 1 part (e.g. 1 mL) of lipid: DNA complex was mixed with 20 parts (e.g. 20 mL) of HEK293 cells in suspension at 2× 105/mL and mixed gently by pipetting up and down 5 times in a sterile, conical tube. Larger or smaller bulk transfections are scaled accordingly, using this ratio. 100 μL cells+lipid: DNA complex was dispensed into a sterile, tissue-culture treated 96-well plate (20,000 cells/well), and incubated at least 16 hours to allow expression. The cells were then incubated in a 37° C.+5% CO2 incubator for >16 hrs. The serially diluted Compound I was prepared at 100× final concentration in 100% DMSO. The serially diluted Compound I stock was prepared in PCR plates. 1 μL per well of 100× serially diluted inhibitor/compound was added to the cells in 96-well plates that have been transiently transfected overnight and mixed by tapping the plate by hand. The plate was incubated at 37° C.+5% CO2 incubator overnight. A 1× solution of substrate mix (500× stock) and appropriate concentration of tracer was prepared in Opti-Mem. The cells were washed by setting a plate washer to the 96 well plate 5× in PBS pH 7.4 by adding 200 μL PBS each time. The cells were incubated at 37° C. for 2 hours. 100 μL of the 1× Substrate-Tracer solution was added and the 96 well plate is gently tapped to mix. The plate on plate reader is read every hour for the next 6 hours. The binding assay results show that Compound I provides greater than 80% inhibition of the PI3K target.

### Example 22: Cell Proliferation Assay

The objective of this study was investigate the effect of Compound I on the cell proliferation of 13 cell lines after 3 days treatment, and determine the IC50 of Compound I in each cell line. The cell lines tested included as follows: UM-UC-3 (bladder), KYSE-410 (HN/Esophagus), SW1463 (rectum), Calu-1 (Lung), NCI-H358 (Lung), SW837 (Rectum), SW756 (Cervix), NCI-H2122 (Lung), NCI-H1373 (Lung), NCI-H1792 (Lung), NCI-H23 (Lung), MIA PaCa-2 (Pancreas), and HC44.

Cells were recovered and maintained in appropriate culture media. The cells were harvested respectively during the logarithmic growth period. The cells were then resuspended and counted using a Vi cell counter (The cell viability be measured by trypan blue exclusion assay.). The cells were then diluted and 90 μL cell suspensions were used in 96-well plates according to plate map with final cell density. Two duplicate plates were set up. One is for day 0 reading (TO) and the other was cultured in incubator for reading at the end point. The incubated plates were incubated overnight in humidified incubator at 37° C. with 5% CO2.

At day 0, 10 μL culture medium to each well for TO reading. 10 μL of culture medium was added to each well for TO reading. Then, 50 μl CellTiter-Glo® Reagent was added to each well. The contents were then mixed for 2 minutes on an orbital shaker to facilitate cell lysis. The plates were then allowed to incubate at room temperature for 10 minutes to stabilize luminescent signal. Luminescence was then recorded using an EnVision Multi Label Reader. The test compound Compound I, and a control cisplatin, where then diluted at various concentrations from a 10 mM stock solution. Compound I was diluted and 10 μL of each 10× compound I from working solutions for a concentration ranging from 10 μM to about 0.005 μM. Cisplatin was diluted to about 3.33 mM to about 150 nM. The screening plates were then placed back into the incubator for the appropriate treatment time (3 days).

For the endpoint CTG reading, 50 μL of CellTiter-Glo® Reagent were added to each well. The contents were then mixed for 2 minutes on an orbital shaker to facilitate cell lysis. The plate was allowed to incubate at room temperature for 10 minutes to stabilize luminescent signal. Luminescence was then recorded using an EnVision Multi Label Reader. IC50's were determined for each cell line. The assay indicates Compound I has inhibitive effect across multiple cancer cell lines, including a substantial inhibitory effect against NCI-H358 (see Table 38).

TABLE 36
IC50 Proliferation Assay

| | Absolute IC50 | Absolute IC50 |
|---|---|---|
| Cell Line Name | (μM) Compound I | (μM) Cisplatin |
| UM-UC-3 (bladder) | >10 | 1.8642 |
| KYSE-410 (HN/Esophagus) | 2.0231 | 15.8034 |
| SW1463 (rectum) | 3.8582 | 12.2245 |
| Calu-1 (Lung) | >10 | 10.1785 |
| NCI-H358 (Lung) | 1.69 | 12.4619 |
| SW837 (Rectum) | >10 | 42.4853 |
| SW756 (Cervix) | 7.2855 | 10.2600 |
| NCI-H2122 (Lung) | 3.7651 | 17.787 |
| NCI-H1373 (Lung) | >10 | 8.7241 |
| NCI-H1792 (Lung) | >10 | 4.8783 |
| NCI-H23 (Lung) | >10 | 3.5848 |
| MIA PaCa-2 (Pancreas) | >10 | 7.0279 |
| HCC44 (Lung) | 7.92 | 7.9621 |

1. A crystalline form of Compound I: 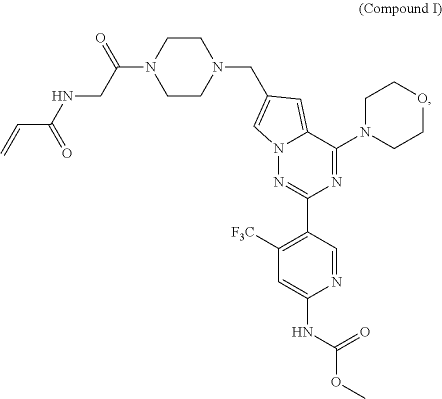 [SMILES: C=CC(=O)NCC(=O)N1CCN(Cc2cc3c(N4CCOCC4)nc(-c4cnc(NC(=O)OC)cc4C(F)(F)F)nn3c2)CC1]
or a pharmaceutically acceptable salt thereof, solvate thereof, or salt solvate thereof.

2. The crystalline form of claim 1, which is a crystalline form of Compound I.

3. The crystalline form of claim 1 or 2, wherein Compound I is anhydrous or non-solvated.

4. The crystalline form of any one of claims 1-3, which exhibits an X-ray powder diffraction (XRPD) pattern comprising peaks at about 9.11±0.2, 22.21±0.2, and 24.99±0.2 degrees two-theta.

5. The crystalline form of claim 4, which exhibits an X-ray powder diffraction (XRPD) pattern comprising peaks at about 9.11±0.2, 16.93-0.2, 18.70-0.2, 22.21±0.2, and 24.99±0.2 degrees two-theta.

6. The crystalline form of claim 5, which exhibits an X-ray powder diffraction (XRPD) pattern comprising peaks at about 9.11±0.2, 16.93-0.2, 18.70-0.2, 20.54=0.2, 20.78=0.2, 22.21±0.2, and 24.99±0.2 degrees two-theta.

7. The crystalline form of any one of claims 4-6, wherein the XRPD pattern further comprises at least two peaks selected from about 4.47±0.2, 12.45±0.2, 14.51±0.2, 22.70=0.2, and 26.54±0.2 degrees two-theta.

8. The crystalline form of any one of claims 4-7 which exhibits an XRPD pattern comprising peaks in Table 1.

9. The crystalline form of any one of claims 1-8, which is Form A exhibiting an XRPD pattern substantially similar to FIG. 1A.

10. The crystalline form of any one of claims 1-9, which exhibits a differential scanning calorimetry (DCS) thermogram comprising an endotherm peak with an onset at about 199° C.

11. The crystalline form of any one of claims 1-10 which exhibits weight percent loss of about 0.6% between about 25° C. to about 160° C. by a thermogravimetric analysis (TGA).

12. The crystalline form of claim 1, which is a crystalline form of a pharmaceutically salt of Compound I or a pharmaceutically acceptable salt solvate of Compound I.

13. The crystalline form of claim 12, which is a crystalline form of a pharmaceutically acceptable salt of Compound I selected from the group consisting of a phosphate salt, a hydrochloride salt, a sulfate salt, a mesylate salt, a benzenesulfonate salt, a tosylate salt, a fumarate salt, a maleate salt, a L-tartrate salt, a citrate salt, and a succinate salt, or a solvate thereof.

14. The crystalline form of claim 12 or 13, which is a crystalline form of a phosphate salt of Compound I or a solvate thereof.

15. The crystalline form of claim 14, wherein the crystalline form is a hydrate.

16. The crystalline form of claim 15, wherein the hydrate is a channel hydrate.

17. The crystalline form of any one of claims 12-16, which exhibits an X-ray powder diffraction (XRPD) pattern comprising peaks at about 5.33±0.2, 15.97±0.2, and 22.97±0.2, degrees two-theta.

18. The crystalline form of any one of claim 17, which exhibits an X-ray powder diffraction (XRPD) pattern comprising peaks at about 5.33±0.2, 10.63±0.2, 15.97±0.2, 20.95±0.2, and 22.97±0.2, degrees two-theta.

19. The crystalline form of any one of claim 18, which exhibits an X-ray powder diffraction (XRPD) pattern comprising peaks at about 5.33±0.2, 10.63±0.2, 15.97±0.2, 20.26±0.2, 20.95±0.2, 22.71±0.2, and 22.97±0.2, degrees two-theta.

20. The crystalline form of any one of claims 17-19, wherein the XRPD pattern further comprises at least two peaks selected from about 7.24±0.2, 14.94±0.2, 18.64±0.2, 18.99±0.2, and 21.34±0.2 degrees two-theta.

21. The crystalline form of any one of claims 14-20 which exhibits an XRPD pattern comprising peaks in Table 2.

22. The crystalline form of any one of claims 14-21, which is Form A* exhibiting an XRPD pattern substantially similar to FIG. 2A.

23. The crystalline form of any one of claims 14-22, which exhibits a differential scanning calorimetry (DCS) thermogram comprising an endotherm peak at about 8.5° C.

24. The crystalline form of any one of claims 14-22, which exhibits a differential scanning calorimetry (DCS) thermogram comprising an endotherm peak with an onset at about 189° C.

25. The crystalline form of any one of claims 14-24 which exhibits a weight percent loss of about 3.75% between about 34° C. to about 170° C. by a thermogravimetric analysis (TGA).

26. The crystalline form of claim 12 or 13, which is a crystalline form of a benzenesulfonate salt of Compound I or a solvate thereof.

27. The crystalline form of claim 26, wherein the Compound I benzenesulfonate salt is anhydrous or non-solvated.

28. The crystalline form of claim 26 or 27, which exhibits an X-ray powder diffraction (XRPD) pattern comprising peaks at about 7.36±0.2, 18.92±0.2 and 19.54±0.2 degrees two-theta.

29. The crystalline form of claim 28, which exhibits an X-ray powder diffraction (XRPD) pattern comprising peaks at about 7.36±0.2, 14.71±0.2, 18.52±0.2, 18.92±0.2 and 19.54±0.2 degrees two-theta.

30. The crystalline form of claim 29, which exhibits an X-ray powder diffraction (XRPD) pattern comprising peaks at about 7.36±0.2, 10.08±0.2, 14.71±0.2, 18.52±0.2, 18.92±0.2, 19.54±0.2, and 21.31±0.2 degrees two-theta.

31. The crystalline form of any one of claims 28-30, wherein the XRPD pattern further comprises at least two peaks selected from about 15.50±0.2, 18.23±0.2, 22.72±0.2, 23.22±0.2, and 24.63±0.2 degrees two-theta.

32. The crystalline form of any one of claims 26-31, which exhibits an XRPD pattern comprising peaks in Table 3.

33. The crystalline form of any one of claims 26-32, which is Form A** exhibiting an XRPD pattern substantially similar to FIG. 3A.

34. The crystalline form of any one of claims 26-33, which exhibits a differential scanning calorimetry (DCS) thermogram which shows decomposition at about 250° C.

35. The crystalline form of any one of claims 26-34, which exhibits a weight percent loss of about 1.1% between about 34° C. to about 190° C. by a thermogravimetric analysis (TGA).

36. The crystalline form of claim 12 or 13, which is a crystalline form of a maleate salt of Compound I or a solvate thereof.

37. The crystalline form of claim 36, wherein the crystalline form is a hydrate.

38. The crystalline form of claim 36 or 37, which exhibits an X-ray powder diffraction (XRPD) pattern comprising peaks at about 4.78±0.2, 7.07±0.2 and 19.83±0.2 degrees two-theta.

39. The crystalline form of claim 38, which exhibits an X-ray powder diffraction (XRPD) pattern comprising peaks at about 4.78±0.2, 7.07±0.2, 12.27±0.2, 19.83±0.2, and 20.84±0.2 degrees two-theta.

40. The crystalline form of claim 39, which exhibits an X-ray powder diffraction (XRPD) pattern comprising peaks at about 4.78±0.2, 7.07±0.2, 12.27±0.2, 19.83±0.2, 20.84±0.2, 20.98±0.2, and 24.35±0.2 degrees two-theta.

41. The crystalline form of any one of claims 38-40, wherein the XRPD pattern further comprises at least two peaks selected from about 18.25±0.2, 18.45±0.2, 22.88±0.2, 23.82±0.2 and 23.84±0.2 degrees two-theta.

42. The crystalline form of any one of claims 36-41, which exhibits an XRPD pattern comprising peaks in Table 4.

43. The crystalline form of any one of claims 36-42, which is Form B* exhibiting an XRPD pattern substantially similar to FIG. 4A.

44. The crystalline form of any one of claims 36-43, which exhibits a differential scanning calorimetry (DCS) thermogram comprising an endotherm peak at about 10° C.

45. The crystalline form of any one of claims 36-44, which exhibits a differential scanning calorimetry (DCS) thermogram comprising an endotherm peak with an onset at about 166° C.

46. The crystalline form of any one of claims 36-45, which exhibits a weight percent loss of about 1.8% between about 34° C. to about 130° C. by a thermogravimetric analysis (TGA).

47. The crystalline form of claim 12 or 13, which is a crystalline form of a hydrochloride salt of Compound I or a solvate thereof.

48. The crystalline form of claim 47, which exhibits an X-ray powder diffraction (XRPD) pattern comprising peaks at about 4.83±0.2, 7.14±0.2 and 9.20±0.2 degrees two-theta.

49. The crystalline form of claim 48, which exhibits an X-ray powder diffraction (XRPD) pattern comprising peaks at about 4.83±0.2, 5.38±0.2, 7.14±0.2, 9.20±0.2, and 22.78±0.2 degrees two-theta.

50. The crystalline form of claim 48 or 49, wherein the XRPD pattern further comprises at least two peaks selected from about 15.03±0.2, 20.32±0.2, 21.12±0.2, 22.45±0.2, 23.57±0.2, 24.66±0.2, and 27.45±0.2 degrees two-theta.

51. The crystalline form of any one of claims 47-50, which exhibits an XRPD pattern comprising peaks in Table 5.

52. The crystalline form of any one of claims 47-51, which is Form A-1 exhibiting an XRPD pattern substantially similar to FIG. 5A.

53. The crystalline form of claim 47, which exhibits an X-ray powder diffraction (XRPD) pattern comprising peaks at about 7.40±0.2, 23.26±0.2 and 24.21±0.2 degrees two-theta.

54. The crystalline form of claim 53, which exhibits an X-ray powder diffraction (XRPD) pattern comprising peaks at about 4.48±0.2, 7.40±0.2, 7.79±0.2, 23.26±0.2 and 24.21±0.2 degrees two-theta.

55. The crystalline form of claim 53 or 54, wherein the XRPD pattern further comprises at least two peaks selected from about 9.73±0.2, 10.12±0.2, 12.93±0.2, 13.96±0.2, 16.08±0.2, 18.93±0.2, 20.79±0.2, and 22.33±0.2 degrees two-theta.

56. The crystalline form of any one of claims 53-55, which exhibits an XRPD pattern comprising peaks in Table 6.

57. The crystalline form of any one of claims 53-56, which is Form B-1 exhibiting an XRPD pattern substantially similar to FIG. 6A.

58. The crystalline form of claim 12 or 13, which is a crystalline form of a sulfate salt of Compound I or a solvate thereof.

59. The crystalline form of claim 58, which exhibits an X-ray powder diffraction (XRPD) pattern comprising peaks at about 6.95±0.2, 9.52±0.2 and 9.82±0.2 degrees two-theta.

60. The crystalline form of claim 59, which exhibits an X-ray powder diffraction (XRPD) pattern comprising peaks at about 6.95±0.2, 9.52±0.2, 9.82±0.2, 12.92±0.2 and 19.46±0.2 degrees two-theta.

61. The crystalline form of any one of claim 59 or 60, wherein the XRPD pattern further comprises at least two peaks selected from about 13.77±0.2, 15.30±0.2, 25.63±0.2 degrees two-theta.

62. The crystalline form of any one of claims 58-61, which exhibits an XRPD pattern comprising peaks in Table 7.

63. The crystalline form of any one of claims 58-62, which is Form A-2 exhibiting an XRPD pattern substantially similar to FIG. 7A.

64. The crystalline form of claim 58, which exhibits an X-ray powder diffraction (XRPD) pattern comprising peaks at about 17.85±0.2, 20.29±0.2, and 24.63±0.2 degrees two-theta.

65. The crystalline form of claim 64, which exhibits an X-ray powder diffraction (XRPD) pattern comprising peaks at about 17.85±0.2, 20.29±0.2, 23.26±0.2, 24.63±0.2, and 24.74±0.2 degrees two-theta.

66. The crystalline form of claim 64 or 65, which exhibits an X-ray powder diffraction (XRPD) pattern further comprising at least two peaks selected from about 8.96±0.2, 12.77±0.2, 15.31±0.2, 16.68±0.2, 19.14±0.2, 20.95±0.2, 20.96±0.2, and 27.78±0.2 degrees two-theta.

67. The crystalline form of any one of claims 64-66, which exhibits an XRPD pattern comprising peaks in Table 8.

68. The crystalline form of any one of claims 64-67, which is Form B-2 exhibiting an XRPD pattern substantially similar to FIG. 8A.

69. The crystalline form of claim 12 or 13, which is a crystalline form of a mesylate salt of Compound I or a solvate thereof.

70. The crystalline form of claim 69, which exhibits an X-ray powder diffraction (XRPD) pattern comprising peaks at about 10.23±0.2, 14.18±0.2 and 18.56±0.2 degrees two-theta.

71. The crystalline form of claim 70, which exhibits an X-ray powder diffraction (XRPD) pattern comprising peaks at about and 5.41±0.2, 7.09±0.2, 10.23±0.2, 14.18±0.2 and 18.56±0.2 degrees two-theta.

72. The crystalline form of claim 70 or 71, which exhibits an X-ray powder diffraction (XRPD) pattern further comprising at least two peaks selected from about 3.55±0.2, 10.78±0.2, 12.45±0.2, 18.76±0.2, 19.82±0.2, 21.89±0.2, 22.32±0.2, and 23.24±0.2 degrees two-theta.

73. The crystalline form of any one of claims 69-72, which exhibits an XRPD pattern comprising peaks in Table 9.

74. The crystalline form of any one of claims 69-73, which is Form A-3 exhibiting an XRPD pattern substantially similar to FIG. 9A.

75. The crystalline form of claim 69, which exhibits an X-ray powder diffraction (XRPD) pattern comprising peaks at about 4.80±0.2, 7.20±0.2 and 19.93±0.2 degrees two-theta.

76. The crystalline form of claim 75, which exhibits an X-ray powder diffraction (XRPD) pattern comprising peaks at about 4.80±0.2, 7.20±0.2, 18.28±0.2, 19.93±0.2, and 21.17±0.2 degrees two-theta.

77. The crystalline form of claim 75 or 76, which exhibits an X-ray powder diffraction (XRPD) pattern further comprising at least two peaks selected from about 12.43±0.2, 14.77±0.2, 16.08±0.2, 18.56±0.2, 22.77±0.2, 23.04±0.2, 23.86±0.2, and 24.43±0.2 degrees two-theta.

78. The crystalline form of any one of claims 75-77, which exhibits an XRPD pattern comprising peaks in Table 10.

79. The crystalline form of any one of claims 75-78, which is Form B-3 exhibiting an XRPD pattern substantially similar to FIG. 10A.

80. The crystalline form of claim 69, which exhibits an X-ray powder diffraction (XRPD) pattern comprising peaks at about 7.09±0.2, 16.73±0.2 and 22.68±0.2 degrees two-theta.

81. The crystalline form of claim 80, which exhibits an X-ray powder diffraction (XRPD) pattern comprising peaks at about 6.89±0.2, 7.09±0.2, 16.73±0.2, 22.34±0.2, and 22.68±0.2 degrees two-theta.

82. The crystalline form of claim 80 or 81, which exhibits an X-ray powder diffraction (XRPD) pattern further comprising at least two peaks selected from about 9.24±0.2, 16.02±0.2, 16.73±0.2, 19.86±0.2, 21.29±0.2, 21.81±0.2, 23.93±0.2, 24.54±0.2, and 27.40±0.2 degrees two-theta.

83. The crystalline form of any one of claims 80-82, which exhibits an XRPD pattern comprising peaks in Table 11.

84. The crystalline form of any one of claims 80-83, which is Form C-3 exhibiting an XRPD pattern substantially similar to FIG. 11A.

85. The crystalline form of claim 12 or 13, which is a crystalline form of a tosylate salt of Compound I or a solvate thereof.

86. The crystalline form of claim 85, which exhibits an X-ray powder diffraction (XRPD) pattern comprising peaks at about 7.46±0.2, 9.99±0.2 and 19.09±0.2 degrees two-theta.

87. The crystalline form of claim 86, which exhibits an X-ray powder diffraction (XRPD) pattern comprising peaks at about 7.46±0.2, 9.99±0.2, 14.89±0.2, 19.09±0.2, and 22.39±0.2 degrees two-theta.

88. The crystalline form of claim 86 or 87, which exhibits an X-ray powder diffraction (XRPD) pattern further comprising at least two peaks selected from about 10.61±0.2, 15.36±0.2, 17.64±0.2, 18.27±0.2, 19.65±0.2, 19.97±0.2, 23.10±0.2, and 25.30±0.2 degrees two-theta.

89. The crystalline form of any one of claims 85-88, which exhibits an XRPD pattern comprising peaks in Table 12.

90. The crystalline form of any one of claims 85-89, which is Form A-4 exhibiting an XRPD pattern substantially similar to FIG. 12A.

91. The crystalline form of claim 12 or 13, which is a crystalline form of a fumarate salt of Compound I or a solvate thereof.

92. The crystalline form of claim 91, which exhibits an X-ray powder diffraction (XRPD) pattern comprising peaks at about 18.12±0.2, 23.11±0.2, and 23.59±0.2 degrees two-theta.

93. The crystalline form of claim 92, which exhibits an X-ray powder diffraction (XRPD) pattern comprising peaks at about 5.31±0.2, 18.12±0.2, 19.79±0.2, 23.11±0.2, and 23.59±0.2 degrees two-theta.

94. The crystalline form of claim 92 or 93, which exhibits an X-ray powder diffraction (XRPD) pattern further comprising at least two peaks selected from about 8.50±0.2, 9.42±0.2, 13.23±0.2, 19.12±0.2, 21.16±0.2, 25.17±0.2, 25.68±0.2 and 28.82±0.2 degrees two-theta.

95. The crystalline form of any one of claims 91-94, which exhibits an XRPD pattern comprising peaks in Table 13.

96. The crystalline form of any one of claims 91-95, which is Form A-5 exhibiting an XRPD pattern substantially similar to FIG. 13A.

97. The crystalline form of claim 36, which exhibits an X-ray powder diffraction (XRPD) pattern comprising peaks at about 3.99±0.2, 23.84±0.2, and 25.40±0.2 degrees two-theta.

98. The crystalline form of claim 97, which exhibits an X-ray powder diffraction (XRPD) pattern comprising peaks at about 3.99±0.2, 23.70±0.2, 23.74±0.2, 23.84±0.2, and 25.40±0.2 degrees two-theta.

99. The crystalline form of claim 97 or 98, which exhibits an X-ray powder diffraction (XRPD) pattern further comprising at least two peaks selected from about 11.84±0.2, 17.48±0.2, 18.85±0.2, 19.59±0.2, 19.97±0.2, 22.75±0.2, 24.86±0.2, and 25.97±0.2 degrees two-theta.

100. The crystalline form of any one of claims 97-99, which exhibits an XRPD pattern comprising peaks in Table 14.

101. The crystalline form of any one of claims 97-100, which is Form A-6 exhibiting an XRPD pattern substantially similar to FIG. 14A.

102. The crystalline form of claim 12 or 13, which is a crystalline form of a L-tartrate salt of Compound I or a solvate thereof.

103. The crystalline form of claim 102, which exhibits an X-ray powder diffraction (XRPD) pattern comprising peaks at about 5.61±0.2, 6.93±0.2, and 19.66±0.2 degrees two-theta.

104. The crystalline form of claim 103, which exhibits an X-ray powder diffraction (XRPD) pattern comprising peaks at about 5.61±0.2, 6.93±0.2, 17.51±0.2, 19.66±0.2, and 21.75±0.2 degrees two-theta.

105. The crystalline form of any one of claim 103 or 104, which exhibits an XRPD pattern comprising peaks in Table 15.

106. The crystalline form of any one of claims 102-105, which is Form A-7 exhibiting an XRPD pattern substantially similar to FIG. 15A.

107. The crystalline form of claim 12 or 13, which is a crystalline form of a citrate salt of Compound I or a solvate thereof.

108. The crystalline form of claim 107, which exhibits an X-ray powder diffraction (XRPD) pattern comprising peaks at about 4.56±0.2, 9.15±0.2, and 12.05±0.2 degrees two-theta.

109. The crystalline form of claim 107 or 108, which exhibits an X-ray powder diffraction (XRPD) pattern comprising peaks at about 4.56±0.2, 9.15±0.2, 12.05±0.2, 17.43±0.2, and 18.63±0.2 degrees two-theta.

110. The crystalline form of claim 108 or 109, which exhibits an X-ray powder diffraction (XRPD) pattern further comprising at least two peaks selected from about 7.94±0.2, 15.77±0.2, 18.27±0.2, 19.10±0.2, 20.23±0.2, 20.88±0.2, 22.31±0.2, and 24.02±0.2 degrees two-theta.

111. The crystalline form of any one of claims 107-110, which exhibits an XRPD pattern comprising peaks in Table 17.

112. The crystalline form of any one of claims 107-111, which is Form A-8 exhibiting an XRPD pattern substantially similar to FIG. 17A.

113. The crystalline form of claim 107, which exhibits an X-ray powder diffraction (XRPD) pattern comprising peaks at about 5.36±0.2, 6.85±0.2, and 20.59±0.2 degrees two-theta.

114. The crystalline form of claim 113, which exhibits an X-ray powder diffraction (XRPD) pattern comprising peaks at about 5.36±0.2, 6.85±0.2, 17.81±0.2, 20.59±0.2, and 22.81±0.2, degrees two-theta.

115. The crystalline form of claim 113 or 114, which exhibits an X-ray powder diffraction (XRPD) pattern further comprising at least two peaks selected from about 16.08±0.2, 21.20±0.2, 25.81±0.2, and 27.02±0.2 degrees two-theta.

116. The crystalline form of any one of claims 113-115, which exhibits an XRPD pattern comprising peaks in Table 18.

117. The crystalline form of any one of claims 113-116, which is Form B-8 exhibiting an XRPD pattern substantially similar to FIG. 18A.

118. The crystalline form of claim 12 or 13, which is a crystalline form of a succinate salt of Compound I or a solvate thereof.

119. The crystalline form of claim 118, which exhibits an X-ray powder diffraction (XRPD) pattern comprising peaks at about 8.73±0.2, 20.05±0.2, and 26.15±0.2 degrees two-theta.

120. The crystalline form of claim 119, which exhibits an X-ray powder diffraction (XRPD) pattern comprising peaks at about 4.08±0.2, 8.16±0.2, 8.73±0.2, 20.05±0.2, and 26.15±0.2 degrees two-theta.

121. The crystalline form of claim 119 or 120, which exhibits an X-ray powder diffraction (XRPD) pattern further comprising at least two peaks selected from about 6.76±0.2, 8.96±0.2, 12.30±0.2, 19.63±0.2, 21.10±0.2, 22.76±0.2, 25.88±0.2, and 31.55±0.2 degrees two-theta.

122. The crystalline form of any one of claims 118-121, which exhibits an XRPD pattern comprising peaks in Table 19.

123. The crystalline form of any one of claims 118-122, which is Form A-9 exhibiting an XRPD pattern substantially similar to FIG. 19A.

124. The crystalline form of claim 14, which exhibits an X-ray powder diffraction (XRPD) pattern comprising peaks at about 5.73±0.2, 17.02±0.2, and 23.23±0.2 degrees two-theta.

125. The crystalline form of claim 124, which exhibits an X-ray powder diffraction (XRPD) pattern comprising peaks at about 5.73±0.2, 11.37±0.2, 17.02±0.2, 22.70±0.2 and 23.23±0.2 degrees two-theta.

126. The crystalline form of claim 124 or 125, which exhibits an X-ray powder diffraction (XRPD) pattern further comprising at least two peaks selected from about 6.85±0.2, 7.39±0.2, 10.90±0.2, 14.65±0.2, 16.13±0.2, 19.77±0.2, 19.99±0.2, and 20.40±0.2 degrees two-theta.

127. The crystalline form of any one of claims 124-126, which exhibits an XRPD pattern comprising peaks in Table 20.

128. The crystalline form of any one of claims 124-127, which is Form B exhibiting an XRPD pattern substantially similar to FIG. 20.

129. The crystalline form of claim 14 or 15, which exhibits an X-ray powder diffraction (XRPD) pattern comprising peaks at about 7.66±0.2, 17.15±0.2, and 22.09±0.2 degrees two-theta.

130. The crystalline form of claim 129, which exhibits an X-ray powder diffraction (XRPD) pattern comprising peaks at about 5.22±0.2, 7.66±0.2, 17.15±0.2, 22.09±0.2, and 24.96±0.2 degrees two-theta.

131. The crystalline form of claim 129 or 130, which exhibits an X-ray powder diffraction (XRPD) pattern further comprising at least two peaks selected from about 10.55±0.2, 11.06±0.2, 16.81±0.2, 17.60±0.2, 19.32±0.2, 20.88±0.2, 21.39±0.2, and 26.46±0.2 degrees two-theta.

132. The crystalline form of any one of claims 129-131, which exhibits an XRPD pattern comprising peaks in Table 21.

133. The crystalline form of any one of claims 129-132, which is Form C exhibiting an XRPD pattern substantially similar to FIG. 21A.

134. The crystalline form of claim 14 or 15, which exhibits an X-ray powder diffraction (XRPD) pattern comprising peaks at about 4.97±0.2, 8.09±0.2, and 23.89±0.2 degrees two-theta.

135. The crystalline form of claim 134, which exhibits an X-ray powder diffraction (XRPD) pattern comprising peaks at about 4.97±0.2, 8.09±0.2, 17.46±0.2, 23.89±0.2, and 30.74±0.2 degrees two-theta.

136. The crystalline form of any one of claim 134 or 135, which exhibits an XRPD pattern comprising peaks in Table 22.

137. The crystalline form of any one of claims 134-136, which is Form J exhibiting an XRPD pattern substantially similar to FIG. 22A.

138. The crystalline form of claim 14 or 15, which exhibits an X-ray powder diffraction (XRPD) pattern comprising peaks at about 4.59±0.2, 13.75±0.2, and 21.37±0.2 degrees two-theta.

139. The crystalline form of claim 138, which exhibits an X-ray powder diffraction (XRPD) pattern comprising peaks at about 4.59±0.2, 8.53±0.2, 13.75±0.2, 21.37±0.2, and 23.02±0.2 degrees two-theta.

140. The crystalline form of claim 138 or 139, which exhibits an X-ray powder diffraction (XRPD) pattern further comprising at least two peaks selected from about 6.58±0.2, 10.08±0.2, 11.08±0.2, 24.21±0.2, and 31.80±0.2 degrees two-theta.

141. The crystalline form of any one of claims 138-140, which exhibits an XRPD pattern comprising peaks in Table 23.

142. The crystalline form of any one of claims 138-141, which is Form K exhibiting an XRPD pattern substantially similar to FIG. 23A.

143. The crystalline form of claim 14 or 15, which exhibits an X-ray powder diffraction (XRPD) pattern comprising peaks at about 10.66±0.2, 21.99±0.2, and 22.38±0.2 degrees two-theta.

144. The crystalline form of claim 143, which exhibits an X-ray powder diffraction (XRPD) pattern comprising peaks at about 10.66±0.2, 18.78±0.2, 21.99±0.2, 22.38±0.2, and 23.56±0.2 degrees two-theta.

145. The crystalline form of claim 143 or 144, which exhibits an X-ray powder diffraction (XRPD) pattern further comprising at least two peaks selected from about 8.49±0.2, 11.51±0.2, 13.62±0.2, 14.02±0.2, 15.37±0.2, 21.35±0.2, 23.20±0.2 and 24.13±0.2 degrees two-theta.

146. The crystalline form of any one of claims 143-145, which exhibits an XRPD pattern comprising peaks in Table 24.

147. The crystalline form of any one of claims 143-146, which is Form H2 exhibiting an XRPD pattern substantially similar to FIG. 24.

148. The crystalline form of claim 14, wherein the crystalline form is a solvate.

149. The crystalline form of claim 148, wherein the solvate is a dimethylsulfoxide (DMSO) solvate.

150. The crystalline form of claim 148 or 149, which exhibits an X-ray powder diffraction (XRPD) pattern comprising peaks at about 20.42±0.2, 20.94±0.2, and 21.65±0.2 degrees two-theta.

151. The crystalline form of claim 150, which exhibits an X-ray powder diffraction (XRPD) pattern comprising peaks at about 7.21±0.2, 10.18±0.2, 20.42±0.2, 20.94±0.2, and 21.65±0.2 degrees two-theta.

152. The crystalline form of claim 150 or 151, which exhibits an X-ray powder diffraction (XRPD) pattern further comprising at least two peaks selected from about 5.30±0.2, 14.41±0.2, 15.95±0.2, 19.76±0.2, 24.22±0.2, 25.28±0.2, 27.56±0.2, and 28.97±0.2 degrees two-theta.

153. The crystalline form of any one of claims 150-152, which exhibits an XRPD pattern comprising peaks in Table 25.

154. The crystalline form of any one of claims 150-153, which is Form E exhibiting an XRPD pattern substantially similar to FIG. 25A.

155. The crystalline form of claim 148 or 149, which exhibits an X-ray powder diffraction (XRPD) pattern comprising peaks at about 11.11±0.2, 20.77±0.2, and 21.32±0.2 degrees two-theta.

156. The crystalline form of claim 155, which exhibits an X-ray powder diffraction (XRPD) pattern comprising peaks at about 10.10±0.2, 11.11±0.2, 20.77±0.2, 21.32±0.2, and 24.20±0.2 degrees two-theta.

157. The crystalline form of claim 155 or 156, which exhibits an X-ray powder diffraction (XRPD) pattern further comprising at least two peaks selected from about 15.84±0.2, 16.81±0.2, 20.19±0.2, 22.57±0.2, 22.71±0.2, 23.15±0.2, 25.23±0.2 and 25.60±0.2 degrees two-theta.

158. The crystalline form of any one of claims 155-157, which exhibits an XRPD pattern comprising peaks in Table 26.

159. The crystalline form of any one of claims 155-158, which is Form F exhibiting an XRPD pattern substantially similar to FIG. 26A.

160. The crystalline form of claim 148, wherein the solvate is a dimethylsulfoxide (DMSO)-water solvate.

161. The crystalline form of claim 148 or 160, which exhibits an X-ray powder diffraction (XRPD) pattern comprising peaks at about 5.28±0.2, 15.77±0.2, and 18.95±0.2 degrees two-theta.

162. The crystalline form of claim 161, which exhibits an X-ray powder diffraction (XRPD) pattern comprising peaks at about 5.28±0.2, 15.77±0.2, 18.39±0.2, 18.95±0.2, and 21.00±0.2 degrees two-theta.

163. The crystalline form of claim 161 or 162, which exhibits an X-ray powder diffraction (XRPD) pattern further comprising at least two peaks selected from about 10.54±0.2, 14.81±0.2, 15.03±0.2, 20.27±0.2, 21.79±0.2, 22.76±0.2, 23.06±0.2, and 25.10±0.2 degrees two-theta.

164. The crystalline form of any one of claims 161-163, which exhibits an XRPD pattern comprising peaks in Table 27.

165. The crystalline form of any one of claims 161-164, which is Form G exhibiting an XRPD pattern substantially similar to FIG. 27A.

166. The crystalline form of claim 148, wherein the solvate is a 2,2,2-trifluoroethanol (TFE) solvate.

167. The crystalline form of claim 166, which exhibits an X-ray powder diffraction (XRPD) pattern comprising peaks at about 8.56±0.2, 9.63±0.2, and 20.48±0.2 degrees two-theta.

168. The crystalline form of claim 167, which exhibits an X-ray powder diffraction (XRPD) pattern comprising peaks at about 8.56±0.2, 9.63±0.2, 17.91±0.2, 20.48±0.2, and 23.87±0.2 degrees two-theta.

169. The crystalline form of claim 166 or 167, which exhibits an X-ray powder diffraction (XRPD) pattern further comprising at least two peaks selected from about 14.85±0.2, 19.61±0.2, 21.10±0.2, 21.60±0.2, 22.68±0.2, 23.31±0.2, 26.98±0.2 and 29.87±0.2 degrees two-theta.

170. The crystalline form of any one of claims 167-169, which exhibits an XRPD pattern comprising peaks in Table 28.

171. The crystalline form of any one of claims 167-170, which is Form I exhibiting an XRPD pattern substantially similar to FIG. 28A.

172. The crystalline form of claim 148, wherein the solvate is a dimethylformamide (DMF)-water hetero solvate.

173. The crystalline form of claim 172, which exhibits an X-ray powder diffraction (XRPD) pattern comprising peaks at about 5.37±0.2, 10.69±0.2, and 22.05±0.2 degrees two-theta.

174. The crystalline form of claim 173, which exhibits an X-ray powder diffraction (XRPD) pattern comprising peaks at about 5.37±0.2, 8.53±0.2, 10.69±0.2, 18.86±0.2, and 22.05±0.2 degrees two-theta.

175. The crystalline form of claim 173 or 174, which exhibits an X-ray powder diffraction (XRPD) pattern further comprising at least two peaks selected from about 3.74±0.2, 15.39±0.2, 17.99±0.2, 18.80±0.2, 21.46±0.2, 22.29±0.2, 23.47±0.2, and 23.61±0.2 degrees two-theta.

176. The crystalline form of any one of claims 173-175, which exhibits an XRPD pattern comprising peaks in Table 29.

177. The crystalline form of any one of claims 173-176, which is Form H1 exhibiting an XRPD pattern substantially similar to FIG. 29A.

178. The crystalline form of claim 148, wherein the solvate is an acetone solvate.

179. The crystalline form of claim 178, which exhibits an X-ray powder diffraction (XRPD) pattern comprising peaks at about 10.67±0.2, 18.77±0.2, and 22.04±0.2 degrees two-theta.

180. The crystalline form of claim 179, which exhibits an X-ray powder diffraction (XRPD) pattern comprising peaks at about 10.67±0.2, 17.95±0.2, 18.77±0.2, 22.04±0.2, and 23.64±0.2 degrees two-theta.

181. The crystalline form of claim 179 or 180, which exhibits an X-ray powder diffraction (XRPD) pattern further comprising at least two peaks selected from about 5.35±0.2, 7.46±0.2, 8.50±0.2, 11.51±0.2, 13.63±0.2, 15.36±0.2, 21.35±0.2, and 25.55±0.2 degrees two-theta.

182. The crystalline form of any one of claims 179-181, which exhibits an XRPD pattern comprising peaks in Table 30.

183. The crystalline form of any one of claims 179-182, which is Form H3 exhibiting an XRPD pattern substantially similar to FIG. 30.

184. The crystalline form of claim 148, wherein the solvate is a tetrahydrofuran (THF) solvate.

185. The crystalline form of claim 184, which exhibits an X-ray powder diffraction (XRPD) pattern comprising peaks at about 5.32±0.2, 22.00±0.2, and 22.34±0.2 degrees two-theta.

186. The crystalline form of claim 185, which exhibits an X-ray powder diffraction (XRPD) pattern comprising peaks at about 5.32±0.2, 10.63±0.2, 18.74±0.2, 22.00±0.2, and 22.34±0.2 degrees two-theta.

187. The crystalline form of claim 185 or 186, which exhibits an X-ray powder diffraction (XRPD) pattern further comprising at least two peaks selected from about 7.41±0.2, 8.47±0.2, 11.48±0.2, 11.75±0.2, 13.62±0.2, 14.01±0.2, 17.90±0.2, 23.05±0.2, and 23.54±0.2 degrees two-theta.

188. The crystalline form of any one of claims 185-187, which exhibits an XRPD pattern comprising peaks in Table 31.

189. The crystalline form of any one of claims 185-188, which is Form H4 exhibiting an XRPD pattern substantially similar to FIG. 31.

190. The crystalline form of claim 148 or 149, which exhibits an X-ray powder diffraction (XRPD) pattern comprising peaks at about 15.55±0.2, 18.84±0.2, and 21.66±0.2 degrees two-theta.

191. The crystalline form of claim 190, which exhibits an X-ray powder diffraction (XRPD) pattern comprising peaks at about 5.33±0.2, 15.55±0.2, 18.84±0.2, 21.30±0.2, and 21.66±0.2 degrees two-theta.

192. The crystalline form of claim 190 or 191, which exhibits an X-ray powder diffraction (XRPD) pattern further comprising at least two peaks selected from about 7.54±0.2, 10.63±0.2, 11.32±0.2, 13.54±0.2, 17.98±0.2, 20.90±0.2, 22.52±0.2, and 23.36±0.2 degrees two-theta.

193. The crystalline form of any one of claims 190-192, which exhibits an XRPD pattern comprising peaks in Table 32.

194. The crystalline form of any one of claims 190-193 which is Form H5 exhibiting an XRPD pattern substantially similar to FIG. 32.

195. The crystalline form of claim 148, wherein the solvate is a benzyl alcohol solvate.

196. The crystalline form of claim 195, which exhibits an X-ray powder diffraction (XRPD) pattern comprising peaks at about 5.27±0.2, 21.64±0.2, and 22.35±0.2 degrees two-theta.

197. The crystalline form of claim 195, which exhibits an X-ray powder diffraction (XRPD) pattern comprising peaks at about 5.27±0.2, 18.67±0.2, 21.64±0.2, 21.99±0.2, and 22.35±0.2 degrees two-theta.

198. The crystalline form of claim 195, which exhibits an X-ray powder diffraction (XRPD) pattern further comprising at least two peaks selected from about 8.38±0.2, 10.55±0.2, 11.47±0.2, 11.71±0.2, 13.44±0.2, 13.96±0.2, 15.45±0.2, and 23.36±0.2 degrees two-theta.

199. The crystalline form of any one of claims 196-198, which exhibits an XRPD pattern comprising peaks in Table 33.

200. The crystalline form of any one of claims 196-199 which is Form H6 exhibiting an XRPD pattern substantially similar to FIG. 33.

201. The crystalline form of any one of claims 1-200 wherein the crystalline form has a purity in the range of about 80% to about 99%.

202. The crystalline form of any one of claims 1-200, wherein the crystalline form has a purity of about 95% or higher.

203. The crystalline form of any one of claims 1-200, wherein the crystalline form has a purity of about 99% or higher.

204. The crystalline form of any one of claims 4-9, 17-22, 28-33, 38-43, 48-57, 59-68, 70-84, 86-90, 92-101, 103-106, 108-117, 119-147, 150-159, 161-165, 167-171, 173-177, 179-183, 185-194, and 196-200, wherein the XRPD pattern was obtained using Cu Kα radiation.

205. The crystalline form of claim 1, wherein the crystalline form is a pharmaceutically acceptable salt of Formula (I-A): 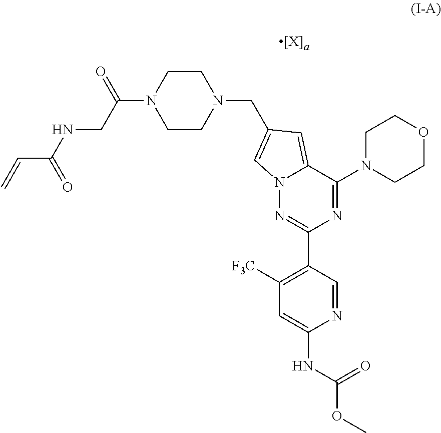
wherein X is a pharmaceutically acceptable acid; and
a is about 0.5 to about 2.

206. The crystalline form of claim 1, wherein the crystalline form is of Formula (I-B): 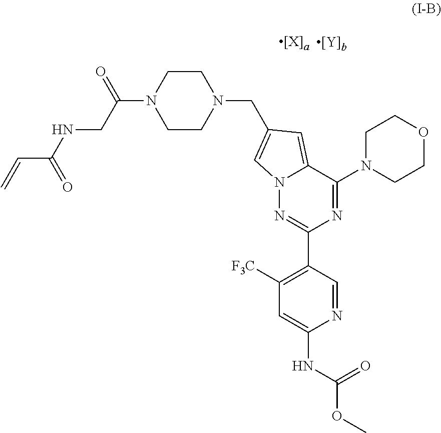
wherein:
Y is a solvent;
X is a pharmaceutically acceptable acid;
a is about 0.5 to about 2; and
b is about 0.5 to about 5.

207. The crystalline form of claim 1, wherein the crystalline form is of Formula (I-C): 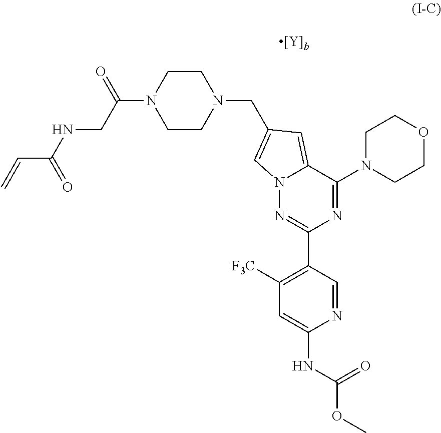
wherein Y is a solvent; and
b is about 0.5 to about 5.

208. The crystalline form of claim 205 or 206, wherein X is phosphoric acid, hydrochloric acid, sulfuric acid, methanesulfonic acid, a benzenesulfonic acid, p-toluenesulfonic acid, fumaric acid, maleic acid, L-tartaric acid, citric acid, and succinic acid.

209. The crystalline form of claim 205, 206, or 208, wherein a is about 1

210. The crystalline form of claim 206 or 207, wherein the solvent is water, acetone, benzyl alcohol, DMF, DMSO, THF, TFE or a combination thereof.

211. The compound of any one of claims 206-210, wherein b is about 0.5, about 1, about 1.5, about 2, about 2.5 about 3, about 3.5, about 4, or about 4.5.

212. The compound of claim 206-210, wherein b is about 1.5 to about 2.

213. A composition comprising a crystalline form of any one of claims 1-212, and a pharmaceutically acceptable carrier.

214. The composition of claim 213, further comprising one or more additional therapeutic agents.

215. A method for treating cancer in a subject in need thereof, comprising administering an effective amount of the crystalline form of any one of claims 1-212, or the pharmaceutical composition of claim 213 or 214.

216. The method of claim 215, wherein the cancer has a mutation in the PIK3CA gene.

217. The method of claim 215 or 216, wherein the cancer is ampullary cancer, anal cancer, bladder cancer, breast cancer, breast cancers, cervical cancer, colon cancer, colorectal cancer, endometrial cancer, esophageal cancer, gastric cancer, glioma, head and neck cancer, hematologic cancer, lung cancer, liver cancer, ovary cancer, pancreatic cancer, penile cancer, prostate cancer, renal cancer, salivary gland cancer, skin cancer, vaginal cancer, and urothelial cancer.

218. The method of claim 217, wherein the hematologic cancer is leukemia, lymphoma, or myeloma.

219. A method for inhibiting phosphoinositide 3-kinase (PI3K) in a subject in need thereof, comprising administering an effective amount of the crystalline form of any one of claims 1-212, or the pharmaceutical composition of claim 213 or 214.

220. A salt of compound I, or a solvate thereof.

221. The salt of claim 220, wherein the salt is a phosphate salt of compound I, or a solvate thereof.

222. A means for inhibiting phosphoinositide 3-kinase (PI3K).

223. A means for covalently binding to phosphoinositide 3-kinase (PI3K) and inhibiting PI3K.

224. The means of claim 222, or claim 223, wherein the means are in the form of a salt.

225. The means of any one of claims 222-224, wherein the means are in a crystalline form.

226. The means of claim 225, wherein the crystalline form may include Form A, Form A*, Form A** Form B*, Form A-1, Form B-1, Form A-2, Form B-2, Form A-3, Form B-3, Form C-3, Form A-4, Form A-5, Form A-6, Form A-7, Form B-7, Form A-8, Form B-8, Form A-9, Form B, Form C, Form J, Form K, Form H2, Form E, Form F, Form G, Form I, Form H1, Form H3, Form H4, Form H5, and/or Form H6.

227. A pharmaceutical composition comprising a means for inhibiting phosphoinositide 3-kinase (PI3K), and a pharmaceutically acceptable carrier.

228. A pharmaceutical composition comprising a means for covalently binding to phosphoinositide 3-kinase (PI3K) and inhibiting PI3K.

229. The pharmaceutical composition of claim 227 or 228, wherein the means is in the form of a salt.

230. The pharmaceutical composition of any one of claims 227-229, wherein the means are in a crystalline form.

231. The pharmaceutical composition of claim 230, wherein the crystalline form may include Form A, Form A*, Form A**, Form B*, Form A-1, Form B-1, Form A-2, Form B-2, Form A-3, Form B-3, Form C-3, Form A-4, Form A-5, Form A-6, Form A-7, Form B-7, Form A-8, Form B-8, Form A-9, Form B, Form C, Form J, Form K, Form H2, Form E, Form F, Form G, Form I, Form H1, Form H3, Form H4, Form H5, and/or Form H6.

232. The pharmaceutical composition of any one of claim 213-215, or 227-231, wherein a crystalline form is at least about 70% pure in the pharmaceutical composition.

233. The pharmaceutical composition of claim 232, wherein the crystalline form is at least about 80% pure in the pharmaceutical composition.

234. The pharmaceutical composition of claim 232, wherein the crystalline form is at least about 90% pure in the pharmaceutical composition.

235. The pharmaceutical composition of claim 232, wherein the crystalline form is at least 95% pure in the pharmaceutical composition.

236. The pharmaceutical composition of claim 232, wherein the crystalline form is at least 99% pure in the pharmaceutical composition.
